SEQUENCE LISTING (230 sequences)

SEQ ID NO: 1 - protein, 8 aa, synthetic construct
GFSLSSYY

SEQ ID NO: 2 - protein, 8 aa, synthetic construct
GIDLSSND

SEQ ID NO: 3 - protein, 8 aa, synthetic construct
GFTISSYH

SEQ ID NO: 4 - protein, 8 aa, synthetic construct
GFSLSTAY

SEQ ID NO: 5 - protein, 8 aa, synthetic construct
GIDLSSND

SEQ ID NO: 6 - protein, 8 aa, synthetic construct
GRSLSYKY

SEQ ID NO: 7 - protein, 8 aa, synthetic construct
GFSLSSNY

SEQ ID NO: 8 - protein, 8 aa, synthetic construct
GIDLNSYD

SEQ ID NO: 9 - protein, 8 aa, synthetic construct
GFSLNIYG

SEQ ID NO: 10 - protein, 8 aa, synthetic construct
GFSLNSYY

SEQ ID NO: 11 - protein, 8 aa, synthetic construct
GFSLSSYY

SEQ ID NO: 12 - protein, 8 aa, synthetic construct
GFSLSSNY

SEQ ID NO: 13 - protein, 8 aa, synthetic construct
GFTISNYH

SEQ ID NO: 14 - protein, 8 aa, synthetic construct
GFSLSSSY

SEQ ID NO: 15 - protein, 8 aa, synthetic construct
GFSLSTAY

SEQ ID NO: 16 - protein, 8 aa, synthetic construct
GFTISTYH

SEQ ID NO: 17 - protein, 8 aa, synthetic construct
GFDLSSNA

SEQ ID NO: 18 - protein, 8 aa, synthetic construct
GFSLIGQY

SEQ ID NO: 19 - protein, 9 aa, synthetic construct
GFSLNNKFW

SEQ ID NO: 20 - protein, 9 aa, synthetic construct
GFSFSSSYY

SEQ ID NO: 21 - protein, 9 aa, synthetic construct
GFSFSSGYY

SEQ ID NO: 22 - protein, 9 aa, synthetic construct
GFSVSSGVY

SEQ ID NO: 23 - protein, 8 aa, synthetic construct
GFDFTSSA

SEQ ID NO: 24 - protein, 6 aa, synthetic construct
IGSRDK

SEQ ID NO: 25 - protein, 7 aa, synthetic construct
IWRGINI

SEQ ID NO: 26 - protein, 8 aa, synthetic construct
LVRGSYNT

SEQ ID NO: 27 - protein, 7 aa, synthetic construct
ISPGGTT

SEQ ID NO: 28 - protein, 7 aa, synthetic construct
IWRGSSI

SEQ ID NO: 29 - protein, 8 aa, synthetic construct
IPASGFRP

SEQ ID NO: 30 - protein, 7 aa, synthetic construct
VDTTGRS

SEQ ID NO: 31 - protein, 7 aa, synthetic construct
IWRGGIA

SEQ ID NO: 32 - protein, 8 aa, synthetic construct
IYAGSGST

SEQ ID NO: 33 - protein, 6 aa, synthetic construct
IGSRDK

SEQ ID NO: 34 - protein, 7 aa, synthetic construct
IEPTDSR

SEQ ID NO: 35 - protein, 7 aa, synthetic construct
VDTSGRS

SEQ ID NO: 36 - protein, 8 aa, synthetic construct
IVAGSDVA

SEQ ID NO: 37 - protein, 7 aa, synthetic construct
IDTAVAT

SEQ ID NO: 38 - protein, 7 aa, synthetic construct
IHLGRST

SEQ ID NO: 39 - protein, 8 aa, synthetic construct
INANSGGT

SEQ ID NO: 40 - protein, 6 aa, synthetic construct
IIYGST

SEQ ID NO: 41 - protein, 7 aa, synthetic construct
IDTTERS

SEQ ID NO: 42 - protein, 9 aa, synthetic construct
IDAGGSGGT

SEQ ID NO: 43 - protein, 9 aa, synthetic construct
IYVGSNNNS

SEQ ID NO: 44 - protein, 8 aa, synthetic construct
ILPGTVAS

SEQ ID NO: 45 - protein, 8 aa, synthetic construct
ILTGSVTS

SEQ ID NO: 46 - protein, 6 aa, synthetic construct
ILNSRI

SEQ ID NO: 47 - protein, 14 aa, synthetic construct
AREVGGERTGAFVL

SEQ ID NO: 48 - protein, 13 aa, synthetic construct
ARDWIAYHSGISF

SEQ ID NO: 49 - protein, 12 aa, synthetic construct
VRDVIGTSGSDL

SEQ ID NO: 50 - protein, 11 aa, synthetic construct
VRSGDCMDFNL

SEQ ID NO: 51 - protein, 13 aa, synthetic construct
ARDWIGYHTGISF

SEQ ID NO: 52 - protein, 10 aa, synthetic construct
AKDLGMDFNL

SEQ ID NO: 53 - protein, 11 aa, synthetic construct
VRSGGCMDFDL

SEQ ID NO: 54 - protein, 13 aa, synthetic construct
ARDWLGYHTGTSL

SEQ ID NO: 55 - protein, 19 aa, synthetic construct
ARDLGYGDSNYYKYYEFGL

SEQ ID NO: 56 - protein, 14 aa, synthetic construct
AREVGGERTGAFVL

SEQ ID NO: 57 - protein, 10 aa, synthetic construct
ARNVGGRPNL

SEQ ID NO: 58 - protein, 11 aa, synthetic construct
VRSGGCMDFDL

SEQ ID NO: 59 - protein, 12 aa, synthetic construct
VRDVAGTTGSDL

SEQ ID NO: 60 - protein, 11 aa, synthetic construct
VRSDDCMAFNF

SEQ ID NO: 61 - protein, 11 aa, synthetic construct
VRSGDCMAFDL

SEQ ID NO: 62 - protein, 13 aa, synthetic construct
VRSGYNSGMAFDP

SEQ ID NO: 63 - protein, 18 aa, synthetic construct
ARGGRSYGDGYAFGYFNI

SEQ ID NO: 64 - protein, 11 aa, synthetic construct
VRSDSCMAFGL

SEQ ID NO: 65 - protein, 17 aa, synthetic construct
ARDLAAAGGGLTGAFNL

SEQ ID NO: 66 - protein, 20 aa, synthetic construct
ARSIVGYAGYTYARYNAFDP

SEQ ID NO: 67 - protein, 14 aa, synthetic construct
ARVNPTYGGHGYDL

SEQ ID NO: 68 - protein, 14 aa, synthetic construct
VRVNPNYGGHGYDL

SEQ ID NO: 69 - protein, 16 aa, synthetic construct
ARVPGGSPYNWWAMDL

SEQ ID NO: 70 - protein, 6 aa, synthetic construct
QSISGY

SEQ ID NO: 71 - protein, 8 aa, synthetic construct
PSVYNNNA

SEQ ID NO: 72 - protein, 8 aa, synthetic construct
QTVYNNNL

SEQ ID NO: 73 - protein, 8 aa, synthetic construct
KSVWRKNC

SEQ ID NO: 74 - protein, 8 aa, synthetic construct
PSVYNNNA

SEQ ID NO: 75 - protein, 8 aa, synthetic construct
QSVYNDNQ

SEQ ID NO: 76 - protein, 8 aa, synthetic construct
KSVWSQNC

SEQ ID NO: 77 - protein, 8 aa, synthetic construct
KSVYSNNC

SEQ ID NO: 78 - protein, 8 aa, synthetic construct
QSVYNNKH

SEQ ID NO: 79 - protein, 6 aa, synthetic construct
QSISGY

SEQ ID NO: 80 - protein, 6 aa, synthetic construct
QSVSNW

SEQ ID NO: 81 - protein, 8 aa, synthetic construct
KSVWSKNC

SEQ ID NO: 82 - protein, 8 aa, synthetic construct
QSVYNKNL

SEQ ID NO: 83 - protein, 8 aa, synthetic construct
RSVWRNNC

SEQ ID NO: 84 - protein, 8 aa, synthetic construct
KSVYSKNC

SEQ ID NO: 85 - protein, 8 aa, synthetic construct
KSVYNNNN

SEQ ID NO: 86 - protein, 6 aa, synthetic construct
QNIYSN

SEQ ID NO: 87 - protein, 8 aa, synthetic construct
KSVWNNNC

SEQ ID NO: 88 - protein, 6 aa, synthetic construct
QSINNL

SEQ ID NO: 89 - protein, 6 aa, synthetic construct
QNIYNN

SEQ ID NO: 90 - protein, 8 aa, synthetic construct
KSVHNNNH

SEQ ID NO: 91 - protein, 8 aa, synthetic construct
KSVYARNH

SEQ ID NO: 92 - protein, 8 aa, synthetic construct
QSVAGTNW

SEQ ID NO: 93-115 - skipped (23 entries)

SEQ ID NO: 116 - protein, 12 aa, synthetic construct
QQGVSGNNVQNV

SEQ ID NO: 117 - protein, 9 aa, synthetic construct
AGGFDSDGA

SEQ ID NO: 118 - protein, 11 aa, synthetic construct
LGGFHNDADTA

SEQ ID NO: 119 - protein, 11 aa, synthetic construct
AGGYSDSSDNA

SEQ ID NO: 120 - protein, 9 aa, synthetic construct
AGGFYSDGV

SEQ ID NO: 121 - protein, 11 aa, synthetic construct
LGAYNDDSETA

SEQ ID NO: 122 - protein, 11 aa, synthetic construct
AGAYYDSSDNG

SEQ ID NO: 123 - protein, 9 aa, synthetic construct
AGGYNSDGA

SEQ ID NO: 124 - protein, 9 aa, synthetic construct
AGGYSDVRA

SEQ ID NO: 125 - protein, 12 aa, synthetic construct
QQGVSGSNIHNP

SEQ ID NO: 126 - protein, 12 aa, synthetic construct
QQAYSGRNVDNV

SEQ ID NO: 127 - protein, 11 aa, synthetic construct
AGAYYDSSDNG

SEQ ID NO: 128 - protein, 11 aa, synthetic construct
LGAYDNDADTA

SEQ ID NO: 129 - protein, 11 aa, synthetic construct
AGGYSDARDNG

SEQ ID NO: 130 - protein, 11 aa, synthetic construct
AGGYSDARDNA

SEQ ID NO: 131 - protein, 11 aa, synthetic construct
AGAFSSASDNA

SEQ ID NO: 132 - protein, 14 aa, synthetic construct
QSAYYSSSTDMANA

SEQ ID NO: 133 - protein, 11 aa, synthetic construct
AGGYSTSSDNG

SEQ ID NO: 134 - protein, 12 aa, synthetic construct
QQGWSGSNVDNV

SEQ ID NO: 135 - protein, 13 aa, synthetic construct
QSVAYSTGAATIA

SEQ ID NO: 136 - protein, 11 aa, synthetic construct
AGAFISSGDNG

SEQ ID NO: 137 - protein, 11 aa, synthetic construct
AGAFSTSGDNG

SEQ ID NO: 138 - protein, 12 aa, synthetic construct
HGAYSSVNVIHT

SEQ ID NO: 139 - protein, 117 aa, synthetic construct
QEQLKESGGRLVTPGTPLTLTCTVSGFSLSSYYMWWVRQAPGKGLEWIGSIGSRDKYYATWANGRVTISRTSTTVDLKITSPTTEDTATYFCAREVGGERTGAFVLWGQGTLVTVSS

SEQ ID NO: 140 - protein, 117 aa, synthetic construct
QAVEESGGRLVTPGTPLVLTCTASGIDLSSNDMAWVRQPPGKGLEFIGAIWRGINIFYANWAKGRLTISKTSSTTVDLRITSPTIEDTATYFCARDWIAYHSGISFWGQGTLVTVSS

SEQ ID NO: 141 - protein, 117 aa, synthetic construct
QSLEESGGRLVTPGTPLTLTCTASGFTISSYHMSWVRQAPGKGLEWIGYLVRGSYNTYYASWAKGRFTISKTSSTTVDLKLTSPTTEDTATYFCVRDVIGTSGSDLWGQGTLVTVSS

SEQ ID NO: 142 - protein, 114 aa, synthetic construct
QSVEESGGRLVTPGTPLTLTCTVSGFSLSTAYMSWVRQAPGEGLEWIGYISPGGTTYYASWAKGRFTISKTSTTVDLKITSPTSEDTATYSCVRSGDCMDFNLWGQGTLVTVSS

SEQ ID NO: 143 - protein, 117 aa, synthetic construct
QAVEESGGRLVTPGTPLVLTCTASGIDLSSNDMAWVRQPPGKGLEFIGGIWRGSSIFYANWAKGRLTISKTSSTTVDLRITSPTIEDTATYFCARDWIGYHTGISFWGQGTLVTVSR

SEQ ID NO: 144 - protein, 114 aa, synthetic construct
QSLEESGGRLVTPGTPLTLTCTASGRSLSYKYMSWVRQAPGKGLERIGYIPASGFRPRYASWAKGRFTISRTSATVDLRITSPTTEDTATYFCAKDLGMDFNLWGQGTLVTVSS

SEQ ID NO: 145 - protein, 114 aa, synthetic construct
QSLEESGGRLVTPGTPLTLTCTVSGFSLSSNYISWVRQAPGEGLEWVGYVDTTGRSYYATWAKGRFTISRTSTTVDLKMTSPTSEDTATYFCVRSGGCMDFDLWGQGTLVTVSS

SEQ ID NO: 146 - protein, 117 aa, synthetic construct
QSVEESGGRLVTPGTPLTLTCTVSGIDLNSYDMIWVRQAPGKGLEFIGWIWRGGIANYASWAKGRFTFSITSSTTVDLRITSPTTEDTATYFCARDWLGYHTGTSLWGQGTLVTVSS

SEQ ID NO: 147 - protein, 123 aa, synthetic construct
QSVEESGGRLVTPGTPLTLTCTASGFSLNIYGVSWVRQAPGKGLEWIGYIYAGSGSTWYASWAKGRFTISKTSTTVDLKITSPTTEDTATYFCARDLGYGDSNYYKYYEFGLWGQGTLVTVSS

SEQ ID NO: 148 - protein, 117 aa, synthetic construct
QEQLKESGGRLVTPGTPLTLTCTASGFSLNSYYMWWVRQAPGKGLEWIGSIGSRDKYYANWAKGRFTISRTSTTVDLKITSPTTDDTATYFCAREVGGERTGAFVLWGQGTLVTVSS

SEQ ID NO: 149 - protein, 113 aa, synthetic construct
QSLEESGGRLVMPGTPLTLTCTASGFSLSSYYMSWVRQAPGKGLQWIGYIEPTDSRDYASWAKGRFTISKTSTTVDLRIISPTTEDTATYFCARNVGGRPNLWGQGTLVTVSS

SEQ ID NO: 150 - protein, 114 aa, synthetic construct
QSLEESGGRLVTPGTPLTLTCAASGFSLSSNYISWVRQAPGEGLEWVGYVDTSGRSYYATWAKGRFTISRTSTTVDLKITSPTSEDTATYFCVRSGGCMDFDLWGQGTLVTVSS

SEQ ID NO: 151 - protein, 117 aa, synthetic construct
QSLEESGGRLVTPGTPLTLTCTASGFTISNYHMSWVRQAPGKGLEWIGYIVAGSDVAYYASWARGRFTISKTSSTTVDLTITRPTTEDTATYFCVRDVAGTTGSDLWGQGTLVTVSS

SEQ ID NO: 152 - protein, 114 aa, synthetic construct
QSVEESGGRLVTPGTPLTLTCTVSGFSLSSSYMSWVRQAPGKGLEWIGYIDTAVATYFASWAKGRFTISKTSTTVDLKITSPTSEDTATYFCVRSDDCMAFNFWGQGTLVTVSS

SEQ ID NO: 153 - protein, 115 aa, synthetic construct
QSLEESGGRLVTPGTPLTLTCTVSGFSLSTAYISWVRQAPGEGLQWIGYIHLGRSTYYASWAQGRFTISKTSSTTVDLKITSPTSEDTATYFCVRSGDCMAFDLWGQGTLVTVSS

SEQ ID NO: 154 - protein, 117 aa, synthetic construct
QSVEESGGRLVTPGTPLTLTCTASGFTISTYHMSWVRQAPGKGLEWIGYINANSGGTWYASWAKGRFTISKTSTTVNLKITSPTTEDTATYFCVRSGYNSGMAFDPWGQGTLVTVSS

SEQ ID NO: 155 - protein, 123 aa, synthetic construct
QEQLGESGGGLVQPGGTLKLSCKGSGFDLSSNAMCWVRQAPGKGLEWVGCIIYGSTYYATWVNGRFTLSRDNAQSSVDLQLSSLTAADTATYFCARGGRSYGDGYAFGYFNIWGQGTLVTVSS

SEQ ID NO: 156 - protein, 116 aa, synthetic construct
QEQLEESGGRLGTPGTPLTLTCTVSGFSLIGQYMSWVRQAPGKGLEWIGYIDTTERSYYASWAKGRFTISKTSSTTVDLKMTSLTTEDTATYFCVRSDSCMAFGLWGQGTLVTVSS

SEQ ID NO: 157 - protein, 125 aa, synthetic construct
QSGGGGEGGLVKPGGSLELCCKASGFSLNNKFWIWWVRQAPGKGLEWIGSIDAGGSGGTYYASWVNGRFTLSRDIDQSTYCLQLNSLTAADTAIYYCARDLAAAGGGLTGAFNLWGQGTLVTVSS

SEQ ID NO: 158 - protein, 127 aa, synthetic construct
QSLEESGGGLVQPEGSLTLTCTASGFSFSSSYYMCWVRQAPGKGLEWIACIYVGSNNNSYYASWAKGRFAISKTSSTTVTLQMGSLTAADTATYFCARSIVGYAGYTYARYNAFDPWGQGTLVTVSS

SEQ ID NO: 159 - protein, 121 aa, synthetic construct
QEQLVESGGGLVKPGASLTLTCKASGFSFSSGYYMCWVRQAPGKGLEWIGCILPGTVASYYASWAKGRSTISKASSTTVDLKMTRLTAADTATYFCARVNPTYGGHGYDLWGQGTLVTVSS

SEQ ID NO: 160 - protein, 121 aa, synthetic construct
QEQLVESGGGLVQPGASLTLTCKASGFSVSSGVYMCWVRQAPGKGLEWIGCILTGSVTSYYATWVNGRFTLSKASSTTLYLQLNSLTVADTATHFCVRVNPNYGGHGYDLWGQGTLVTVSS

SEQ ID NO: 161 - protein, 119 aa, synthetic construct
RSLEESGGGLVKPGETLTLTCKASGFDFTSSAACWVRQAPGKGLEWIGCILNSRIYYAAWAKGRFTISRASSITVTLRMTSLTAADTATYFCARVPGGSPYNWWAMDLWGQGTLVTVSS

SEQ ID NO: 162 - protein, 110 aa, synthetic construct
ALVMTQSPSSVSAAVGDTVTIKCQASQSISGYLAWYQQKPGQAPKLLIYKASTLASGVSSRFKGRGSGTEFTLTISDLECADAATYYCQQGVSGNNVQNVFGGGTEVVVK

SEQ ID NO: 163 - protein, 109 aa, synthetic construct
AQVLTQTPSSVSAAVGGTVTINCQSSPSVYNNNALSWFQQKPGQPPKVLISRASTRASGVPSRFKGSGSGTQFTLTISGVECVDAATYYCAGGFDSDGAFGGGTEVVVK

SEQ ID NO: 164 - protein, 111 aa, synthetic construct
AAVLTQTPSPVSAAVGGTVTIKCQSSQTVYNNNLLSWYQQKPGQPPKLLIYLASTLASGVPDRFSGSGSGTQFTLTISGVQCDDAATYYCLGGFHNDADTAFGGGTEVVVK

SEQ ID NO: 165 - protein, 111 aa, synthetic construct
AAVLTQTPSPVSTAVGGTVSISCQSTKSVWRKNCLVWYQQKPGQPPKRLIYQASSLASGVPSRFKGSGSGTDFTLTIGDVQCDDAATYYCAGGYSDSSDNAFGGGTEVVVK

SEQ ID NO: 166 - protein, 109 aa, synthetic construct
AQVLTQTPASVSAAVGGTVTINCQSSPSVYNNNALAWYQQKPGQPPKLLISRASTLASGVPSRFKGSGSGTQFTLTISGVECVDAATYYCAGGFYSDGVFGGGTEVVVK

SEQ ID NO: 167 - protein, 111 aa, synthetic construct
AAVLTQTPSPVSAAVGGTVTITCQSSQSVYNDNQLSWYQQKSGQPPKILIYEASKLASGVPSRFSGSGSGTQFTLTIVGVQCDDAASYYCLGAYNDDSETAFGGGTEVVVK

SEQ ID NO: 168 - protein, 111 aa, synthetic construct
AQVLTQTPSSVSAAVGGSVSISCQSTKSVWSQNCLVWYQQKPGQPPKRLIYQASKLASGVPSRFKGSGSGTQFTLTIGDVQCDDAATYYCAGAYYDSSDNGFGGGTEVVVK

SEQ ID NO: 169 - protein, 109 aa, synthetic construct
AQVLTQTPASVSAAVGGTVTISCQSSKSVYSNNCLSWFQQKPGQPPKLLIYRASTLASGVPSRFKGSGSGAQFILTISDVQCDDAATYYCAGGYNSDGAFGGGTEVVVK

SEQ ID NO: 170 - protein, 109 aa, synthetic construct
AQVLTQTPSPVSAAVGGTVTIACQSSQSVYNNKHLAWYQQKLGQPPKLLIYSASKVASGVSSRFSGRGSGTQFTLTISGVDCDDAATYYCAGGYSDVRAFGGGTEVVVK

SEQ ID NO: 171 - protein, 110 aa, synthetic construct
ALVMTQTPSSVSEPVGGTVTIKCQASQSISGYLAWYQQKPGQPPKLLIYKASTLASGVSSRFKGSGSGTEFTLTISDLECADAATYYCQQGVSGSNIHNPFGGGTEVVVK

SEQ ID NO: 172 - protein, 110 aa, synthetic construct
AYDVTQTPASVEAAVGGTVTIKCQVSQSVSNWLAWYQQKPGQPPKLLIYSASTLASGVPSRFKGSGSGTQFTLTISDLECADAATYYCQQAYSGRNVDNVFGGGTEVVVK

SEQ ID NO: 173 - protein, 111 aa, synthetic construct
AQVLTQTPSSVSTTVGGTVSISCQSTKSVWSKNCLVWYQQKPGQPPKRLIYQASKLASGVPSRFKGSGSGTQFTLTIGDVQCDDAATYYCAGAYYDSSDNGFGGGTEVVVK

SEQ ID NO: 174 - protein, 111 aa, synthetic construct
AAVLTQTPSPVSAAVGGTVTIKCQSSQSVYNKNLLSWYQQKPGQPPRVLIYKASTLASGVPSRFSGSGSGTQFTLTIRGVQCDDAATYYCLGAYDNDADTAFGGGTEVVVK

SEQ ID NO: 175 - protein, 111 aa, synthetic construct
AAVLTQTPASVSAAVGGTVTISCQSTRSVWRNNCLAWYQQKPGQPPKLLIYRASSLASGVPSRFRGSGSGTQFTLTIGDVQCDDAATYYCAGGYSDARDNGFGGGTEVVVK

SEQ ID NO: 176 - protein, 111 aa, synthetic construct
AAVLTQTPSPVSAAVGGTVSISCQSTKSVYSKNCLAWYQQKPGQPPKLLIYKASTLASGVPSRFKGSGSGTQFTLTIGDVQCDDVATYYCAGGYSDARDNAFGGGTEVVVK

SEQ ID NO: 177 - protein, 111 aa, synthetic construct
AAVLTQTPSPVSAAVGGTVSISCQSSKSVYNNNNLAWFQQKPGQPPKLLIYGASMLASGVPSRFKGSGSGTQFTLTISDVQCDDAATYYCAGAFSSASDNAFGGGTEVVVK

SEQ ID NO: 178 - protein, 112 aa, synthetic construct
ALVMTQTPSSVSAAVGGTVTINCQASQNIYSNLAWYQQKPGQRPKLLIYGTSNLESGVPSRFKGSGSGTEYTLTISDLECDDAATYYCQSAYYSSSTDMANAFGGGTEVVVK

SEQ ID NO: 179 - protein, 111 aa, synthetic construct
VQVLTQTPSSVSAAVGGTVTMSCQSTKSVWNNNCLAWYQQKPGQPPKLLIYKASTLAAGVSSRFKGNGSGTQSTLTISDVQCDDAATYYCAGGYSTSSDNGFGGGTEVVVK

SEQ ID NO: 180 - protein, 110 aa, synthetic construct
AYDMTQTPASVSAAVGGTVTINCQASQSINNLLAWYQQKPGQPPKLLIYKASTLASGVPSRFKGSGSGTQFTLTISGVQCADAATYYCQQGWSGSNVDNVFGGGTEVVVK

SEQ ID NO: 181 - protein, 111 aa, synthetic construct
ALVMTQTPSSVSGAVGGTVTINCQASQNIYNNLAWYQQKPGQPPKLLIYAASNLASGVSSRFKGSGSGTEYTLTISDLECDDAATYYCQSVAYSTGAATIAFGGGTEVVVK

SEQ ID NO: 182 - protein, 111 aa, synthetic construct
AAVLTQTPSPVSAAVGGTVSISCQSSKSVHNNNHLAWFQQKPGQPPKLLIYKASTLASGVPSRFKGSGSGTQFTLTISDVQCDDAATYYCAGAFISSGDNGFGGGTEVVVK

SEQ ID NO: 183 - protein, 111 aa, synthetic construct
AQVLTQTPASVSAAVGGTVSISCQSSKSVYARNHLAWFQQKPGQPPKLLIYKASNLPTGVPSRFSGSGSGTQFTLTISDVQCDDAATYYCAGAFSTSGDNGFGGGTEVVVK

SEQ ID NO: 184 - protein, 112 aa, synthetic construct
AQVLTQTPSSVSAAVGGTVTINCQSSQSVAGTNWLSWYQQKPGQPPKLLIYKASILEAGVSSRFKGSRSGTQFTLTINDMQCDDAATYYCHGAYSSVNVIHTFGGGTEVVVK

SEQ ID NO: 185 - DNA, 351 bp, synthetic construct
  source 1..351: mol_type = other DNA
caggagcagctgaaggagtccgggggtcgcctggtcacgcctgggacacccctgacactcacctgcacagtctctggattctccctcagtagctactacatgtggtgggtccgccaggctccaggaaagggcctggaatggatcggaagtattgggagtcgtgataaatattacgcgacctgggcgaatggtcgagtcaccatctccagaacctcgaccacggtggatctgaagatcaccagtccgacaaccgaggacacggccacctatttctgtgccagagaagttggtggtgagaggactggtgcgtttgtcttgtggggccagggcaccctggtcaccgtctcgagc

SEQ ID NO: 186 - DNA, 351 bp, synthetic construct
  source 1..351: mol_type = other DNA
caggcggtggaggaatccgggggtcgcctggtcacgcctgggacacccctggtactcacctgcacagcctctggaatcgacctcagtagcaacgacatggcgtgggtccgccagcctccagggaaggggctggaattcatcggagccatttggaggggtattaatatattctacgcgaactgggcgaaaggccgactcaccatctccaaaacctcgtcgaccacggtggatctgagaatcaccagtccgacaatcgaggacacggccacctatttctgtgccagagattggattgcttatcatagtggtataagtttctggggccagggcaccctggtcaccgtctcgagc

SEQ ID NO: 187 - DNA, 351 bp, synthetic construct
  source 1..351: mol_type = other DNA
cagtcgctggaggagtccgggggtcgcctggtcacgcctgggacacccctgacactcacctgcacagcctctggattcaccatcagtagctaccatatgagctgggtccgccaggctccagggaaggggctggagtggatcggatatcttgttcgtggtagttataacacatactacgcgagctgggcgaaaggccgattcaccatctccaaaacctcgtcgaccacggtggatctgaagctcaccagtccgacaaccgaggacacggccacctatttctgtgtcagagatgttattggtactagtggttctgacttgtggggccagggcaccctggtcaccgtctcgagc

SEQ ID NO: 188 - DNA, 342 bp, synthetic construct
  source 1..342: mol_type = other DNA
cagtcggtggaggagtccgggggtcgcctggtcacgcctgggacacccctgacactcacctgcacagtctctggcttctccctcagtaccgcctacatgagctgggtccgccaggctccaggggaggggctggaatggatcggatatattagtcctggtggtaccacatactacgcgagctgggcgaaaggccgattcaccatctccaaaacctcgaccacggtggatctgaaaatcaccagtccgacaagcgaggacacggccacctatagctgtgtcaggagtggtgactgtatggactttaatttgtggggccagggcaccctggtcaccgtctcgagc

SEQ ID NO: 189 - DNA, 351 bp, synthetic construct
  source 1..351: mol_type = other DNA
caggcggtggaggagtccgggggtcgcctggtcacgcctgggacacccctggtactcacctgcacagcctctggaatcgacctcagtagcaacgacatggcgtgggtccgccagcctccagggaaggggctggaattcatcggaggcatttggaggggtagtagtatattctacgcgaactgggcgaaaggccgactcaccatctccaaaacctcgtcgaccacggtggatctgagaatcaccagtccgacaatcgaggacacggccacgtatttctgtgccagagattggattggttatcatactggtatcagtttctggggccagggcaccctggtcaccgtctcgagg

SEQ ID NO: 190 - DNA, 342 bp, synthetic construct
  source 1..342: mol_type = other DNA
cagtcgctggaggagtccgggggccgcctggtcacgcctgggacacccctgacactcacctgcacagcctctggacgctccctcagttataagtatatgagttgggtccggcaggctccagggaaggggctggaacggatcggttacattcctgctagtggttttcgcccgaggtatgcgagttgggcgaaaggccgattcaccatctccagaacctcggccacagtggatctgagaatcaccagtccgacaaccgaggacacggccacctatttctgtgccaaagatttgggaatggactttaatttgtggggccagggcaccctggtcaccgtctcgagc

SEQ ID NO: 191 - DNA, 342 bp, synthetic construct
  source 1..342: mol_type = other DNA
cagtcgctggaggagtccgggggtcgcctggtcacgcctgggacacccctgacactcacctgcacagtctctggcttctccctcagtagcaactacatcagctgggtccgccaggctccaggggaggggctggagtgggtcggatatgtcgatactactggccgctcatattacgcgacctgggcaaaaggccgattcaccatctccagaacctcgaccacggtggatctgaaaatgaccagtccgacaagcgaggacacggccacctatttttgtgtcaggagtggtggctgtatggactttgacttgtggggccagggcaccctggtcaccgtctcgagc

SEQ ID NO: 192 - DNA, 351 bp, synthetic construct
  source 1..351: mol_type = other DNA
cagtcggtggaggagtccgggggtcgcctggtcacgcctgggacacccctgacactcacctgcacagtctctgggatcgacctcaatagctacgacatgatctgggtccgccaggctccagggaaggggctggaattcatcggatggatttggaggggtggtattgcgaactacgcgagctgggcgaaaggccgattcaccttctccataacctcgtcgaccacggtggatctcagaatcaccagtccgacaaccgaggacacggccacctatttctgtgccagagattggcttggatatcatactggtacaagtttgtggggccagggcaccctggtcaccgtctcgagc

SEQ ID NO: 193 - DNA, 369 bp, synthetic construct
  source 1..369: mol_type = other DNA
cagtcggtggaggagtccgggggtcgcctggtcacgcctgggacacccctgacactcacctgcacagcctctggattctccctcaatatctatggagtgagctgggtccgccaggctccagggaaggggctggagtggatcggatatatttatgctggtagtggtagcacatggtacgcgagctgggcgaaaggccgattcaccatctccaaaacctcgaccacggtggatctgaaaatcaccagtccgacaaccgaggacacggccacctatttctgtgccagagatctcggttacggtgatagtaattattataaatattatgaatttggtttgtggggccagggcaccctggtcaccgtctcgagc

SEQ ID NO: 194 - DNA, 351 bp, synthetic construct
  source 1..351: mol_type = other DNA
caggagcagctgaaggagtccgggggtcgcctggtcacgcctgggacacccctgacactcacctgcacagcctctggattctccctcaatagctactatatgtggtgggtccgccaggctccaggaaaggggctggaatggatcgggagcattggtagtcgtgataaatattacgcgaactgggcgaaaggccgattcaccatctccagaacctcgaccacggtggatctgaagatcaccagtccgacaaccgacgacacggccacctatttctgtgccagagaagttggtggtgagaggactggtgcgtttgtcttgtggggccagggcaccctggtcaccgtctcgagc

SEQ ID NO: 195 - DNA, 339 bp, synthetic construct
  source 1..339: mol_type = other DNA
cagtcgctggaggagtccgggggtcgcctggtcatgcctgggacacccctgacactcacctgcacagcctctggattctccctcagttcttactatatgagttgggtccgccaggctccagggaaggggctgcagtggatcggatatattgagcctactgattccagggactacgcgagctgggcgaaaggccgattcaccatctccaaaacctcgaccacggtggatctgaggatcatcagtccgacaaccgaggacacggccacctatttttgtgcccgaaatgttggtggtaggcctaatttgtggggccagggcaccctggtcaccgtctcgagc

SEQ ID NO: 196 - DNA, 342 bp, synthetic construct
  source 1..342: mol_type = other DNA
cagtcgctggaggagtccgggggtcgcctggtcacgcctgggacacccctgacactcacctgcgcagcctctggattctccctcagtagcaactacatcagctgggtccgccaggctccaggggaggggctggagtgggtcggatatgtagatactagtggccgctcatattacgcgacctgggcaaaaggccgattcaccatctccagaacctcgaccacggtggatctgaaaatcaccagtccgacaagcgaggacacggccacctatttttgtgttaggagtggtggctgtatggactttgacttgtggggccagggcaccctggtcaccgtctcgagc

SEQ ID NO: 197 - DNA, 351 bp, synthetic construct
  source 1..351: mol_type = other DNA
cagtcgctggaggagtccgggggtcgcctggtcacgcctgggacaccgctgacactcacctgcacagcctctggattcaccatcagtaactaccacatgagctgggtccgccaggctccagggaaggggctggagtggatcggatacattgttgctggtagtgatgtcgcatactacgcgagctgggcgagaggccgattcaccatctccaaaacctcgtcgaccacggtggatctgacaatcaccaggccgacaaccgaggacacggccacctatttctgtgtcagagatgttgctggtactactggttctgacttgtggggccagggcaccctggtcaccgtctcgagc

SEQ ID NO: 198 - DNA, 342 bp, synthetic construct
  source 1..342: mol_type = other DNA
cagtcggtggaggagtccgggggtcgcctggtcacgcctgggacacccctgacactcacctgcacagtctctggcttctccctcagtagcagctacatgagctgggtccgccaggctccagggaaggggctggaatggatcggatatattgatactgctgttgccacttacttcgcgagctgggcaaaaggccgattcaccatctccaaaacctcgaccacggtggatctgaaaatcaccagtccgacaagcgaggacacggccacgtatttctgtgtcaggagtgatgactgtatggcctttaacttctggggccagggcaccctggtcaccgtctcgagc

SEQ ID NO: 199 - DNA, 345 bp, synthetic construct
  source 1..345: mol_type = other DNA
cagtcgctggaggagtccgggggtcgcctggtcacgcctgggacacccctgacactcacctgcacagtctctggcttctccctcagtaccgcctacattagttgggtccgccaggctccaggggaggggctgcaatggatcggatatattcatcttggtcgtagtacatactacgcgagctgggcgcaaggccgattcaccatctccaaaacctcgtcgaccacggtggatctgaaaatcaccagtccgacaagcgaggacacggccacctatttctgtgtcaggagtggtgactgtatggcctttgatttgtggggccagggcaccctggtcaccgtctcgagc

SEQ ID NO: 200 - DNA, 351 bp, synthetic construct
  source 1..351: mol_type = other DNA
cagtcggtggaggagtccgggggtcgcctggtcacgcctgggacacccctgacactcacctgcacagcctctggattcaccatcagtacctaccacatgagctgggtccgccaggctccagggaaggggctggaatggatcggatacattaatgctaatagtggtggcacatggtacgcgagctgggcgaaaggccgattcaccatctccaaaacctcgaccacggtgaatctgaaaatcaccagtccgacaaccgaggacacggccacctatttctgtgtcagaagtggttataatagtggtatggcttttgatccctggggccagggcaccctggtcaccgtctcgagc

SEQ ID NO: 201 - DNA, 369 bp, synthetic construct
  source 1..369: mol_type = other DNA
caggagcagctgggggagtccggaggaggcctggtccagcctgggggaaccctgaaactctcctgcaaaggctctggattcgacctcagtagcaatgcaatgtgctgggtccgccaggctccagggaaggggctggagtgggtcggatgcattatttatggtagcacatactacgcgacctgggtgaatggccgattcactctctccagagacaacgcccagagctcggtggatctgcaactgagcagtctgacagccgcggacacggccacctatttctgtgccagaggggggcgtagttatggtgatggttatgcttttggatactttaacatctggggccagggcaccctggtcaccgtctcgagc

SEQ ID NO: 202 - DNA, 348 bp, synthetic construct
  source 1..348: mol_type = other DNA
caggagcagctggaggagtccgggggtcgcctgggcacgcctgggacacccctgacactcacctgcacagtctctggattctccctcattggccaatatatgtcctgggtccgccaggctccagggaaggggctggaatggatcggatatattgatactactgaaaggtcatactacgcgagctgggcgaaaggccgattcaccatctccaaaacctcgtcgaccacggtggatctgaaaatgaccagtctgacaaccgaggacacggccacctatttttgtgtcagatccgatagttgtatggcgtttggcttatggggccagggcaccctggtcaccgtctcgagc

SEQ ID NO: 203 - DNA, 375 bp, synthetic construct
  source 1..375: mol_type = other DNA
cagtccggaggaggaggcgaaggaggcctggtcaagcctgggggatccctggaactctgctgcaaggcctctggattctctctcaataacaaattctggatatggtgggtccgccaggctccagggaaggggctggagtggattggaagcattgatgctggtggtagtggtggcacttactacgcgagctgggtgaatggccgattcactctctccagagacatcgaccagagcacttattgcctacaactgaacagtctgacagccgcggacacggccatatattactgtgcgagagatttagcggctgctggtggtggtctaacgggcgcctttaacttgtggggccagggcaccctggtcaccgtctcgagc

SEQ ID NO: 204 - DNA, 381 bp, synthetic construct
  source 1..381: mol_type = other DNA
cagtcgttggaggagtccgggggaggcctggtccagcctgagggatccctgacactcacctgcacagcttctggattctccttcagtagcagctactacatgtgctgggtccgccaggctccagggaaggggctggagtggatcgcatgcatttatgttggcagtaataataattcttactacgcgagctgggcgaaaggccgattcgccatctccaaaacctcgtcgaccacggtgactctgcaaatgggcagtctgacagccgcggacacggccacctatttctgtgcgaggagtattgttggttatgctggttatacttatgctcggtacaatgcttttgatccctggggccagggcaccctggtcaccgtctcgagc

SEQ ID NO: 205 - DNA, 363 bp, synthetic construct
  source 1..363: mol_type = other DNA
caggagcaactggtggagtccgggggaggcctggtcaagcctggggcatccctgacactcacctgcaaagcctctggattctccttcagtagcggctactacatgtgctgggtccgccaggctccagggaaggggctggagtggatcggatgcattcttcccggcactgtggcctcttactacgcgagctgggcgaaaggccggtccaccatctccaaagcctcgtcgaccacggtggatctgaaaatgaccaggctgacagcggcggatacggccacttatttctgtgcgagagttaatcctacttatggtggccatggatatgacttgtggggccagggcaccctggtcaccgtctcgagc

SEQ ID NO: 206 - DNA, 363 bp, synthetic construct
  source 1..363: mol_type = other DNA
caggagcagctggtggagtccgggggaggcctggtccagcctggggcatccctgacactcacctgcaaagcctctggattctccgtcagtagcggcgtttacatgtgctgggtccgccaagctccagggaaggggctggagtggatcggatgcattcttactggcagtgttacctcttactacgcgacctgggtgaatggccgattcaccctctccaaagcctcgtcgaccacactatatctacaactgaacagtctgacagtcgcggacacggccacccatttctgtgtaagagttaatcccaattatggtggccatggttatgacttgtggggccagggcaccctggtcaccgtctcgagc

SEQ ID NO: 207 - DNA, 357 bp, synthetic construct
  source 1..357: mol_type = other DNA
cggtcgttggaggagtccgggggaggcctggtcaagcctggagaaaccctgacactcacctgcaaagcctctggattcgacttcactagcagtgcagcgtgctgggtccgccaggctccagggaaggggctggagtggatcggatgtatacttaatagtaggatctattatgcggcctgggcgaaaggccgattcaccatttccagggcctcgtcgatcacggtgactctacgaatgaccagtctgacagccgcggacacggccacctatttctgtgcgagagtccctggtggcagtccttataattggtgggccatggacctctggggccagggcaccctggtcaccgtctcgagc

SEQ ID NO: 208 - DNA, 330 bp, synthetic construct
  source 1..330: mol_type = other DNA
gcccttgtgatgacccagtctccatcctccgtgtctgcagctgtgggagacacagtcaccatcaagtgccaggccagtcagagcattagtggctacttagcctggtatcagcagaaaccagggcaggctcccaagctcctgatctacaaggcttccactctggcatctggggtctcatcgcgattcaaaggccgtggatctgggacagagttcactctcaccatcagcgacctggagtgtgccgatgctgccacttactattgtcaacagggtgttagtggtaataatgttcaaaatgttttcggcggagggaccgaggtggtggtcaaa

SEQ ID NO: 209 - DNA, 327 bp, synthetic construct
  source 1..327: mol_type = other DNA
gcccaagtgctgacccagactccatcctccgtgtctgcagctgtgggaggcacagtcaccatcaattgccagtccagtccgagcgtttataataacaatgcattatcctggtttcagcagaaaccagggcagcctcccaaggtcctgatctccagggcatccactcgggcatctggggtcccatcgcggttcaagggcagtggatctgggacacagttcactctcaccatcagcggcgtggagtgtgtcgatgctgccacttactactgtgcaggcggttttgatagtgatggtgctttcggcggagggaccgaggtggtggtcaaa

SEQ ID NO: 210 - DNA, 333 bp, synthetic construct
  source 1..333: mol_type = other DNA
gcagccgtgctgacccagacaccatcgcccgtgtctgcagctgtgggaggcacagtcaccatcaagtgccagtccagtcagactgtttataataacaacctcttatcctggtatcagcagaaaccagggcagcctcccaagctcctgatctatctggcgtccactctggcatctggggtcccagataggttcagcggcagtggatctgggacacagttcactctcaccatcagcggcgtgcagtgtgacgatgctgccacttactactgtctaggcggttttcataatgatgctgatacggctttcggcggagggaccgaggtggtggtcaaa

SEQ ID NO: 211 - DNA, 333 bp, synthetic construct
  source 1..333: mol_type = other DNA
gccgccgtgctgacccagactccatctcccgtgtctacagctgtgggaggcacagtcagcatcagttgccagtccactaagagtgtttggcgtaagaactgcttagtctggtatcaacagaaaccagggcagcctcccaagcgcctgatttaccaggcatccagtctggcatctggggtcccatcgcggttcaaaggcagtggatctgggacagatttcactctcaccatcggcgacgtgcagtgtgacgatgctgccacttactactgtgcgggcggttatagtgatagtagtgataatgctttcggcggagggaccgaggtggtggtcaaa

SEQ ID NO: 212 - DNA, 327 bp, synthetic construct
  source 1..327: mol_type = other DNA
gcccaagtgctgacccagactccagcctccgtgtctgcagctgtgggaggcacagtcaccatcaattgccagtccagtccgagtgtttataataacaatgctttagcctggtatcaacagaaaccagggcagcctcccaagctcctgatctccagggcatccactctggcatctggggtcccatcgcggttcaagggcagtggatctgggacacaattcactctcaccatcagcggcgtggagtgtgtcgatgctgccacttactactgtgcaggcggtttttatagtgatggtgttttcggcggagggaccgaggtggtggtcaaa

SEQ ID NO: 213 - DNA, 333 bp, synthetic construct
  source 1..333: mol_type = other DNA
gcagccgtactgacccagacaccatcacccgtgtctgcagctgtgggaggcacagtcaccatcacttgccagtccagtcagagtgtttataatgacaaccaattatcctggtatcagcagaaatcagggcagcctcccaagatcctgatctacgaagcatccaaactggcatctggggtcccatcgcggttcagtggcagtggatctgggacacagttcactctcaccatcgtcggtgtgcagtgtgacgatgctgcctcttactactgcctaggcgcttataatgatgattctgagactgctttcggcggagggaccgaggtggtggtcaaa

SEQ ID NO: 214 - DNA, 333 bp, synthetic construct
  source 1..333: mol_type = other DNA
gcccaagtgctgactcagactccatcctccgtgtctgcagctgtgggaggctcagtcagcatcagttgccagtccactaagagtgtttggagtcagaattgcttagtctggtatcagcagaaaccagggcagcctcccaagcgcctcatttaccaggcatcgaaactggcatctggggtcccgtcgcggttcaaaggcagtggctctgggacacagttcactctcaccatcggcgacgtgcagtgtgacgatgctgccacttactactgtgcaggcgcttattatgatagtagtgataatggtttcggcggagggaccgaggtggtggtcaaa

SEQ ID NO: 215 - DNA, 327 bp, synthetic construct
  source 1..327: mol_type = other DNA
gcccaagtgctgacccagactcctgcctccgtgtctgcagctgtgggaggcacagtcaccatcagttgccagtccagtaagagtgtttatagtaacaactgcttatcctggtttcaacagaaaccaggccagcctcctaagctcctgatctacagggcatccactctggcatctggggtcccatcgcggttcaagggcagtggatctggggcacagtttattctcaccattagcgacgtgcagtgtgacgatgctgccacttactactgtgcaggcggttataatagtgatggtgctttcggcggagggaccgaggtggtggtcaaa

SEQ ID NO: 216 - DNA, 327 bp, synthetic construct
  source 1..327: mol_type = other DNA
gcccaagtgctgacccagactccatcccctgtgtctgcagctgtgggaggcacagtcaccatcgcttgccagtccagtcagagtgtttataataataaacacttagcctggtatcagcagaaactaggacagcctcccaaactcctgatctattctgcgtccaaagtggcatctggggtctcatcgcggttcagtggccgtggatctgggacacagttcactctcaccatcagtggcgtggactgtgacgatgctgccacttactactgtgcaggcggttatagtgacgttcgtgctttcggcggagggaccgaggtggtggtcaaa

SEQ ID NO: 217 - DNA, 330 bp, synthetic construct
  source 1..330: mol_type = other DNA
gcccttgtgatgacccagactccatcctccgtgtctgaacctgtgggaggcacagtcaccatcaagtgccaggccagtcagagcattagtggctacttagcctggtatcagcagaaaccagggcagcctcccaagctcctgatctacaaggcatccactttggcatctggggtctcatcgcgattcaaaggcagtggatctgggacagagttcactctcaccatcagcgacctggagtgtgccgatgctgccacttactactgtcaacagggtgttagtggtagtaatattcataatcctttcggcggagggaccgaggtggtggtcaaa

SEQ ID NO: 218 - DNA, 330 bp, synthetic construct
  source 1..330: mol_type = other DNA
gcctatgatgtgacccagactccagcctctgtggaggcagctgtgggaggcacagtcaccatcaagtgccaggtcagtcagagcgttagcaattggttagcctggtatcagcagaaaccagggcagcctcccaagctcctgatctattctgcatccactctggcatctggggtcccatcgcggttcaaaggcagtggatctgggacacagttcactctcaccatcagcgacctggagtgtgccgatgctgccacttattattgtcaacaggcttatagtggtaggaatgttgataatgttttcggcggagggaccgaggtggtggtcaaa

SEQ ID NO: 219 - DNA, 333 bp, synthetic construct
  source 1..333: mol_type = other DNA
gcccaagtgctgacccagactccatcctccgtgtctacaactgtgggaggcacagtcagcatcagttgccagtccactaagagtgtttggagtaagaactgcttagtctggtatcagcagaaaccagggcagcctcccaagcgcctgatttaccaggcatcgaaactggcatctggggtcccgtcgcggttcaaaggcagtggatctgggacacagttcactctcaccatcggcgacgtgcagtgtgacgatgctgccacttactactgtgcaggcgcttattatgatagtagtgataatggtttcggcggagggaccgaggtggtggtcaaa

SEQ ID NO: 220 - DNA, 333 bp, synthetic construct
  source 1..333: mol_type = other DNA
gcagccgtgctgacccagacaccatcgcccgtgtctgcagctgtgggaggcacagtcaccatcaagtgccagtccagtcagagtgtttataataaaaacctcttatcctggtatcagcagaaacccgggcagcctcccagggtcctgatctacaaggcatccactctggcatctggggtcccatcacggttcagcggcagtgggtctgggacacagttcactctcaccatccgcggcgtgcagtgtgacgatgctgccacttactactgtctaggcgcttatgataatgatgctgatacggcgttcggcggagggaccgaggtggtggtcaaa

SEQ ID NO: 221 - DNA, 333 bp, synthetic construct
  source 1..333: mol_type = other DNA
gccgccgtgctgacccagactccagcctccgtgtctgcagctgtgggaggcacagtcaccatcagttgccagtccaccaggagtgtttggcgtaacaactgcttagcctggtatcagcagaaaccagggcagcctcccaagctcctgatctacagggcatccagtctggcatctggggtcccatcgcgattcagaggcagtggatctgggacacagttcactctcaccatcggcgacgtgcagtgtgacgatgctgccacttactactgtgcaggcgggtatagtgatgcacgtgataatggtttcggcggagggaccgaggtggtggtcaaa

SEQ ID NO: 222 - DNA, 333 bp, synthetic construct
  source 1..333: mol_type = other DNA
gccgccgtgctgacccagactccatcccccgtgtctgcagctgtgggaggcacagtcagcatcagttgccagtccactaagagtgtttatagtaagaactgcttagcctggtatcagcagaaaccagggcagcctcccaagctcctgatctacaaggcttccactctggcatctggggtcccatcgcggttcaaaggcagtggatctgggacacagttcactctcaccatcggcgacgtgcagtgtgacgatgttgccacttactactgtgcaggcggctatagtgatgctcgagataatgctttcggcggagggaccgaggtggtggtcaaa

SEQ ID NO: 223 - DNA, 333 bp, synthetic construct
  source 1..333: mol_type = other DNA
gccgccgtgctgacccagactccatctcccgtgtctgcagctgtgggaggcacagtcagcatcagttgccagtccagtaagagtgtttataataacaacaatttagcctggtttcagcagaaaccagggcagcctcccaagctcctgatctatggtgcatccatgctggcatctggggtcccatcgcgattcaaaggcagtggatctgggacgcagttcactctcaccatcagtgacgtgcagtgtgacgatgctgccacttactactgtgcaggcgcttttagtagtgccagtgacaatgctttcggcggagggaccgaggtggtggtcaaa

SEQ ID NO: 224 - DNA, 336 bp, synthetic construct
  source 1..336: mol_type = other DNA
gcccttgtgatgacccagactccgtcctccgtgtctgcagctgtgggaggcacagtcaccatcaattgccaggccagtcagaacatttacagcaatttagcctggtatcaacagaaaccagggcagcgtcccaagctcctgatctatggtacatccaatctggaatctggggtcccatcgcggttcaaaggcagtggatctgggacagagtacactctcaccatcagcgacctggagtgtgacgatgccgccacttactactgtcaaagtgcttattatagtagtagtactgatatggctaatgctttcggcggagggaccgaggtggtggtcaaa

SEQ ID NO: 225 - DNA, 333 bp, synthetic construct
  source 1..333: mol_type = other DNA
gtccaagtgctgacccagactccatcctccgtgtctgcagctgtgggaggcacagtcaccatgagttgccagtccactaagagtgtttggaataataattgtttagcctggtatcagcagaaaccagggcagcctcccaagctcctgatctacaaggcttccactctggcagctggggtctcatcgcggttcaaaggcaatggatctgggacacagtccactctcaccatcagcgacgtgcagtgtgacgatgctgccacttactactgtgcaggcggttatagtactagtagtgataatggttttggcggagggaccgaggtggtggtcaaa

SEQ ID NO: 226 - DNA, 330 bp, synthetic construct
  source 1..330: mol_type = other DNA
gcctatgatatgacccagactccagcctccgtgtctgcagctgtgggaggcacagtcaccatcaattgccaggccagtcagagcattaacaacctcttagcctggtatcagcagaaaccagggcagcctcccaagctcctgatctacaaggcatccactctggcatctggggtcccatcgcggttcaaaggcagtggatctgggacacagttcactctcaccatcagcggcgtgcagtgtgccgatgctgccacttactattgtcaacagggttggagtggtagtaatgttgataatgttttcggcggagggaccgaggtggtggtcaaa

SEQ ID NO: 227 - DNA, 333 bp, synthetic construct
  source 1..333: mol_type = other DNA
gcccttgtgatgacccagactccatcctccgtgtctggagctgtgggaggcacagtcaccatcaattgccaggccagtcagaacatttacaacaatttagcctggtatcagcagaaaccagggcagcctcccaagctcctgatctatgctgcatccaatctggcatctggggtctcatcaaggttcaaaggcagtggatctgggacagagtacactctcaccatcagcgacctggagtgtgacgatgctgccacttactactgtcaaagtgttgcttatagtactggtgctgctacgatcgctttcggcggagggaccgaggtggtggtcaaa

SEQ ID NO: 228 - DNA, 333 bp, synthetic construct
  source 1..333: mol_type = other DNA
gccgccgtgctgacccagactccatctcccgtgtctgcagctgtgggaggcacagtcagcatcagttgccagtccagtaagagtgttcataataataaccacttagcctggtttcagcagaaaccagggcagcctcccaagctcctgatctacaaggcttccactctggcatctggggtcccatcgcggttcaaaggcagtggatctgggacacagttcactctcaccatcagcgacgtgcagtgtgacgatgctgccacttactactgtgcaggcgctttcatttctagtggtgataatggcttcggcggagggaccgaggtggtggtcaaa

SEQ ID NO: 229 - DNA, 333 bp, synthetic construct
  source 1..333: mol_type = other DNA
gcccaagtgctgacccagactccagcctccgtgtctgcagctgtgggaggcacagtcagcatcagttgccagtccagtaagagtgtttatgctcgcaaccacttagcctggtttcagcagaaaccagggcagcctcccaagctcctgatctacaaggcatccaacctgccaactggggtcccatcgcggttcagcggcagtggatctgggacacagttcactctcaccatcagcgacgtgcagtgtgacgatgctgccacgtactactgtgcaggcgctttcagtactagtggtgataatggtttcggcggagggaccgaggtggtggtcaaa

SEQ ID NO: 230 - DNA, 336 bp, synthetic construct
  source 1..336: mol_type = other DNA
gcgcaagtgctgacccagactccatcgtccgtgtctgcagctgtgggaggcacagtcaccatcaattgtcagtccagtcagagcgttgctggtaccaactggttatcctggtatcagcagaaaccagggcagcctcccaagctcctgatctacaaggcatccattctggaggccggggtctcatcgcggttcaaaggcagtagatctgggacgcagttcactctcaccatcaacgacatgcagtgtgacgatgctgccacttactactgtcatggcgcctatagtagtgttaatgtgattcatactttcggcggagggaccgaggtggtggtcaaa

(19) United States
(12) Patent Application Publication
(10) Pub. No.: US 2026/0243783 A1
(43) Pub. Date: Aug. 20, 2026

(54) Immunoassays for Buprenorphine and Metabolites

(71) Applicant: Ark Diagnostics, Inc., Fremont, CA (US)

(72) Inventors: Rajendra Singh, San Jose, CA (US); Soon Jin Oh, Sunnyvale, CA (US); Jie Liang, Newark, CA (US)

(21) Appl. No.: 19/540,475

(22) Filed: Feb. 13, 2026

Related U.S. Application Data
(60) Provisional application No. 63/761,006, filed on Feb. 20, 2025.

(51) Int. Cl. G01N 33/94; C07D 491/08; C07H 17/00; C07K 16/44; G01N 33/58

(52) U.S. Cl. CPC G01N 33/9486; C07D 491/08; C07H 17/00; C07K 16/44; G01N 33/581; G01N 2333/904

(57) ABSTRACT

Disclosed herein are antibodies that specifically bind to buprenorphine and its metabolites norbuprenorphine, buprenorphine glucuronide and norbuprenorphine glucuronide analytes. Such antibodies can be used to detect a buprenorphine analyte in a sample, such as in a homogeneous enzyme immunoassay method. Hapten structures to elicit such antibodies and conjugates useful in immunoassays are also described.

### CROSS-REFERENCE TO RELATED APPLICATIONS

This application claims the benefit of U.S. Provisional Application No. 63/761,006, filed Feb. 20, 2025, the disclosure of which is incorporated herein by reference in its entirety.

### FIELD OF THE INVENTION

The present invention relates to methods and systems for the detection of buprenorphine (BUP) and its metabolites norbuprenorphine (N-BUP), buprenorphine glucuronide (BUP-G) and norbuprenorphine glucuronide (NBUP-G) using immunoassays. In particular, the invention relates to antibodies and haptens used in immunoassays for the detection and quantification of BUP, N-BUP, BUP-G and NBUP-G in biological samples.

### SEQUENCE LISTING

This application hereby incorporates by reference the material of the electronic Sequence Listing filed concurrently herewith. The material in the electronic Sequence Listing is submitted as an xml (.xml) file entitled “ARKD-010WO_Seq_Listing.xml” created on Feb. 9, 2026, which has a file size of approximately 208,896 bytes, and is herein incorporated by reference in its entirety.

### INTRODUCTION

Buprenorphine, N-cyclopropylmethyl-7a-[1-(5)-hydroxy-1,2,2-trimethylpropyl]-6,14-endo-ethano-6,7,8,14-tetrahydrooripavine, is a highly lipophilic opiate analog with both agonist and antagonist properties at the μ-opiate receptor (Martin et al., J. Pharmacol. Exp. Ther., 197:517 (1976); Cowan et al., Br. J. Pharmacol., 60:537 (1977); Heel et al., Drugs, 17:81 (1979)). As an analgesic, it has a potency about 30 times higher than that of morphine and 75 times higher than that of pentazocine when administered intravenously or intramuscularly and is effective in the treatment of acute and chronic pain. In last couple of decades buprenorphine a semi-synthetic narcotic derived from thebaine, has attracted attention as a potential pharmacotherapy for opiate abuse and dependence. Recently with the supply shortage of heroin, buprenorphine, has been used as the substitute drug of choice. BUP's unique effects and pharmacology make it an attractive and clinically helpful treatment option. The FDA has approved several buprenorphine formulations to treat opioid use disorders. The unique pharmacology of buprenorphine at the mu-opioid receptor (i.e. high affinity, low intrinsic activity and slow dissociation) results in buprenorphine having: (1) a good safety profile, (2) low physical dependence, and (3) flexibility in dose scheduling. Further, BUP produces less euphoria than morphine and heroin. When compared with other opiates, it also causes a significantly lower degree of sedation and respiratory depression, the slowing down of breathing that makes heroin overdoses so dangerous.

On the other hand, BUP has been chosen by the National Institute on Drug Abuse as one of the medications for the treatment of opiate dependence. Norbuprenorphine, NBUP hereinafter, is a major dealkylated metabolite of BUP and displays similar but distinct properties. These limited side effects can be attributed to the fact that BUP and NBUP are partial mu agonists. Agonists are chemicals that bind to and stimulate opiate receptors by releasing second messengers. Antagonists block the effects of opiates by binding to receptors without stimulating them. By stimulating mu opiate receptors in the brain, mu agonists produce the effects associated with morphine: analgesia, euphoria, sedation, and respiratory depression. Because BUP is a partial mu agonist, it also readily binds to mu opiate receptors. However, BUP activates these receptors to a lesser degree than full mu receptor agonists such as morphine and heroin. Both N-BUP and BUP are potent partial agonists, with NBUP having moderate efficacy and BUP having low efficacy. BUP also is released slowly from the mu receptor, producing a long-lasting effect. Therefore, it may be possible to give BUP to patients every other day, rather than in the daily doses that methadone patients must receive. Some studies also suggest that withdrawal effects are less severe with BUP than with methadone. From both a drug of abuse and therapeutic drug monitoring view point, it is important to have available reliable immunoassays for BUP and its metabolites, NBUP, BUP-G and NBUP-G. HPLC-MS methods for the detection of buprenorphine, norbuprenorphine and their glucuronides are disclosed in the literature [Kronstrand, R.; Selden G.; Josefsson, M. “Analysis of buprenorphine, norbuprenorphine, and their glucuronides in urine by liquid chromatography-mass spectrometry”, J. Anal. Toxicol, 2003, 27, (7), p 464-70.], and two immunoassays have also been disclosed [Cirimele, V.; Kintz, P.; Lohner, S; Ludes, B. “Enzyme immunoassay validation for the detection of buprenorphine in urine”, J. Anal. Toxicol. 2003, 27, (2)ₙ p 103-5, and Tiong, G. K. L.; Olley, J. E. “Enzyme immunoassay of buprenorphine”, Naunyn-Schmiedeberg's Arch Pharmacol 1988, 338, 202]. However, these assays are not suitable for use in automated clinical analyzers. There is, therefore, a need for assays for the detection of the forementioned drugs and, in some instances, their metabolites. The assays should be able to detect these drugs in order to monitor and treat addicted patients (Roger L Bertholf, and Gary M Reisfield, MD, Laboratory Medicine, Volume 48, Issue 4, November 2017, Pages e57-e61).

Buprenorphine has a long duration of action because it dissociates slowly from the buprenorphine-receptor complex (Hambrook et al., Opiates and Endogenous Opiate Peptides, 295-301 (1976)). Another important feature of the drug is its limited effect on respiration, even in overdose (Hand et al., Ann. Clin. Biochem., 23:47-53 (1986); Banks et al., N.Z. Med. J., 89:256-257 (1979)). Due to its potency and other beneficial properties, buprenorphine is used broadly for pain management in, for example, cancer and postoperative patients. Besides its beneficial use, the abuse of buprenorphine by patients necessitates monitoring in addition to compliance.

Further, due to its long duration of action, its ability to antagonize opiates, its low dependence liability and lack of significant withdrawal symptoms compared to heroin, cocaine, and other narcotics, buprenorphine is also useful in the management of opiate dependency, including the rehabilitation of opiate addicts (Jasinski et al., Arch. Gen. Psychiatry, 35:501 (1978); Mello et al., Science, 207:657 (1980)). Nevertheless, despite its lower physical dependence liability, buprenorphine abuse has been reported (Strang, Lancet, 25:725 (1985); Robertson et al., Br. Med. J., 292:1465 (1986); Chowhurdy et al., Br. J. Addiction, 85:1349 (1990)).

The chemistry of buprenorphine metabolism in man and other animals (e.g., canine and equine) has been well-studied, with the kinetics reported in plasma (Hand et al., Ann. Clin. Biochem., 23:47-53 (1986); McQuay et al., Advances in Pain Research and Therapy, pp. 271-278 (1986); Bullingham et al., Clin. Pharmacokinet., 8:332-343 (1983)) and in urine (Cone et al., Drug Metab. Dispos., 12:577-581 (1984); Hand et al., J Anal. Tox., 13:100-104 (1989); Heel et al., Drugs, 17:81 (1979)) after intravenous, intramuscular, and sublingual administration. The parent drug is nearly completely metabolized to norbuprenorphine N-BUP, norbuprenorphine glucuronide, NBUP-G, and buprenorphine glucuronide BUP-G, and is consequently present in urine at extremely low concentrations. After sublingual administration, buprenorphine plasma concentrations rise slowly and are maintained at low concentrations for several hours (Bullingham et al., Br. J. Clin. Pharmacol., 13:665-673 (1982)). Buprenorphine has a long half-life of about 8 hours, and the norbuprenorphine metabolite appears to have an even slower elimination (Hand et al., Ann. Clin. Biochem., 23:47-53 (1986); McQuay et al., Advances in Pain Research and Therapy, pp. 271-278 (1986); Bullingham et al., Clin. Pharmacokinet., 8:332-343 (1983)). This accords with the appearance of buprenorphine and its buprenorphine glucuronide metabolite in urine in 1-2 days and norbuprenorphine and norbuprenorphine glucuronide in 1-4 days (Cone et al., Drug Metab. Dispos., 12:577-581 (1984); Blom et al., J Chromatogr., 338:89-98 (1985)). Heel et al. reports that approximately 15-27% of a dose of buprenorphine appears in the urine, mainly in the form of glucuronide metabolites of the parent compound and the norbuprenorphine metabolite (Heel et al., Drugs, 17:81 (1979)). Buprenorphine monitoring thus requires the measurement of the parent drug BUP, as well as the three metabolites NBUP, BUP-G and NBUP-G.

### SUMMARY OF THE INVENTION

The accurate detection of buprenorphine and its metabolites in a biological sample, such as plasma or urine, is useful for several purposes, including determining the illicit use or abuse of buprenorphine, monitoring the dose and efficacy of buprenorphine during clinical treatment for pain, and confirming the prescriptive use of buprenorphine, for example, confirming its use in a drug rehabilitation program. Furthermore, given that the criteria of assay performance for one use may not be applicable for other uses, the differential detection of buprenorphine and its metabolites, i.e., an assay that distinguishes between buprenorphine and buprenorphine metabolites, is useful. For example, confirmation of appropriate rehabilitative use of buprenorphine requires the accurate detection of buprenorphine metabolites rather than buprenorphine alone in urine, as the presence of significant amounts of the unmetabolized parent drug in a urine sample indicates patient tampering, i.e., adulteration of the sample with buprenorphine to feign compliance with the rehabilitation program. Thus, an assay for the sensitive detection of buprenorphine and its metabolites is required to accurately confirm rehabilitative use of buprenorphine to get a complete status. In some cases, it is critical to accurately measure only the parent drug. For example, when buprenorphine is used for the management of pain under supervised conditions, and plasma levels of the parent drug used in testing there is minimal risk of tampering. (D. Moody et al., J. Anal. Toxicol. 21:406-414, 1997). In other instances, it is appropriate to measure either the parent drug or its metabolites, or both the parent drug and its metabolites. For example, the determination of buprenorphine abuse in humans and other mammalian subjects can be made by detecting the presence of buprenorphine and/or one or more of its urine drug screening enhance workplace safety, monitor medication compliance, and detect drug abuse. Employment screening, federal regulations, unusual patient behavior, and risk patterns may prompt urine drug screening. Compliance testing may be necessary for patients taking controlled substances. Standard immunoassay testing is fast, inexpensive, and the preferred initial test for urine drug screening.

Urine immunoassay screening methods are available for monitoring BUP compliance and misuse; however, these screens have poor sensitivity or specificity. Pretreatment of urine with β-glucuronidase (BG) improves the sensitivity and overall accuracy of three commercialized BUP enzyme immunoassays when compared with liquid chromatography-tandem mass spectrometry (LC-MS-MS) (Snyder et al, Journal of Analytical Toxicology, Volume 38, Issue 6, 2014, Pages 375-379). However, this extra step not only lowers throughput but the preanalytical step, for example the hydrolytic step has to be compatible with the downstream immunoassay conditions particularly for a homogeneous format with no wash steps or buffer exchange.

Urine beta-glucuronidase (BG) pretreatment improved EIA, HEIA and CEDIA sensitivities from 70, 82 and 94%, respectively, to 97% for each of the three methods, when compared with LC-MS-MS. While the specificity of the EIA and HEIA remained 100% after BG pretreatment, the specificity of the CEDIA decreased from 74 to 67% (Snyder et al, Journal of Analytical Toxicology, Volume 38, Issue 6, 2014, Pages 375-379).

This invention improves the sensitivity of the EIA and HEIA Buprenorphine and its metabolites screening methods without any pretreatment by using antibodies capable of detecting all four analytes, BUP, NBUP, BUP-G and NBUP-G when used in conjunction with the enzyme conjugates derived from the novel haptens.

Treatment with medications for opioid use disorder such as buprenorphine improves patient morbidity and mortality as well as treatment adherence, an important component of patient care. Buprenorphine is combined with naloxone to reduce misuse; and, when taken sublingually, naloxone is poorly absorbed. Buprenorphine and Naloxone are structurally similar, with some antibodies showing considerable cross reactivity, therefore, urine testing for some medications containing Naloxone could result as false positives for buprenorphine. Some patients who want to appear adherent may directly tamper with their urine by adding buprenorphine to their urine to allow for the detection without ingestion. Practitioners may rely upon the concentration of buprenorphine and the metabolite, norbuprenorphine, and utilize the ratio of metabolite to parent compound (norbuprenorphine:buprenorphine—N:B ratio) to discern possible evidence of tampering; however, there remains debate as to what specific ratio may signify this practice.

Urinary sample adulteration with exogenous buprenorphine (6,952 ng/ml), which has led to a false-positive immunoassay test result (14.9 ng/ml) on a subsequent sample due to a phenomenon of instrumental carry-over. This unusual case confirms the importance to take into account adulteration when screening urines for buprenorphine in patients undergoing substitution therapy for opioid dependence, routinely perform a confirmation assay on positive samples, and rule out instrumental carry-over of metabolic products.

Analytical methods for the detection of buprenorphine and its metabolites include chromatographic methods, including thin layer chromatography, gas chromatography and high-performance liquid chromatography, which can be used to detect the parent drug buprenorphine (Hackett et al., J Chromatography, 374:400-404 (1986)). Other commercialized methods include fluorescence polarization immunoassay (FPIA), enzyme linked immunoassay (ELISA), radioimmunoassay (RIA), High pressure liquid chromatography (HPLC), HPLC coupled to tandem mass spectrometry (LC-MS/MS) and enzyme multiplied immunoassay technique (EMIT), cloned enzyme donor immunoassay (CEDIA). However, these methods such as ELISA can be time-consuming and expensive or require technical expertise for the accurate quantitation of buprenorphine and/or its metabolites by chromatographic methods coupled to mass spectrometry particularly for preliminary screening campaigns.

Prior methods now obsoleted in clinical chemistry analyzers such as fluorometric or radiometric immunoassays employing the use of a polyclonal antisera that binds to buprenorphine and/or its metabolites used for the detection of these compounds in biological samples. However, current immunoassays are limited by the sensitivity and/or cross-reactivity of the polyclonal antisera used. Cross-reaction of antisera particularly with structurally similar compounds can cause lack of specificity in the measurement of buprenorphine in the absence of extraction steps to overcome interference, which may result in an overestimate of buprenorphine concentration (Bartlett et al., Eur. J. Clin. Pharmacol., 18:339-345 (1980); Debrabandere et al., Analyst, 118:137-143 (1993)). Further, the polyclonal antibody fails to recognize the buprenorphine glucuronide or the norbuprenorphine glucuronide metabolites.

Homogeneous immunoassays have been used for over the last 50 years for measuring drugs of abuse as urine drug tests for example in employment screening using SAMSHA guidelines in the clinical laboratory and hospital. Some advantages of immunoassays are that such assays are accurate, sensitive, and in many commercial assay formats, easy to use. Immunoassays to measure BUP have been commercialized and their availability has enabled the routine measurement of drug levels in patient samples. Such assays have some limitations with respect to sensitivity and specificity and detect only buprenorphine plus norbuprenorphine. Immunoassays depend on the antibody reagent selection to provide specificity which is challenging as it is usually difficult or impossible to construct a drug analog suitable for conjugation to a large molecule (such as a protein) to develop an immunogen that induces an antibody that reacts with the drug. Often, the derivatization necessary to create an immunogen sufficiently alters the drug such that the resulting antibodies recognize the analog, but not the drug. Therefore, preparation of analogs that are suitable for conjugation to a protein and induce antibodies that recognize both the analog and the drug is required to develop an immunoassay.

The structure of BUP and its three major metabolites NBUP, BUP-G and NBUP-G are also provided in FIG. 1.

Siemens Syva® EMIT® technology employs a competitive enzyme immunoassay principle based on competition between drug in the specimen and BUP hapten labeled with the enzyme glucose-6-phosphate dehydrogenase (G6PD) for binding to the antibody reagent. As the latter binds antibody, enzyme activity decreases. In the presence of drug from the specimen, enzyme activity increases and is directly proportional to the drug concentration. Active enzyme converts the coenzyme nicotinamide adenine dinucleotide (NAD⁺) to NADH that is measured spectrophotometrically as a rate of change in absorbance. Endogenous serum G6PD does not interfere with the results because the coenyzme NAD⁺ functions only with the bacterial enzyme used in the assay. Thermo Fisher (DRI) and Lin-Zhi have commercialized BUP assay using similar EMIT technology with G6PD as the enzyme label, however these assays have limitations that are overcome by the current invention. Another homogeneous format the cloned enzyme donor immunoassay (CEDIA) is also commercially available from Thermo Fisher. Free buprenorphine and norbuprenorphine are excreted in urine at low concentrations with assays targeting either BUP or NBUP have higher false negatives as the glucuronides are not detected. The CEDIA method has higher sensitivity compared to the DRI method, attributed to differing antibody specificities (Melanson S E F et al, J. Anal Toxicol, 201-6, 36, 2012). The same study showed that the DRI assay although less sensitive had few false positives due to the presence of structurally similar compounds as the antibodies lacked specificity. There is therefore a need for an assay with not only higher sensitivity but superior specificity compared to methods covered in the prior art. Buprenorphine immunoassay performance has been improved after treatment of the urine sample with beta-glucuronidase (Snyder M L et al, J Anal Toxicol, 375-9, 38, 2014). However this additional step slows throughput, adds cost and creates additional pH, dilution plus buffer compatibility issues downstream with enzyme immunoassays. Thermo Scientific™ CEDIA Buprenorphine II immunoassay (Bup2) detects the presence of free as well as glucuronidated buprenorphine and norbuprenorphine, however the cutoff value of 10 μg/L of this second generation assay is less sensitive than the cut-off value of 5 μg/L of the first generation Thermo Scientific™ CEDIA Buprenorphine immunoassay (Bup1) that detected no glucuronides.

In order for immunoassays to be specific for BUP and its metabolites NBUP, BUP-G and NBUP-G and not cross react with the structurally similar opioid agonist and antagonists, the design of haptens for immunization to select antibodies and generation of enzyme conjugates to yield sensitivities and specificities simultaneously is challenging and the subject of this invention.

Conventional immunoassays, including those described in U.S. Pat. Nos. 7,220,842 and 7,863,427, describe compounds and methods for detecting BUP and NBUP in biological samples use antibodies specific for BUP that have minimal cross reactivity to the glucuronides. The antibodies used in conventional immunoassays have a high cross reactivity to NBUP enabling BUP and NBUP detection and the dynamic range is limited. There is a need for superior specificity and higher dynamic range as well as the capability to detect the glucuronide BUP-G and NBUP-G.

The present disclosure provides methods for immunoassay of BUP and its metabolites NBUP, BUP-G and N-BUP-G analyte. In some embodiments, the present disclosure relates to the use of derivatives of NBUP, BUP-G and NBUP-G haptens, tracers and conjugates in a signal producing immunoassay system. The present disclosure also relates to the use of immunogens of BUP analogs used for producing antibodies for capture of such analytes.

In some embodiments, the present disclosure provides BUP derivatives acylated and/or alkylated on the pyramidal N at the bridgehead (N-17, FIG. 1), O- of the phenolic group (C3, FIG. 1) as well as carboxyl of the glucuronide. In certain embodiments, such derivatives are used to produce immunogens to generate antibodies and conjugates useful in the immunoassays described herein.

In some embodiments, the present disclosure provides a polyclonal or monoclonal antibodies that specifically binds to BUP, NBUP, BUP-G and NBUP-G with less than 0.1% cross reactivity to structurally similar opioids naloxone, morphine, codeine, heroin, oxycodone, oxymorphone and oxycodone.

In some embodiments, the present disclosure provides a polyclonal or monoclonal antibodies that specifically bind to BUP, and NBUP, BUP-G and NBUP-G.

In some embodiments, the antibodies may specifically bind to one or more of BUP, N-BUP, BUP-G and NBUP-G.

In some embodiments, the present disclosure provides methods for the syntheses of haptens, immunogens and conjugates starting from buprenorphine or thebaine. In some embodiments, the synthesis includes coupling through pyramidal bridgehead nitrogen (N-17) of the BUP, NBUP with a linking group to a protein or a label (e.g., a label enzyme).

In some embodiments, the synthesis includes coupling through oxygen of the phenyl group (C-3) of the BUP, NBUP with a linking group to a protein or a label (e.g., a label enzyme).

In some embodiments, the synthesis includes coupling through carboxyl group of the glucuronide BUP-G and NBUP-G with a linking group to a protein or a label (e.g., a label enzyme).

In some aspects, these haptens are conjugated to proteins through various linkers, and thus provide monoclonal antibodies and enzyme conjugates that further reduce the cross reactivity to structurally related opioids.

Further, this disclosure provides antibodies having binding specificity to BUP, NBUP, BUP-G and NBUP-G. In some cases, BUP antibodies comprise a variable heavy chain and a variable light chain, wherein the variable heavy chain amino acid sequence comprises three heavy chain complementarity-determining regions (HCDR1, HCDR2, and HCDR3) and the variable light chain amino acid sequence comprises three light chain complementarity-determining regions (LCDR1, LCDR2, and LCDR3), wherein HCDR1 is selected from one of SEQ ID NOs: 1-23, HCDR2 is selected from one of SEQ ID NOs: 24-46, HCDR3 is selected from one of SEQ ID NOs: 47-69, LCDR1 is selected from one of SEQ ID NOs: 70-92, LCDR2 is selected from one of SEQ ID NOs: 93-115, and LCDR3 is selected from one of SEQ ID NOs: 116-138.

The present disclosure also describes antibodies having binding specificity to BUP, NBUP, BUP-G and NBUP-G and have a variable heavy chain and a variable light chain, wherein the variable heavy chain amino acid sequence comprises a sequence selected from one of SEQ ID NOs: 139-161, and wherein the variable light chain amino acid sequence comprises a sequence selected from one of SEQ ID NOs: 162-184.

The present disclosure relates generally to antibodies having binding specificity to BUP, NBUP, BUP-G and NBUP-G and a variable heavy chain and a variable light chain, wherein the variable heavy chain is encoded by a nucleotide comprising a sequence selected from one of SEQ ID NOs: 185-207, and wherein the variable light chain amino acid is encoded by a nucleotide comprising a sequence selected from one of SEQ ID NOs: 208-230.

The present disclosure further describes methods of detecting BUP, NBUP, BUP-G and NBUP-G in a sample, comprising the steps of combining in a solution the sample with a capture molecule and a labeled BUP, labeled NBUP, labeled BUP-G and/or labeled NBUP-G wherein the capture molecule is capable of binding the labeled BUP, NBUP, BUP-G and/or NBUP-G and wherein BUP, NBUP, BUP-G and NBUP-G when present in the sample competes with the labeled BUP, labeled NBUP, labeled BUP-G and/or labeled NBUP-G for binding to the capture molecule. The methods comprise detecting an amount of labeled BUP, labeled NBUP, labeled BUP-G and/or labeled NBUP-G bound to the capture molecule through signal produced by a label on the bound labeled BUP, labeled NBUP, labeled BUP-G and labeled NBUP-G, wherein the signal is inversely proportional to the amount of BUP, NBUP, BUP-G and NBUP-G present in the sample.

Further, the present disclosure provides kits for detecting BUP, NBUP, BUP-G and NBUP-G in a sample, the kit comprising a capture molecule; a labeled BUP, labeled NBUP, labeled BUP-G, or labeled NBUP-G wherein the capture molecule is capable of binding the labeled BUP labeled labeled NBUP, labeled BUP-G, or labeled NBUP-G and wherein BUP, NBUP, BUP-G or NBUP-G competitively inhibits binding of the labeled BUP. labeled NBUP, labeled BUP-G, or labeled NBUP-G to the capture molecule. The kits may also comprise instructions for performing the detection assay, including combining in a solution the sample with a capture molecule and a labeled BUP, labeled NBUP, labeled BUP-G, or labeled NBUP-G wherein the capture molecule is capable of binding the labeled BUP, labeled NBUP, labeled BUP-G, or labeled NBUP-G and wherein BUP. NBUP, BUP-G and/or NBUP-G when present in the sample competes with the labeled BUP, labeled NBUP, labeled BUP-G, or labeled NBUP-G for binding to the capture molecule; and detecting an amount of labeled BUP, labeled NBUP, labeled BUP-G, or labeled NBUP-G bound to the capture molecule through signal produced by a label on the bound labeled BUP, labeled NBUP, labeled BUP-G, or labeled NBUP-G wherein the signal is inversely proportional to the amount of BUP, NBUP, BUP-G and/or NBUP-G present in the sample.

### BRIEF DESCRIPTION OF THE DRAWINGS

The present disclosure will be more fully understood from the following detailed description thereof taken in connection with the accompanying drawings which form a part of this application and in which:

FIG. 1 shows the chemical structures for BUP and the 3 metabolites (Norbuprenorphine (NBUP), Buprenorphine Glucuronide (BUP-G), and Norbuprenorphine Glucuronide (NBUP-G)) and the numbering system with an alternative nomenclature in accordance with various embodiments.

FIG. 2 shows the chemical structures of structurally related opiates (natural) and opioids (synthetic) in accordance with various embodiments.

FIG. 3 shows the chemical structures of BUP, NBUP and alkyl NBUP-G haptens in accordance with various embodiments.

FIG. 4 shows the synthesis of Hapten BUP-G (Hapten 5) and NBUP-G (Hapten 6), in accordance with various embodiments.

FIG. 5 shows the synthesis scheme for the NBUP N-ethyl bromoacetamide, Hapten 1, according to embodiments of the present disclosure.

FIG. 6 shows the synthesis scheme for the NBUP-G, N-ethyl chloroacetamide, Hapten 2, according to embodiments of the present disclosure.

FIG. 7 shows the synthesis scheme for the BUP O-ethyl bromoacetamide, Hapten 3, according to embodiments of the present disclosure.

FIG. 8 shows the synthesis scheme for the NBUP O-ethyl bromoacetamide, Hapten 4, according to embodiments of the present disclosure.

FIG. 9 shows an exemplary 1H NMR of Hapten 1, in accordance with various embodiments.

FIG. 10 shows an exemplary LC-MS chromatogram of Hapten 1 in accordance with various embodiments.

FIG. 11 shows an exemplary 1H NMR of Hapten 2 in accordance with various embodiments.

FIG. 12 shows an exemplary LC-MS chromatogram of Hapten 2 in accordance with various embodiments.

FIG. 13 shows an exemplary 1H NMR of Hapten 3 in accordance with various embodiments.

FIG. 14 shows an exemplary LC-MS chromatogram of Hapten 3 in accordance with various embodiments.

FIG. 15 shows an exemplary 1H NMR of Hapten 4 in accordance with various embodiments.

FIG. 16 shows an exemplary LC-MS chromatogram of Hapten 4 in accordance with various embodiments.

FIG. 17 shows an exemplary 1H NMR of Buprenorphine Glucuronide, BUP-G Hapten 5 in accordance with various embodiments.

FIG. 18 shows an exemplary LC-MS chromatogram of Buprenorphine Glucuronide, BUP-G Hapten 5 in accordance with various embodiments.

FIG. 19 shows an exemplary 1H NMR of Norbuprenorphine Glucuronide, NBUP-G, Hapten 6 in accordance with various embodiments.

FIG. 20 shows an exemplary LC-MS chromatogram of Norbuprenorphine Glucuronide, NBUP-G, Hapten 6 in accordance with various embodiments.

FIG. 21 shows the conjugation reaction scheme for the haloacetamide Haptens 1, 2, 3 and 4 with KLH to generate immunogens, according to embodiments of the present disclosure.

FIG. 22 shows the conjugation reaction scheme for the BUP-G (Hapten 5) and NBUP-G (Hapten 6) with KLH to generate immunogens, according to embodiments of the present disclosure.

FIG. 23 shows the conjugation reaction scheme for the haloacetamide Haptens 1, 2, 3 and 4 with BSA, according to embodiments of the present disclosure.

FIG. 24 shows the conjugation reaction scheme for the BUP-G (Hapten 5) and NBUP-G (Hapten 6) with BSA, according to embodiments of the present disclosure.

FIG. 25 shows generation of G6PD labeled enzyme conjugates from haloacetamide Haptens 1, 2, 3 and 4, according to embodiments of the present disclosure.

FIG. 26 shows principle of the homogeneous enzyme immunoassay and the antibody screening technique for detecting BUP, NBUP, BUP-G and/or NBUP-G, according to embodiments of the present disclosure.

FIG. 27 shows Buprenorphine response curve in the homogeneous immunoassay format on the Beckman AU 480 Clinical Analyzer, according to embodiments of the present disclosure.

FIG. 28 shows an exemplary histogram for preliminary precision (Qualitative) with cutoff 5.0 ng/ml, in accordance with various embodiments.

FIG. 29 shows exemplary calibration curves of Buprenorphine (BUP), Norbuprenorphine (NBUP), Buprenorphine Glucuronide (BUP-G), and Norbuprenorphine Glucuronide (NBUP-G) generated on the Beckman AU 480 Clinical Analyzer, in accordance with various embodiments.

FIG. 30 illustrates the sequences identified in Tables 1 and 2 providing SEQ ID NOs for amino acid sequences and nucleic acid sequences representing complementarity determining regions (CDRs) and variable chains (V_{H} and V_{L}), and nucleic acid sequences encoding variable chains (V_{H} and V_{L}), in accordance with various embodiments.

### BRIEF DESCRIPTION OF THE SEQUENCES

SEQ ID NOs: 1-23 represent amino acid sequences of the first complementarity determining region of the variable heavy chain (HCDR1) of the antibody clones demonstrated in Table 1.

SEQ ID NOs: 24-46 represent amino acid sequences of the second complementarity determining region of the variable heavy chain (HCDR2) of the antibody clones demonstrated in Table 1.

SEQ ID NOs: 47-69 represent amino acid sequences of the third complementarity determining region of the variable heavy chain (HCDR3) of the antibody clones demonstrated in Table 1.

SEQ ID NOs: 70-92 represent amino acid sequences of the first complementarity determining region of the variable light chain (LCDR1) of the antibody clones demonstrated in Table 1.

SEQ ID NOs: 93-115 represent amino acid sequences of the second complementarity determining region of the variable light chain (LCDR2) of the antibody clones demonstrated in Table 1.

SEQ ID NOs: 116-138 represent amino acid sequences of the third complementarity determining region of the variable light chain (LCDR3) of the antibody clones demonstrated in Table 1.

SEQ ID NOs: 139-161 represent amino acid sequences of the variable heavy chain of the antibody clones demonstrated in Table 1.

SEQ ID NOs: 162-184 represent amino acid sequences of the variable light chain of the antibody clones demonstrated in Table 1.

SEQ ID NOs: 185-207 represent nucleotide sequences encoding the amino acid sequences of the variable heavy chain of the antibody clones demonstrated in Table 1.

SEQ ID NOs: 208-230 represent nucleotide sequences encoding the amino acid sequences of the variable light chain of the antibody clones demonstrated in Table 1.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. It should also be understood that the precise numerical values used in the specification and claims form additional embodiments of the invention, and are intended to include any ranges which can be narrowed to any two end points within the example ranges and values provided. Efforts have been made to ensure the accuracy of the numerical values disclosed herein. Any measured numerical value, however, can inherently contain certain errors resulting from the standard deviation found in its respective measuring technique.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. Also incorporated by reference is any supplemental information that was published along with any of the aforementioned publications, patents and patent applications. For example, some journal articles are published with supplemental information that is typically available online.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications, patents and patents applications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

Before proceeding further with the description of the specific embodiments of the present disclosure, a number of terms will be defined.

### Analyte

A compound or composition to be measured, the material of interest. The analyte is a member of a specific binding pair (sbp) and may be a ligand, which is mono- or polyvalent, usually antigenic or haptenic, and is a single compound or plurality of compounds which share at least one common epitopic or determinant site.

### BUP Analyte

As used herein, the term “BUP analyte” refers to analytes having an antibody binding epitope which is common to BUP. Analytes included in the term “BUP analyte” include (BUP), with low cross reactivity to structurally similar analogs.

### NBUP Analyte

As used herein, the term “NBUP analyte” refers to analytes having an antibody binding epitope which is common to norbuprenorphine. Analytes included in the term “NBUP analyte” include (NBUP), with low cross reactivity to structurally similar analogs.

### BUP-G Analyte

As used herein, the term “BUP-G analyte” refers to analytes having an antibody binding epitope which is common to buprenorphine glucuronide. Analytes included in the term “BUP analyte” include (BUP-G), with low cross reactivity to structurally similar analogs.

### NBUP-G Analyte

As used herein, the term “NBUP-G analyte” refers to analytes having an antibody binding epitope which is common to Norbuprenorphine glucuronide. Analytes included in the term “N-BUP-G analyte” include (NBUP-G), with low cross reactivity to structurally similar analogs.

### Sample Suspected of Containing Analyte

Any sample which is reasonably suspected of containing analyte can be analyzed by the methods of the present disclosure. Such samples can include human, animal or man-made samples. The sample can be prepared in any convenient medium which does not interfere with the assay. Typically, the sample is an aqueous solution or a natural fluid, such as, but not limited to, urine, whole blood, serum, plasma, cerebral-spinal fluid, or saliva. In some instances, the sample is urine.

### Measuring the Amount of Analytes.

The drug buprenorphine (BUP) is metabolized into three major metabolites: norbuprenorphine (NBUP), buprenorphine glucuronide (BUP-G), and norbuprenorphine glucuronide (NBUP-G). Various embodiments described herein may measure one or more of the foregoing compounds (e.g., BUP, NBUP, BUP-G, and/or NBUP-G) as analytes of an assay.

Quantitative, semiquantitative, and qualitative methods as well as all other methods for determining analyte are considered to be methods of measuring the amount of one or more of BUP, NBUP, BUP-G, and NBUP-G as analytes. For example, a method which merely detects the presence or absence of analyte in a sample suspected of containing an analyte is considered to be included within the scope of the present disclosure.

Synonyms for the phrase “measuring the amount of analyte(s)” which are contemplated within the scope of the present disclosure include, but are not limited to, detecting, measuring, or determining analyte; detecting, measuring, or determining the presence of analyte; detecting, or determining the amount of analyte; and detecting, measuring or determining the concentration of analyte.

### Member of a Specific Binding Pair

A member of a specific binding pair (sbp member) is one of two different molecules, having an area on the surface or in a cavity which specifically binds to and is thereby defined as complementary with a particular spatial and polar organization of the other molecule. The members of the specific binding pair are referred to as ligand and receptor (antiligand), sbp member and sbp partner, or the like. These will usually be members of an immunological pair such as antigen-antibody or an antigen-aptamer.

### Ligand

Any organic compound for which a receptor naturally exists or can be prepared. For example, in one context of the present disclosure, the analyte is a ligand and the present disclosure provides methods for determining the amount or concentration of the analyte which is a ligand.

### Receptor

A receptor is any compound or composition capable of recognizing a particular spatial and polar organization of a molecule. These organized areas of a molecule are referred to as epitopic or determinant sites. Illustrative naturally occurring receptors include antibodies and enzymes.

### Epitope

“Epitope” is a molecular region on the surface of an antigen capable of eliciting an immune response and of combining with the specific antibody produced by such a response, also called determinant, antigenic determinant. In reference to a hapten (such as BUP, N-BUP, BUP-G and N-BUP-G) an antibody can be generated against the non-antigenic hapten molecules by conjugating the hapten to an immunogenic carrier. An antibody is then generated which recognizes an “epitope” defined by the hapten.

### Linking Group

A linking group is a portion of a structure which connects two or more substructures. A linking group has at least one uninterrupted chain of atoms extending between the substructures. The atoms of a linking group are themselves connected by chemical bonds. The number of atoms in a linking group is determined by counting the atoms other than hydrogen.

### Conjugate

A conjugate is a molecule comprised of two or more substructures bound together through a linking group to form a single structure. The binding can be made by connecting the subunits through a linking group. Within the context of the present disclosure, a conjugate can include a glucose-6-phosphate dehydrogenase (G6PD) enzyme attached to a hapten, sbp member or analyte analog, such as a conjugate where a G6PD mutant enzyme is used (e.g., recombinant G6PD as described in U.S. Pat. Nos. 6,455,288, 6,090,567, and 6,033,890). Within the context of the present disclosure, G6PD may also be referred to as an enzyme, such as a G6PD enzyme, or a label, such as a G6PD label. In some cases, a conjugate can include a label (e.g., a label protein) including, but not limited to, G6PD, alkaline phosphatase, β-galactosidase, and horse radish peroxidase, or a chemical label such as a fluorescent, luminescent or colorimetric molecule attached to a hapten, sbp member or analyte analog.

### Conjugation

Conjugation is any process where two subunits are linked together to form a conjugate. The conjugation process can be comprised of any number of steps, for example as described herein.

### Hapten

Haptens are capable of binding specifically to corresponding antibodies, but usually do not themselves function as immunogens for preparation of the antibodies. Antibodies which recognize a hapten can be prepared against compounds comprised of the hapten linked to an immunogenic carrier.

### Derivative

The term “derivative” refers to a chemical compound or molecule made from a parent compound by one or more chemical reactions.

### Analog

The term “analog” is a compound having a structure similar to that of another compound, but differing from it in respect to a certain component. It can differ in one or more atoms, functional groups, or substructures, which are replaced with other atoms, groups, or substructures.

### Label

A “label,” “detector molecule,” “reporter” or “detectable marker” is any molecule which produces, or can be induced to produce, a detectable signal. The label can be conjugated to an analyte, immunogen, antibody, or to another molecule such as a receptor or a molecule that can bind to a receptor such as a ligand, particularly a hapten or antibody. A label can be attached directly or indirectly by a linking group. Non-limiting examples of labels include radioactive isotopes (e.g., ¹²⁵I), enzymes (e.g. β-galactosidase, peroxidase), G6PD (e.g., mutant G6PD, such as recombinant G6PD as described in U.S. Pat. Nos. 6,455,288, 6,090,567, and 6,033,890), enzyme fragments, enzyme substrates, enzyme inhibitors, coenzymes, catalysts, fluorophores (e.g., rhodamine, fluorescein isothiocyanate or FITC, or Dylight 649), dyes, chemiluminescers and luminescers (e.g., dioxetanes, luciferin, acridan esters, luminol), or sensitizers. Labels include particles such as, gold, modified latex or quantum dots. Modified latex particles could be dyed with rare earth chelates or organic dyes.

### Immunogen

The term “immunogen” refers to a substance capable of eliciting, producing, or generating an immune response in an organism.

### Immunogenic Carrier

An “immunogenic carrier,” as used herein, is an immunogenic substance, commonly a protein, which can join at one or more positions with haptens, thereby enabling the production of antibodies that can specifically bind with these haptens. Examples of immunogenic carrier substances include, but are not limited to, proteins, glycoproteins, complex polyamino-polysaccharides, particles, and nucleic acids that are recognized as foreign and thereby elicit an immunologic response from the host. The polyamino-polysaccharides may be prepared from polysaccharides using any of the conventional means known for this preparation. Specific examples are KLH (keyhole limpet hemocyanin), BTG (Bovine thryroglobulin), and serum albumins.

### Protein

The terms “protein”, “polypeptide” and “peptide” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, polypeptides having modified peptide backbones, and fusion proteins.

### Signal Producing System

The “signal producing system” is utilized in assays for analytes and may have one or more components, at least one component being a detectable label (e.g., G6PD, such as a mutant G6PD). The signal producing system generates a signal that relates to the presence or amount of analyte in a sample. The signal producing system includes all of the reagents required to produce a measurable signal. For purposes of the present disclosure, typically, the G6PD or a label protein (e.g., alkaline phosphatase, β-galactosidase or horse radish peroxidase) is conjugated to a sbp member analogous to the analyte.

Other components of the signal producing system can include substrates, enhancers, activators, chemiluminescent compounds, cofactors, inhibitors, scavengers, metal ions, specific binding substances required for binding of signal generating substances, coenzymes, substances that react with enzymic products, other enzymes and catalysts, and the like.

The signal producing system provides a signal detectable by external means, such as by measurement of electromagnetic radiation, e.g., by visual examination or spectrophotometry. In some instances, the signal producing system includes a chromophoric substrate and an enzyme label (e.g., mutant G6PD enzyme), where chromophoric substrates are enzymatically converted to dyes which absorb light in the ultraviolet or visible region. The product of the enzymatic reaction can also be interrogated by fluorescence.

### Isolated

“Isolated” when used in the context of an antibody means altered “by the hand of man” from any natural state; i.e., that, if it occurs in nature, it has been changed or removed from its original environment, or both. For example, a naturally occurring antibody naturally present in a living animal in its natural state is not “isolated”, but the same antibody separated from the coexisting materials of its natural state is “isolated”, as the term is employed herein. Antibodies may occur in a composition, such as an immunoassay reagent, which are not naturally occurring compositions, and therein remain isolated antibodies within the meaning of that term as it is employed herein.

### Cross-Reactivity

“Cross-reactivity” refers to the reaction of an antibody with an antigen that was not used to induce that antibody. “Cross-reactivity” may be determined in a quantitative immunoassay by establishing a standard curve using known dilutions of the target analyte. The standard curve is then used to calculate the apparent concentration of the interfering substance present in various known amounts in samples assayed under similar condition. The cross-reactivity is the apparent concentration divided by the actual concentration multiplied by 100.

### Calibration and Control Material

The phrase “calibration and control material” refers to any standard or reference material containing a known amount of an analyte. A sample suspected of containing an analyte and the corresponding calibration material are assayed under similar conditions. The concentration of analyte is calculated by comparing the results obtained for the unknown specimen or sample containing known concentration of analyte with the results obtained for the standard. This is commonly done by constructing or generating a calibration curve.

### Sensitivity

Is used in the sense of detection limit, i.e., the smallest amount of an analyte giving a signal that is distinguishable from the signal obtained in the absence of analyte.

### Spike-Recovery

“Spike-recovery” refers to an assay measuring the amount of analyte (recovery) in a sample mixture compared to a known amount of the analyte added (spiked) to the sample mixture. The measuring the amount of analyte may be expressed in terms of concentration (ng/ml) or a percentage (%).

### Substantial Change in Enzyme Activity

A change in activity of an enzyme sufficient to allow detection of an analyte when the enzyme is used as a label in an assay for the analyte. Typically, the enzyme's activity is reduced 10 to 100%, such as 20 to 99%, or 30 to 95%.

### Inhibitory Antibody

An antibody capable of inhibiting the activity of an enzyme or an enzyme-ligand conjugate upon binding an epitope present on the enzyme. Such antibodies are distinguished from antiligand antibodies capable of inhibiting the enzyme activity of enzyme-ligand conjugates upon binding to the ligand.

### Modulation

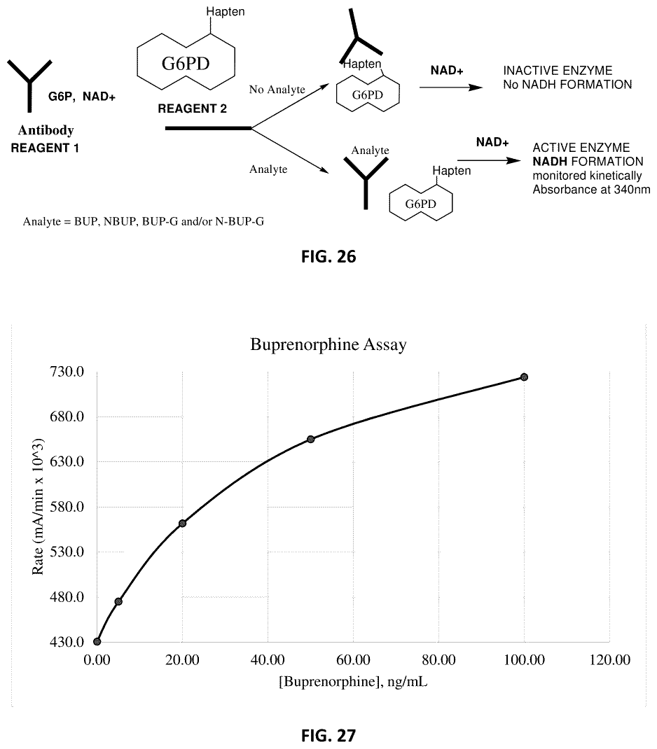

In an assay experiment “modulation” refers to hapten or analyte attached to a label such as an enzyme and an analyte in a sample suspected of containing the analyte competing for analyte-antibody binding sites, thus modulating the amount of enzymatic product formed (see FIG. 26).

### Maximum Inhibition

“Maximum inhibition” refers to an antibody capable of inhibiting the activity of an enzyme or an enzyme-ligand conjugate upon binding an epitope present on the enzyme when excess antibody is added to the assay and the signal obtained in the absence of analyte.

### Ancillary Materials

Various ancillary materials will frequently be employed in an assay in accordance with the present disclosure. For example, buffers will normally be present in the assay medium, as well as stabilizers for the assay medium and the assay components. Frequently, in addition to these additives, additional proteins may be included, such as albumins, or surfactants, particularly non-ionic surfactants, binding enhancers, e.g., polyalkylene glycols, or the like.

In certain aspects, the disclosure provides anti-BUP antibodies, anti-NBUP antibodies, anti BUP-G antibodies and anti NBUP-G induced using immunogens of this invention. Such antibodies or combinations thereof could be used in detecting BUP and its metabolites N-BUP, BUP-G and NBUP-G in an immunoassay.

An immunoassay of the disclosure to detect BUP and its metabolites is based on competition between BUP and its metabolites in the sample and tracers, which are labeled BUP and/or its metabolite analogues of the disclosure, for anti-BUP, anti-N-BUP, anti-BUP-G and anti-N-BUP-G antibodies. In one embodiment, the immunoassay is a homogeneous enzyme immunoassay. Immunoassay kits are also provided.

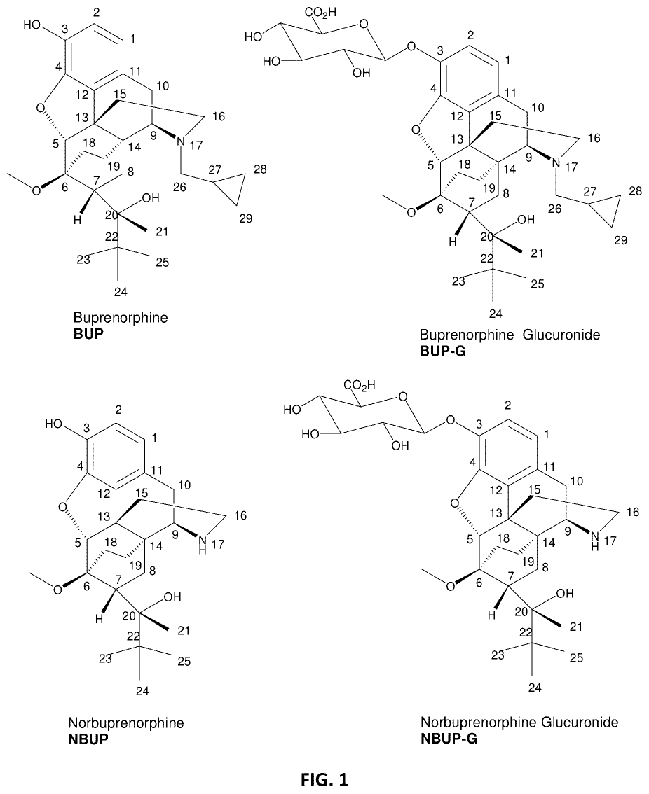

A BUP or NBUP analog of this invention is BUP or N-BUP derivatized to include a chemical moiety that facilitates attachment of a carrier or a label to pyramidal nitrogen at the bridgehead of BUP or NBUP that usually has the linker appended to N. The BUP and N-BUP analogs of this invention are also derivatized through the phenolic oxygen through linkers for conjugation to carrier proteins and signal generating labels. The structure of BUP and N-BUP showing the carbon numbering can be found hereinafter in, for example, FIG. 1.

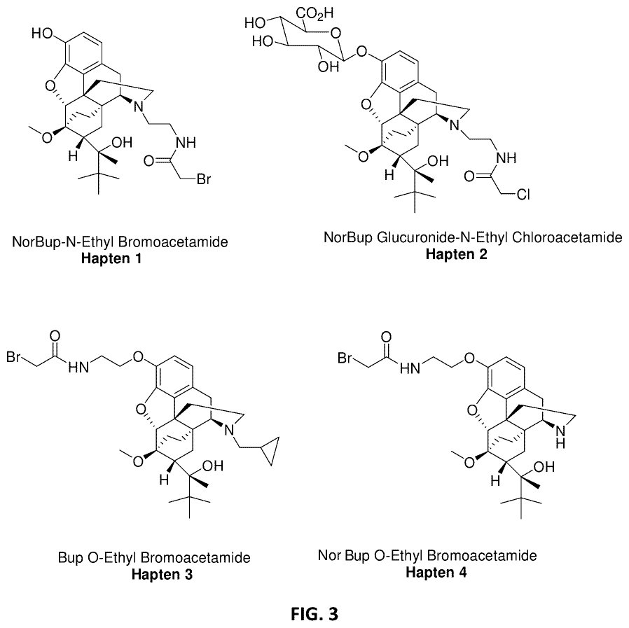

A BUP-G or NBUP-G analogs of this invention is BUP-G or N-BUPG derivatized to include a chemical moiety that facilitates attachment of a carrier or a label to pyramidal nitrogen at the bridgehead of BUP-G or NBUPG that usually has the linker appended to N. The BUP-G and NBUP-G analogs of this invention are also derivatized through the carboxyl group of the glucuronide for conjugation to carrier proteins and signal generating labels. The structure of BUP-G and NBUP-G derivatives showing the carbon numbering and the location of the substituents, can be found hereinafter in, for example, FIG. 3.

U.S. Pat. Nos. 7,220,842 and 7,863,427 describe immunoassays for buprenorphine and norbuprenorphine that utilize immunogens and enzyme conjugates derived from buprenorphine and norburenorphine derivative at the pyramidal N-17 or the phenolic oxygen at C-3 (FIG. 1). Prior art assays do not detect the glucuronides and can yield false negatives when the concentrations of the glucuronides are higher than the cutoff used for buprenorphine and norbuprenorphine. U.S. Pat. Nos. 7,220,842 and 7,863,427 also describe acylated N-17 derivatives that do not mimic the alkylated buprenorphine. The O-alkylated C3 derivatives of buprenorphine and norbuprenorphine used are thus effective in generating immunoassays for buprenorphine and norbuprenorphine only, but not the glucuronides. U.S. Pat. No. 7,195,882 describes the generation of monoclonal antibodies for buprenorphine and metabolites using alkylated buprenorphine both at C3 and N-17. Although some glucuronide antibodies were generated they lacked the sensitivity as the haptens with the free phenolic OH at C-3 were claimed to be glucuronidated in vivo. The present invention describes the synthesis of novel haptens with the glucuronide that generated antibodies with higher cross reactivity to the parent buprenorphine and all the metabolites.

The novel haptens of the present invention provide immunogens and enzyme conjugates that when linked through the carboxyl group of the glucuronide in BUP-G and N-BUPG as well as the phenolic O at C3 and N-17 of the buprenorphine moiety to provide derivatives covering all the epitopic possibilities. The hapten structure provides all the components similar to buprenorphine and the metabolites, their immunological identity induced antibodies resulting in antibodies that react with buprenorphine as well as the metabolites but not with structurally similar opiates. In addition, the BUP haptens can be labeled for use as a tracer in an immunoassay, as described more fully hereinafter.

As is well known, drugs or other haptens can be derivatized to include a linking group with a chemical moiety that facilitates attachment of the hapten to a carrier or a label. Linking groups for preparing haptens are described in, Bioconjugation ed. Mohammed Aslam and Alastair Dent (McMillan References, London 1998).

Briefly, the haloacetamides can be prepared as follows: an appropriately substituted phenyl group carrying a protected amine or carboxyl is appended to the moiety to yield protected amine derivatives with varying linkers. Deprotection followed by acylation with an activated halo acetic derivative yields the haloacetamides of the current invention.

The linking group could be a maleimide, a vinyl sulfone that function as Michael acceptors to the thiol group acting as a nucleophile. The linking group can include a leaving group or a group that reacts with a nucleophile to yield an adduct. The leaving group is a chemical moiety that is active in conjugating the BUP and its metabolite analogs to a label or a carrier. As part of the conjugation process, one or more atoms of the leaving group are given up. Furthermore, conjugation of a label or a carrier generally results in modifying the leaving group so that the linking group in the conjugate includes the residue following such modifications. For convenience herein, the term “linking group” refers to the linking group attached to form a BUP or NBUP, BUP-G or BUP-G metabolite analog and to the residue of the linking group following conjugation to a label or a carrier.

In several embodiments exemplified herein, BUP hapten and analogs are derivatized with a linking group that includes a carboxyl group that is used to attach the analogs to a label or a carrier. In an exemplary conjugation process, the carboxyl group on the BUP-G or NBUP-G analog is reacted with N-hydroxysuccinimide (NHS) to form an active ester. This active ester reacts with amino groups to form BUP analog conjugates. The amino groups can be present in small molecules such as fluorescein or biotin derivatives or in macromolecules such as proteins, for example, bovine serum albumin, keyhole limpet cyanin or peroxidase. In some cases, BUP, N-BUP, BUP-G and NBUP-G conjugates containing a carrier or a label can be used as an immunogen or as a tracer.

In certain aspects, the present disclosure provides antibodies or antigen-binding fragments thereof that bind BUP, BUP-G, NBUP and NBUP-G derivatives conjugated to another molecule. In some embodiments, the antibodies have a variable heavy chain amino acid sequence of any one of SEQ ID NOs: 139-161, or an amino acid sequence having at least 80%, at least 85%, at least 90%, at least 95%, or at least 99% sequence identity to any one of SEQ ID NOs: 139-161, as shown in Table 1. In some embodiments, the antibodies have a variable light chain amino acid sequence of any one of SEQ ID NOs: 162-184, as shown in Table 1, or an amino acid sequence having at least 80%, at least 85%, at least 90%, at least 95%, or at least 99% sequence identity to the sequence of any one of SEQ ID NOs: 162-184, as shown in Table 1.

In some embodiments, the variable heavy chain includes a first CDR (HCDR1) amino acid sequence selected from any one of SEQ ID NOs: 1-23, as shown in Table 1, or an amino acid sequence having at least 80%, at least 85%, at least 90%, at least 95%, or at least 99% sequence identity to the sequence of any one of SEQ ID NOs: 1-23, as shown in Table 1.

In some embodiments, the variable heavy chain includes a second CDR (HCDR2) amino acid sequence selected from any one of SEQ ID NOs: 24-46, as shown in Table 1, or an amino acid sequence having at least 80%, at least 85%, at least 90%, at least 95%, or at least 99% sequence identity to the sequence of any one of SEQ ID NOs: 24-46, as shown in Table 1.

In some embodiments, the variable heavy chain includes a third CDR (HCDR3) amino acid sequence selected from any one of SEQ ID NOs: 47-69, as shown in Table 1, or an amino acid sequence having at least 80%, at least 85%, at least 90%, at least 95%, or at least 99% sequence identity to the sequence of any one of SEQ ID NOs: 47-69, as shown in Table 1.

In some embodiments, the variable light chain includes a first CDR (LCDR1) amino acid sequence selected from any one of SEQ ID NOs: 70-92, as shown in Table 1, or an amino acid sequence having at least 80%, at least 85%, at least 90%, at least 95%, or at least 99% sequence identity to the sequence of any one of SEQ ID NOs: 70-92, as shown in Table 1.

In some embodiments, the variable light chain includes a second CDR (LCDR2) amino acid sequence selected from any one of SEQ ID NOs: 93-115, as shown in Table 1, or an amino acid sequence having at least 80%, at least 85%, at least 90%, at least 95%, or at least 99% sequence identity to the sequence of any one of SEQ ID NOs: 93-115, as shown in Table 1.

In some embodiments, the variable light chain includes a third CDR (LCDR3) amino acid sequence selected from any one of SEQ ID NOs: 116-138, as shown in Table 1, or an amino acid sequence having at least 80%, at least 85%, at least 90%, at least 95%, or at least 99% sequence identity to the sequence of any one of SEQ ID NOs: 116-138, as shown in Table 1.

In some embodiments, the variable heavy chain is encoded by a nucleotide sequence selected from any one of SEQ ID NOs: 185-207, as shown in Table 1, or a nucleotide sequence having at least 80%, at least 85%, at least 90%, at least 95%, or at least 99% sequence identity to the sequence of any one of SEQ ID NOs: 185-207, as shown in Table 1.

In some embodiments, the variable light chain is encoded by a nucleotide sequence selected from any one of SEQ ID NOs: 208-230, as shown in Table 1, or a nucleotide sequence having at least 80%, at least 85%, at least 90%, at least 95%, or at least 99% sequence identity to the sequence of any one of SEQ ID NOs: 208-230, as shown in Table 1.

In still other embodiments, an antibody or fragment thereof may be a monoclonal or polyclonal antibody. Depending on the methods of preparation, in certain embodiments, the antibodies of the present disclosure can be in a lyophilized state.

The antibodies used in the present disclosure can include immunoglobulin molecules and portions of immunoglobulin molecules capable of binding the desired binding site. The immunoglobulin molecules of the present disclosure can be essentially of any class or isotype (e.g., IgG, IgE, IgM, IgD, IgA and IgY) or subclass (e.g., IgG1, IgG2, IgG3, IgG4, IgA1 and IgA2) of an immunoglobulin molecule. Additionally, structures known as nanobodies and domain antibodies can be used, including polypeptides comprising a single or multiple CDRs of an antibody known to bind the cognate binding site, provided an effective amount of the binding ability is retained.

In certain aspects, an immunogen of the disclosure is a BUP analog that includes a carrier. The term “carrier” as used herein refers to a substance that is immunogenic in a selected host animal. Preparation of immunogens by linking a hapten to a carrier is well known. Selection of the carrier and administration route to induce an immune response varies depending on the host animal.

Carriers are generally large molecules, for example, polymers. In some cases, carriers are large proteins from a species other than that of the host animal. Bovine serum albumin (BSA) and keyhole limpet hemocyanin (KLH) are frequently used as carriers for inducing antibodies in mice, rats, goats, rabbits, chickens, and sheep. Additional carriers, such as proteins that are used to conjugate immunogens are well-known in the art and such embodiments are within the purview of the disclosure.

Exemplary preparations of immunogenic BUP, N-BUP, BUP-G and NBUP-G analogs for inducing antibodies to BUP, N-BUP, BUP-G and NBUP-G are described in the examples. In certain such exemplary immunogen preparations, R—Y is (CH₂)ₙ, CO—NH-(carrier), where n is from 1 to 12 and has one or more hetero atoms such as O, N, S. In some cases, n is 1 to 8, and the carrier is BSA.

The term “tracer” as used herein refers to a labeled analyte analog that can be used in a competitive immunoassay format. A tracer of disclosed herein is a BUP, N-BUP, BUP-G and/or NBUP-G analog that includes a label which is attached to BUP, N-BUP, BUP-G and/or NBUP-G through a linking group.

The term “label” is used to refer to substances that can be detected directly or indirectly. Labels that can be detected directly include, for example, a radionuclide or a fluorochrome. Labels can also be detected indirectly through one or more reactions. Such labels include enzymes that are detected by production of a signal, such as a colored product, chemiluminescence, fluorescence, or a radioactive product. Such enzyme labels and their signal development systems are well known. Other such labels include use of a member of a specific binding pair such as biotin/avidin. additional labels suitable for use in immunoassay procedures are well known and include, for example, enzymes, radionuclides, fluorochromes, dioxetanes, acridinium esters, lanthanides and metal chelates, biotin, and the like. In some cases, the label is a fluorochrome, acridinium ester, biotin, dyed latex particle, gold particles or an enzyme such as HRP and G6PD.

Suitable fluorochromes include dyes from the xanthene family e.g., fluoresceins and rhodamines (e.g., tetramethylrhodamine isothiocyanate-TRITC), phycoerythrin (PE), allophycocyanin (APC), Texas Red (Thermo Fisher, Waltham), and preferably fluorescein. Although allophycocyanin and phycoerythrin are suitable fluorochromes, they cannot be used for fluorescence polarization immunoassays, because they are too large. Suitable fluoresceins include fluorescein isothiocyanate (FITC), (2-aminoethyl)-thioureido-fluorescein (FTED), fluorescein-thiosemicarbazide (FTSC), (2-aminoethyl)-ureido-fluorescein (FAMCO-E), erythrocin (tetra-iodo-fluorescein), fluoresceinamine (FAM) and their derivatives such as Oregon Green and Tokyo Green.

In certain embodiments, tracers having a fluorescein residue attached to the linking group through the 5-position of the fluorescein moiety are designated isomer I. Tracers having a fluorescein residue attached to the linking group through the 6-position of fluorescein are designated isomer II. For fluorescein and rhodamine-labeled tracers, little or none of the lactone form exists during fluorescence measurements and the carboxylated forms exist primarily as salts. The fluorochrome can be a homogeneous composition or a mixture of isomers. In addition, the fluorochrome can be used in its lactone form or as a biologically acceptable salt (e.g., Na, K, ammonium and similar salts) so that the fluorochrome can exist in its ionized state in the immunoassay.

Alternatively, carboxylic acids can be condensed with amines using other methods known in the art. Synthetic methods for formation of the amides of carboxylic acids are well known and are described in, The practice of peptide synthesis by M. Bodansky and A. Bodansky, (2nd ed. Springer-Verlag, New York 1995). Methods of making immunogenic conjugates are also described in Methods in Immunology and Immunochemistry (Cutris Williams, Academic Press 1977). Those references are incorporated by reference herein in their entireties. In addition, exemplary methods to produce BUP analogs useful as tracers or as immunogens are described in detail in the Examples section below. Certain exemplary BUP, N-BUP, BUP-G and NBUP-G analogs useful as immunogens, tracers and specifically as enzyme conjugates are listed below in Table 3.

Broadly they are label free methods such as mass spectrometry or label based ones that are immunoassays using an antibody directed towards the analyte. Mass spectrometry method when coupled with chromatography, such as GCMS or LC-MS/MS provide high specificity. However, the GCMS or LC-MS/MS methods are time consuming, require specialized equipment, highly trained analysts, and extensive sample preparation, and are expensive. The methods also require sample volumes that are too large to be used in pediatric testing unless BUP concentrations are abnormally high. Briefly, mass spectrometry methods are not routinely used for BUP therapeutic drug monitoring in a typical clinical chemistry or hospital lab. Immunoassays have been developed for BUP based on heterogeneous requiring multiple wash steps or homogeneous formats that require a simple mix and read.

More recently, a competitive immunoassay (U.S. Pat. Nos. 7,195,882, 7,220,642 and 7,863,427) has been developed for free BUP using antibodies that detect BUP and NBUP but do not cross-react with BUP-G or NBUP-G but still have some cross reactivity to isolated BUP-G. These antibodies were used in the competitive immunoassay and were developed against a particular immunogen linked through the N-17 or phenolic O at C3 (FIG. 1). Haptens were used that are derivatives involving the chemical modification of the N linked derivative of BUP. Antibodies were used to detect BUP and some selected with minimal cross reactivity to BUP-G, the lower sensitivity necessitating a glucuronidase treatment to convert the BUP-G and NBUP-G to BUP and NBUP that could be detected.

An embodiment of the present disclosure provides compounds of Formula 1 and Formula 2, shown below:

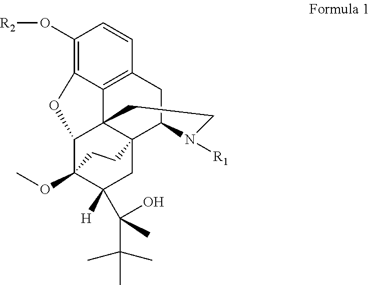

wherein:
R₁ is —Y—Z;
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—; and
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R₂ is H, glucuronic acid, glucuronide salt, or a protecting group,
and salts thereof.

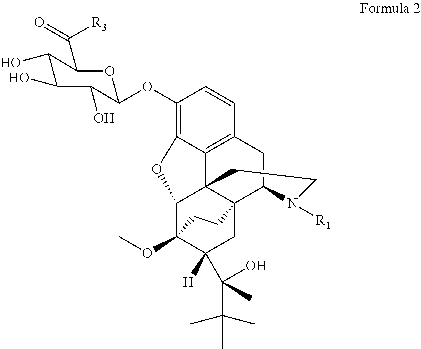

wherein:
R₁ is selected from H, -cyclopropyl methyl, and Y—Z;
R₃ is selected from OH, NH2, NHR4, and Y—Z;
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—;
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R4 is selected from an immunogenic carrier, a protein, an enzyme, and a label.

Aspects of the present disclosure include antibodies. In some embodiments, an antibody of the present disclosure specifically binds to any of the compounds of the present disclosure, including any of the compounds of Formula 1 described elsewhere herein.

Aspects of the present disclosure include antibodies. In some embodiments, an antibody of the present disclosure specifically binds to any of the compounds of the present disclosure, including any of the compounds of Formula 2 described elsewhere herein.

Nucleic acids that encode any of the antibodies of the present disclosure are also provided, as are expression vectors comprising such nucleic acids, and cells comprising such nucleic acids and expression vectors.

The antibodies and enzymes disclosed herein may be used in various assays and methods that measure, quantify, and/or detect the presence of BUP, NBUP, BUP-G and NBUP-G in a sample. In some embodiments, a competitive assay format is used. In certain such formats, antibodies or enzymes may be attached to a solid support and are used to capture labeled and unlabeled BUP, NBUP, BUP-G and NBUP-G (when present in the sample). In the absence of BUP, NBUP, BUP-G and NBUP-G in the sample, the labeled BUP, NBUP, BUP-G and NBUP-G would bind to the antibody/enzyme on the solid support, generating a signal. Free BUP, NBUP, BUP-G and NBUP-G in the sample would compete with the labeled BUP, NBUP, BUP-G and NBUP-G for the antibody/enzyme binding site, resulting in a decrease in signal. The signal is inversely proportional to the binding of the unlabeled BUP, NBUP, BUP-G and NBUP-G from the sample.

The antibodies and enzymes described herein can be combined with a sample to perform the assays. The sample may be a biological sample, such as tissue extracts, tissues used in immunohistochemistry, or fluids. The fluid samples may be derived from blood, plasma, serum, or buffer.

The antibodies and enzymes described herein may be linked or bound to various components or moieties in order to perform assay functions. For example, in some embodiments, the antibodies and enzymes discussed herein may be bound directly through covalent or non-covalent attachment, or indirectly to a solid support or carrier to form a capture molecule. When bound indirectly, intermediate linkers may be used to bind the components. Suitable intermediate linkers include, but are not limited to, an amino group or a carboxylate group or a thiol, biotin, ligands, or other chemical bonds. Suitable solid supports or carriers include, but are not limited to, glass surfaces (e.g., a glass slide or bead), plastic surfaces, metal surfaces, polystyrene surfaces (e.g., a bead or a plate), nitrocellulose surfaces, microparticles, nano-particle surfaces, plates, wells, and paramagnetic or magnetic beads that may be coated with avidin or streptavidin or have other surface functionalities to promote binding affinity.

In some embodiments, labeled BUP is used in the competitive assay format. In certain embodiments, some of the BUP may be linked or bound, directly through covalent or non-covalent attachment, or indirectly, to a label to form a labeled BUP. When bound indirectly, intermediate linkers may be used as discussed herein.

As stated, in some embodiments, an antibody or fragment thereof or an aptamer may be bound to a solid support to form a capture molecule during an assay or method of the present disclosure. This binding can be performed before or after contacting the sample with the antibody or fragment thereof or aptamer. The antibody or fragment thereof or aptamer can be bound to a solid support directly (e.g., covalently) or indirectly (e.g., using binding partners).

Similarly, in some embodiments, BUP molecule may be bound to a label to form a labeled BUP during an assay or method of the present disclosure. This binding can be performed before or after contacting BUP with the sample. BUP can be bound to a label directly (e.g., covalently) or indirectly (e.g., using binding partners).

Examples of suitable binding partners include, but are not limited to, biotin/streptavidin; antibody/antigen; antibody/Fc receptor; an antibody of a first species and an antibody of a second species against first species antibodies; Fc/Fc receptor; 6-His/Ni²⁺; 6-His/cobalt; and 6-His/divalent cation resin.

In other embodiments, binding pairs can be streptavidin and biotin or two antibodies that bind each other such as an antibody that binds an Fc portion of another antibody. In other embodiments, the binding may occur through the interaction between numerous binding pairs. It is contemplated that essentially any method can be used that results in the binding of the antibody to the solid support or binding BUP to a label, e.g., directly or indirectly. In some embodiments, an antibody comprises biotin and a solid support comprises streptavidin or vice versa.

In some embodiments, the label may be any label that corresponds to a suitable detection method. Suitable detection modes include, but are not limited to, absorbance, fluorescence or luminescence using labels or compounds, chemiluminescent compounds, enzyme labels, fluorophores, chromogenic compounds, radiolabels, catalysts, colorimetric compounds or labels, labeled antibodies, latex particle, a magnetic particle, a radioactive element, fluorescent dyes, phosphorescent dyes, lanthanides, gold particles, silver colloidal particles, selenium colloidal particles, metal chelates, ferrocenes, coenzymes, electro active groups, oligonucleotides or stable radicals. The metal chelate may be a ruthenium for electrochemiluminescence, an osmium metal chelate or a lanthanide such as europium, samarium or terbium chelate for time resolved fluorescence. The detection method may include any known detection method including, but not limited to, chromogenic, radioisotopic, fluorescence, immunofluorescence, luminescence, bioluminescence, electrochemiluminescence (ECL), amperometric measurements involving current or impedance, Surface Enhanced Raman Scatter (SERS) and surface plasmon resonance (SPR).

In some embodiments, the detection method may be absorbance that is read in an endpoint or kinetic mode. A colorimetric compound may serve as the label that may be detected or quantified within a cuvette, reaction chamber, such as in a flow cell, or on a disposable container or a lateral flow strip. The solid support may serve to hold the antibody bound to the label near an electrode in the electrochemical readout reaction chamber during detection. The solid support could be a lateral flow device or a surface plasmon generating surface.

In some embodiments, the solid support and/or the label may be from a lyophilized composition that is rehydrated with the sample for use in an assay. The lyophilized composition may contain standard and/or other necessary assay specific components of an assay, such as buffers, reagents, detergents, preservatives, salts, proteins, antibodies, etc. It is contemplated that the solid support and the label may be lyophilized in separate compositions, and then rehydrated with the sample. It is also contemplated that the solid support and the label may be lyophilized in the same composition, and then rehydrated with the sample.

The antibodies and enzymes of the present disclosure may be used in various assay formats, including, for example, heterogeneous immunoassays such as enzyme-linked immunosorbent assays (ELISA) or ECL assays for detecting the presence of BUP, NBUP, BUP-G and NBUP-G or homogeneous immunoassays such as EMIT, CEDIA, LOCI or turbidimetric formats such as QMS (Thermo Fisher), PETINIA (Abbott) or KIMs (Roche) that require no wash steps to separate bound and free moieties. In one aspect of the present disclosure, the assay method steps for detecting and/or quantifying BUP, NBUP, BUP-G and NBUP-G in a sample may include combining in a solution the sample with a capture molecule and a labeled BUP, wherein the capture molecule is capable of binding the labeled BUP, NBUP, BUP-G and NBUP-G, and wherein BUP, NBUP, BUP-G and NBUP-G, when present in the sample, competes with the labeled BUP, NBUP, BUP-G and NBUP-G for binding to the capture molecule; and detecting an amount of labeled BUP, NBUP, BUP-G and NBUP-G bound to the capture molecule through signal produced by a label on the bound labeled BUP, NBUP, BUP-G and NBUP-G, wherein the signal is inversely proportional to the amount of BUP, NBUP, BUP-G and NBUP-G present in the sample.

In some embodiments, the capture molecule comprises an antibody or fragment thereof attached to a solid support. In some embodiments, a capture molecule can be an antibody or fragment thereof attached to a solid support, where the antibody or fragment thereof binds BUP and/or NBUP, BUP-G and NBUP-G conjugated to another molecule(s). For example, the antibody may selectively bind to a molecule conjugated to BUP, NBUP, BUP-G or NBUP-G as compared to the same molecule without conjugation to BUP, NBUP, BUP-G and NBUP-G. When an antibody is described as binding to BUP, NBUP, BUP-G and NBUP-G it is understood that this also includes an antibody that selectively binds a molecule conjugated to BUP, NBUP, BUP-G and NBUP-G as compared to the same molecule without conjugation to BUP, NBUP, BUP-G and NBUP-G

In some embodiments, the labeled BUP, NBUP, BUP-G and NBUP-G comprise a BUP, NBUP, BUP-G and NBUP-G molecule, or BUP, NBUP, BUP-G and NBUP-G conjugated to another molecule, covalently linked to a label. In some embodiments, the labeled BUP, NBUP, BUP-G and NBUP-G comprises a BUP, NBUP, BUP-G and NBUP-G conjugate covalently linked to a label.

In some embodiments, the conjugate of the BUP and NBUP, analog derivative is modified at the phenyl O at C3. In some embodiments, the conjugate of the BUP and NBUP, analog derivative is modified at the pyramidal nitrogen, N17 (FIG. 1).

In some embodiments, the conjugate of the BUP-G and NBUP-G, analog derivative is modified at the carboxyl group of the glucuronide and/or the pyramidal nitrogen N17.

It is contemplated that the steps of the methods of the present disclosure do not have to be completed in the order provided herein, and may be performed in different orders. Additionally, the sample may be incubated for a period of time before a washing step and removal of any unbound or excess materials. It is further contemplated that additional washing steps to remove materials during the assay may be performed at additional times during the method, such as after the addition of each assay component and/or before the detecting step.

In other embodiments, a capture molecule and sample are combined prior to the addition of a labeled BUP, NBUP, BUP-G and/or NBUP-G. For example, a solution comprising a sample and a capture molecule may be incubated for a period of time prior to the addition of a labeled BUP, NBUP, BUP-G and/or NBUP-G.

Components/reagents used in embodiments of the assays disclosed herein can be lyophilized using standard lyophilization methods. For example, the components and reagents can be lyophilized by creating a solution containing the desired component(s), such as a labeled BUP or capture molecule. Then the solution can be used to form drops that are allowed to fall into a freezing medium (e.g., liquid nitrogen), typically forming frozen spheres, and then lyophilizing the frozen spheres or pellets.

In some embodiments of the assays, a lyophilized composition containing a capture molecule or a labeled BUP, NBUP, BUP-G and NBUP-G combination thereof, or both is rehydrated with the sample. This embodiment may be advantageous in that the sample is essentially undiluted during the assay, which may result in higher levels of sensitivity because more BUP or one of the three metabolites is present in an undiluted sample as compared to a diluted sample of the same volume. In some embodiments, a sample is diluted prior to combining with the other reagents. In some embodiments, a sample is not diluted prior to combining with the other reagents.

Also provided are methods of making the antibodies of the present disclosure. The methods include culturing a cell of the present disclosure under conditions suitable for the cell to express the antibody, wherein the antibody is produced.

Aspects of the present disclosure further include compositions. A composition of the present disclosure may include any of the antibodies, nucleic acids, expression vectors, and/or cells of the present disclosure.

Also provided are methods for determining an amount of at least one BUP analyte in a medium. In certain embodiments, such methods include combining in a medium a sample suspected of containing at least one BUP analyte, and an antibody of the present disclosure. Such methods further include determining the presence or absence of a complex comprising the BUP, NBUP, BUP-G and/or NBUP-G analyte and the antibody, wherein the presence of the complex indicates the presence of the BUP, NBUP, BUP-G and/or NBUP-G analyte in the sample.

Aspects of the present disclosure further include kits. According to some embodiments, the kits find use in determining an amount of at least one BUP, NBUP, BUP-G and/or NBUP-G analyte in a sample. In certain embodiments, a kit of the present disclosure includes any of the antibodies of the present disclosure, and instructions for using the antibody to determine an amount of at least one BUP, NBUP, BUP-G and/or NBUP-G analyte in a sample. Such kits may further include any of the compounds represented in FIG. 1 of the present disclosure.

According to some embodiments, a kit of the present disclosure includes any of the compounds of FIG. 1 of the present disclosure, and instructions for using the compound to determine an amount of at least one BUP, NBUP, BUP-G and/or NBUP-G analyte in a sample. Such kits may further include any of the antibodies of the present disclosure.

### Compounds, Conjugates and Syntheses Thereof

Homogeneous enzyme immunoassays depend on the availability of enzyme-sbp member conjugates whose enzyme activity can be strongly modulated on binding of the sbp partner. The present disclosure provides enzyme-sbp member conjugates and antibodies for conducting assays that are useful in homogeneous immunoassays.

In certain embodiments, protein immunogens are synthesized and used to prepare antibodies specific for compounds, such as (but not limited to) BUP analyte. The antibodies may be used in methods for detecting BUP analyte in a sample suspected of containing the analyte. Label conjugates are prepared and may be employed in the above methods. Effective quantitation of samples for the presence of BUP analyte as referred to above may be realized.

The immunogens and label conjugates may involve an analog of BUP linked through the phenyl group to a protein or a label. In some instances, the conjugate may be referred to herein as a protein conjugate or a label conjugate, respectively.

Compounds of the present disclosure include compounds useful for producing antibodies according to the present disclosure. In addition, compounds of the present disclosure include conjugates useful for the immunoassays described herein. In certain embodiments, the compounds include those derived from Formula 1 or Formula 2:

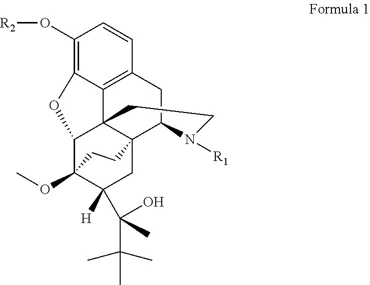

wherein:
R₁ is —Y—Z;
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—;
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R₂ is H, glucuronic acid, glucuronide salt, or a protecting group,
and salts thereof.

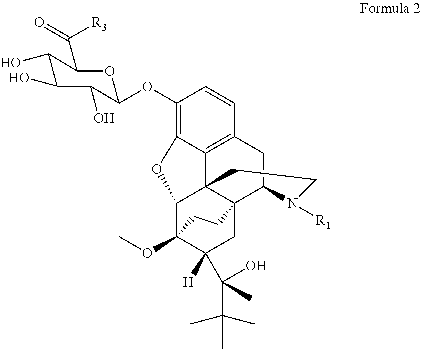

wherein:
R₁ is selected from H, -cyclopropyl methyl, and Y—Z;
R₃ is selected from OH, NH2, NHR4, and Y—Z—R4;
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—;
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R4 is selected from an immunogenic carrier, a protein, an enzyme, and a label,
and salts thereof.

In some embodiments, Z is a protein. For example, the protein can be an immunogenic carrier. The immunogenic carrier can be conjugated to a compounds derived from Formula 1 and Formula 2 to yield haptens of the current invention, thereby enabling the production of antibodies that can specifically bind with the analytes BUP, NBUP, BUP-G and/or NBUP-G. For example, the immunogenic carrier can be selected from a hemocyanin, a globulin, an albumin, and a polysaccharide. In some instances, the immunogenic carrier is bovine serum albumin (BSA). In some instances, the immunogenic carrier is keyhole limpet hemocyanin (KLH). In certain embodiments, the immunogenic carrier may be modified to include one or more functional groups. The functional group on the modified immunogenic carrier can be a reactive functional group that facilitates attachment of the immunogenic carrier to the linking group in the compounds of Formula 1 and Formula 2.

In some embodiments, a BUP, NBUP, BUP-G and NBUP-G hapten is linked through an alkyl, ether, thioether, azo, keto or alkene functionality. As such, in some cases, the linking group comprises an alkyl or substituted alkyl group attached to the pyramidal nitrogen at N-17 or through the phenolic O at C-3 or through the carboxyl group of the glucuronides.

In certain embodiments, such haptens are used to produce antibodies specific for BUP, NBUP, BUP-G and NBUP-G

In certain embodiments, Z is a label. The label is a molecule which produces, or can be induced to produce, a detectable signal. For example, the label can be an enzyme, such as an enzyme selected from an alkaline phosphatase, a β-galactosidase and a horse radish peroxidase. In some embodiments, the label is an enzyme, where the enzyme is glucose-6-phosphate dehydrogenase (G6PD). In some instances, the G6PD is a mutant G6PD, which includes one or more amino acid residue substitutions relative to the wild-type form. For example, the mutant G6PD can include a cysteine substitution, e.g., a cysteine substitution in each subunit of the G6PD enzyme. In some cases, the linking group can be attached to the G6PD enzyme at the cysteine residue. In certain embodiments, the label may be modified to include one or more functional groups. The functional group on the modified label can be a reactive functional group that facilitates attachment of the label to the linking group in the compound of Formula 1 and Formula 2.

In certain embodiments, Z is a protein. The protein can be any suitable protein, which includes amino acid residues, such as a dipeptide, tripeptide, and the like, in any number of such amino acid residues, such as 2 or more, 3 or more, 4 or more, 5 or more, 6 or more, 7 or more, 8 or more, 9 or more, 10 or more, etc. In certain embodiments, the protein may be modified to include one or more functional groups. The functional group on the modified protein can be a reactive functional group that facilitates attachment of the protein to the linking group in the compounds of Formula 1 and Formula 2. In some instances, the protein is acylated. In some instances, the protein is alkylated.

The linking group may include about 1 to 25 atoms (excluding hydrogen atoms) and may include a chain of from 2 to 15 atoms (excluding hydrogen atoms), each independently selected from carbon, oxygen, sulfur, nitrogen, halogen and phosphorous. In some embodiments, the linking group includes 1 to 15 carbon atoms and/or 0 to 6 heteroatoms. Examples of linking groups include, but are not limited to, —(CH₂)ₙC(O)—, —C(O)(CH₂)ₙ—, —C(O)(CH₂)ₙNHC(O)—, —C(O)(CH₂)ₙNHC(O)(CH₂)ₙ—, —(CH₂)ₙSCH₂C(O)—, —(CH₂) DC(O)NH(CH₂)ₙ—, —(CH₂) NHC(O)—, —(CH₂)ₙNHC(O)(CH₂)ₙ—, —(CH₂)ₙNHC(O)(CH₂)NO(CH₂)ₙNHC(O)(CH₂)ₙ—, —NH(CH₂)ₙC(O)—, —(CH₂)ₙ—, —(CH₂)ₙNHC(O)(CH₂)ₙNHC(O)(CH₂)ₙ—, —C(O)NH(CH₂CH₂O)ₘ(CH₂)ₙNHC(O)(CH₂)ₙ—, and —(CH₂)ₙ (heterocyclyl)S(CH₂)ₙC(O)—, each m is independently an integer from 1 to 10, and each n is independently an integer from 1 to 10, and including salts thereof. In certain embodiments, the linking group is —C(O)NH(CH₂CH₂O)₂(CH₂)₂NHC(O)CH₂—. In certain embodiments, the linking group is —CH₂NHC(O)(CH₂)₇NHC(O)CH₂—. In certain embodiments, the linking group is —CH₂NHC(O)(CH₂)₂O(CH₂)₄NHC(O)CH₂—. In certain embodiments, the linking group is —CH₂NHC(O)(CH₂)₂NHC(O)CH₂—. In certain embodiments, the linking group is —CH₂NHC(O)CH₂—. In certain embodiments, the linking group is —C(O)(CH₂) NHC(O)(CH₂)ₙ—, such as —C(O)(CH₂CH₂)NHC(O)(CH₂)—. In certain embodiments, the linking group is
(CH₂)ₙ (heterocyclyl)S(CH₂)ₙC(O)—, such as —(CH₂CH₂CH₂CH₂)(2,5-dioxopyrrolidin-1-yl)S(CH₂)C(O)—.

The number of heteroatoms in the linking group may range from 0 to 6, such as from about 1 to 5, or from 2 to 5, or from 3 to 5. The linking agents may be aliphatic or aromatic. When heteroatoms are present, oxygen may be present as oxo or oxy, bonded to carbon, sulfur, nitrogen or phosphorous; nitrogen may be present as nitro, nitroso or amino, bonded to carbon, oxygen, sulfur or phosphorous; sulfur can be analogous to oxygen; phosphorous can be bonded to carbon, sulfur, oxygen or nitrogen, such as phosphonate and phosphate mono or di-ester. Common functionalities in forming a covalent bond between the linking group and the molecule to be conjugated are alkylamine, amidine, thioamide, ether, urea, thiourea, guanidine, azo, thioether and carboxylate, sulfonate, and phosphate esters, amides and thioesters.

In certain embodiments, when a linking group has a non-oxocarbonyl group including nitrogen and sulfur analogs, a phosphate group, an amino group, alkylating agent such as halo or tosylalkyl, oxy (hydroxyl or the sulfur analog, mercapto) oxocarbonyl (e.g., aldehyde or ketone), or active olefin such as a vinyl sulfone or α-, β-unsaturated ester, these functionalities can be linked to amine groups, carboxyl groups, active olefins, alkylating agents, e.g., bromoacetyl. Where an amine and carboxylic acid or its nitrogen derivative or phosphoric acid is linked, amides, amidines and phosphoramides can be formed. Where mercaptan and activated olefin are linked, thioethers can be formed. Where a mercaptan and an alkylating agent are linked, thioethers can be formed. Where aldehyde and an amine are linked under reducing conditions, an alkylamine can be formed. Where a carboxylic acid or phosphoric acid and an alcohol are linked, esters can be formed. Various linking groups and conjugation methods are described in, for example, Cautrecasas, J. Biol. Chem. (1970) 245:3059 and Greg Hermanson, Bioconjugate Techniques: Third Edition (2013).

To develop an assay for BUP and its metabolites, the chemical structure of BUP that conserves the morphinan ring system common to most opiates is used. For example, BUP has several ring systems fused together with a bridged pyramidal nitrogen N-17 as part of the opiate structure as well as phenolic oxygen for labelling as used in U.S. Pat. No. 7,863,427. The alkyl and acyl linked derivatives from the phenyl moiety preserve the core morphinan ring system of the opiates. The specific linkage through the phenyl group generates chemical structures that retain significant BUP complementarity to prepare immunogens and raise antibodies accordingly. Linkage through the phenolic O at C3 with alkyl and acyl linkers blocks glucuronidation as well as precludes formation of antibodies to glucuronidated BUP and NBUP. The antibodies derived from haptens described in U.S. Pat. Nos. 7,220,842 and 7,863,427 thus yield immunoassays for BUP and NBUP but not BUP-G and NBUP-G.

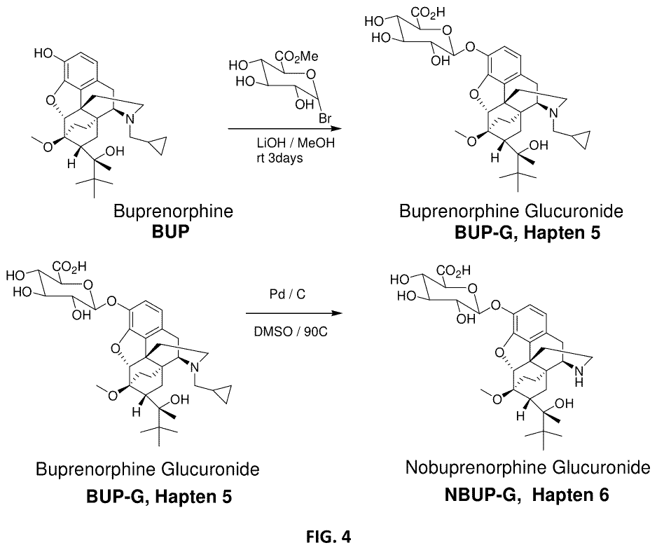

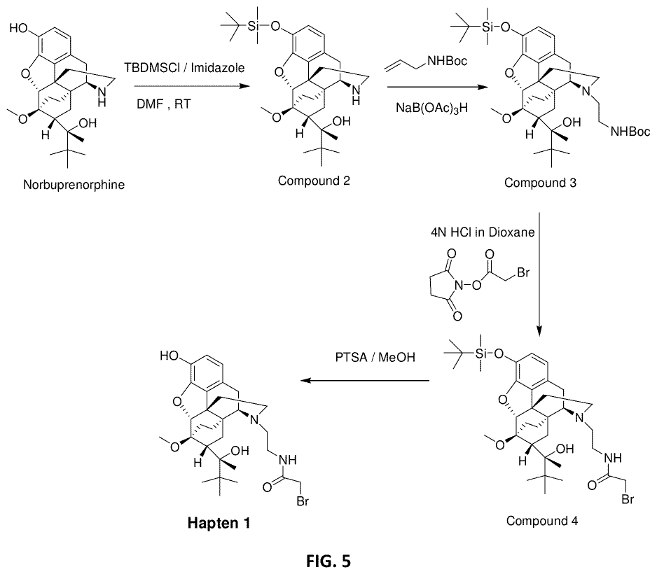

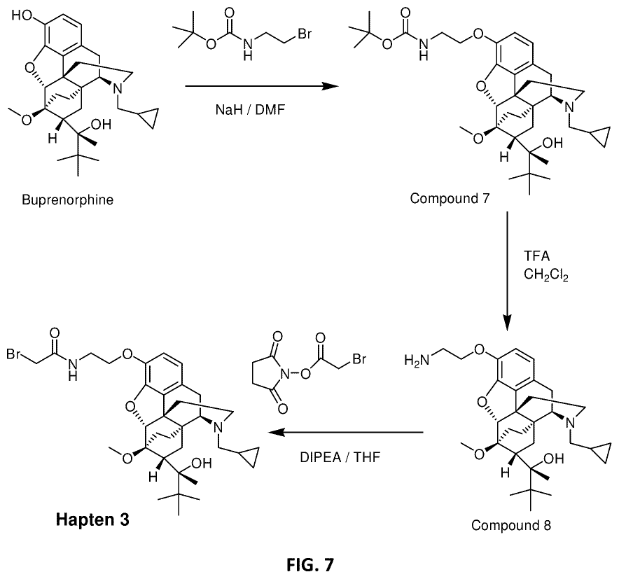

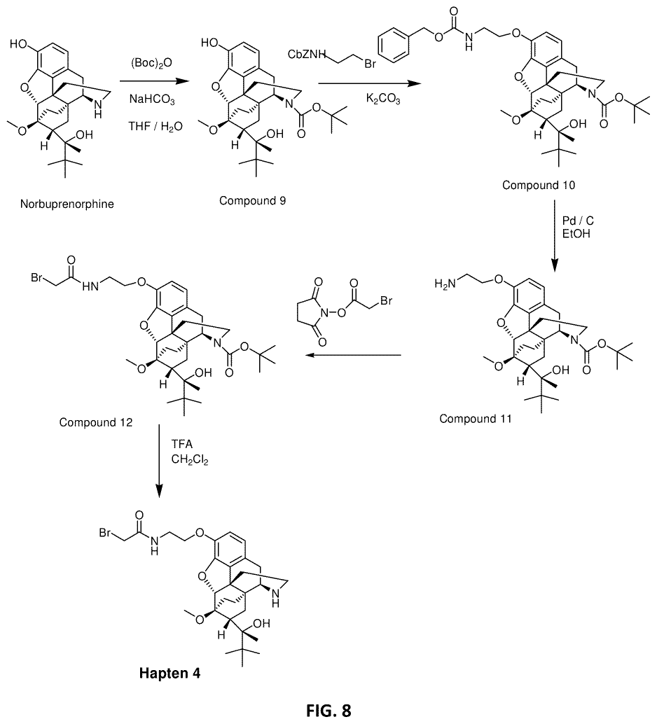

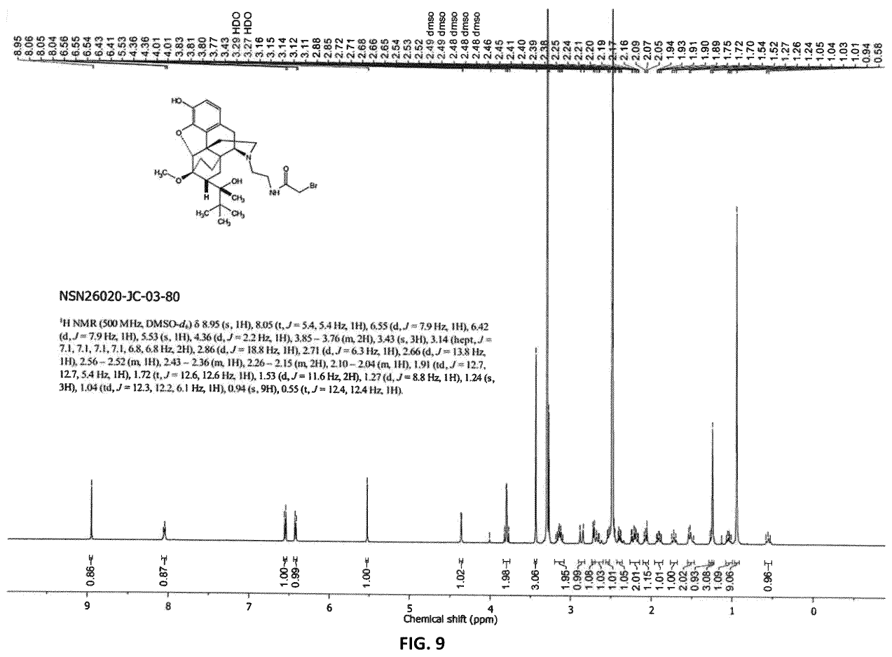

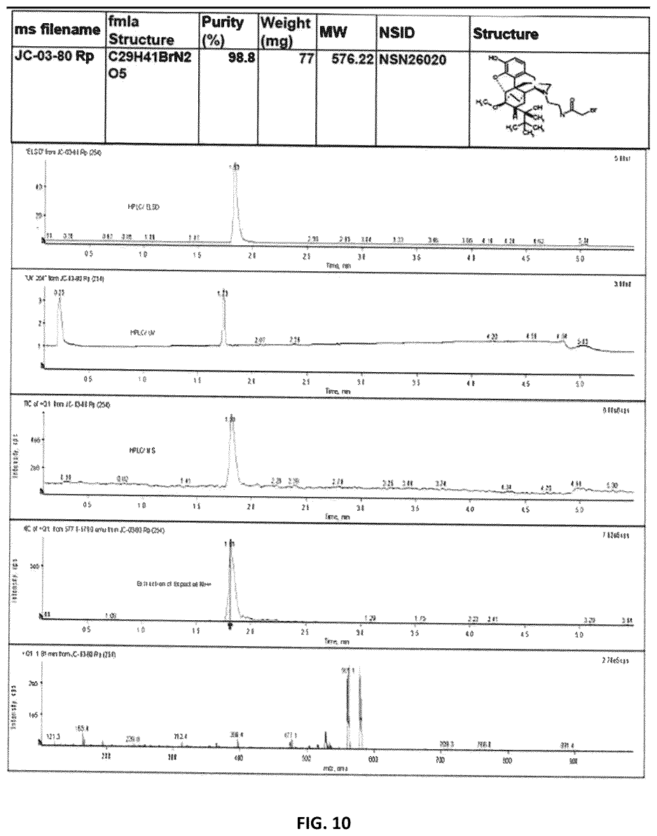

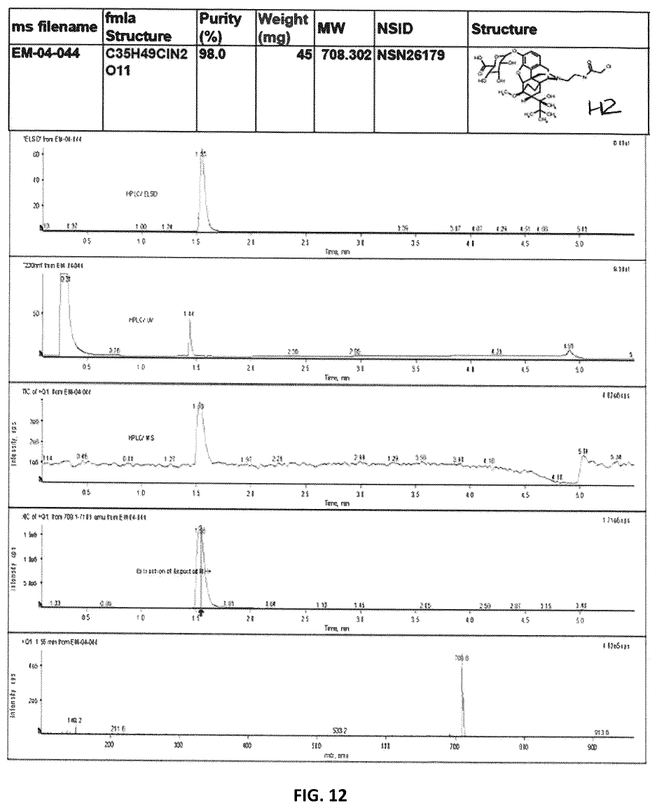

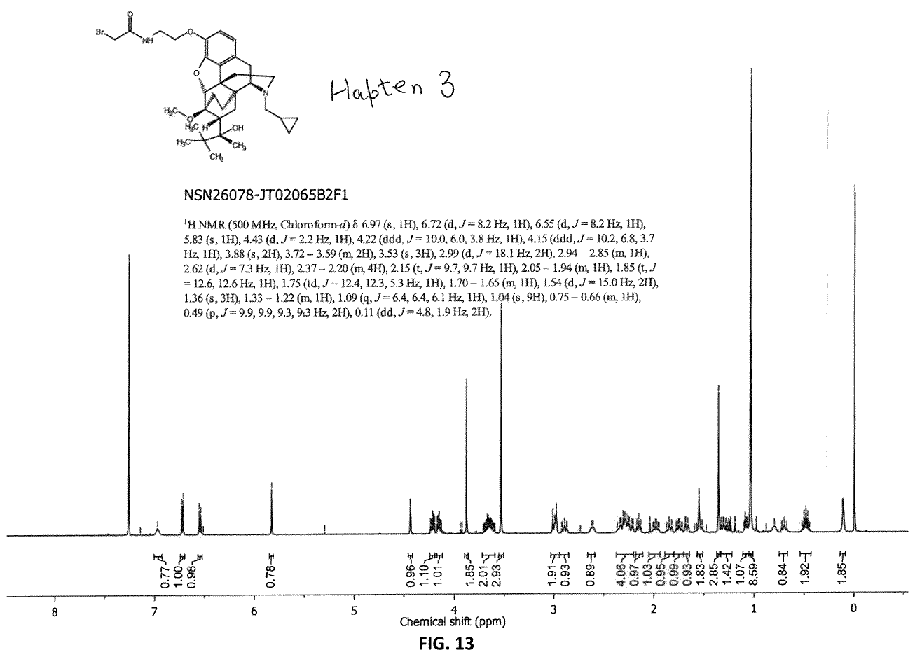

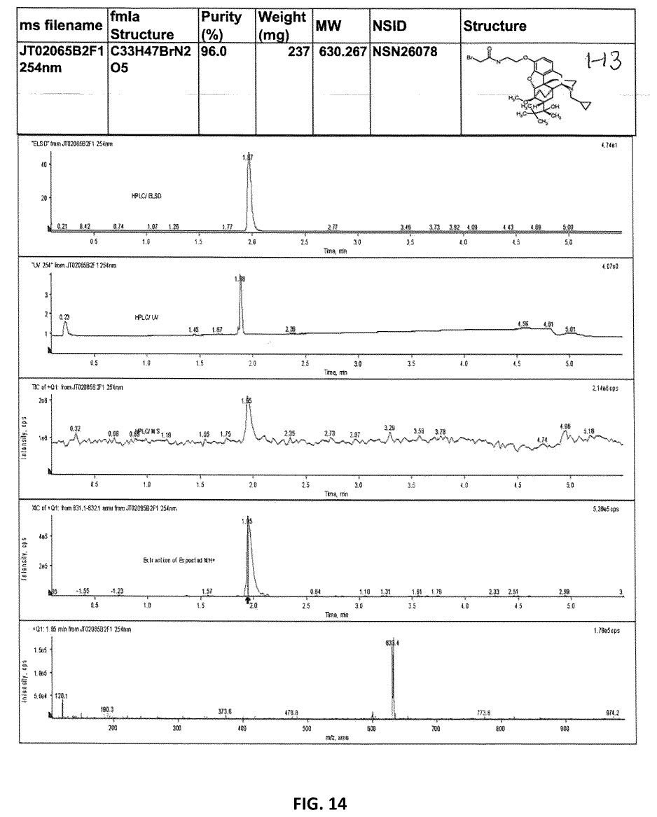

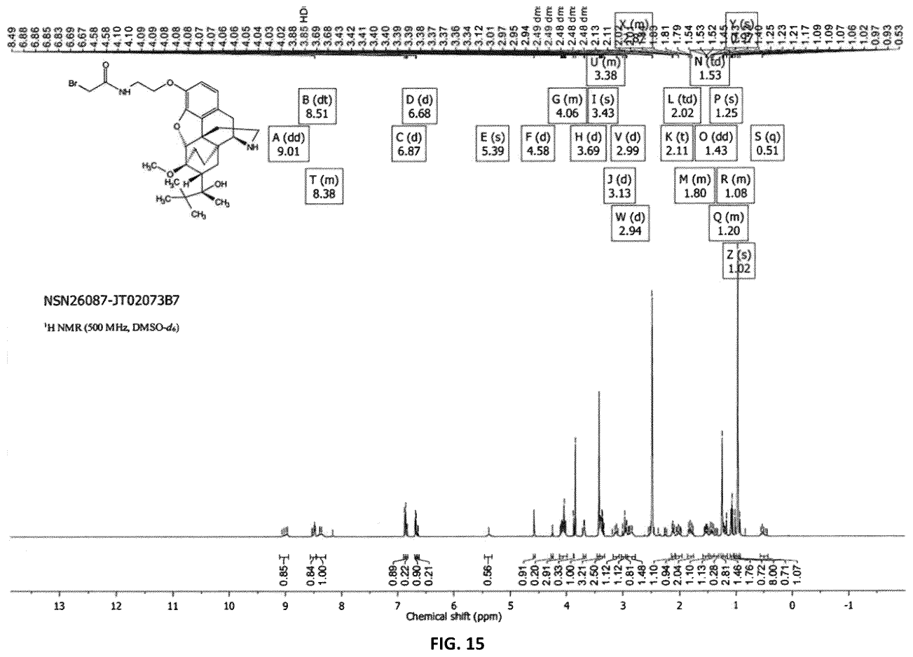

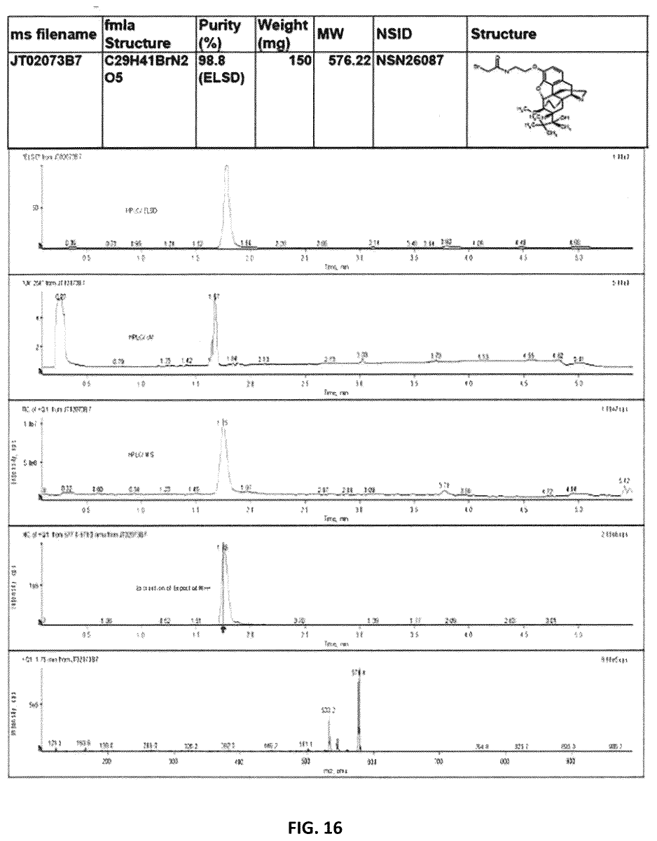

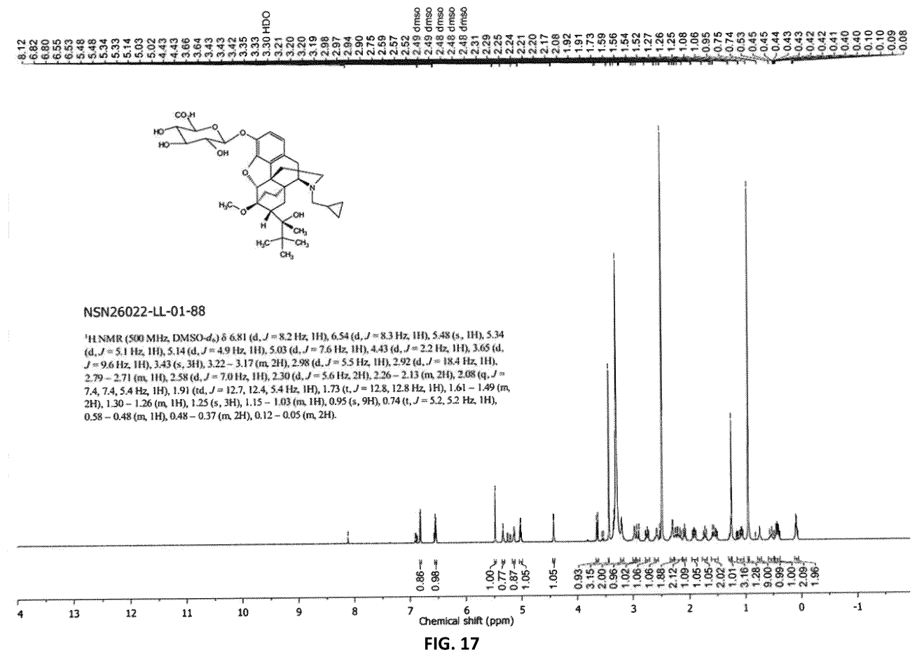

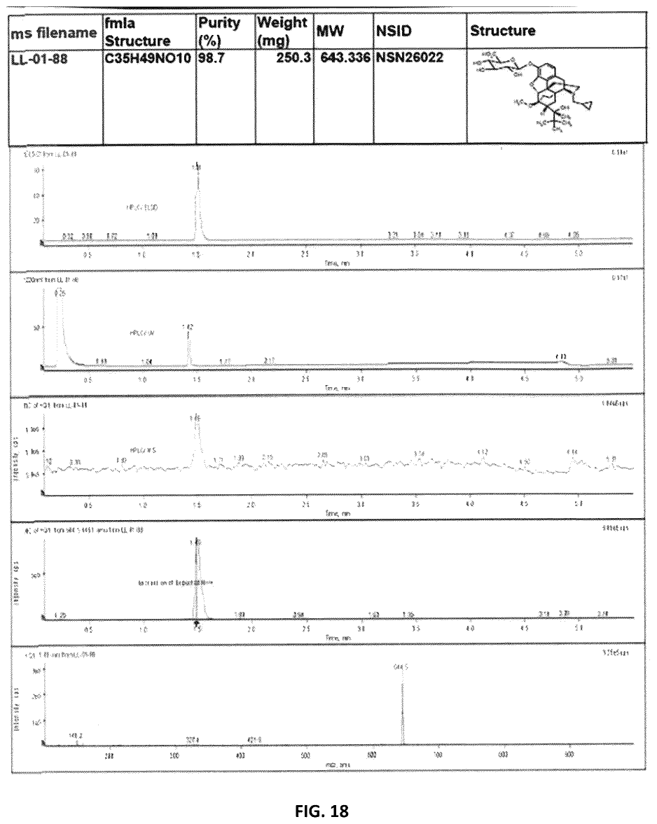

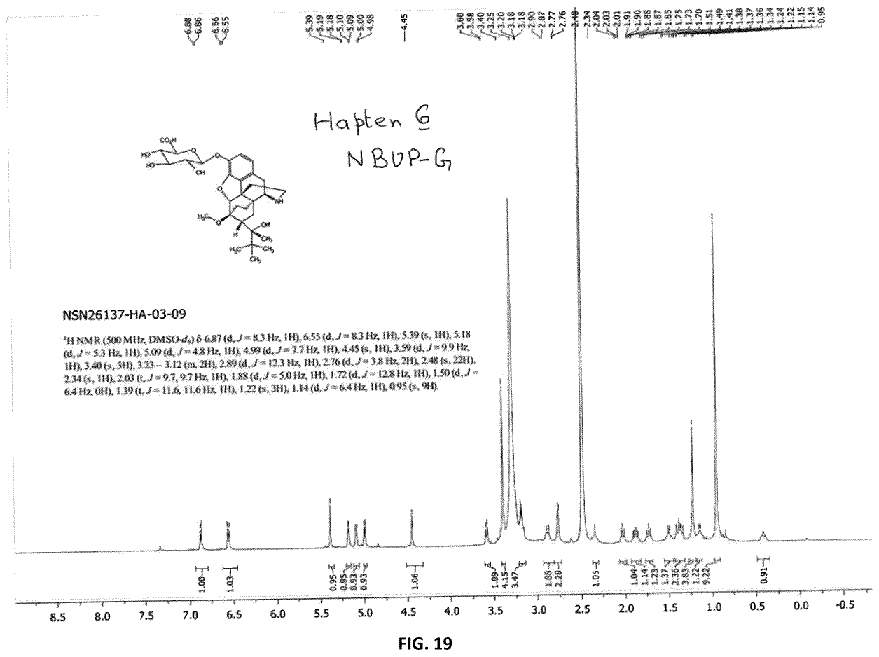

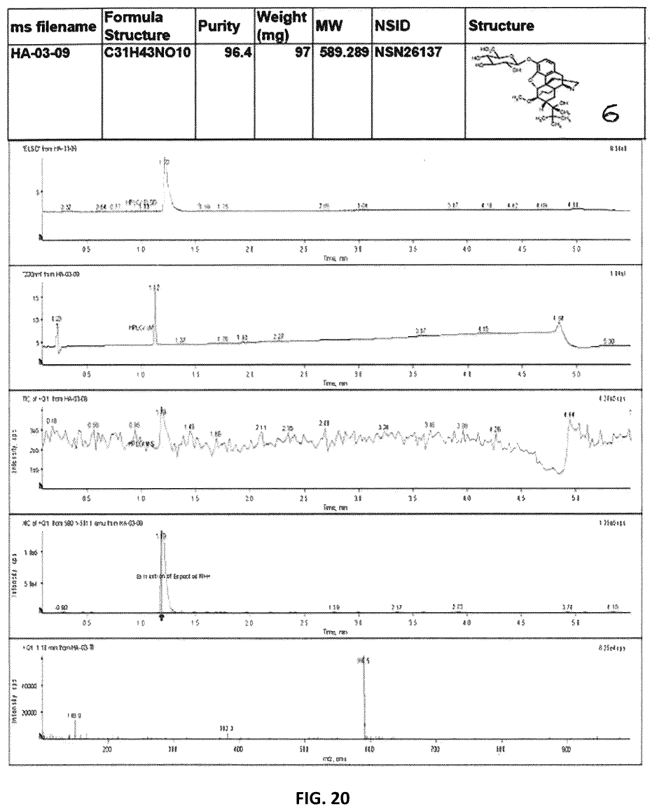

The present disclosure provides for the design of BUP, NBUP, BUP-G and NBUP-G haptens and immunogens by modification of the 1) pyramidal N at N17, 2) phenolic O at C3 and 3) carboxyl of the glucuronide of BUP-G and NBUP-G. Haptens derived from the BUP, NBUP, BUP-G and NBUP-G analogs are shown as Hapten1, Hapten 2, Hapten 3, Hapten 4, Hapten 5 and Hapten 6 of the present disclosure and their synthesis plus analytical data are shown in FIGS. 4-20. Placement of a linking group at the phenyl ring provides for antibodies that may specifically react with BUP analyte because the analytes share the pyramidal nitrogen group. The present disclosure thus provides BUP analogs and immunogens useful with the various types of immunoassays described herein.

Compounds useful for producing antibodies and conjugates according to the present disclosure can be synthesized in accordance with the general synthetic methods described below. Compounds and intermediates for synthesizing the Haptens 1-6 can be prepared by standard methods. The following reaction schemes are only meant to represent examples of the methods and are in no way meant to limit the present disclosure.

### a) Haptens

The synthesis of haptens involves functional chemistry manipulations to manipulate the various groups of atoms in an organic molecule. Protecting and deprotecting some functionality while transforming others are routinely used in organic synthesis.

Addition and removal of protecting group, such as tertbutoxy carbonyl (Boc) is usually performed to protect an amino group for subsequent acylation with an activated haloacetic derivative. Suitable protecting groups are described in detail in patents and articles in the technical literature. See, for example, “Principles of Peptide Synthesis” (M. Bodanszky, Springer Verlag, Berlin, Heidelberg, New York, Tokyo (1984)). Examples of such protecting groups, by way of example and not limitation, are t-butoxycarbonyl (t-Boc), fluorenylmethyloxycarbonyl (Fmoc), acetaminomethyl (Acm), triphenyl methyl (Trt), benzyloxycarbonyl (Cbz) biphenylisopropyloxycarbonyl, 1-amyloxycarbonyl, isobornyloxycarbonyl, alpha-dimethyl-3,5-dimethoxybenxyloxycarbonyl, o-nitrophenylsulfenyl, 2-cyano-1,1-dimentylethoxycarbonyl, bromobenzyloxy, carbamyl, formyl, and the like. Various silyl protecting groups such as tertbutyldimethyl silyl group are used to block the OH group and deprotected with fluoride containing reagents. The particular protecting group chosen may depend on the nature of the reaction to be performed and the conditions of such reaction such as temperature, pH, oxidation and reduction and so forth.

### b) Immunogen

Functionalized haptens (e.g. Haptens 1-6) may be conjugated to proteins. Haptens containing a carboxyl group can be directly coupled to the epsilon amino groups of lysine in proteins. The haloacetamides can be coupled to thiols directly with cysteinyl groups after reduction or usually by a two-step procedure when a lysine needs to converted to a thiol. Activation of protein lysine residues by acylation of the epsilon-nitrogen with N-succinimidyl S-acetylthioacetate (SATA), followed by subsequent hydrolysis of the S-acetyl group with hydroxylamine produces a nucleophilic sulfhydryl group. Conjugation of the sulfhydryl activated protein with the haloacetamide derivatized hapten proceeds via nucleophilic displacement of the bromide to create a thioether linked conjugate. Suitable proteins (immunogenic carriers) include, but are not limited to, keyhole limpet hemocyanin (KLH), bovine thyroglobulin (BTG), and ovalbumin (OVA).

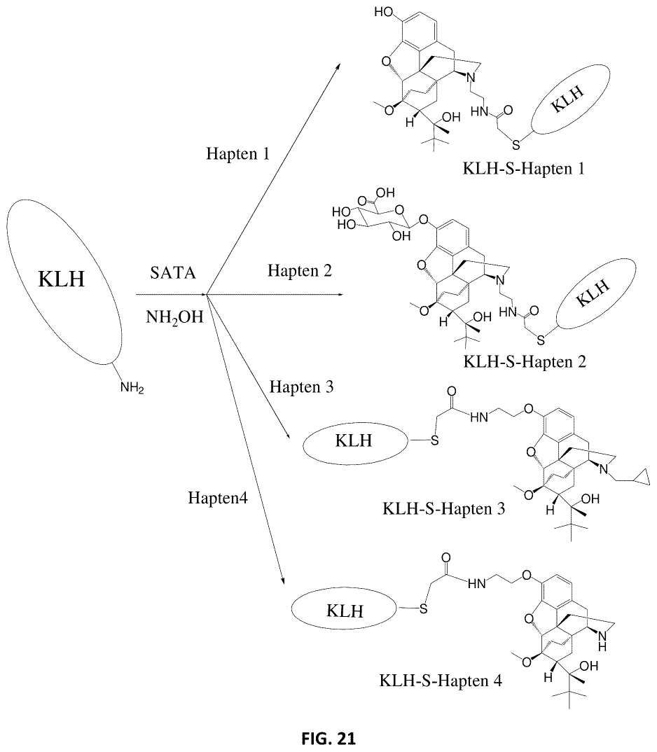

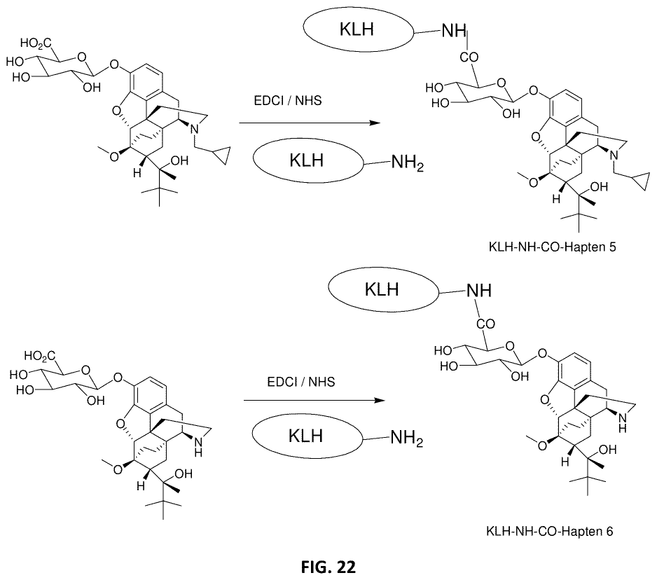

The BUP, NBUP, BUP-G and NBUP-G analogs includes the haloacetamide functionality for conjugating to thiol containing proteins. The synthesis of various haptens are shown in FIGS. 4-8, while the protein conjugates derived from them: KLH-Hapten1, KLH-Hapten2, KLH-Hapten3, KLH-Hapten4, KLH-Hapten5, and KLH-Hapten6 are shown in FIGS. 21 and 22, the preparation of which is described in Example 8. Reaction of amines from keyhole limpet hemocyanin (KLH) with N-succinimidyl S-acetylthioacetate can produce protected sulfhydryls that can be subsequently deprotected by hydroxylamine for reaction with haloacetamides of the current invention. Reaction of thiol modified KLH-SH with halooacetamides in sodium phosphate (0.1 M, pH 8.0) buffer solution can produce the desired immunogen -KLH as show in FIG. 22. The immunogens can be purified by chromatography, such as on a Sephadex G-25 column with buffer solution. The concentration of immunogens can be measured using a protein assay, such as, but not limited to a Pierce™ Rapid Gold BCA protein assay kit. The KLH immunogens can be used for the immunization of rabbits for antibody production. KLH immunogens are also generated by reaction of the carboxyl groups on glucuronides through carbodiimide chemistry via N-hydroxysuccinimide activation of the carboxyl group.

### c) Enzyme Conjugate

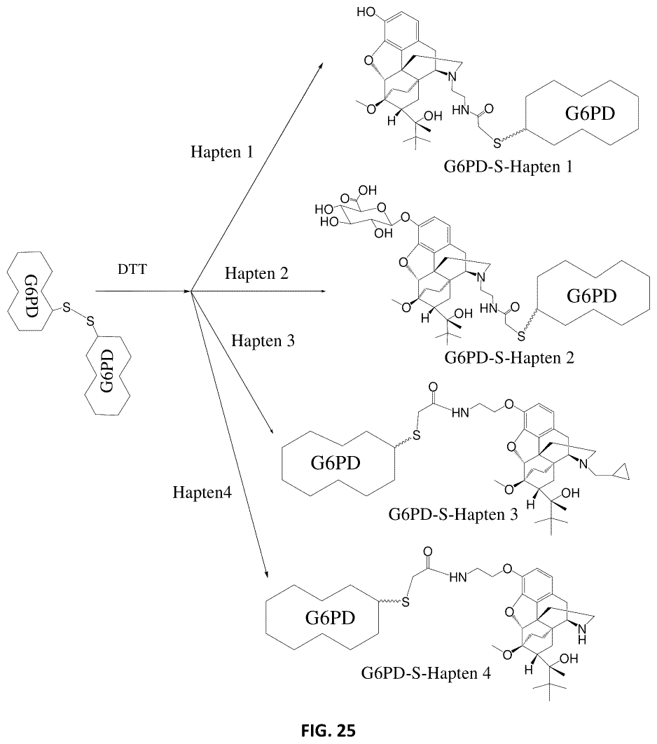

Haptens derived from BUP, NBUP, BUP-G and NBUP-G of the present invention with halocetamide functionality can be used for reaction with proteins containing a thiol group. Conjugation of the halocetamides to cysteine containing G6PD is shown in FIG. 25, the preparation of which is described in Example 9.

Haptens derived from BUP-X-Y can be used to prepare immunogen. Hapten BUP-X-Y (1-6) can be used to prepare a G6PD conjugate. The immunogen BUP-X-L-S-KLH can be used for elicitation of antibodies. In certain embodiments, in an enzyme-based assay format, antibodies produced can show good modulation with a BUP analyte. In some embodiments, the immunogen BUP-X-Y-S-KLH can be used to successfully raise antibodies, which may provide an indication that such antibodies have potential use in an enzyme-based BUP immunoassay as described hereinafter.

### Antibodies and Preparation Thereof

Aspects of the present disclosure include antibodies, which specifically bind to BUP. In some embodiments, an antibody of the present disclosure specifically binds to any of the compounds of the present disclosure, including any of the compounds of Formula 1 described elsewhere herein.

The term “antibody” (also used interchangeably with “immunoglobulin”) encompasses polyclonal (e.g., rabbit polyclonal) and monoclonal antibody preparations where the antibody may be an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the compound, including, but not limited to single chain Fv (scFv), Fab, (Fab′)₂, (scFv′)₂, and diabodies; chimeric antibodies; monoclonal antibodies, human antibodies; and fusion proteins comprising an antigen-binding portion of an antibody and a non-antibody protein. In some embodiments, the antibody is selected from an IgG, Fv, single chain antibody, scFv, Fab, F(ab′)2, or Fab′. The antibodies may be further conjugated to other moieties, such as members of specific binding pairs, e.g., biotin (member of biotin-avidin specific binding pair), and the like.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region is composed of a “framework” region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, (1991 and Lefranc et al. IMGT, the international ImMunoGeneTics information System®. Nucl. Acids Res., 2005, 33, D593-D597)). A detailed discussion of the IMGT system, including how the IMGT system was formulated and how it compares to other systems, is provided on the World Wide Web at imgt.cines.fr/textes/IMGT ScientificChart/Numbering/IMGTnumberingsTable.html. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to IMGT, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies encompass intact immunoglobulins as well as a number of well characterized fragments which may be genetically encoded or produced by digestion with various peptidases. Thus, for example, pepsin digests an antibody below the disulfide linkages in the hinge region to produce F(ab)′₂, a dimer of Fab which itself is a light chain joined to VH—CHI by a disulfide bond. The F(ab)′₂ may be reduced under mild conditions to break the disulfide linkage in the hinge region thereby converting the (Fab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies, including, but are not limited to, Fab′₂, IgG, IgM, IgA, scFv, dAb, nanobodies, unibodies, and diabodies. In certain embodiments, an antibody of the present disclosure is selected from an IgG, Fv, single chain antibody, scFv, Fab, F(ab′)₂, and Fab′.

The phrases “specifically binds,” “specific for,” “immunoreactive,” “immunoreactivity,” and “antigen binding specificity,” when referring to an antibody, refer to a binding reaction with an antigen which is highly preferential to the antigen or a fragment thereof, so as to be determinative of the presence of the antigen in the presence of a heterogeneous population of antigens. Thus, under designated immunoassay conditions, the specified antibodies bind to a particular antigen and do not bind in a significant amount to other antigens present in the sample. Specific binding to an antigen under such conditions may require an antibody that is selected for its specificity for a particular antigen. For example, the antibodies may specifically bind to the compound, and do not exhibit comparable binding to other molecules present in a sample.

In some embodiments, an antibody of the present disclosure “specifically binds” to the compound if it binds to or associates with the compound with an affinity or Kₐ (that is, an equilibrium association constant of a particular binding interaction with units of 1/M) of, for example, greater than or equal to about 10⁵ M⁻¹. In certain embodiments, the antibody binds to the compound with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the antibody binds to the compound with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the antibody for the compound can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by Bioluminescence Interferometry (BLI, ForteBio), by radioimmunoassay; or the like.

Whether a first antibody “competes with” a second antibody for binding to the compound may be readily determined using competitive binding assays known in the art. Competing antibodies may be identified, for example, via an antibody competition assay. For example, a sample of a first antibody can be bound to a solid support. Then, a sample of a second antibody suspected of being able to compete with such first antibody is then added. One of the two antibodies is labelled. If the labeled antibody and the unlabeled antibody bind to separate and discrete sites on the compound, the labeled antibody will bind to the same level whether or not the suspected competing antibody is present. However, if the sites of interaction are identical or overlapping, the unlabeled antibody will compete, and the amount of labeled antibody bound to the compound will be lowered. If the unlabeled antibody is present in excess, very little, if any, labeled antibody will bind.

For purposes of the present disclosure, competing antibodies are those that decrease the binding of an antibody to the compound by about 50% or more, about 60% or more, about 70% or more, about 80% or more, about 85% or more, about 90% or more, about 95% or more, or about 99% or more. Details of procedures for carrying out such competition assays are well known in the art and can be found, for example, in Harlow and Lane, Antibodies, A Laboratory Manual, Cold Spring Harbor Laboratory Press, Cold Spring Harbor, New York, 1988, 567-569, 1988, ISBN 0-87969-314-2. Such assays can be made quantitative by using purified antibodies. A standard curve may be established by titrating one antibody against itself, i.e., the same antibody is used for both the label and the competitor. The capacity of an unlabeled competing antibody to inhibit the binding of the labeled antibody to the plate may be titrated. The results may be plotted, and the concentrations necessary to achieve the desired degree of binding inhibition may be compared.

According to some embodiments, an antibody of the present disclosure competes for binding to a compound of Formula 1 or Formula 2 for BUP with an antibody

Having cross reactivity of 50-100% with NBUP, BUP-G or NBUP-G, comprising:
A variable heavy chain (V_{H}) polypeptide comprising:
   a V_{H} CDR1 comprising the amino acid sequence of any one of SEQ ID NOs: 1-23,
   a V_{H} CDR2 comprising the amino acid sequence of any one of SEQ ID NOs: 24-46, and
   a V_{H} CDR3 comprising the amino acid sequence of any one of SEQ ID NOs: 47-69 and
a variable light chain (V_{L}) polypeptide comprising:
   a V_{L} CDR1 comprising the amino acid sequence of any one of SEQ ID NOs: 70-92,
   a V_{L} CDR2 comprising the amino acid sequence of any one of SEQ ID NOs: 93-115, and
   a V_{L} CDR3 comprising the amino acid sequence of any one of SEQ ID NOs: 116-138.

In certain embodiments, such an antibody comprises one each of the CDRs set forth in Table 1—e.g., one HCDR1, one HCDR2, one HCDR3, one LCDR1, one LCDR2, and one LCDR3. According to some embodiments, the antibody comprises: a variable heavy chain (V_{H}) polypeptide comprising an amino acid sequence having 70% or greater, 75% or greater, 80% or greater, 85% or greater, 90% or greater, or 95% or greater identity to the amino acid sequence set forth in SEQ ID NOs: 139-161, as described in Table 1; and a variable light chain (V_{L}) polypeptide comprising an amino acid sequence having 70% or greater, 75% or greater, 80% or greater, 85% or greater, 90% or greater, or 95% or greater identity to the amino acid sequence set forth in SEQ ID Nos: 162-184, as described in Table 1.

In some cases, antibodies comprise the three heavy chain CDRs as set forth in SEQ ID covered in Table 1 and have amino acid substitutions in the sequences described outside the CDRs such that the resulting V_{H} polypeptide comprises an amino acid sequence having 70% or greater, 75% or greater, 80% or greater, 85% or greater, 90% or greater, 95% or greater, or 99% or greater sequence identity to the amino acid sequence set forth in SEQ ID in Table 1. Similarly, in some cases, antibodies comprise the three heavy chain CDRs as set forth in SEQ ID covered in Table 1 and have amino acid substitutions in the sequence outside the CDRs such that the resulting V_{L} polypeptide comprises an amino acid sequence having 70% or greater, 75% or greater, 80% or greater, 85% or greater, 90% or greater, 95% or greater, or 99% or greater sequence identity to the amino acid sequence set forth in Table 1.

Also provided are expression vectors that include any of the nucleic acids of the present disclosure. The expression vectors find use, e.g., for expressing a V_{H} and/or a V_{L} of an antibody of the present disclosure in a host cell. The expression of natural or synthetic nucleic acids encoding a V_{H} and/or a V_{L} of an antibody of the present disclosure will typically be achieved by operably linking a nucleic acid encoding the V_{H} and/or V_{L} to a promoter (which is either constitutive or inducible), and incorporating the construct into an expression vector. The vectors can be suitable for replication and integration in prokaryotes, eukaryotes, or both. Typical cloning vectors contain transcription and translation terminators, initiation sequences, and promoters useful for regulation of the expression of the nucleic acid encoding the V_{H} and/or V_{L}. The vectors optionally comprise generic expression cassettes containing at least one independent terminator sequence, sequences permitting replication of the cassette in both eukaryotes and prokaryotes, i.e., shuttle vectors, and selection markers for both prokaryotic and eukaryotic systems. See Sambrook et al (1989). To obtain high levels of expression of a cloned nucleic acid it is common to construct expression plasmids which typically contain a strong promoter to direct transcription, a ribosome binding site for translational initiation, and a transcription/translation terminator.

Accordingly, aspects of the present disclosure further include cells, e.g., recombinant host cells. In certain embodiments, provided are cells that include any of the nucleic acids and/or expression vectors of the present disclosure. According to some embodiments, provided are cells that include a first nucleic acid encoding a variable heavy chain (V_{H}) polypeptide of an antibody of the present disclosure, and a second nucleic acid encoding a variable light chain (V_{L}) polypeptide of the antibody. In certain embodiments, provided are cells that include a first expression vector comprising the first nucleic acid, and a second expression vector comprising the second nucleic acid. Cells of the present disclosure may be produced by introducing one or more nucleic acids and/or expression vectors of the present disclosure into host cells via methods known in the art, e.g., electroporation, lipofection, microinjection, or the like.

Also provided are methods of making the antibodies of the present disclosure. In certain embodiments, such methods include culturing a cell (e.g., recombinant host cell) of the present disclosure under conditions suitable for the cell to express the antibody, wherein the antibody is produced. The suitable conditions for culturing the cell such that the antibody is expressed may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%).

Also provided are methods of preparing polyclonal antibodies that specifically bind any of the new immunogens derived from Formula 1 and Formula 2. Antiserum containing antibodies is obtained by well-established techniques involving immunization of an animal, such as rabbits and sheep, with an appropriate immunogen derived from Formula 1 and/or Formula 2 and obtaining antisera from the blood of the immunized animal after an appropriate waiting period. Reviews are provided by Parker, Radioimmunoassay of Biologically Active Compounds, Prentice-Hall (Englewood Cliffs, N.J., U.S., 1976), Butler, J. Immunol. Meth. 7: 1 24 (1975); Broughton and Strong, Clin. Chem. 22:726 732 (1976); and Playfair, et al., Br. Med. Bull. 30: 24 31 (1974). The immunization procedures are well established in the art and are set forth in numerous treatises and publications including “The Immunoassay Handbook,” ^{4t}h Edition, edited by David Wild (Nature Publishing Group, 2013) and the references cited therein. The degree of the antibody purification required depends on the desired application. For many purposes there is no requirement for purification.

Serum harvested may be tested for the presence of antibodies that specifically bind BUP, NBUP, BUP-G and NBUP-G analyte using a BUP protein conjugate or other BUP, NBUP, BUP-G and NBUP-G conjugates in either an ELISA format or homogeneous enzyme immunoassay format. This technique is generally applicable to produce polyclonal antibodies to BUP, NBUP, BUP-G and NBUP-G analytes as described herein and to assess their utility. The specific antibodies prepared are useful as reagents for immunoassays for the detection or determination (optionally including quantification) of BUP and its metabolites NBUP, BUP-G and NBUP-G.

The following procedure may be employed to prepare monoclonal antibodies, in particular for monoclonal antibodies that specifically bind the immunogens of Formula 1 and/or Formula 2. Monoclonal antibodies may be produced according to the standard techniques of Kohler and Milstein, Nature 265:495 497, 1975. Reviews of monoclonal antibody techniques are found in Lymphocyte Hybridomas, ed. Melchers, et al. Springer-Verlag (New York 1978), Nature 266:495 (1977), Science 208:692 (1980), and Methods of Enzymology 73 (Part B): 3 46 (1981). Samples of an appropriate immunogen preparation are injected into an animal such as a rabbit or mouse and, after a sufficient time, the animal is sacrificed and spleen cells obtained. Alternatively, the spleen cells of a non-immunized animal can be sensitized to the immunogen in vitro. The spleen cell chromosomes encoding the base sequences for the desired immunoglobulins can be compressed by fusing the spleen cells, generally in the presence of a non-ionic detergent, for example, polyethylene glycol, with a myeloma cell line. The resulting cells, which include fused hybridomas, are allowed to grow in a selective medium, such as HAT-medium, and the surviving immortalized cells are grown in such medium using limiting dilution conditions. The cells are grown in a suitable container, e.g., microtiter wells, and the supernatant is screened for monoclonal antibodies having the desired specificity. Various techniques exist for enhancing yields of monoclonal antibodies, such as injection of the hybridoma cells into the peritoneal cavity of a mammalian host, which accepts the cells, and harvesting the ascites fluid. Where an insufficient amount of the monoclonal antibody collects in the ascites fluid, the antibody may be harvested from the blood of the host. Alternatively, the cell producing the desired antibody can be grown in a hollow fiber cell culture device or a spinner flask device, both of which are well known in the art. Various conventional ways exist for isolation and purification of the monoclonal antibodies from other proteins and other contaminants (see Kohler and Milstein, supra).

The following procedure may be employed to prepare recombinant monoclonal antibodies, in particular monoclonal antibodies that specifically bind the immunogens of Formula 1. Single B-cell screen, cloning and expression was performed. Peripheral blood mononuclear cells (PBMCs) were isolated from whole blood of rabbit and cultured the same day and plating single B-cells onto 40×96 well plates. The 40×96-well plates were incubated at 37° C./5% CO₂ for seven days in B cell culturing media and the supernatants were then screened by indirect ELISA against-BSA antigen to determine antigen-positive wells. Antigen-positive wells were preserved in RNA lysis buffer and stored at −80° C. mRNA was isolated from selected B cell well (BUP—SH-BSA antigen-positive wells) by Dynabeads mRNA DIRECT purification kit (Ambion, catalog #61012). cDNA was synthesized and 2 rounds of PCR performed to prepare the antibody variable region cDNA for cloning. Rabbit IgG heavy and kappa light chain variable region cDNAs were cloned into mammalian expression vectors with a rabbit heavy and a light chain constant region, respectively. Expression constructs were co-transfected into HEK 293 cells and cell culture supernatants assayed by indirect ELISA against BUP—S-BSA antigen. The antibodies were purified according to standard approaches for antibody purification from supernatants. In general, antibodies can be purified by known techniques such as chromatography, e.g., DEAE chromatography, aBx chromatography, and the like, filtration, and so forth. Antibodies may be screened using any of several techniques, for example using a homogeneous enzyme immunoassay format and considering such properties as, conjugate inhibition, curve size and cross-reactivity, and so forth.

DNA sequencing was performed for selected positive rabbit monoclonal antibodies. The rabbit IgG heavy chain sequence is approximately 1200 bp and can be sequenced from the 5′ ends to obtain a reliable full-length variable sequence. The rabbit kappa light chain is approximately 700 bp and full-length variable sequence can be reliably obtained from sequencing in the 5′ direction. All heavy chain and kappa chain variable region sequences were translated. The resulting amino acid sequences of the V_{H} and V_{L} of example antibodies are provided in Table 1 herein, while the corresponding nucleotide sequences are in Table 2 herein.

The amino acid sequences of embodiments of the variable heavy chain (V_{H}) polypeptides, variable light chain (V_{L}) polypeptides, and CDRs are provided in Table 1 below.

TABLE 1
V_{H}, V_{L}, and CDR Amino Acid Sequences

| | |
|---|---|
| 01H3-1K1 | QEQLKESGGRLVTPGTPLTLTCTVSGFSLSSYYMWWVRQAPGKGLEWIGSIGSRDKY |
| V_{H} | YATWANGRVTISRTSTTVDLKITSPTTEDTATYFCAREVGGERTGAFVLWGQGTLVT |
| (SEQ ID NO: 139) | VSS |
| 01H3-1K1 | GFSLSSYY |
| V_{H} CDR1 | |
| (SEQ ID NO: 1) | |
| 01H3-1K1 | IGSRDK |
| V_{H} CDR2 | |
| (SEQ ID NO: 24) | |
| 01H3-1K1 | AREVGGERTGAFVL |
| V_{H} CDR3 | |
| (SEQ ID NO: 47) | |
| 01H3-1K1 | ALVMTQSPSSVSAAVGDTVTIKCQASQSISGYLAWYQQKPGQAPKLLIYKASTLASGV |
| V_{L} (SEQ ID | SSRFKGRGSGTEFTLTISDLECADAATYYCQQGVSGNNVQNVFGGGTEVVVK |
| NO: 162) | |
| 01H3-1K1 | QSISGY |
| V_{L}CDR1 | |
| (SEQ ID NO: 70) | |
| 01H3-1K1 | KAS |
| V_{L}CDR2 | |
| (SEQ ID NO: 93) | |
| 01H3-1K1 | QQGVSGNNVQNV |
| V_{L}CDR3 | |
| (SEQ ID NO: 116) | |
| 02H3-2K2 | QAVEESGGRLVTPGTPLVLTCTASGIDLSSNDMAWVRQPPGKGLEFIGAIWRGINIFY |
| V_{H} (SEQ ID | ANWAKGRLTISKTSSTTVDLRITSPTIEDTATYFCARDWIAYHSGISFWGQGTLVTVSS |
| NO: 140) | |
| 02H3-2K2 | GIDLSSND |
| V_{H} CDR1 | |
| (SEQ ID NO: 2) | |
| 02H3-2K2 | IWRGINI |
| V_{H} CDR2 | |
| (SEQ ID NO: 25) | |
| 02H3-2K2 | ARDWIAYHSGISF |
| V_{H} CDR3 | |
| (SEQ ID NO: 48) | |
| 02H3-2K2 | AQVLTQTPSSVSAAVGGTVTINCQSSPSVYNNNALSWFQQKPGQPPKVLISRASTRA |
| V_{L} | SGVPSRFKGSGSGTQFTLTISGVECVDAATYYCAGGFDSDGAFGGGTEVVVK |
| (SEQ ID NO: 163) | |
| 02H3-2K2 | PSVYNNNA |
| V_{L} CDR1 | |
| (SEQ ID NO: 71) | |
| 02H3-2K2 | RAS |
| V_{L}CDR2 | |
| (SEQ ID NO: 94) | |
| 02H3-2K2 | AGGFDSDGA |
| V_{L}CDR3 | |
| (SEQ ID NO: 117) | |
| 03H3-3K2 | QSLEESGGRLVTPGTPLTLTCTASGFTISSYHMSWVRQAPGKGLEWIGYLVRGSYNTY |
| V_{H} (SEQ ID | YASWAKGRFTISKTSSTTVDLKLTSPTTEDTATYFCVRDVIGTSGSDLWGQGTLVTVSS |
| NO: 141) | |
| 03H3-3K2 | GFTISSYH |
| V_{H} CDR1 | |
| (SEQ ID NO: 3) | |
| 03H3-3K2 | LVRGSYNT |
| V_{H} CDR2 | |
| (SEQ ID NO: 26) | |
| 03H3-3K2 | VRDVIGTSGSDL |
| V_{H} CDR3 | |
| (SEQ ID NO: 49) | |
| 03H3-3K2 | AAVLTQTPSPVSAAVGGTVTIKCQSSQTVYNNNLLSWYQQKPGQPPKLLIYLASTLAS |
| V_{L} (SEQ ID | GVPDRFSGSGSGTQFTLTISGVQCDDAATYYCLGGFHNDADTAFGGGTEVVVK |
| NO: 164) | |
| 03H3-3K2 | QTVYNNNL |
| V_{L} CDR1 | |
| (SEQ ID NO: 72) | |
| V03H3-3K2 | LAS |
| V_{L} CDR2 | |
| (SEQ ID NO: 95) | |
| 03H3-3K2 | LGGFHNDADTA |
| V_{L}CDR3 | |
| (SEQ ID NO: 118) | |
| 04H3-4K1 | QSVEESGGRLVTPGTPLTLTCTVSGFSLSTAYMSWVRQAPGEGLEWIGYISPGGTTYY |
| V_{H} | ASWAKGRFTISKTSTTVDLKITSPTSEDTATYSCVRSGDCMDFNLWGQGTLVTVSS |
| (SEQ ID NO: 142) | |
| 04H3-4K1 | GFSLSTAY |
| V_{H} CDR1 | |
| (SEQ ID NO: 4) | |
| 04H3-4K1 | ISPGGTT |
| V_{H} CDR2 | |
| (SEQ ID NO: 27) | |
| 04H3-4K1 | VRSGDCMDFNL |
| V_{H} CDR3 | |
| (SEQ ID NO: 50) | |
| 04H3-4K1 | AAVLTQTPSPVSTAVGGTVSISCQSTKSVWRKNCLVWYQQKPGQPPKRLIYQASSLA |
| V_{L} | SGVPSRFKGSGSGTDFTLTIGDVQCDDAATYYCAGGYSDSSDNAFGGGTEVVVK |
| (SEQ ID NO: 165) | |
| 04H3-4K1 | KSVWRKNC |
| V_{L}CDR1 | |
| (SEQ ID NO: 73) | |
| 04H3-4K1 | QAS |
| V_{L}CDR2 | |
| (SEQ ID NO: 96) | |
| 04H3-4K1 | AGGYSDSSDNA |
| V_{L}CDR3 | |
| (SEQ ID NO: 119) | |
| 05H1-5K2 | QAVEESGGRLVTPGTPLVLTCTASGIDLSSNDMAWVRQPPGKGLEFIGGIWRGSSIF |
| V_{H} (SEQ ID | YANWAKGRLTISKTSSTTVDLRITSPTIEDTATYFCARDWIGYHTGISFWGQGTLVTVS |
| NO: 143) | R |
| 05H1-5K2 | GIDLSSND |
| V_{H} CDR1 | |
| (SEQ ID NO: 5) | |
| 05H1-5K2 | IWRGSSI |
| V_{H} CDR2 | |
| (SEQ ID NO: 28) | |
| 05H1-5K2 | ARDWIGYHTGISF |
| V_{H} CDR3 | |
| (SEQ ID NO: 51) | |
| 05H1-5K2 | AQVLTQTPASVSAAVGGTVTINCQSSPSVYNNNALAWYQQKPGQPPKLLISRASTLA |
| V_{L} | SGVPSRFKGSGSGTQFTLTISGVECVDAATYYCAGGFYSDGVFGGGTEVVVK |
| (SEQ ID NO: 166) | |
| 05H1-5K2 V_{L} | PSVYNNNA |
| CDR1 | |
| (SEQ ID NO: 74) | |
| 05H1-5K2 | RAS |
| V_{L}CDR2 | |
| (SEQ ID NO: 97) | |
| 05H1-5K2 | AGGFYSDGV |
| V_{L}CDR3 | |
| (SEQ ID NO: 120) | |
| 06H1-6K2 | QSLEESGGRLVTPGTPLTLTCTASGRSLSYKYMSWVRQAPGKGLERIGYIPASGFRPR |
| V_{H} | YASWAKGRFTISRTSATVDLRITSPTTEDTATYFCAKDLGMDFNLWGQGTLVTVSS |
| (SEQ ID NO: 144) | |
| 06H1-6K2 | GRSLSYKY |
| V_{H} CDR1 | |
| (SEQ ID NO: 6) | |
| 06H1-6K2 | IPASGFRP |
| V_{H} CDR2 | |
| (SEQ ID NO: 29) | |
| 06H1-6K2 | AKDLGMDFNL |
| V_{H} CDR3 | |
| (SEQ ID NO: 4452 | |
| 06H1-6K2 | AAVLTQTPSPVSAAVGGTVTITCQSSQSVYNDNQLSWYQQKSGQPPKILIYEASKLAS |
| V_{L} | GVPSRFSGSGSGTQFTLTIVGVQCDDAASYYCLGAYNDDSETAFGGGTEVVVK |
| (SEQ ID NO: 167) | |
| 06H1-6K2 | QSVYNDNQ |
| V_{L}CDR1 | |
| (SEQ ID NO: 75) | |
| 06H1-6K2 | EAS |
| V_{L}CDR2 | |
| (SEQ ID NO: 98) | |
| 06H1-6K2 | LGAYNDDSETA |
| V_{L} CDR3 | |
| (SEQ ID NO: 121) | |
| 07H3-7K1 | QSLEESGGRLVTPGTPLTLTCTVSGFSLSSNYISWVRQAPGEGLEWVGYVDTTGRSYY |
| V_{H} | ATWAKGRFTISRTSTTVDLKMTSPTSEDTATYFCVRSGGCMDFDLWGQGTLVTVSS |
| (SEQ ID NO: 145) | |
| 07H3-7K1 | GFSLSSNY |
| V_{H} CDR1 | |
| (SEQ ID NO: 7) | |
| 07H3-7K1 | VDTTGRS |
| V_{H} CDR2 | |
| (SEQ ID NO: 30) | |
| 07H3-7K1 | VRSGGCMDFDL |
| V_{H} CDR3 | |
| (SEQ ID NO: 53) | |
| 07H3-7K1 | AQVLTQTPSSVSAAVGGSVSISCQSTKSVWSQNCLVWYQQKPGQPPKRLIYQASKLA |
| V_{L} | SGVPSRFKGSGSGTQFTLTIGDVQCDDAATYYCAGAYYDSSDNGFGGGTEVVVK |
| (SEQ ID NO: 168) | |
| 07H3-7K1 | KSVWSQNC |
| V_{L}CDR1 | |
| (SEQ ID NO: 76) | |
| 07H3-7K1 | QAS |
| V_{L}CDR2 | |
| (SEQ ID NO: 99) | |
| 07H3-7K1 | AGAYYDSSDNG |
| V_{L} CDR3 | |
| (SEQ ID NO: 122) | |
| 08H1-8K1 | QSVEESGGRLVTPGTPLTLTCTVSGIDLNSYDMIWVRQAPGKGLEFIGWIWRGGIAN |
| V_{H} (SEQ ID | YASWAKGRFTFSITSSTTVDLRITSPTTEDTATYFCARDWLGYHTGTSLWGQGTLVTV |
| NO: 146) | SS |
| 08H1-8K1 | GIDLNSYD |
| V_{H} CDR1 | |
| (SEQ ID NO: 8) | |
| 08H1-8K1 | IWRGGIA |
| V_{H} CDR2 | |
| (SEQ ID NO: 31) | |
| 08H1-8K1 | ARDWLGYHTGTSL |
| V_{H} CDR3 | |
| (SEQ ID NO: 54) | |
| 08H1-8K1 | AQVLTQTPASVSAAVGGTVTISCQSSKSVYSNNCLSWFQQKPGQPPKLLIYRASTLAS |
| V_{L} | GVPSRFKGSGSGAQFILTISDVQCDDAATYYCAGGYNSDGAFGGGTEVVVK |
| (SEQ ID NO: 169) | |
| 08H1-8K1 | KSVYSNNC |
| V_{L}CDR1 | |
| (SEQ ID NO: 77) | |
| 08H1-8K1 | RAS |
| V_{L}CDR2 | |
| (SEQ ID NO: 100) | |
| 08H1-8K1 | AGGYNSDGA |
| V_{L}CDR3 | |
| (SEQ ID NO: 123) | |
| 09H1-9K1 | QSVEESGGRLVTPGTPLTLTCTASGFSLNIYGVSWVRQAPGKGLEWIGYIYAGSGST |
| V_{H} | WYASWAKGRFTISKTSTTVDLKITSPTTEDTATYFCARDLGYGDSNYYKYYEFGLWG |
| (SEQ ID NO: 147) | QGTLVTVSS |
| 09H1-9K1 | GFSLNIYG |
| V_{H} CDR1 | |
| (SEQ ID NO: 9) | |
| 09H1-9K1 | IYAGSGST |
| V_{H} CDR2 | |
| (SEQ ID NO: 32) | |
| 09H1-9K1 | ARDLGYGDSNYYKYYEFGL |
| V_{H} CDR3 | |
| (SEQ ID NO: 55) | |
| 09H1-9K1 | AQVLTQTPSPVSAAVGGTVTIACQSSQSVYNNKHLAWYQQKLGQPPKLLIYSASKVA |
| V_{L} (SEQ ID | SGVSSRFSGRGSGTQFTLTISGVDCDDAATYYCAGGYSDVRAFGGGTEVVVK |
| NO: 170) | |
| 09H1-9K1 | QSVYNNKH |
| V_{L}CDR1 | |
| (SEQ ID NO: 78) | |
| 09H1-9K1 | SAS |
| V_{L}CDR2 | |
| (SEQ ID NO: 101) | |
| 09H1-9K1 | AGGYSDVRA |
| V_{L} CDR3 | |
| (SEQ ID NO: 124) | |
| 10H3-10K3 | QEQLKESGGRLVTPGTPLTLTCTASGFSLNSYYMWWVRQAPGKGLEWIGSIGSRDK |
| V_{H} | YYANWAKGRFTISRTSTTVDLKITSPTTDDTATYFCAREVGGERTGAFVLWGQGTLVT |
| (SEQ ID NO: 148) | VSS |
| 10H3-10K3 | GFSLNSYY |
| V_{H} CDR1 | |
| (SEQ ID NO: 10) | |
| 10H3-10K3 | IGSRDK |
| V_{H} CDR2 | |
| (SEQ ID NO: 33) | |
| 10H3-10K3 | AREVGGERTGAFVL |
| V_{H} CDR3 | |
| (SEQ ID NO: 56) | |
| 10H3-10K3 | ALVMTQTPSSVSEPVGGTVTIKCQASQSISGYLAWYQQKPGQPPKLLIYKASTLASGV |
| V_{L} | SSRFKGSGSGTEFTLTISDLECADAATYYCQQGVSGSNIHNPFGGGTEVVVK |
| (SEQ ID NO: 171) | |
| 10H3-10K3 | QSISGY |
| V_{L} CDR1 | |
| (SEQ ID NO: 79) | |
| 10H3-10K3 | KAS |
| V_{L} CDR2 | |
| (SEQ ID NO: 102) | |
| 10H3-10K3 | QQGVSGSNIHNP |
| V_{L} CDR3 | |
| (SEQ ID NO: 125) | |
| 11H1-11K1 | QSLEESGGRLVMPGTPLTLTCTASGFSLSSYYMSWVRQAPGKGLQWIGYIEPTDSRD |
| V_{H} (SEQ ID | YASWAKGRFTISKTSTTVDLRIISPTTEDTATYFCARNVGGRPNLWGQGTLVTVSS |
| NO: 149) | |
| 11H1-11K1 | GFSLSSYY |
| V_{H} CDR1 | |
| (SEQ ID NO: 11) | |
| 11H1-11K1 | IEPTDSR |
| V_{H} CDR2 | |
| (SEQ ID NO: 34) | |
| 11H1-11K1 | ARNVGGRPNL |
| V_{H} CDR3 | |
| (SEQ ID NO: 57) | |
| 11H1-11K1 | AYDVTQTPASVEAAVGGTVTIKCQVSQSVSNWLAWYQQKPGQPPKLLIYSASTLAS |
| V_{L} | GVPSRFKGSGSGTQFTLTISDLECADAATYYCQQAYSGRNVDNVFGGGTEVVVK |
| (SEQ ID NO: 172) | |
| 11H1-11K1 | QSVSNW |
| V_{L}CDR1 | |
| (SEQ ID NO: 80) | |
| 11H1-11K1 | SAS |
| V_{L}CDR2 | |
| (SEQ ID NO: 103) | |
| 11H1-11K1 | QQAYSGRNVDNV |
| V_{L}CDR3 | |
| (SEQ ID NO: 126) | |
| 12H3-12K2 | QSLEESGGRLVTPGTPLTLTCAASGFSLSSNYISWVRQAPGEGLEWVGYVDTSGRSYY |
| V_{H} | ATWAKGRFTISRTSTTVDLKITSPTSEDTATYFCVRSGGCMDFDLWGQGTLVTVSS |
| (SEQ ID NO: 150) | |
| 12H3-12K2 | GFSLSSNY |
| V_{H} CDR1 | |
| (SEQ ID NO: 12) | |
| 12H3-12K2 | VDTSGRS |
| V_{H} CDR2 | |
| (SEQ ID NO: 35) | |
| 12H3-12K2 | VRSGGCMDFDL |
| V_{H} CDR3 | |
| (SEQ ID NO: 58) | |
| 12H3-12K2 | AQVLTQTPSSVSTTVGGTVSISCQSTKSVWSKNCLVWYQQKPGQPPKRLIYQASKLA |
| V_{L} | SGVPSRFKGSGSGTQFTLTIGDVQCDDAATYYCAGAYYDSSDNGFGGGTEVVVK |
| (SEQ ID NO: 173) | |
| 12H3-12K2 | KSVWSKNC |
| V_{L}CDR1 | |
| (SEQ ID NO: 81) | |
| 12H3-12K2 | QAS |
| V_{L}CDR2 | |
| (SEQ ID NO: 104) | |
| 12H3-12K2 | AGAYYDSSDNG |
| V_{L} CDR3 | |
| (SEQ ID NO: 127) | |
| 13H1-13K2 | QSLEESGGRLVTPGTPLTLTCTASGFTISNYHMSWVRQAPGKGLEWIGYIVAGSDVA |
| V_{H} (SEQ ID | YYASWARGRFTISKTSSTTVDLTITRPTTEDTATYFCVRDVAGTTGSDLWGQGTLVTV |
| NO: 151) | SS |
| 13H1-13K2 | GFTISNYH |
| V_{H} CDR1 | |
| (SEQ ID NO: 13) | |
| 13H1-13K2 | IVAGSDVA |
| V_{H} CDR2 | |
| (SEQ ID NO: 36) | |
| 13H1-13K2 | VRDVAGTTGSDL |
| V_{H} CDR3 | |
| (SEQ ID NO: 59) | |
| 13H1-13K2 | AAVLTQTPSPVSAAVGGTVTIKCQSSQSVYNKNLLSWYQQKPGQPPRVLIYKASTLAS |
| V_{L} | GVPSRFSGSGSGTQFTLTIRGVQCDDAATYYCLGAYDNDADTAFGGGTEVVVK |
| (SEQ ID NO: 174) | |
| 13H1-13K2 | QSVYNKNL |
| V_{L}CDR1 | |
| (SEQ ID NO: 82) | |
| 13H1-13K2 | KAS |
| V_{L}CDR2 | |
| (SEQ ID NO: 105) | |
| 13H1-13K2 | LGAYDNDADTA |
| V_{L}CDR3 | |
| (SEQ ID NO: 128) | |
| 14H1-14K2 | QSVEESGGRLVTPGTPLTLTCTVSGFSLSSSYMSWVRQAPGKGLEWIGYIDTAVATYF |
| V_{H} | ASWAKGRFTISKTSTTVDLKITSPTSEDTATYFCVRSDDCMAFNFWGQGTLVTVSS |
| (SEQ ID NO: 152) | |
| 14H1-14K2 | GFSLSSSY |
| V_{H} CDR1 | |
| (SEQ ID NO: 14) | |
| 14H1-14K2 | IDTAVAT |
| V_{H} CDR2 | |
| (SEQ ID NO: 37) | |
| 14H1-14K2 | VRSDDCMAFNF |
| V_{H} CDR3 | |
| (SEQ ID NO: 60) | |
| 14H1-14K2 | AAVLTQTPASVSAAVGGTVTISCQSTRSVWRNNCLAWYQQKPGQPPKLLIYRASSLA |
| V_{L} (SEQ ID | SGVPSRFRGSGSGTQFTLTIGDVQCDDAATYYCAGGYSDARDNGFGGGTEVVVK |
| NO: 175) | |
| 14H1-14K2 | RSVWRNNC |
| V_{L}CDR1 | |
| (SEQ ID NO: 83) | |
| 14H1-14K2 | RAS |
| V_{L}CDR2 | |
| (SEQ ID NO: 106) | |
| 14H1-14K2 | AGGYSDARDNG |
| V_{L}CDR3 | |
| (SEQ ID NO: 129) | |
| 15H3-15K2 | QSLEESGGRLVTPGTPLTLTCTVSGFSLSTAYISWVRQAPGEGLQWIGYIHLGRSTYYA |
| V_{H} (SEQ ID | SWAQGRFTISKTSSTTVDLKITSPTSEDTATYFCVRSGDCMAFDLWGQGTLVTVSS |
| NO: 153) | |
| 15H3-15K2 | GFSLSTAY |
| V_{H} CDR1 | |
| (SEQ ID NO: 15) | |
| 15H3-15K2 | IHLGRST |
| V_{H} CDR2 | |
| (SEQ ID NO: 38) | |
| 15H3-15K2 | VRSGDCMAFDL |
| V_{H} CDR3 | |
| (SEQ ID NO: 61) | |
| 15H3-15K2 | AAVLTQTPSPVSAAVGGTVSISCQSTKSVYSKNCLAWYQQKPGQPPKLLIYKASTLAS |
| V_{L} | GVPSRFKGSGSGTQFTLTIGDVQCDDVATYYCAGGYSDARDNAFGGGTEVVVK |
| (SEQ ID NO: 176) | |
| 15H3-15K2 | KSVYSKNC |
| V_{L}CDR1 | |
| (SEQ ID NO: 84) | |
| 15H3-15K2 | KAS |
| V_{L}CDR2 | |
| (SEQ ID NO: 107) | |
| 15H3-15K2 | AGGYSDARDNA |
| V_{L}CDR3 | |
| (SEQ ID NO: 130) | |
| 16H2-16K3 | QSVEESGGRLVTPGTPLTLTCTASGFTISTYHMSWVRQAPGKGLEWIGYINANSGGT |
| V_{H} | WYASWAKGRFTISKTSTTVNLKITSPTTEDTATYFCVRSGYNSGMAFDPWGQGTLVT |
| (SEQ ID NO: 154) | VSS |
| 16H2-16K3 | GFTISTYH |
| V_{H} CDR1 | |
| (SEQ ID NO: 16) | |
| 16H2-16K3 | INANSGGT |
| V_{H} CDR2 | |
| (SEQ ID NO: 39) | |
| 16H2-16K3 | VRSGYNSGMAFDP |
| V_{H} CDR3 | |
| (SEQ ID NO: 62) | |
| 16H2-16K3 | AAVLTQTPSPVSAAVGGTVSISCQSSKSVYNNNNLAWFQQKPGQPPKLLIYGASML |
| V_{L} (SEQ ID | ASGVPSRFKGSGSGTQFTLTISDVQCDDAATYYCAGAFSSASDNAFGGGTEVVVK |
| NO: 177) | |
| 16H2-16K3 | KSVYNNNN |
| V_{L}CDR1 | |
| (SEQ ID NO: 85) | |
| 16H2-16K3 | GAS |
| V_{L}CDR2 | |
| (SEQ ID NO: 108) | |
| 16H2-16K3 | AGAFSSASDNA |
| V_{L} CDR3 | |
| (SEQ ID NO: 131) | |
| 17H1-17K2 | QEQLGESGGGLVQPGGTLKLSCKGSGFDLSSNAMCWVRQAPGKGLEWVGCIIYGST |
| V_{H} | YYATWVNGRFTLSRDNAQSSVDLQLSSLTAADTATYFCARGGRSYGDGYAFGYFNI |
| (SEQ ID NO: 155) | WGQGTLVTVSS |
| 17H1-17K2 | GFDLSSNA |
| V_{H} CDR1 | |
| (SEQ ID NO: 17) | |
| 17H1-17K2 | IIYGST |
| V_{H} CDR2 | |
| (SEQ ID NO: 40) | |
| 17H1-17K2 | ARGGRSYGDGYAFGYFNI |
| V_{H} CDR3 | |
| (SEQ ID NO: 63) | |
| 17H1-17K2 | ALVMTQTPSSVSAAVGGTVTINCQASQNIYSNLAWYQQKPGQRPKLLIYGTSNLESG |
| V_{L} (SEQ ID | VPSRFKGSGSGTEYTLTISDLECDDAATYYCQSAYYSSSTDMANAFGGGTEVVVK |
| NO: 178) | |
| 17H1-17K2 | QNIYSN |
| V_{L}CDR1 | |
| (SEQ ID NO: 86) | |
| 17H1-17K2 | GTS |
| V_{L}CDR2 | |
| (SEQ ID NO: 109) | |
| 17H1-17K2 | QSAYYSSSTDMANA |
| V_{L}CDR3 | |
| (SEQ ID NO: 132) | |
| 18H2-18K3 | QEQLEESGGRLGTPGTPLTLTCTVSGFSLIGQYMSWVRQAPGKGLEWIGYIDTTERSY |
| V_{H} | YASWAKGRFTISKTSSTTVDLKMTSLTTEDTATYFCVRSDSCMAFGLWGQGTLVTVS |
| (SEQ ID NO: 156) | S |
| 18H2-18K3 | GFSLIGQY |
| V_{H} CDR1 | |
| (SEQ ID NO: 18) | |
| 18H2-18K3 | IDTTERS |
| V_{H} CDR2 | |
| (SEQ ID NO: 41) | |
| 18H2-18K3 | VRSDSCMAFGL |
| V_{H} CDR3 | |
| (SEQ ID NO: 64) | |
| 18H2-18K3 | VQVLTQTPSSVSAAVGGTVTMSCQSTKSVWNNNCLAWYQQKPGQPPKLLIYKASTL |
| V_{L} (SEQ ID | AAGVSSRFKGNGSGTQSTLTISDVQCDDAATYYCAGGYSTSSDNGFGGGTEVVVK |
| NO: 179) | |
| 18H2-18K3 | KSVWNNNC |
| V_{L}CDR1 | |
| (SEQ ID NO: 87) | |
| 18H2-18K3 | KAS |
| V_{L}CDR2 | |
| (SEQ ID NO: 110) | |
| 18H2-18K3 | AGGYSTSSDNG |
| V_{L}CDR3 | |
| (SEQ ID NO: 133) | |
| 22H2-22K1 | QSGGGGEGGLVKPGGSLELCCKASGFSLNNKFWIWWVRQAPGKGLEWIGSIDAGG |
| V_{H} | SGGTYYASWVNGRFTLSRDIDQSTYCLQLNSLTAADTAIYYCARDLAAAGGGLTGAF |
| (SEQ ID NO: 157) | NLWGQGTLVTVSS |
| 22H2-22K1 | GFSLNNKFW |
| V_{H} CDR1 | |
| (SEQ ID NO: 19) | |
| 22H2-22K1 | IDAGGSGGT |
| V_{H} CDR2 | |
| (SEQ ID NO: 42) | |
| 22H2-22K1 | ARDLAAAGGGLTGAFNL |
| V_{H} CDR3 | |
| (SEQ ID NO: 65) | |
| 22H2-22K1 | AYDMTQTPASVSAAVGGTVTINCQASQSINNLLAWYQQKPGQPPKLLIYKASTLASG |
| V_{L} (SEQ ID | VPSRFKGSGSGTQFTLTISGVQCADAATYYCQQGWSGSNVDNVFGGGTEVVVK |
| NO: 180) | |
| 22H2-22K1 | QSINNL |
| V_{L} CDR1 | |
| (SEQ ID NO: 88) | |
| 22H2-22K1 | KAS |
| V_{L} CDR2 | |
| (SEQ ID NO: 111) | |
| 22H2-22K1 | QQGWSGSNVDNV |
| V_{L} CDR3 | |
| (SEQ ID NO: 134) | |
| 23H4-23K4 | QSLEESGGGLVQPEGSLTLTCTASGFSFSSSYYMCWVRQAPGKGLEWIACIYVGSNN |
| V_{H} (SEQ ID | NSYYASWAKGRFAISKTSSTTVTLQMGSLTAADTATYFCARSIVGYAGYTYARYNAF |
| NO: 158) | DPWGQGTLVTVSS |
| 23H4-23K4 | GFSFSSSYY |
| V_{H} CDR1 | |
| (SEQ ID NO: 20) | |
| 23H4-23K4 | IYVGSNNNS |
| V_{H} CDR2 | |
| (SEQ ID NO: 43) | |
| 23H4-23K4 | ARSIVGYAGYTYARYNAFDP |
| V_{H} CDR3 | |
| (SEQ ID NO: 66) | |
| 23H4-23K4 | ALVMTQTPSSVSGAVGGTVTINCQASQNIYNNLAWYQQKPGQPPKLLIYAASNLAS |
| V_{L} | GVSSRFKGSGSGTEYTLTISDLECDDAATYYCQSVAYSTGAATIAFGGGTEVVVK |
| (SEQ ID NO: 181) | |
| 23H4-23K4 | QNIYNN |
| V_{L}CDR1 | |
| (SEQ ID NO: 89) | |
| 23H4-23K4 | AAS |
| V_{L}CDR2 | |
| (SEQ ID NO: 112) | |
| 23H4-23K4 | QSVAYSTGAATIA |
| V_{L}CDR3 | |
| (SEQ ID NO: 135) | |
| 25H4-25K1 | QEQLVESGGGLVKPGASLTLTCKASGFSFSSGYYMCWVRQAPGKGLEWIGCILPGTV |
| V_{H} | ASYYASWAKGRSTISKASSTTVDLKMTRLTAADTATYFCARVNPTYGGHGYDLWGQ |
| (SEQ ID NO: 159) | GTLVTVSS |
| 25H4-25K1 | GFSFSSGYY |
| V_{H} CDR1 | |
| (SEQ ID NO: 21) | |
| 25H4-25K1 | ILPGTVAS |
| V_{H} CDR2 | |
| (SEQ ID NO: 44) | |
| 25H4-25K1 | ARVNPTYGGHGYDL |
| V_{H} CDR3 | |
| (SEQ ID NO: 67) | |
| 25H4-25K1 | AAVLTQTPSPVSAAVGGTVSISCQSSKSVHNNNHLAWFQQKPGQPPKLLIYKASTLA |
| V_{L} | SGVPSRFKGSGSGTQFTLTISDVQCDDAATYYCAGAFISSGDNGFGGGTEVVVK |
| (SEQ ID NO: 182) | |
| 25H4-25K1 | KSVHNNNH |
| V_{L} CDR1 | |
| (SEQ ID NO: 90) | |
| 25H4-25K1 | KAS |
| V_{L} CDR2 | |
| (SEQ ID NO: 113) | |
| 25H4-25K1 | AGAFISSGDNG |
| V_{L}CDR3 | |
| (SEQ ID NO: 136) | |
| 26H6-26K5 | QEQLVESGGGLVQPGASLTLTCKASGFSVSSGVYMCWVRQAPGKGLEWIGCILTGS |
| V_{H} | VTSYYATWVNGRFTLSKASSTTLYLQLNSLTVADTATHFCVRVNPNYGGHGYDLWG |
| (SEQ ID NO: 160) | QGTLVTVSS |
| 26H6-26K5 | GFSVSSGVY |
| V_{H} CDR1 | |
| (SEQ ID NO: 22) | |
| 26H6-26K5 | ILTGSVTS |
| V_{H} CDR2 | |
| (SEQ ID NO: 45) | |
| 26H6-26K5 | VRVNPNYGGHGYDL |
| V_{H} CDR3 | |
| (SEQ ID NO: 68) | |
| 26H6-26K5 | AQVLTQTPASVSAAVGGTVSISCQSSKSVYARNHLAWFQQKPGQPPKLLIYKASNLP |
| V_{L} | TGVPSRFSGSGSGTQFTLTISDVQCDDAATYYCAGAFSTSGDNGFGGGTEVVVK |
| (SEQ ID NO: 183) | |
| 26H6-26K5 | KSVYARNH |
| V_{L}CDR1 | |
| (SEQ ID NO: 91) | |
| 26H6-26K5 | KAS |
| V_{L}CDR2 | |
| (SEQ ID NO: 114) | |
| 26H6-26K5 | AGAFSTSGDNG |
| V_{L}CDR3 | |
| (SEQ ID NO: 137) | |
| 27H6-27K5 | RSLEESGGGLVKPGETLTLTCKASGFDFTSSAACWVRQAPGKGLEWIGCILNSRIYYA |
| V_{H} | AWAKGRFTISRASSITVTLRMTSLTAADTATYFCARVPGGSPYNWWAMDLWGQGT |
| (SEQ ID NO: 161) | LVTVSS |
| 27H6-27K5 | GFDFTSSA |
| V_{H} CDR1 | |
| (SEQ ID NO: 23) | |
| 27H6-27K5 | ILNSRI |
| V_{H} CDR2 | |
| (SEQ ID NO: 46) | |
| 27H6-27K5 | ARVPGGSPYNWWAMDL |
| V_{H} CDR3 | |
| (SEQ ID NO: 69) | |
| 27H6-27K5 | AQVLTQTPSSVSAAVGGTVTINCQSSQSVAGTNWLSWYQQKPGQPPKLLIYKASILE |
| V_{L} | AGVSSRFKGSRSGTQFTLTINDMQCDDAATYYCHGAYSSVNVIHTFGGGTEVVVK |
| (SEQ ID NO: 184) | |
| 27H6-27K5 | QSVAGTNW |
| V_{L} CDR1 | |
| (SEQ ID NO: 92) | |
| 27H6-27K5 | KAS |
| V_{L} CDR2 | |
| (SEQ ID NO: 115) | |
| 27H6-27K5 | HGAYSSVNVIHT |
| V_{L} CDR3 | |
| (SEQ ID NO: 138) | |

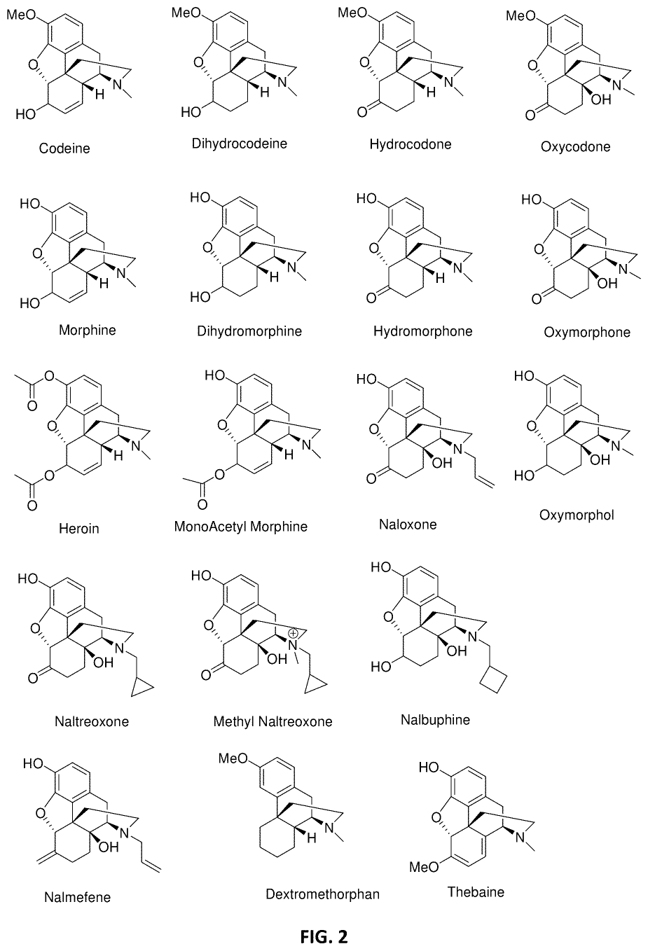

In certain embodiments, antibodies of the present disclosure further specifically binds to BUP and metabolites N-BUP, BUP-G and N-BUP-G. According to some embodiments, antibodies of the present disclosure have cross reactivity of structurally similar opiates (FIG. 2) of less than 1%, less than 0.1%, less than 0.01%, of its reactivity for BUP, N-BUP, BUP-G and N-BUP-G.

Aspects of the present disclosure further include nucleic acids. In certain embodiments, a nucleic acid of the present disclosure encodes a variable heavy chain (V_{H}) polypeptide, a variable light chain (V_{L}) polypeptide, or both, of any of the antibodies of the present disclosure, including but not limited to a V_{H} and/or a V_{L} that includes the CDRs of any of the antibodies set forth in Table 1. Examples of nucleic acids having nucleotide sequences that encode example antibodies of the present disclosure are provided in Table 2 below.

TABLE 2
Nucleotide Sequences

| | |
|---|---|
| 01H3-1K1V_{H} | CAGGAGCAGCTGAAGGAGTCCGGGGGTCGCCTGGTCACGCCTGGGACACCCCT |
| (SEQ ID NO: 185) | GACACTCACCTGCACAGTCTCTGGATTCTCCCTCAGTAGCTACTACATGTGGTGG |
| | GTCCGCCAGGCTCCAGGAAAGGGCCTGGAATGGATCGGAAGTATTGGGAGTCG |
| | TGATAAATATTACGCGACCTGGGCGAATGGTCGAGTCACCATCTCCAGAACCTC |
| | GACCACGGTGGATCTGAAGATCACCAGTCCGACAACCGAGGACACGGCCACCTA |
| | TTTCTGTGCCAGAGAAGTTGGTGGTGAGAGGACTGGTGCGTTTGTCTTGTGGGG |
| | CCAGGGCACCCTGGTCACCGTCTCGAGC |
| 01H3-1K1 V_{L} | GCCCTTGTGATGACCCAGTCTCCATCCTCCGTGTCTGCAGCTGTGGGAGACACAG |
| (SEQ ID NO: 208) | TCACCATCAAGTGCCAGGCCAGTCAGAGCATTAGTGGCTACTTAGCCTGGTATCA |
| | GCAGAAACCAGGGCAGGCTCCCAAGCTCCTGATCTACAAGGCTTCCACTCTGGC |
| | ATCTGGGGTCTCATCGCGATTCAAAGGCCGTGGATCTGGGACAGAGTTCACTCT |
| | CACCATCAGCGACCTGGAGTGTGCCGATGCTGCCACTTACTATTGTCAACAGGGT |
| | GTTAGTGGTAATAATGTTCAAAATGTTTTCGGCGGAGGGACCGAGGTGGTGGTC |
| | AAA |
| 02H3-2K2 V_{H} | CAGGCGGTGGAGGAATCCGGGGGTCGCCTGGTCACGCCTGGGACACCCCTGGT |
| (SEQ ID NO: 186) | ACTCACCTGCACAGCCTCTGGAATCGACCTCAGTAGCAACGACATGGCGTGGGT |
| | CCGCCAGCCTCCAGGGAAGGGGCTGGAATTCATCGGAGCCATTTGGAGGGGTA |
| | TTAATATATTCTACGCGAACTGGGCGAAAGGCCGACTCACCATCTCCAAAACCTC |
| | GTCGACCACGGTGGATCTGAGAATCACCAGTCCGACAATCGAGGACACGGCCAC |
| | CTATTTCTGTGCCAGAGATTGGATTGCTTATCATAGTGGTATAAGTTTCTGGGGC |
| | CAGGGCACCCTGGTCACCGTCTCGAGC |
| 02H3-2K2 V_{L} | GCCCAAGTGCTGACCCAGACTCCATCCTCCGTGTCTGCAGCTGTGGGAGGCACA |
| (SEQ ID NO: 209) | GTCACCATCAATTGCCAGTCCAGTCCGAGCGTTTATAATAACAATGCATTATCCT |
| | GGTTTCAGCAGAAACCAGGGCAGCCTCCCAAGGTCCTGATCTCCAGGGCATCCA |
| | CTCGGGCATCTGGGGTCCCATCGCGGTTCAAGGGCAGTGGATCTGGGACACAGT |
| | TCACTCTCACCATCAGCGGCGTGGAGTGTGTCGATGCTGCCACTTACTACTGTGC |
| | AGGCGGTTTTGATAGTGATGGTGCTTTCGGCGGAGGGACCGAGGTGGTGGTCA |
| | AA |
| 03H3-3K2 V_{H} | CAGTCGCTGGAGGAGTCCGGGGGTCGCCTGGTCACGCCTGGGACACCCCTGAC |
| (SEQ ID NO: 187) | ACTCACCTGCACAGCCTCTGGATTCACCATCAGTAGCTACCATATGAGCTGGGTC |
| | CGCCAGGCTCCAGGGAAGGGGCTGGAGTGGATCGGATATCTTGTTCGTGGTAG |
| | TTATAACACATACTACGCGAGCTGGGCGAAAGGCCGATTCACCATCTCCAAAACC |
| | TCGTCGACCACGGTGGATCTGAAGCTCACCAGTCCGACAACCGAGGACACGGCC |
| | ACCTATTTCTGTGTCAGAGATGTTATTGGTACTAGTGGTTCTGACTTGTGGGGCC |
| | AGGGCACCCTGGTCACCGTCTCGAGC |
| 03H3-3K2 V_{L} | GCAGCCGTGCTGACCCAGACACCATCGCCCGTGTCTGCAGCTGTGGGAGGCACA |
| (SEQ ID NO: 210) | GTCACCATCAAGTGCCAGTCCAGTCAGACTGTTTATAATAACAACCTCTTATCCTG |
| | GTATCAGCAGAAACCAGGGCAGCCTCCCAAGCTCCTGATCTATCTGGCGTCCACT |
| | CTGGCATCTGGGGTCCCAGATAGGTTCAGCGGCAGTGGATCTGGGACACAGTTC |
| | ACTCTCACCATCAGCGGCGTGCAGTGTGACGATGCTGCCACTTACTACTGTCTAG |
| | GCGGTTTTCATAATGATGCTGATACGGCTTTCGGCGGAGGGACCGAGGTGGTG |
| | GTCAAA |
| 04H3-4K1 V_{H} | CAGTCGGTGGAGGAGTCCGGGGGTCGCCTGGTCACGCCTGGGACACCCCTGAC |
| (SEQ ID NO: 188) | ACTCACCTGCACAGTCTCTGGCTTCTCCCTCAGTACCGCCTACATGAGCTGGGTC |
| | CGCCAGGCTCCAGGGGAGGGGCTGGAATGGATCGGATATATTAGTCCTGGTGG |
| | TACCACATACTACGCGAGCTGGGCGAAAGGCCGATTCACCATCTCCAAAACCTC |
| | GACCACGGTGGATCTGAAAATCACCAGTCCGACAAGCGAGGACACGGCCACCTA |
| | TAGCTGTGTCAGGAGTGGTGACTGTATGGACTTTAATTTGTGGGGCCAGGGCAC |
| | CCTGGTCACCGTCTCGAGC |
| 04H3-4K1 V_{L} | GCCGCCGTGCTGACCCAGACTCCATCTCCCGTGTCTACAGCTGTGGGAGGCACA |
| (SEQ ID NO: 211) | GTCAGCATCAGTTGCCAGTCCACTAAGAGTGTTTGGCGTAAGAACTGCTTAGTCT |
| | GGTATCAACAGAAACCAGGGCAGCCTCCCAAGCGCCTGATTTACCAGGCATCCA |
| | GTCTGGCATCTGGGGTCCCATCGCGGTTCAAAGGCAGTGGATCTGGGACAGATT |
| | TCACTCTCACCATCGGCGACGTGCAGTGTGACGATGCTGCCACTTACTACTGTGC |
| | GGGCGGTTATAGTGATAGTAGTGATAATGCTTTCGGCGGAGGGACCGAGGTGG |
| | TGGTCAAA |
| 05H1-5K2 V_{H} | CAGGCGGTGGAGGAGTCCGGGGGTCGCCTGGTCACGCCTGGGACACCCCTGGT |
| (SEQ ID NO: 189) | ACTCACCTGCACAGCCTCTGGAATCGACCTCAGTAGCAACGACATGGCGTGGGT |
| | CCGCCAGCCTCCAGGGAAGGGGCTGGAATTCATCGGAGGCATTTGGAGGGGTA |
| | GTAGTATATTCTACGCGAACTGGGCGAAAGGCCGACTCACCATCTCCAAAACCTC |
| | GTCGACCACGGTGGATCTGAGAATCACCAGTCCGACAATCGAGGACACGGCCAC |
| | GTATTTCTGTGCCAGAGATTGGATTGGTTATCATACTGGTATCAGTTTCTGGGGC |
| | CAGGGCACCCTGGTCACCGTCTCGAGG |
| 05H1-5K2 V_{L} | GCCCAAGTGCTGACCCAGACTCCAGCCTCCGTGTCTGCAGCTGTGGGAGGCACA |
| (SEQ ID NO: 212) | GTCACCATCAATTGCCAGTCCAGTCCGAGTGTTTATAATAACAATGCTTTAGCCT |
| | GGTATCAACAGAAACCAGGGCAGCCTCCCAAGCTCCTGATCTCCAGGGCATCCA |
| | CTCTGGCATCTGGGGTCCCATCGCGGTTCAAGGGCAGTGGATCTGGGACACAAT |
| | TCACTCTCACCATCAGCGGCGTGGAGTGTGTCGATGCTGCCACTTACTACTGTGC |
| | AGGCGGTTTTTATAGTGATGGTGTTTTCGGCGGAGGGACCGAGGTGGTGGTCA |
| | AA |
| 06H1-6K2 V_{H} | CAGTCGCTGGAGGAGTCCGGGGGCCGCCTGGTCACGCCTGGGACACCCCTGAC |
| (SEQ ID NO: 190) | ACTCACCTGCACAGCCTCTGGACGCTCCCTCAGTTATAAGTATATGAGTTGGGTC |
| | CGGCAGGCTCCAGGGAAGGGGCTGGAACGGATCGGTTACATTCCTGCTAGTGG |
| | TTTTCGCCCGAGGTATGCGAGTTGGGCGAAAGGCCGATTCACCATCTCCAGAAC |
| | CTCGGCCACAGTGGATCTGAGAATCACCAGTCCGACAACCGAGGACACGGCCAC |
| | CTATTTCTGTGCCAAAGATTTGGGAATGGACTTTAATTTGTGGGGCCAGGGCACC |
| | CTGGTCACCGTCTCGAGC |
| 06H1-6K2 V_{L} | GCAGCCGTACTGACCCAGACACCATCACCCGTGTCTGCAGCTGTGGGAGGCACA |
| (SEQ ID NO: 213) | GTCACCATCACTTGCCAGTCCAGTCAGAGTGTTTATAATGACAACCAATTATCCT |
| | GGTATCAGCAGAAATCAGGGCAGCCTCCCAAGATCCTGATCTACGAAGCATCCA |
| | AACTGGCATCTGGGGTCCCATCGCGGTTCAGTGGCAGTGGATCTGGGACACAGT |
| | TCACTCTCACCATCGTCGGTGTGCAGTGTGACGATGCTGCCTCTTACTACTGCCTA |
| | GGCGCTTATAATGATGATTCTGAGACTGCTTTCGGCGGAGGGACCGAGGTGGTG |
| | GTCAAA |
| 07H3-7K1 V_{H} | CAGTCGCTGGAGGAGTCCGGGGGTCGCCTGGTCACGCCTGGGACACCCCTGAC |
| (SEQ ID NO: 191) | ACTCACCTGCACAGTCTCTGGCTTCTCCCTCAGTAGCAACTACATCAGCTGGGTC |
| | CGCCAGGCTCCAGGGGAGGGGCTGGAGTGGGTCGGATATGTCGATACTACTGG |
| | CCGCTCATATTACGCGACCTGGGCAAAAGGCCGATTCACCATCTCCAGAACCTCG |
| | ACCACGGTGGATCTGAAAATGACCAGTCCGACAAGCGAGGACACGGCCACCTAT |
| | TTTTGTGTCAGGAGTGGTGGCTGTATGGACTTTGACTTGTGGGGCCAGGGCACC |
| | CTGGTCACCGTCTCGAGC |
| 07H3-7K1 V_{L} | GCCCAAGTGCTGACTCAGACTCCATCCTCCGTGTCTGCAGCTGTGGGAGGCTCA |
| (SEQ ID NO: 214) | GTCAGCATCAGTTGCCAGTCCACTAAGAGTGTTTGGAGTCAGAATTGCTTAGTCT |
| | GGTATCAGCAGAAACCAGGGCAGCCTCCCAAGCGCCTCATTTACCAGGCATCGA |
| | AACTGGCATCTGGGGTCCCGTCGCGGTTCAAAGGCAGTGGCTCTGGGACACAGT |
| | TCACTCTCACCATCGGCGACGTGCAGTGTGACGATGCTGCCACTTACTACTGTGC |
| | AGGCGCTTATTATGATAGTAGTGATAATGGTTTCGGCGGAGGGACCGAGGTGG |
| | TGGTCAAA |
| 08H1-8K1 V_{H} | CAGTCGGTGGAGGAGTCCGGGGGTCGCCTGGTCACGCCTGGGACACCCCTGAC |
| (SEQ ID NO: 192) | ACTCACCTGCACAGTCTCTGGGATCGACCTCAATAGCTACGACATGATCTGGGTC |
| | CGCCAGGCTCCAGGGAAGGGGCTGGAATTCATCGGATGGATTTGGAGGGGTGG |
| | TATTGCGAACTACGCGAGCTGGGCGAAAGGCCGATTCACCTTCTCCATAACCTCG |
| | TCGACCACGGTGGATCTCAGAATCACCAGTCCGACAACCGAGGACACGGCCACC |
| | TATTTCTGTGCCAGAGATTGGCTTGGATATCATACTGGTACAAGTTTGTGGGGCC |
| | AGGGCACCCTGGTCACCGTCTCGAGC |
| 08H1-8K1 V_{L} | GCCCAAGTGCTGACCCAGACTCCTGCCTCCGTGTCTGCAGCTGTGGGAGGCACA |
| (SEQ ID NO: 215) | GTCACCATCAGTTGCCAGTCCAGTAAGAGTGTTTATAGTAACAACTGCTTATCCT |
| | GGTTTCAACAGAAACCAGGCCAGCCTCCTAAGCTCCTGATCTACAGGGCATCCAC |
| | TCTGGCATCTGGGGTCCCATCGCGGTTCAAGGGCAGTGGATCTGGGGCACAGTT |
| | TATTCTCACCATTAGCGACGTGCAGTGTGACGATGCTGCCACTTACTACTGTGCA |
| | GGCGGTTATAATAGTGATGGTGCTTTCGGCGGAGGGACCGAGGTGGTGGTCAA |
| | A |
| 09H1-9K1 V_{H} | CAGTCGGTGGAGGAGTCCGGGGGTCGCCTGGTCACGCCTGGGACACCCCTGAC |
| (SEQ ID NO: 193) | ACTCACCTGCACAGCCTCTGGATTCTCCCTCAATATCTATGGAGTGAGCTGGGTC |
| | CGCCAGGCTCCAGGGAAGGGGCTGGAGTGGATCGGATATATTTATGCTGGTAG |
| | TGGTAGCACATGGTACGCGAGCTGGGCGAAAGGCCGATTCACCATCTCCAAAAC |
| | CTCGACCACGGTGGATCTGAAAATCACCAGTCCGACAACCGAGGACACGGCCAC |
| | CTATTTCTGTGCCAGAGATCTCGGTTACGGTGATAGTAATTATTATAAATATTATG |
| | AATTTGGTTTGTGGGGCCAGGGCACCCTGGTCACCGTCTCGAGC |
| 09H1-9K1 V_{L} | GCCCAAGTGCTGACCCAGACTCCATCCCCTGTGTCTGCAGCTGTGGGAGGCACA |
| (SEQ ID NO: 216) | GTCACCATCGCTTGCCAGTCCAGTCAGAGTGTTTATAATAATAAACACTTAGCCT |
| | GGTATCAGCAGAAACTAGGACAGCCTCCCAAACTCCTGATCTATTCTGCGTCCAA |
| | AGTGGCATCTGGGGTCTCATCGCGGTTCAGTGGCCGTGGATCTGGGACACAGTT |
| | CACTCTCACCATCAGTGGCGTGGACTGTGACGATGCTGCCACTTACTACTGTGCA |
| | GGCGGTTATAGTGACGTTCGTGCTTTCGGCGGAGGGACCGAGGTGGTGGTCAA |
| | A |
| 10H3-10K3 V_{H} | CAGGAGCAGCTGAAGGAGTCCGGGGGTCGCCTGGTCACGCCTGGGACACCCCT |
| (SEQ ID NO: 194) | GACACTCACCTGCACAGCCTCTGGATTCTCCCTCAATAGCTACTATATGTGGTGG |
| | GTCCGCCAGGCTCCAGGAAAGGGGCTGGAATGGATCGGGAGCATTGGTAGTCG |
| | TGATAAATATTACGCGAACTGGGCGAAAGGCCGATTCACCATCTCCAGAACCTC |
| | GACCACGGTGGATCTGAAGATCACCAGTCCGACAACCGACGACACGGCCACCTA |
| | TTTCTGTGCCAGAGAAGTTGGTGGTGAGAGGACTGGTGCGTTTGTCTTGTGGGG |
| | CCAGGGCACCCTGGTCACCGTCTCGAGC |
| 10H3-10K3 V_{L} | GCCCTTGTGATGACCCAGACTCCATCCTCCGTGTCTGAACCTGTGGGAGGCACA |
| (SEQ ID NO: 217) | GTCACCATCAAGTGCCAGGCCAGTCAGAGCATTAGTGGCTACTTAGCCTGGTAT |
| | CAGCAGAAACCAGGGCAGCCTCCCAAGCTCCTGATCTACAAGGCATCCACTTTG |
| | GCATCTGGGGTCTCATCGCGATTCAAAGGCAGTGGATCTGGGACAGAGTTCACT |
| | CTCACCATCAGCGACCTGGAGTGTGCCGATGCTGCCACTTACTACTGTCAACAGG |
| | GTGTTAGTGGTAGTAATATTCATAATCCTTTCGGCGGAGGGACCGAGGTGGTGG |
| | TCAAA |
| 11H1-11K1 V_{H} | CAGTCGCTGGAGGAGTCCGGGGGTCGCCTGGTCATGCCTGGGACACCCCTGAC |
| (SEQ ID NO: 195) | ACTCACCTGCACAGCCTCTGGATTCTCCCTCAGTTCTTACTATATGAGTTGGGTCC |
| | GCCAGGCTCCAGGGAAGGGGCTGCAGTGGATCGGATATATTGAGCCTACTGATT |
| | CCAGGGACTACGCGAGCTGGGCGAAAGGCCGATTCACCATCTCCAAAACCTCGA |
| | CCACGGTGGATCTGAGGATCATCAGTCCGACAACCGAGGACACGGCCACCTATT |
| | TTTGTGCCCGAAATGTTGGTGGTAGGCCTAATTTGTGGGGCCAGGGCACCCTGG |
| | TCACCGTCTCGAGC |
| 11H1-11K1 V_{L} | GCCTATGATGTGACCCAGACTCCAGCCTCTGTGGAGGCAGCTGTGGGAGGCACA |
| (SEQ ID NO: 218) | GTCACCATCAAGTGCCAGGTCAGTCAGAGCGTTAGCAATTGGTTAGCCTGGTAT |
| | CAGCAGAAACCAGGGCAGCCTCCCAAGCTCCTGATCTATTCTGCATCCACTCTGG |
| | CATCTGGGGTCCCATCGCGGTTCAAAGGCAGTGGATCTGGGACACAGTTCACTC |
| | TCACCATCAGCGACCTGGAGTGTGCCGATGCTGCCACTTATTATTGTCAACAGGC |
| | TTATAGTGGTAGGAATGTTGATAATGTTTTCGGCGGAGGGACCGAGGTGGTGGT |
| | CAAA |
| 12H3-12K2 V_{H} | CAGTCGCTGGAGGAGTCCGGGGGTCGCCTGGTCACGCCTGGGACACCCCTGAC |
| (SEQ ID NO: 196) | ACTCACCTGCGCAGCCTCTGGATTCTCCCTCAGTAGCAACTACATCAGCTGGGTC |
| | CGCCAGGCTCCAGGGGAGGGGCTGGAGTGGGTCGGATATGTAGATACTAGTGG |
| | CCGCTCATATTACGCGACCTGGGCAAAAGGCCGATTCACCATCTCCAGAACCTCG |
| | ACCACGGTGGATCTGAAAATCACCAGTCCGACAAGCGAGGACACGGCCACCTAT |
| | TTTTGTGTTAGGAGTGGTGGCTGTATGGACTTTGACTTGTGGGGCCAGGGCACC |
| | CTGGTCACCGTCTCGAGC |
| 12H3-12K2 V_{L} | GCCCAAGTGCTGACCCAGACTCCATCCTCCGTGTCTACAACTGTGGGAGGCACA |
| (SEQ ID NO: 219) | GTCAGCATCAGTTGCCAGTCCACTAAGAGTGTTTGGAGTAAGAACTGCTTAGTCT |
| | GGTATCAGCAGAAACCAGGGCAGCCTCCCAAGCGCCTGATTTACCAGGCATCGA |
| | AACTGGCATCTGGGGTCCCGTCGCGGTTCAAAGGCAGTGGATCTGGGACACAGT |
| | TCACTCTCACCATCGGCGACGTGCAGTGTGACGATGCTGCCACTTACTACTGTGC |
| | AGGCGCTTATTATGATAGTAGTGATAATGGTTTCGGCGGAGGGACCGAGGTGG |
| | TGGTCAAA |
| 13H1-13K2 V_{H} | CAGTCGCTGGAGGAGTCCGGGGGTCGCCTGGTCACGCCTGGGACACCGCTGAC |
| (SEQ ID NO: 197) | ACTCACCTGCACAGCCTCTGGATTCACCATCAGTAACTACCACATGAGCTGGGTC |
| | CGCCAGGCTCCAGGGAAGGGGCTGGAGTGGATCGGATACATTGTTGCTGGTAG |
| | TGATGTCGCATACTACGCGAGCTGGGCGAGAGGCCGATTCACCATCTCCAAAAC |
| | CTCGTCGACCACGGTGGATCTGACAATCACCAGGCCGACAACCGAGGACACGGC |
| | CACCTATTTCTGTGTCAGAGATGTTGCTGGTACTACTGGTTCTGACTTGTGGGGC |
| | CAGGGCACCCTGGTCACCGTCTCGAGC |
| 13H1-13K2 V_{L} | GCAGCCGTGCTGACCCAGACACCATCGCCCGTGTCTGCAGCTGTGGGAGGCACA |
| (SEQ ID NO: 220) | GTCACCATCAAGTGCCAGTCCAGTCAGAGTGTTTATAATAAAAACCTCTTATCCT |
| | GGTATCAGCAGAAACCCGGGCAGCCTCCCAGGGTCCTGATCTACAAGGCATCCA |
| | CTCTGGCATCTGGGGTCCCATCACGGTTCAGCGGCAGTGGGTCTGGGACACAGT |
| | TCACTCTCACCATCCGCGGCGTGCAGTGTGACGATGCTGCCACTTACTACTGTCT |
| | AGGCGCTTATGATAATGATGCTGATACGGCGTTCGGCGGAGGGACCGAGGTGG |
| | TGGTCAAA |
| 14H1-14K2 V_{H} | CAGTCGGTGGAGGAGTCCGGGGGTCGCCTGGTCACGCCTGGGACACCCCTGAC |
| (SEQ ID NO: 198) | ACTCACCTGCACAGTCTCTGGCTTCTCCCTCAGTAGCAGCTACATGAGCTGGGTC |
| | CGCCAGGCTCCAGGGAAGGGGCTGGAATGGATCGGATATATTGATACTGCTGTT |
| | GCCACTTACTTCGCGAGCTGGGCAAAAGGCCGATTCACCATCTCCAAAACCTCGA |
| | CCACGGTGGATCTGAAAATCACCAGTCCGACAAGCGAGGACACGGCCACGTATT |
| | TCTGTGTCAGGAGTGATGACTGTATGGCCTTTAACTTCTGGGGCCAGGGCACCCT |
| | GGTCACCGTCTCGAGC |
| 14H1-14K2 V_{L} | GCCGCCGTGCTGACCCAGACTCCAGCCTCCGTGTCTGCAGCTGTGGGAGGCACA |
| (SEQ ID NO: 221) | GTCACCATCAGTTGCCAGTCCACCAGGAGTGTTTGGCGTAACAACTGCTTAGCCT |
| | GGTATCAGCAGAAACCAGGGCAGCCTCCCAAGCTCCTGATCTACAGGGCATCCA |
| | GTCTGGCATCTGGGGTCCCATCGCGATTCAGAGGCAGTGGATCTGGGACACAGT |
| | TCACTCTCACCATCGGCGACGTGCAGTGTGACGATGCTGCCACTTACTACTGTGC |
| | AGGCGGGTATAGTGATGCACGTGATAATGGTTTCGGCGGAGGGACCGAGGTGG |
| | TGGTCAAA |
| 15H3-15K2 V_{H} | CAGTCGCTGGAGGAGTCCGGGGGTCGCCTGGTCACGCCTGGGACACCCCTGAC |
| (SEQ ID NO: 199) | ACTCACCTGCACAGTCTCTGGCTTCTCCCTCAGTACCGCCTACATTAGTTGGGTCC |
| | GCCAGGCTCCAGGGGAGGGGCTGCAATGGATCGGATATATTCATCTTGGTCGTA |
| | GTACATACTACGCGAGCTGGGCGCAAGGCCGATTCACCATCTCCAAAACCTCGTC |
| | GACCACGGTGGATCTGAAAATCACCAGTCCGACAAGCGAGGACACGGCCACCTA |
| | TTTCTGTGTCAGGAGTGGTGACTGTATGGCCTTTGATTTGTGGGGCCAGGGCAC |
| | CCTGGTCACCGTCTCGAGC |
| 15H3-15K2 V1 | GCCGCCGTGCTGACCCAGACTCCATCCCCCGTGTCTGCAGCTGTGGGAGGCACA |
| (SEQ ID NO: 222) | GTCAGCATCAGTTGCCAGTCCACTAAGAGTGTTTATAGTAAGAACTGCTTAGCCT |
| | GGTATCAGCAGAAACCAGGGCAGCCTCCCAAGCTCCTGATCTACAAGGCTTCCA |
| | CTCTGGCATCTGGGGTCCCATCGCGGTTCAAAGGCAGTGGATCTGGGACACAGT |
| | TCACTCTCACCATCGGCGACGTGCAGTGTGACGATGTTGCCACTTACTACTGTGC |
| | AGGCGGCTATAGTGATGCTCGAGATAATGCTTTCGGCGGAGGGACCGAGGTGG |
| | TGGTCAAA |
| 16H2-16K3 V_{H} | CAGTCGGTGGAGGAGTCCGGGGGTCGCCTGGTCACGCCTGGGACACCCCTGAC |
| (SEQ ID NO: 200) | ACTCACCTGCACAGCCTCTGGATTCACCATCAGTACCTACCACATGAGCTGGGTC |
| | CGCCAGGCTCCAGGGAAGGGGCTGGAATGGATCGGATACATTAATGCTAATAG |
| | TGGTGGCACATGGTACGCGAGCTGGGCGAAAGGCCGATTCACCATCTCCAAAAC |
| | CTCGACCACGGTGAATCTGAAAATCACCAGTCCGACAACCGAGGACACGGCCAC |
| | CTATTTCTGTGTCAGAAGTGGTTATAATAGTGGTATGGCTTTTGATCCCTGGGGC |
| | CAGGGCACCCTGGTCACCGTCTCGAGC |
| 16H2-16K3 V_{L} | GCCGCCGTGCTGACCCAGACTCCATCTCCCGTGTCTGCAGCTGTGGGAGGCACA |
| (SEQ ID NO: 223) | GTCAGCATCAGTTGCCAGTCCAGTAAGAGTGTTTATAATAACAACAATTTAGCCT |
| | GGTTTCAGCAGAAACCAGGGCAGCCTCCCAAGCTCCTGATCTATGGTGCATCCAT |
| | GCTGGCATCTGGGGTCCCATCGCGATTCAAAGGCAGTGGATCTGGGACGCAGTT |
| | CACTCTCACCATCAGTGACGTGCAGTGTGACGATGCTGCCACTTACTACTGTGCA |
| | GGCGCTTTTAGTAGTGCCAGTGACAATGCTTTCGGCGGAGGGACCGAGGTGGT |
| | GGTCAAA |
| 17H1-17K2 V_{H} | CAGGAGCAGCTGGGGGAGTCCGGAGGAGGCCTGGTCCAGCCTGGGGGAACCC |
| (SEQ ID NO: 201) | TGAAACTCTCCTGCAAAGGCTCTGGATTCGACCTCAGTAGCAATGCAATGTGCTG |
| | GGTCCGCCAGGCTCCAGGGAAGGGGCTGGAGTGGGTCGGATGCATTATTTATG |
| | GTAGCACATACTACGCGACCTGGGTGAATGGCCGATTCACTCTCTCCAGAGACA |
| | ACGCCCAGAGCTCGGTGGATCTGCAACTGAGCAGTCTGACAGCCGCGGACACG |
| | GCCACCTATTTCTGTGCCAGAGGGGGGCGTAGTTATGGTGATGGTTATGCTTTTG |
| | GATACTTTAACATCTGGGGCCAGGGCACCCTGGTCACCGTCTCGAGC |
| 17H1-17K2 V_{L} | GCCCTTGTGATGACCCAGACTCCGTCCTCCGTGTCTGCAGCTGTGGGAGGCACA |
| (SEQ ID NO: 224) | GTCACCATCAATTGCCAGGCCAGTCAGAACATTTACAGCAATTTAGCCTGGTATC |
| | AACAGAAACCAGGGCAGCGTCCCAAGCTCCTGATCTATGGTACATCCAATCTGG |
| | AATCTGGGGTCCCATCGCGGTTCAAAGGCAGTGGATCTGGGACAGAGTACACTC |
| | TCACCATCAGCGACCTGGAGTGTGACGATGCCGCCACTTACTACTGTCAAAGTGC |
| | TTATTATAGTAGTAGTACTGATATGGCTAATGCTTTCGGCGGAGGGACCGAGGT |
| | GGTGGTCAAA |
| 18H2-18K3 V_{H} | CAGGAGCAGCTGGAGGAGTCCGGGGGTCGCCTGGGCACGCCTGGGACACCCCT |
| (SEQ ID NO: 202) | GACACTCACCTGCACAGTCTCTGGATTCTCCCTCATTGGCCAATATATGTCCTGG |
| | GTCCGCCAGGCTCCAGGGAAGGGGCTGGAATGGATCGGATATATTGATACTACT |
| | GAAAGGTCATACTACGCGAGCTGGGCGAAAGGCCGATTCACCATCTCCAAAACC |
| | TCGTCGACCACGGTGGATCTGAAAATGACCAGTCTGACAACCGAGGACACGGCC |
| | ACCTATTTTTGTGTCAGATCCGATAGTTGTATGGCGTTTGGCTTATGGGGCCAGG |
| | GCACCCTGGTCACCGTCTCGAGC |
| 18H2-18K3 V_{L} | GTCCAAGTGCTGACCCAGACTCCATCCTCCGTGTCTGCAGCTGTGGGAGGCACA |
| (SEQ ID NO: 225) | GTCACCATGAGTTGCCAGTCCACTAAGAGTGTTTGGAATAATAATTGTTTAGCCT |
| | GGTATCAGCAGAAACCAGGGCAGCCTCCCAAGCTCCTGATCTACAAGGCTTCCA |
| | CTCTGGCAGCTGGGGTCTCATCGCGGTTCAAAGGCAATGGATCTGGGACACAGT |
| | CCACTCTCACCATCAGCGACGTGCAGTGTGACGATGCTGCCACTTACTACTGTGC |
| | AGGCGGTTATAGTACTAGTAGTGATAATGGTTTTGGCGGAGGGACCGAGGTGG |
| | TGGTCAAA |
| 22H2-22K1 V_{H} | CAGTCCGGAGGAGGAGGCGAAGGAGGCCTGGTCAAGCCTGGGGGATCCCTGG |
| (SEQ ID NO: 203) | AACTCTGCTGCAAGGCCTCTGGATTCTCTCTCAATAACAAATTCTGGATATGGTG |
| | GGTCCGCCAGGCTCCAGGGAAGGGGCTGGAGTGGATTGGAAGCATTGATGCTG |
| | GTGGTAGTGGTGGCACTTACTACGCGAGCTGGGTGAATGGCCGATTCACTCTCT |
| | CCAGAGACATCGACCAGAGCACTTATTGCCTACAACTGAACAGTCTGACAGCCG |
| | CGGACACGGCCATATATTACTGTGCGAGAGATTTAGCGGCTGCTGGTGGTGGTC |
| | TAACGGGCGCCTTTAACTTGTGGGGCCAGGGCACCCTGGTCACCGTCTCGAGC |
| 22H2-22K1 V_{L} | GCCTATGATATGACCCAGACTCCAGCCTCCGTGTCTGCAGCTGTGGGAGGCACA |
| (SEQ ID NO: 226) | GTCACCATCAATTGCCAGGCCAGTCAGAGCATTAACAACCTCTTAGCCTGGTATC |
| | AGCAGAAACCAGGGCAGCCTCCCAAGCTCCTGATCTACAAGGCATCCACTCTGG |
| | CATCTGGGGTCCCATCGCGGTTCAAAGGCAGTGGATCTGGGACACAGTTCACTC |
| | TCACCATCAGCGGCGTGCAGTGTGCCGATGCTGCCACTTACTATTGTCAACAGG |
| | GTTGGAGTGGTAGTAATGTTGATAATGTTTTCGGCGGAGGGACCGAGGTGGTG |
| | GTCAAA |
| 23H4-23K4 V_{H} | CAGTCGTTGGAGGAGTCCGGGGGAGGCCTGGTCCAGCCTGAGGGATCCCTGAC |
| (SEQ ID NO: 204) | ACTCACCTGCACAGCTTCTGGATTCTCCTTCAGTAGCAGCTACTACATGTGCTGG |
| | GTCCGCCAGGCTCCAGGGAAGGGGCTGGAGTGGATCGCATGCATTTATGTTGG |
| | CAGTAATAATAATTCTTACTACGCGAGCTGGGCGAAAGGCCGATTCGCCATCTCC |
| | AAAACCTCGTCGACCACGGTGACTCTGCAAATGGGCAGTCTGACAGCCGCGGAC |
| | ACGGCCACCTATTTCTGTGCGAGGAGTATTGTTGGTTATGCTGGTTATACTTATG |
| | CTCGGTACAATGCTTTTGATCCCTGGGGCCAGGGCACCCTGGTCACCGTCTCGAG |
| | C |
| 23H4-23K4 V_{L} | GCCCTTGTGATGACCCAGACTCCATCCTCCGTGTCTGGAGCTGTGGGAGGCACA |
| (SEQ ID NO: 227) | GTCACCATCAATTGCCAGGCCAGTCAGAACATTTACAACAATTTAGCCTGGTATC |
| | AGCAGAAACCAGGGCAGCCTCCCAAGCTCCTGATCTATGCTGCATCCAATCTGGC |
| | ATCTGGGGTCTCATCAAGGTTCAAAGGCAGTGGATCTGGGACAGAGTACACTCT |
| | CACCATCAGCGACCTGGAGTGTGACGATGCTGCCACTTACTACTGTCAAAGTGTT |
| | GCTTATAGTACTGGTGCTGCTACGATCGCTTTCGGCGGAGGGACCGAGGTGGTG |
| | GTCAAA |
| 25H4-25K1 V_{H} | CAGGAGCAACTGGTGGAGTCCGGGGGAGGCCTGGTCAAGCCTGGGGCATCCCT |
| (SEQ ID NO: 205) | GACACTCACCTGCAAAGCCTCTGGATTCTCCTTCAGTAGCGGCTACTACATGTGC |
| | TGGGTCCGCCAGGCTCCAGGGAAGGGGCTGGAGTGGATCGGATGCATTCTTCC |
| | CGGCACTGTGGCCTCTTACTACGCGAGCTGGGCGAAAGGCCGGTCCACCATCTC |
| | CAAAGCCTCGTCGACCACGGTGGATCTGAAAATGACCAGGCTGACAGCGGCGG |
| | ATACGGCCACTTATTTCTGTGCGAGAGTTAATCCTACTTATGGTGGCCATGGATA |
| | TGACTTGTGGGGCCAGGGCACCCTGGTCACCGTCTCGAGC |
| 25H4-25K1 V_{L} | GCCGCCGTGCTGACCCAGACTCCATCTCCCGTGTCTGCAGCTGTGGGAGGCACA |
| (SEQ ID NO: 228) | GTCAGCATCAGTTGCCAGTCCAGTAAGAGTGTTCATAATAATAACCACTTAGCCT |
| | GGTTTCAGCAGAAACCAGGGCAGCCTCCCAAGCTCCTGATCTACAAGGCTTCCAC |
| | TCTGGCATCTGGGGTCCCATCGCGGTTCAAAGGCAGTGGATCTGGGACACAGTT |
| | CACTCTCACCATCAGCGACGTGCAGTGTGACGATGCTGCCACTTACTACTGTGCA |
| | GGCGCTTTCATTTCTAGTGGTGATAATGGCTTCGGCGGAGGGACCGAGGTGGTG |
| | GTCAAA |
| 26H6-26K5 V_{H} | CAGGAGCAGCTGGTGGAGTCCGGGGGAGGCCTGGTCCAGCCTGGGGCATCCCT |
| (SEQ ID NO: 206) | GACACTCACCTGCAAAGCCTCTGGATTCTCCGTCAGTAGCGGCGTTTACATGTGC |
| | TGGGTCCGCCAAGCTCCAGGGAAGGGGCTGGAGTGGATCGGATGCATTCTTACT |
| | GGCAGTGTTACCTCTTACTACGCGACCTGGGTGAATGGCCGATTCACCCTCTCCA |
| | AAGCCTCGTCGACCACACTATATCTACAACTGAACAGTCTGACAGTCGCGGACAC |
| | GGCCACCCATTTCTGTGTAAGAGTTAATCCCAATTATGGTGGCCATGGTTATGAC |
| | TTGTGGGGCCAGGGCACCCTGGTCACCGTCTCGAGC |
| 26H6-26K5 V_{L} | GCCCAAGTGCTGACCCAGACTCCAGCCTCCGTGTCTGCAGCTGTGGGAGGCACA |
| (SEQ ID NO: 229) | GTCAGCATCAGTTGCCAGTCCAGTAAGAGTGTTTATGCTCGCAACCACTTAGCCT |
| | GGTTTCAGCAGAAACCAGGGCAGCCTCCCAAGCTCCTGATCTACAAGGCATCCA |
| | ACCTGCCAACTGGGGTCCCATCGCGGTTCAGCGGCAGTGGATCTGGGACACAGT |
| | TCACTCTCACCATCAGCGACGTGCAGTGTGACGATGCTGCCACGTACTACTGTGC |
| | AGGCGCTTTCAGTACTAGTGGTGATAATGGTTTCGGCGGAGGGACCGAGGTGG |
| | TGGTCAAA |
| 27H6-27K5 V_{H} | CGGTCGTTGGAGGAGTCCGGGGGAGGCCTGGTCAAGCCTGGAGAAACCCTGAC |
| (SEQ ID NO: 207) | ACTCACCTGCAAAGCCTCTGGATTCGACTTCACTAGCAGTGCAGCGTGCTGGGTC |
| | CGCCAGGCTCCAGGGAAGGGGCTGGAGTGGATCGGATGTATACTTAATAGTAG |
| | GATCTATTATGCGGCCTGGGCGAAAGGCCGATTCACCATTTCCAGGGCCTCGTC |
| | GATCACGGTGACTCTACGAATGACCAGTCTGACAGCCGCGGACACGGCCACCTA |
| | TTTCTGTGCGAGAGTCCCTGGTGGCAGTCCTTATAATTGGTGGGCCATGGACCTC |
| | TGGGGCCAGGGCACCCTGGTCACCGTCTCGAGC |
| 27H6-27K5 V_{L} | GCGCAAGTGCTGACCCAGACTCCATCGTCCGTGTCTGCAGCTGTGGGAGGCACA |
| (SEQ ID NO: 230) | GTCACCATCAATTGTCAGTCCAGTCAGAGCGTTGCTGGTACCAACTGGTTATCCT |
| | GGTATCAGCAGAAACCAGGGCAGCCTCCCAAGCTCCTGATCTACAAGGCATCCA |
| | TTCTGGAGGCCGGGGTCTCATCGCGGTTCAAAGGCAGTAGATCTGGGACGCAGT |
| | TCACTCTCACCATCAACGACATGCAGTGTGACGATGCTGCCACTTACTACTGTCA |
| | TGGCGCCTATAGTAGTGTTAATGTGATTCATACTTTCGGCGGAGGGACCGAGGT |
| | GGTGGTCAAA |

### Compositions

The present disclosure also provides compositions. According to some embodiments, a composition of the present disclosure includes any of the compounds, antibodies, nucleic acids, expression vectors, and/or cells of the present disclosure.

In certain aspects, a composition of the present disclosure includes any of the compounds, antibodies, nucleic acids, expression vectors, and/or cells of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

In certain embodiments, the compositions of the present disclosure find use as reagents in performing any of the immunoassays of the present disclosure, including any of the homogeneous enzyme immunoassays described herein. For example, a composition that includes any of the compounds of Formula 1-4 of the present disclosure, any of the antibodies of the present disclosure, or both, may be employed to perform such immunoassays. According to some embodiments, the reagents are provided in lyophilized form, e.g., to increase stability, convenience, and/or the like. As just one example, a composition that includes any of the compounds of Formula 1, 2, 3 or 4 of the present disclosure, any of the antibodies of the present disclosure, or both, may be provided in the form of lyophilized reagent spheres as described in U.S. Pat. No. 5,413,732, the disclosure of which is incorporated herein by reference in its entirety for all purposes. Briefly, such spheres may be made, e.g., by forming a homogeneous solution of a reagent; measuring uniform drops of the solution (e.g., 2 to 50 μL); dispensing the uniform, measured drops into an unagitated cryogenic liquid (e.g., liquid nitrogen), whereby the drops are frozen; collecting the frozen drops from the cryogenic liquid; and lyophilizing the frozen drops, thereby forming a plurality of lyophilized reagent spheres.

### Immunoassays

Aspects of the present disclosure further include methods of using any of the compounds of Formula 1, and 2 of the present disclosure and/or any of the antibodies of the present disclosure. In certain embodiments, such compounds and/or antibodies may be used for detecting (including determining an amount of) at least one BUP analyte and/or metabolites in a medium, e.g., a medium that includes a biological sample of interest. For example, according to some embodiments, provided are methods for determining an amount of at least one BUP analyte in a medium, the methods including combining in a medium a sample suspected of containing at least one BUP analyte, and any of the antibodies of the present disclosure. Such methods further include determining the presence or absence of a complex comprising the BUP analyte and/or metabolites and the antibodies, wherein the presence of the complex indicates the presence of the BUP analyte and/or metabolites in the sample. In certain embodiments, the medium further includes any of the compounds of Formula 1 and 2 of the present disclosure. For example, the medium may further include a BUP, NBUP, BUP-G and/or NBUP-G conjugate that has a BUP moiety and a detectable label (e.g., enzyme, such as G6PD).

The sample suspected of containing at least one BUP analyte and/or metabolites or a combination may be any sample of interest. In certain embodiments, the sample is whole blood, blood serum, blood plasma, urine, sputum, semen, saliva, ocular lens fluid, cerebral fluid, spinal fluid, amniotic fluid, tissue culture media, hair, bile, or the like, and including preanalytical steps like dilutions, alkali treatment or derivatizations thereof.

The present disclosure provides immunoassay methods for assessing the presence or absence at least one BUP analyte and/or metabolite NBUP, BUP-G and NBUP-G in a sample suspected of containing the analytes. Immunoassays of the present disclosure can be of a variety of formats. The immunoassays may be separation immunoassays (also known as heterogeneous immunoassays) or homogeneous immunoassays. Furthermore, the immunoassays may be qualitative or quantitative. Assays of this disclosure include both sandwich and competitive assays. The immunoassays may embody other types of assays that are neither sandwich nor competitive assays, as in certain assays involving immunoprecipitation. In certain embodiments, the immunoassay is a homogeneous immunoassay, where the assay reagents and sample are mixed together to form a homogeneous assay mixture.

In certain embodiments, the immunoassay is a homogeneous enzyme immunoassay system used for the analysis of at least one BUP analyte and/or metabolites in a biological fluid sample. In some instances, the immunoassay is based on competition between at least one BUP analyte and/or metabolites in the sample and labeled BUP, N-BUP, BUP-G and/or N-BUP-G for antibody binding sites. In some embodiments, the label is a protein, such as an enzyme. For example, the label may be an enzyme the activity of which may be measured spectrophotometrically. In one non-limiting example, an assay of the present disclosure employs BUP and/or acylated or alkylated BUP, NBUP, BUP-G and NBUP-G labeled with the enzyme glucose-6-phosphate dehydrogenase (G6PD) for antibody binding sites. In certain embodiments, enzyme activity decreases upon binding to the antibody, such that the concentration of the BUP analyte in the sample can be measured in terms of enzyme activity. In some cases, active enzyme converts nicotinamide adenine dinucleotide (NAD⁺) to NADH, resulting in an absorbance change that is measured spectrophotometrically. In certain instances, endogenous serum G6PD does not interfere with the immunoassay because the coenzyme NAD⁺ functions only with the bacterial (Leuconostoc mesenteroides) enzyme employed in the assay.

In general, the immunoassays of the present disclosure for detecting the presence (or absence) of at least one BUP analyte and/or metabolites NBUP, BUP-G and NBUP-G in a sample can be conducted by adding, to a reaction mixture, (i) a sample suspected of containing at least one BUP analyte and/or metabolites NBUP, BUP-G and NBUP-G and (ii) antibodies that specifically bind to at least one BUP analyte and/or metabolites NBUP, BUP-G and NBUP-G to form a complex between the antibody and BUP, NBUP, BUP-G and NBUP-G analyte that may be present in the sample. The method also includes detecting the presence or absence of the complex. The presence (or absence) of the complex may be indicative of the presence (or absence) of BUP analyte in the sample. Moreover, the amount of complex formed can be assessed to determine the concentration of BUP, NBUP, BUP-G and NBUP-G analyte present in the sample (e.g., to provide an assessment of serum or tissue concentration of BUP, NBUP, BUP-G and NBUP-G analyte in a subject from whom the sample was obtained). The presence and/or amount of complex can be assessed directly (e.g., by detecting bound antibody in the complex) or indirectly (e.g., by assessing activity of an enzyme in a BUP, NBUP, BUP-G and NBUP-G enzyme conjugate, where when the BUP, NBUP, BUP-G and NBUP-G enzyme conjugate is not bound to antibody, a detectable signal is generated, indicating that the BUP analyte antibody in the reaction mixture has been bound by BUP analyte from the sample.

In general, the immunoassays of the present disclosure entail combining in a medium (e.g., assay medium or assay reaction mixture), the sample with a BUP, NBUP, BUP-G and NBUP-G analyte antibody under conditions that permit the formation of a stable complex between the analyte in the sample and the antibody.

Assays may be performed in solution or may use a solid (insoluble) support (e.g., polystyrene, nitrocellulose, particles or beads), using any standard methods (e.g., as described in Current Protocols in Immunology, Coligan et al., ed.; John Wiley & Sons, New York, 1992). Such methods include ELISAs (enzyme-linked immunosorbent assays), IRMAs (immunoradiometric assays), and RIAs (radioimmunoassays). In certain embodiments, the assay is performed in solution, e.g., the assay is performed without the assay reagents attached to or associated with a solid support.

Where the assay is performed in solution, the test sample (and, optionally a control sample) may be incubated with an anti-BUP, anti-NBUP, anti-BUP-G and/or anti-NBUP-G analyte antibody for a time period sufficient to allow formation of analyte and affinity reagent complexes. As previously noted, the BUP, NBUP, BUP-G and NBUP-G analyte antibody may include a detectable label, e.g., radionuclide, fluorescer, or enzyme. The sample may then be treated to separate the BUP analyte antibody complexes from excess, unreacted BUP analyte antibody (e.g., by addition of a secondary antibody (e.g., anti-immunoglobulin antiserum)) followed by centrifugation to precipitate the secondary complexes, or by binding to an affinity surface such as a second, unlabeled antibody fixed to a solid substrate such as Sepharose® or a plastic well). Detection of BUP analyte antibody bound to a BUP, NBUP, BUP-G and NBUP-G analyte may be achieved in a variety of ways. If necessary, a substrate for the detectable label may be added to the sample.

Where the assay uses a solid support, the support can have a BUP analyte antibody (or conjugate) bound to a support surface. Binding of the assay reagent may facilitate the stable, wash-resistant binding of BUP, NBUP, BUP-G and NBUP-G analyte which may be present in the sample (or antibody that is not bound to BUP, NBUP, BUP-G and NBUP-G analyte from the sample, and is present in the reaction mixture, as in a competitive binding assay) to the solid support via specific binding to the antibody. The insoluble support may be any composition to which antibodies or suitable BUP, NBUP, BUP-G and NBUP-G conjugates can be bound, which can be separated from soluble material, and which is otherwise compatible with the overall method of detection of BUP, NBUP, BUP-G and NBUP-G analyte in a sample.

The surface of such supports may be solid or porous and of any convenient shape. Examples of suitable insoluble supports to which the BUP, NBUP, BUP-G and NBUP-G analyte antibody or conjugate is bound include beads, e.g., magnetic beads, fluorescent particles, membranes and microtiter plates. These can be composed of glass, plastic (e.g., polystyrene), polysaccharides, nylon or nitrocellulose.

Assay reagents can include the BUP, NBUP, BUP-G and NBUP-G analyte antibody as disclosed herein, as well as secondary antibodies, which may be optionally detectably labeled. After binding of an assay reagent to the support, the support may be treated with a blocking agent, which binds to the support in areas not occupied by the assay reagent. Suitable blocking agents include non-interfering proteins such as bovine serum albumin, casein, gelatin, and the like. Alternatively, detergents at non-interfering concentrations, such as Tween, NP40, TX100, and the like may be used. Such blocking treatment may reduce nonspecific binding.

Assays of the present disclosure include both qualitative and quantitative assays. Typical quantitative methods involve mixing an analyte with a pre-determined amount of the reagent antibody, and correlating the amount of complex formed with the amount of analyte in the original sample using a relationship determined using standard samples containing known amounts of analyte in the range expected for the sample to be tested. In a qualitative assay, sufficient complex above or below a threshold level established by samples known to contain or be free of analyte can be used to establish the assay result. Unless otherwise stated, the act of “measuring” or “determining” in this disclosure encompasses both qualitative and quantitative determination.

Immunoassay reagents that find use alone or in combination in the assays described herein include, but are not limited to, a BUP, NBUP, BUP-G and NBUP-G analyte antibody, a BUP, NBUP, BUP-G and NBUP-G conjugate, and a BUP analyte (e.g., as a control or in competitive binding assays). Immunoassay reagents can be provided in a buffered aqueous solution. Such solutions may include additional components such as surface active additives, organic solvents, defoamers, buffers, surfactants, and anti-microbial agents. Surface active additives can be introduced to maintain hydrophobic or low-solubility compounds in solution, and stabilize components in the solution. Examples include bulking agents such as β-lactoglobulin (BLG) or polyethyleneglycol (PEG); defoamers and surfactants such as Tween-20, Plurafac A38, Triton X-100, Pluronic 25R2, rabbit serum albumin (RSA), bovine serum albumin (BSA), and carbohydrates. Examples of organic solvents can include methanol and other alcohols. Various buffers may be used to maintain the pH of the solution during storage. Illustrative buffers include HEPES, borate, phosphate, carbonate, tris, barbital and the like. Anti-microbial agents also extend the storage life of the immunoassay reagent.

The BUP, NBUP, BUP-G and NBUP-G analogs haptens and conjugates and/or the BUP, NBUP, BUP-G and NBUP-G analog analyte antibodies to be used as reagents in an assay can be insolubilized by attachment to a solid support. This can be, for example, a wall of a vessel containing the reagent, to a particulate, or to a large molecular weight carrier that can be kept in suspension but removable by physicochemical means, such as centrifugation or microfiltration. In some cases, the attachment is through one or more covalent bonds. The attachment need not be covalent, but is at least of sufficient strength and/or permanence to withstand a separation technique (including a wash) that may be part of the assay procedure. In some cases, the solid support may be functionalized to include a reactive group to facilitate attachment of the BUP conjugate and/or the BUP analyte antibody to the solid support. Nonlimiting examples of reactive groups that may be used include —COOH, —NH₂, —C(O) H, —SH, and the like. In some embodiments, the BUP, NBUP, BUP-G and NBUP-G analog conjugate and/or the BUP, NBUP, BUP-G and NBUP-G analog analyte antibody can be conjugated to a protein carrier and the protein carrier can be conjugated to the solid support, thus indirectly attaching the BUP conjugate and/or the BUP analyte antibody to the solid support. Certain particulate materials include, but are not limited to, agarose, polystyrene, cellulose, polyacrylamide, latex particles, magnetic particles, and fixed red cells. Examples of commercially available matrices include, but are not limited to, Sepharose® (Pharmacia), Poros® resins (Roche Molecular Biochemicals), Actigel Superflow™ resins (Sterogene Bioseparations Inc.), and Dynabeads™ (Dynal Inc, Thermo Fisher Scientific). In certain embodiments, the choice of the solid support may depend on one or more of stability, capacity, accessibility of the coupled antibody, flow rate (or the ability to disperse the resin in the reaction mixture), and ease of separation.

As noted above, immunoassays for detection of at least one BUP analyte and/or metabolites can be of a variety of formats. In general, the immunoassays involve combining one or more immunoassay reagents (e.g., at least an anti-BUP analyte antibody) with a test sample (i.e., a sample suspected of containing at least one BUP analyte and/or metabolites) in a medium (e.g., a reaction mixture or an assay mixture). “Reaction mixture” or “assay mixture” generally refers to the combination of a sample suspected of containing at least one BUP analyte and/or metabolites NBUP, BUP-G and NBUP-G and one or more immunoassay reagents as exemplified in the present disclosure to facilitate detection of the presence or absence of at least one BUP analyte and/or metabolites in the sample, where the detection may be qualitative or quantitative. The reaction mixture is usually an aqueous solution, although the immunoassay reagent(s) may be in solution or immobilized on a support (e.g., a substrate, such as a bead). The reaction mixture can include other components compatible with the immunoassay, e.g., buffers, reagents, and the like.

Immunoassays usually are classified in one of several ways. For example, immunoassays can be classified according to the mode of detection used, i.e., enzyme immunoassays, radio immunoassays, fluorescence polarization immunoassays, chemiluminescence immunoassays, turbidimetric assays, etc. Another grouping method is according to the assay procedure used, i.e., competitive assay formats, sandwich-type assay formats as well as assays based on precipitation or agglutination principles. In certain instances, a further distinction is made depending on whether washing steps are included in the procedure (so-called heterogeneous assays) or whether reaction and detection are performed without a washing step (so-called homogeneous assays). Certain assays are described in more detail below.

Immunoassays may be described as heterogeneous or homogeneous. “Homogeneous immunoassay,” as used herein, refers to an assay method where the complex is not separated from unreacted reaction components, but instead the presence of the complex is detected by a property which at least one of the reactants acquires or loses as a result of being incorporated into the complex. Homogeneous assays include systems involving fluorochrome and fluorochrome quenching pairs on different reagents; enzyme and enzyme inhibitor pairs on different reagents; chromophore and chromophore modifier pairs on different reagents; and latex agglutination assays.

A certain homogeneous assay is the quantitative homogeneous enzyme immunoassay in which a BUP derivative of the current invention is conjugated to an active enzyme. In some embodiments, the conjugation is arranged so that the binding of at least one BUP analyte and/or metabolites antibody to BUP or derivative conjugate affects enzymatic activity of the conjugate in a qualitative or quantitative fashion. If a sample containing at least one BUP analyte and/or metabolites NBUP, BUP-G and NBUP-G is premixed with the antibody, the antibodies may complex with the BUP, NBUP, BUP-G and NBUP-G analytes and thus be prevented from binding to the enzyme conjugate. In this way, the activity of the enzyme in the conjugate can be correlated with the amount of BUP, NBUP, BUP-G and NBUP-G analyte present in the sample.

G6PD is a certain enzyme useful in such assays. In some embodiments, the G6PD is a variant of a naturally occurring G6PD in which one or more lysine residues are deleted or substituted, or one or more cysteine residues are introduced. For example, Leuconostoc mesenteroides G6PD are dimeric enzymes that have the ability to catalyze the oxidation of D-glucose-6-phosphate to D-glucono-delta-lactone-6-phosphate by utilizing either NAD⁺ or NADP⁺. This property of using NAD⁺ differentiates these enzymes from human G6PD, which utilizes only NADP⁺ effectively, and allows L. mesenteroides-specific G6PD activity to be measured in the presence of human G6PD, as for example in human-derived samples. Two certain genera of bacteria from which to select G6PD are Leuconostoc and Zymomonas. Within these genera L. mesenteroides, L. citreum, L. lactis, L. dextranicum, and Z. mobilis are of interest, where L. mesenteroides, L. citreum, and L. lactis are specific examples.

Another example of a homogeneous assay system is the cloned enzyme donor immunoassay (CEDIA). The CEDIA Buprenorphine Assay uses recombinant DNA technology (U.S. Pat. No. 4,708,929) to produce a unique homogeneous enzyme immunoassay system. The assay is based on the bacterial enzyme-galactosidase, which has been genetically engineered into two inactive fragments. These fragments spontaneously re-associate to form fully active enzymes that, in the assay format, cleave a substrate, generating a color change that can be measured spectrophotometrically. In the assay, analyte in the sample competes with analyte conjugated to one inactive fragment (enzyme donor) of -galactosidase for a limited number of antibody binding sites. If analyte is present in the sample, it binds to antibody, leaving the inactive enzyme fragment free to form active enzyme. If the analyte is not present in the sample, antibody binds to analyte conjugated on the inactive fragment, inhibiting the re-association of inactive -galactosidase fragments, and no active enzyme is formed. The amount of active enzyme formed and resultant absorbance change are directly proportional to the amount of analyte present in the sample. This assay does not detect any glucuronides as the antibodies are specific for buprnoprphnie and norbuprenorphine only.

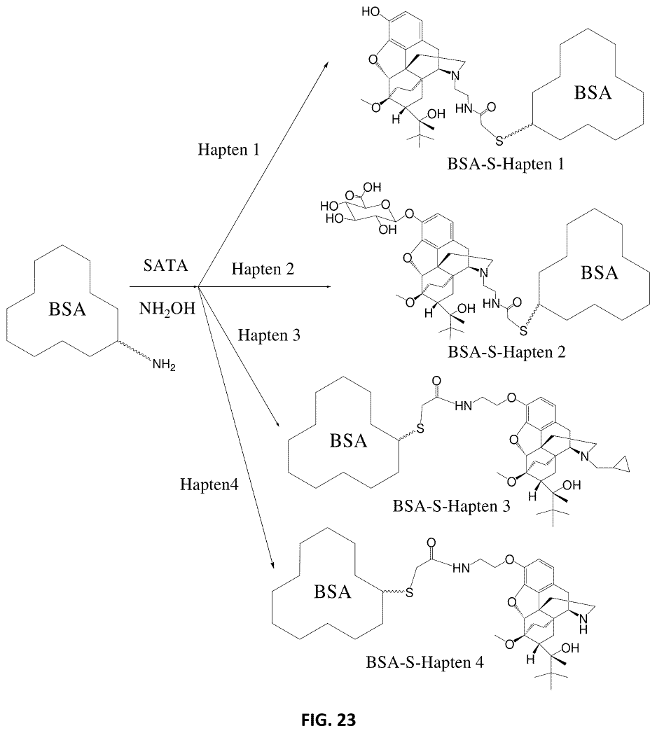

BUP, NBUP, BUP-G and NBUP-G derivatives with thiol reactive groups can be prepared as described above, and may be allowed to react with a glucose-6-phosphate dehydrogenase (G6PD) mutant enzyme to form the respective enzyme conjugates (see, e.g., FIGS. 23-25). The mutant enzyme may be obtained by the procedure described in U.S. Pat. Nos. 6,090,567 and 6,033,890, the disclosures of which are incorporated herein by reference.

In some embodiments, the immunoassay further includes adding a BUP, NBUP, BUP-G and/or NBUP-G analog conjugate that has a BUP moiety and a detectable label to the sample. The presence or absence of BUP or its metabolites NBUP, BUP-G and NBUP-G analytes in the sample can be detected by detecting the detectable label. The detectable label may include an enzyme and the detecting may be performed by assaying activity of the enzyme. In certain embodiments, the enzyme is a dehydrogenase, such as G6PD.

Luminescence oxygen channeling assay (LOCI) is a chemiluminescence homogeneous immunoassay whereby a biotinylated analyte competes with the analyte for antibody binding on the acceptor particles. The biotinylated analyte is bound to the donor particles through streptavidin present on the donor particles. In the presence of the analyte, the two particles come into close proximity through the biotin-analyte binding interactions. The excitation of the donor beads at 680 nm generates singlet oxygen molecules that trigger a series of chemical reactions in the LOCI acceptor beads resulting in a detectable peak of light emission at 615 nm (Ullman, E. F. et al. (1996) Clin. Chem. 42, 1518-1526). In the presence of the analyte the relative light units (RLUS) emitted is inversely proportional to the concentration of the analyte.

In a separation-based or “heterogeneous” assay, the detection of a complex of at least one BUP analyte and/or metabolites antibodies and an analyte involves a process where the complex formed is physically separated from either unreacted analyte, unreacted antibody, or both.

In a heterogeneous immunoassay, a complex of an antibody and a BUP analyte may be first formed in the fluid phase, and then subsequently captured by a solid phase reagent or separated on the basis of an altered physical or chemical property, such as by gel filtration or precipitation. Alternatively, one of the reagents may be attached to a solid phase before contacting with other reagents, and then the complex may be recovered by washing the solid phase free of unreacted reagents. Separation-based assays typically involve use of a labeled derivative or labeled antibody to facilitate detection or quantitation of the complex. Suitable labels include radioisotopes such as ¹²⁵I, enzymes such as peroxidase and β-galactosidase, and fluorescent labels such as fluorescein isothiocyanate. The separation step involves removing labeled reagent present in complex form from unreacted labeled reagent. The amount of label in the complex can be measured directly or inferred from the amount left unreacted.

Assays of the present disclosure include both sandwich and competition assays. Sandwich assays typically involve forming a complex in which the analyte to be measured is sandwiched between one reagent, such as a first antibody used ultimately for separation of the complex, and another reagent, such as a second antibody used as a marker for the separated complex. Competition assays involve a system in which the analyte to be measured competes with a derivative of the analyte for binding to another reagent, such as an antibody. An example of a competition assay using EMIT® is described in U.S. Pat. No. 3,817,837. Immunoassay design for screening drugs of abuse are described in Datta, Pradip. (2019). Critical Issues in Alcohol and Drugs of Abuse Testing 121-128. doi: 10.1016/B978-0-12-815607-0.00009-5.

The compounds and methods of the presently disclosed embodiments also encompass the use of these materials in lateral flow chromatography technologies. Lateral flow chromatography involves a membrane strip which includes a detection device, such as a non-isotopic signal generating moiety, for BUP and/or its metabolites NBUP, BUP-G and NBUP-G analytes. A sample from a patient may then be applied to the membrane strip. The sample may interact with the detection device, producing a result. The results can signify several things, including the absence of the BUP and/or its metabolites NBUP, BUP-G and NBUP-G analytes in the sample, the presence of BUP and/or its metabolites NBUP, BUP-G and NBUP-G analytes in the sample, and/or the concentration of the BUP and/or its metabolites NBUP, BUP-G and NBUP-G analytes in the sample.

Certain embodiments provide a method of qualitatively determining the presence or absence of a BUP and/or its metabolites NBUP, BUP-G and NBUP-G analytes in a sample, through the use of lateral flow chromatography. In certain embodiments, the basic design of the qualitative lateral flow device is as follows: 1) The sample pad is where the sample is applied. The sample pad is treated with chemicals such as buffers or salts, which, when re-dissolved, optimize the chemistry of the sample for reaction with the conjugate, test, and control reagents; 2) Conjugate release pad is typically a polyester or glass fiber material that is treated with a conjugate reagent such as an antibody colloidal gold conjugate. A typical process for treating a conjugate pad is to use impregnation followed by drying. In use, the liquid sample added to the test will re-dissolve the conjugate so that it will flow into the membrane; 3) The membrane substrate is usually made of nitrocellulose or a similar material whereby antibody capture components are immobilized; 4) A wicking pad is used in tests where blood plasma must be separated from whole blood. An impregnation process is usually used to treat this pad with reagents intended to condition the sample and promote cell separation; 5) The absorbent pad acts as a reservoir for collecting fluids that have flowed through the device; and 6) The above layers and membrane system are laminated onto a plastic backing with adhesive material which serves as a structural member.

Certain embodiments provide a method of qualitatively determining the presence of a BUP analyte in a sample, through the use of lateral flow chromatography. In these embodiments, the membrane strip includes a sample pad, which is a conjugate release pad that has an antibody that is specific for the BUP and/or its metabolites NBUP, BUP-G and NBUP-G analytes. This antibody may be conjugated to a non-isotopic signal-generating moiety, such as a colloidal gold particle. Other detection moieties useful in a lateral flow chromatography environment include dyes, colored latex particles, fluorescently labeled latex particles, non-isotopic signal generating moieties such as gold or quantum dots, etc. In some instances, the membrane strip further includes a capture line, in which the BUP analyte antigen or analyte conjugate is immobilized on the strip. In some embodiments, this immobilization is through covalent attachment to the membrane strip, optionally through a linking group. In other embodiments, the immobilization is through non-covalent attachment to the membrane strip. In still other embodiments, the immobile BUP analyte in the capture line is attached to a reactive partner, such as an immunogenic carrier like BSA.

Sample from a patient may be applied to the sample pad, where it can combine with the antibody in the conjugate release pad, thus forming a solution. This solution may then migrate chromatographically by capillary action across the membrane. When BUP and/or its metabolites NBUP, BUP-G and NBUP-G analytes are present in the sample, a BUP and/or its metabolites NBUP, BUP-G and NBUP-G analyte antibody complex may be formed, which migrates across the membrane by capillary action. When the solution reaches the capture line, the BUP, NBUP, BUP-G and NBUP-G analyte antibody complex may compete with the immobile BUP and/or its metabolites NBUP, BUP-G and NBUP-G analytes for the limited binding sites of the antibody. When a sufficient concentration of BUP, NBUP, BUP-G and/or NBUP-G analyte is present in the sample, it may fill the limited antibody binding sites. In certain instances, this will prevent the formation of a colored antibody-immobile BUP, NBUP, BUP-G and NBUP-G analyte complex in the capture line. Therefore, absence of color in the capture line indicates the presence of BUP and/or its metabolites NBUP, BUP-G, NBUP-G analytes in the sample.

In the absence of BUP, NBUP, BUP-G and NBUP-G analyte in the sample, a colored antibody-immobile BUP, NBUP, BUP-G and/or NBUP-G analyte complexes may form once the solution reaches the capture line of the membrane strip. In some instances, the formation of this complex in the capture line is evidence of the absence of BUP, NBUP, BUP-G and/or NBUP-G analyte in the sample.

Certain embodiments provide a method of quantitatively determining the amount of a BUP analyte in a sample, through the use of lateral flow chromatography. This technology is further described in U.S. Pat. Nos. 4,391,904; 4,435,504; 4,959,324; 5,264,180; 5,340,539; and 5,416,000, 8,399,261, the disclosures of which are incorporated herein by reference. In some embodiments, the antibody may be immobilized along the entire length of the membrane strip. In general, if the membrane strip is made from paper, the antibody may be covalently bound to the membrane strip. If the membrane strip is made from nitrocellulose, then the antibody can be non-covalently attached to the membrane strip through, for example, hydrophobic and electrostatic interactions. The membrane strip may include a conjugate release pad that includes the BUP analyte attached to a detector moiety. In certain embodiments, the detector moiety is an enzyme, such as horseradish peroxidase (HRP).

In certain embodiments, sample from a patient is applied to the membrane strip, where it can combine with the BUP, NBUP, BUP-G and/or NBUP-G analyte/detector molecule in the conjugate release pad, thus forming a solution. This solution may then be allowed to migrate chromatographically by capillary action across the membrane. When BUP, NBUP, BUP-G and NBUP_G analyte is present in the sample, both the sample BUP, NBUP, BUP-G and NBUP-G analytes and the BUP, NBUP, BUP-G and/or NBUP-G analyte/detector molecule compete for the limited number of binding sites of the antibody. When a sufficient concentration of BUP, NBUP, BUP-G and NBUP-G analyte is present in the sample, it may fill the limited antibody binding sites. In some instances, this forces the BUP analyte/detector molecule to continue to migrate in the membrane strip. The shorter the distance of migration of the BUP, NBUP, BUP-G and NBUP-G analyte/detector molecule in the membrane strip, the lower the concentration of BUP, NBUP, BUP-G and/or NBUP-G analyte in the sample, and vice versa. When the BUP, NBUP, BUP-G and NBUP-G analyte/detector molecule includes an enzyme, the length of migration of the BUP, NBUP, BUP-G and NBUP-G analyte/detector molecule can be detected by applying an enzyme substrate to the membrane strip. Detection of the product of the enzyme reaction may then be utilized to determine the concentration of the BUP, NBUP, BUP-G and NBUP-G analyte in the sample. In certain embodiments, the enzyme's color producing substrate such as a modified N,N-dimethylaniline is immobilized to the membrane strip and 3-methyl-2-benzothiazolinone hydrazone is passively applied to the membrane, thus alleviating the need for a separate reagent to visualize the color producing reaction.

Fluorescence polarization immunoassay (FPIA) technology is based upon competitive binding. FPIA technology is described in, for example, U.S. Pat. Nos. 4,593,089, 4,492,762, 4,668,640, and 4,751,190, the disclosures of which are incorporated herein by reference.

The FPIA technology can be used to identify the presence of BUP, NBUP, BUP-G and NBUP-G analyte and can be used in assays that quantify the amount of BUP, NBUP, BUP-G and NBUP-G analyte in a sample. In part, the rotational properties of molecules in solution allow for the degree of polarization to be directly proportional to the size of the molecule. Accordingly, polarization may increase as molecular size increases. That is, when linearly polarized light is used to excite a fluorescent-labeled or other luminescent-labeled BUP analyte thereof, which is small and rotates rapidly in solution, the emitted light may be significantly depolarized. When the fluorescent-labeled BUP analyte interacts with or is bound to an antibody, the rotation may be slowed and the emitted light may be highly polarized. In some cases, this is because the antibody significantly and measurably increases the size of the complex. Also, increasing the amount of unlabeled BUP, NBUP, BUP-G and NBUP-G analyte in the sample can result in decreased binding of the fluorescent-labeled BUP, NBUP, BUP-G and NBUP-G analyte by the BUP, NBUP, BUP-G and NBUP-G analyte antibody, and thereby decrease the polarization of light emitted from sample. The quantitative relationship between polarization and concentration of the unlabeled BUP, NBUP, BUP-G and NBUP-G analyte in the sample can be established by measuring the polarization values of calibrations with known concentrations of BUP, NBUP, BUP-G and NBUP-G analyte. Thus, FPIA can be used to identify the presence and concentration of BUP, NBUP, BUP-G and NBUP-G analyte in a sample. WO95/16026 provides a method for detecting BUP, NBUP, BUP-G and NBUP-G using FPIA and murine IgG.

Homogeneous microparticles immunoassay technology, which can be referred to as immunoturbidimetric assays, is based on the agglutination of particles and compounds in solution. When particles and/or chemical compounds agglutinate, particle sizes can increase and increase the turbidity of a solution. Accordingly, BUP, NBUP, BUP-G and NBUP-G analyte antibodies can be used with microparticles in order to assess the presence, and optionally the amount, of BUP analyte in a sample. Homogeneous microparticles immunoassay may be useful because the immunoassays can be performed on blood, blood hemolysate, serum, plasma, tissue, and/or other samples. Homogeneous microparticles immunoassay assays can be configured to be performed with BUP analyte and loaded onto a microparticle, or with a BUP, NBUP, BUP-G and NBUP-G analyte antibody loaded onto a microparticle. Homogeneous microparticles immunoassay or immunoturbidimetric assays find use for measuring agglutination of substances in a sample. Immunoturbidimetric assay technologies are described in, e.g., U.S. Pat. Nos. 5,571,728, 4,847,209, 6,514,770, and 6,248,597, the disclosures of which are incorporated herein by reference. Such assays involve light attenuation, nephelometric, or turbidimetric methods.

Cloned Enzyme Donor Immunoassays (“CEDIA®”, ThermoFisher), as are based upon the competition of BUP, NBUP analyte in the biological sample with a BUP or NBUP conjugate containing an inactive genetically engineered enzyme-donor (“ED”) fragment such as from β-D-galactoside galactohydrolase or β-galactosidase (“β-gal”) from E. coli, for binding to an antibody capable of binding BUP analyte. Thiol containing ED can be conjugated to the haloacetamides of the current invention to yield ED conjugates. If BUP and/or NBUP analyte is present in the sample it may bind to the antibody, leaving the ED portion of the ED-derivative conjugate free to restore enzyme activity of β-D-galactoside galactohydrolase or b-gal in the reaction mixture so as to be capable of association with enzyme acceptor (“EA”) fragments. The active enzyme, which includes the ED and EA, may then be capable of producing a quantifiable reaction product when exposed to an appropriate substrate. An example of a substrate is chlorophenol red-β-D-galactopyranoside (“CPRG”), which can be cleaved by the active enzyme into galactose and CPR, where CPR is measured by absorbency at about wavelength 570 nm. If BUP analyte is not present in the sample, the antibody may bind to the ED-derivative conjugate, thereby inhibiting association of the ED fragments with the EA fragments and inhibiting restoration of enzyme activity. The amount of reaction product and resultant absorbance change are proportional to the amount of BUP analyte in the sample. Alternatively a fluorescent substrate such as umbelliferone galactoside can be used to give a fluorescent signal. Alternatively a chemiluminescent substrate such as dioxetane galactosides or luciferin galactoside substrates could be used for a luminescent signal. The CEDIA assay is limited to detecting buprenorphine and norbuprenorphine only,

A competitive assay using chemiluminescent microparticle immunoassay (“CMIA”) technology can also be used to assess whether or not BUP analyte is present in a sample (Regis Bouquie et al., Am J. Clin Path 2016). Various types of CMIA technologies may be used for determining the presence and/or amount of an analyte in a sample. CMIA assays can include the use of BUP analyte antibodies, which are capable of binding to BUP analyte, which are coupled to particles, such as magnetic particles or particles suitable for separation by filtration, sedimentation, and/or other means. Additionally, a tracer, which can include a BUP or derivative linked to a suitable chemiluminescent moiety, can be used to compete with free BUP analyte in the patient's sample for the limited amount of BUP analyte antibody on the particle. After the sample, tracer, and antibody particles interact and a routine wash step has removed unbound tracer, the amount of tracer bound to antibody particles can be measured by chemiluminescence, wherein chemiluminescence is expressed in Relative Light Units (RLUs). The amount of chemiluminescence is inversely related to the amount of free analyte in the patient's sample and concentration is determined by constructing a standard curve using known values of the analyte. Chips with different analytical reagents (antigens, antibodies, nucleic acid probes) arrayed on have been developed and commercialized among these technologies, the Evidence MultiStat in combination with Biochip Array Technology by Randox (Crumlin, UK) uses a chemiluminescence reaction as output signal and allows one to perform several laboratory based assays such as drugs of abuse for example buprenorphine and other opioids. This platform has poor sensitivity for the metabolites.

According to some embodiments, provided is a homogenous enzyme immunoassay for the analysis of BUP, NBUP, BUP-G and/or NBUP-G in biological fluids (e.g., whole blood, blood serum, blood plasma, urine, sputum, semen, saliva, ocular lens fluid, cerebral fluid, spinal fluid, amniotic fluid, tissue culture media, or the like, and including dilutions thereof). The assay is based on competition between BUP, NBUP, BUP-G and/or NBUP-G present in the biological fluid and a BUP, NBUP, BUP-G and/or NBUP-G labeled with an enzyme (e.g., glucose-6-phosphate dehydrogenase (G6PD)) for antibody binding sites. Enzyme activity decreases upon binding to the antibody, so the BUP, NBUP, BUP-G and/or NBUP-G concentration in the biological fluid can be measured in terms of enzyme activity. For example, active G6PD converts nicotinamide adenine dinucleotide (NAD⁺) to NADH, resulting in an absorbance change that may be measured spectrophotometrically. A bacterial (Leuconostoc mesenteroides) enzyme may be employed in the assay so that endogenous serum G6PD does not interfere because the coenzyme NAD⁺ functions only with the bacterial enzyme.

In certain embodiments, in an immunoassay of the present disclosure, including in any of the homogenous enzyme immunoassays described herein, one or more reagents are provided in lyophilized form. As just one example, a composition that includes any of the compounds of Formula 1 of the present disclosure, any of the antibodies of the present disclosure, or both, may be provided in the form of lyophilized reagent spheres (or “beads”) as described in U.S. Pat. No. 5,413,732, the disclosure of which is incorporated herein by reference in its entirety for all purposes. Briefly, such spheres may be made, e.g., by forming a homogeneous solution of a reagent; measuring uniform drops of the solution (e.g., 2 to 50 μL); dispensing the uniform, measured drops into an unagitated cryogenic liquid (e.g., liquid nitrogen), whereby the drops are frozen; collecting the frozen drops from the cryogenic liquid; and lyophilizing the frozen drops, thereby forming a plurality of lyophilized reagent spheres.

According to some embodiments, e.g., including embodiments in which one or more reagents are provided in lyophilized form, a centrifugal analyzer that includes a microfluidic rotor (or “disc”) is employed in an immunoassay of the present disclosure. For example, an immunoassay of the present disclosure may employ an analyzer that includes a centrifugal rotor for separating plasma from whole blood that includes a plurality of internal chambers and passages for combining blood plasma or serum with one or more reagents (e.g., lyophilized spheres as described above) and distributing the plasma or serum to a plurality of individual test wells. The chambers and passages necessary for separating the whole blood into plasma are located radially outward from metering chambers that deliver precisely measured volumes of blood and/or diluent to a separation chamber. The separation chamber includes a radially-outward cell trap. Spinning of the rotor causes the cellular components of the whole blood to be sequestered in the cell trap. The separated plasma is then delivered to a plurality of test wells or cuvettes. The above separation and aliquoting steps typically occur as a result of centrifugal force generated by the spinning rotor. The lyophilized reagent spheres described above in combination with the rotors described above are particularly suitable for analyzing blood plasma or diluted blood plasma. They are also useful with a wide variety of other biological fluids, such as urine, sputum, semen, saliva, ocular lens fluid, cerebral fluid, spinal fluid, amniotic fluid, and tissue culture media, as well as food and industrial chemicals, and the like. Details regarding such centrifugal analyzers that include a microfluidic rotor may be found, e.g., in U.S. Pat. Nos. 5,061,381, 5,173,193; 5,122,284 and 5,186,844, the disclosures of which are incorporated herein in their entireties for all purposes.

In some embodiments, the immunoassay employs a centrifugal analyzer that includes a microfluidic rotor comprising siphons for delivering a premeasured volume of liquid (e.g., a biological sample such as whole blood, blood serum, or blood plasma) between a first and a second chamber in the rotor. The siphons may include an elbow that is radially inward of the radially most inward point of the fluid in the first chamber. As the rotor is spinning, the fluid does not flow past the elbow. Once the rotor stops, capillary forces “prime” the siphon by pulling fluid just around the elbow. When the rotor is restarted, centrifugal force draws the remaining fluid out of the metering chamber into the receiving chamber until the level of the fluid in the metering chamber is at the same radial distance as the outlet of the siphon. The siphons may be designed such that the inlet of the siphon on the first chamber is radially outward of the siphon outlet on the second chamber. The positioning of the inlets and outlets of the siphons provides certain advantages. For example, the inlet of the siphon may always be positioned radially outward of the final position of the meniscus of the fluid in the first chamber, after fluid has been transferred to the second chamber. Thus, inaccuracy in measurement associated with different shaped menisci in different fluids is minimized since the meniscus is minimized. In addition, as will be appreciated by one of skill in the art, all siphons are semi-stable because the train of fluid in a siphon is stable but easily broken if the rotor is perturbed. When the train of fluid is broken, under centrifugal force, the fluid contained in the siphon will flow to the radially most outward point. In previous siphons, this point is the siphon outlet. Thus, the potential exists for the delivery of unmetered volumes of fluid to the receiving chamber. In the siphons described herein, the radially most outward point in the siphon is the siphon inlet. In this design, the problem of delivering unmetered volumes of fluid is avoided because the fluid flows back into the first chamber when the train of fluid is broken. Further details regarding analyzers that include a centrifugal rotor comprising siphons for delivering premeasured volumes of liquid, which may be employed in any of the methods/immunoassays of the present disclosure, may be found in U.S. Pat. No. 7,998,411, the disclosure of which is incorporated herein by reference in its entirety for all purposes.

The BUP derivatives, conjugates, antibodies, immunogens, and/or other conjugates described herein are also suitable for any of a number of other heterogeneous immunoassays with a range of detection systems including but not limited to enzymatic or fluorescent, and/or homogeneous immunoassays including but not limited to rapid lateral flow assays, and antibody arrays, as well as formats yet to be developed.

While various immunodiagnostic assays have been described herein that utilize the BUP derivatives, conjugates, antibodies, and immunogens, such assays can also be modified. As such, various modifications of steps or acts for performing such immunoassays can be made within the scope of the embodiments described herein. Additional information related to assay format are described, among other places, in David Wild (The Immunoassay Handbook, 4th Edition Published Date: 31 Jan. 2013, Elsevier Science).

### Kits

Aspects of the present disclosure further include kits. In some embodiments, the kits find use in determining an amount of at least one BUP analyte in a sample. Such kits may include any of the compounds of Formula 1 and Formula 2 of the present disclosure, any of the antibodies of the present disclosure, or both.

In certain embodiments, provided are kits for determining an amount of BUP, NBUP, BUP-G and/or NBUP-G analyte in a sample, such kits including any of the antibodies of the present disclosure, and instructions for using the antibody to determine an amount of at least one BUP, NBUP, BUP-G and/or NBUP-G analyte in a sample. According to some embodiments, the antibody specifically binds to BUP, NBUP, BUP-G and/or NBUP-G with less than 0.1% cross reactivity to structurally similar opioids shown in FIG. 2. In certain embodiments, the kits that include an antibody of the present disclosure further include any of the compounds used as haptens of the present disclosure. In some embodiments, the compound is one where Z is a label, such as an enzyme, e.g., glucose-6-phosphate dehydrogenase (G6PD).

Also provided are kits for determining an amount of at least one BUP, NBUP, BUP-G and/or NBUP-G analyte in a sample, the kits including any of the compounds of Formula 1 of the present disclosure, and instructions for using the compound to determine an amount of at least one BUP analyte in a sample. In certain aspects, the compound is one where Z is a label, such as an enzyme, e.g., glucose-6-phosphate dehydrogenase (G6PD). Such kits may further include any of the antibodies of the present disclosure. According to some embodiments, the antibody specifically binds to BUP, NBUP, BUP-G and/or NBUP-G but with less than 0.1% cross reactivity to structurally similar opioids in FIG. 2, and the kit may further include instructions for determining the amount of BUP, NBUP, BUP-G and/or NBUP-G in the biological sample.

According to some embodiments, the kits of the present disclosure are useful for conveniently performing an assay for the determination of a BUP, NBUP, BUP-G and/or NBUP-G analyte in a sample. The kit may include: (a) an antibody raised that specifically binds to BUP, NBUP, BUP-G and/or NBUP-G and a compound of Formula 1 described herein (e.g., a BUP-X conjugate); and (b) instructions for determining the amount of the BUP, NBUP, BUP-G and/or NBUP-G analyte in the sample. In some embodiments, the kit also includes a conjugate of a compound of Formula 1, where the conjugate includes a label (e.g., a detectable label, such as G6PD). In certain instances, the kit also includes ancillary reagents for determining the analyte. The antibody of the kit may be an antibody raised against a compound of Formula 1 described herein.

To enhance the versatility of the immunoassay, the kit reagents can be provided in packaged combination, in the same or separate containers, in liquid or lyophilized form so that the ratio of the reagents provides for substantial optimization of the method and assay. In certain embodiments, the kits include an antibody of the present disclosure, a compound of the present disclosure, or both, provided as lyophilized reagent spheres as described in U.S. Pat. No. 5,413,732, the disclosure of which is incorporated herein by reference in its entirety for all purposes. The reagents provided in the kits may each be in separate containers or various reagents can be combined in one or more containers, e.g., depending on the cross-reactivity and stability of the reagents. In some embodiments, compounds derived from Formula 1 and Formula 2 described herein (e.g., conjugates of hapten 1,2,3,4 5 and 6) is present in lyophilized form. In some embodiments, the antibody is present in lyophilized form. For example, compounds derived from Formula 1 and 2 can be present in a first lyophilized composition (which may further include one or more excipients, buffers, stabilizers, etc.), and the antibody can be present in a second lyophilized composition (which may further include an enzyme substrate and one or more excipients, buffers, stabilizers, etc.). The first lyophilized composition and the second lyophilized composition may be provided in a single kit, such as for example in a packaging or container for single use.

The kit can further include other separately packaged reagents for conducting an assay such as ancillary reagents such as an ancillary enzyme substrate, and so forth. The relative amounts of the various reagents in the kits can be varied widely to provide for concentrations of the reagents that substantially optimize the reactions that need to occur when performing a method/immunoassay (e.g., homogenous enzyme immunoassay) and further to optimize substantially the sensitivity of the assay. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. The kit can further include a written description of a method in accordance with the present invention as described above.

The description of certain exemplary embodiments of kits uses the language “and/or,” which means that the kit may or may not contain each item mentioned. This language is used for the sake of brevity. In general, an immunoassay kit will include at least one antibody for an immunogen of an analyte, e.g., BUP, and at least one enzyme conjugate (e.g., label conjugate) that corresponds to that analyte, e.g., an enzyme conjugate of a derivative of BUP.

In certain embodiments, a kit is provided for an assay for the analyte BUP and/or metabolites of BUP. The kit may include, in packaged combination: (i) an antibody raised against a compound of Formula 1; and (ii) a conjugate of a derivative of the analyte. Another embodiment of the presently disclosure is a kit for an assay for the analyte BUP and/or metabolites of BUP that includes, in packaged combination: (i) an antibody raised against a derivative of the analyte; and (ii) a conjugate of a hapten of the analyte, where the haptens are compounds derived from Formula 1 and 2.

The compounds, methods and kits of the present disclosure find use in routine monitoring of by immunoassays. In certain embodiments, these immunoassays provide simple automated tests adapted to standard laboratory equipment with a quick turn-around time. As described herein, in order to provide such immunoassays, antibodies to BUP, NBUP, BUP-G and/or NBUP-G are produced. The derivatives and immunogens are designed to impart, through the corresponding antibodies, specific reactivity to BUP, NBUP, BUP-G and/or NBUP-G and less than 0.1% cross reactivity to structurally similar opioids (FIG. 2) as well as detectability of BUP to 5 ng/ml.

The instructions included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate or scanned from a QR code for directions to download or provide instructions for use.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

In relation to the compounds and conjugates and immunogens, the following abbreviations are used: DCM is dichloromethane; DMF is N,N-dimethylformamide; DMSO is dimethyl sulfoxide; EDTA is ethylenediaminetetraacetic acid; KLH is keyhole limpet hemocyanin; SATA is N-succinimidyl S-acetylthioacetate; TFA is trifluoroacetic acid; EDCI is 1-ethyl-3 (3-dimethylaminopropyl) carbodiimidehydrochloride; NHS is N-hydroxysuccinimide; DIPEA is diisopropyl ethylamine; THF is tetrahydrofuran; DTT is dithioerythritol; G6PD is Glucose-6-Phosphate Dehydrogenase; EtOAc is ethyl acetate; BSA is bovine serum albumin; MeCN is Acetonitrile; t-Boc is tert-butyloxycarbonyl protecting group; TLC is thin layer chromatography; MeOH is methanol; AcOH is acetic acid; PBST is phosphate buffered saline with Tween-20; TMB is 3,3′,5,5′-tetramethylbenzidine; PBMC is peripheral blood mononuclear cell.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978; R. C. Larock Comprehensive Organic Transformations, Second Edition, Wiley-VCH 1999).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969. Analytical techniques such as LC-MS is Liquid chromatography with mass spectrometry detection, 1H-NMR means proton nuclear magnetic resonance with shifts denoted in ppm downfield of tetramethyl silane (TMS), ELSD is evaporative light scatter detection.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4th edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

Notwithstanding the appended claims, the present disclosure is also defined by the following clauses:

Clause 1. A compound of Formula 1:

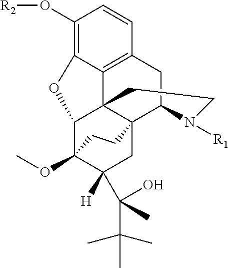

wherein:
R₁ is —Y—Z;
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—;
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R₂ is H, glucuronic acid, glucuronide salt, or a protecting group,
and salts thereof.
   Clause 2. The compound of Clause 1, wherein the linking group comprises 1 to 15 carbon atoms and/or 0 to 6 heteroatoms.
   Clause 3. The compound of Clause 2, wherein the linking group is selected from the group consisting of —(CH₂)ₙC(O)—, —C(O)(CH₂)ₙ—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ—, —(CH₂)ₙNHC(O)—, —(CH₂)ₙNHC(O)(CH₂)ₙ—, —(CH₂)ₙNHC(O)(CH₂)NO(CH₂)ₙNHC(O)(CH₂)ₙ—, —(CH₂)ₙNHC(O)(CH₂)ₙNHC(O)(CH₂)ₙ—, —NH(CH₂)ₙC(O)—, —(CH₂)ₙ—, —C(O)NH(CH₂CH₂O)ₘ(CH₂)ₙNHC(O)(CH₂)ₙ—, and —(CH₂)ₙ (heterocyclyl)S(CH₂)ₙC(O)—, each m is independently an integer from 1 to 10, and each n is independently an integer from 1 to 10, and salts thereof.
   Clause 4. The compound of any one of Clauses 1-3, wherein Z is a protein.
   Clause 5. The compound of Clause 1, wherein the protein is an immunogenic carrier selected from the group consisting of a hemocyanin, a globulin, and an albumin.
   Clause 6. The compound of Clause 5, wherein the immunogenic carrier is bovine serum albumin (BSA) or keyhole limpet hemocyanin (KLH).
   Clause 7. The compound of Clause 1, wherein Z is an immunogenic carrier and the immunogenic carrier is a polysaccharide.
   Clause 8. The compound of any one of Clauses 1-6, wherein Z is a label.
   Clause 9. The compound of Clause 7, wherein the label is an enzyme.
   Clause 10. The compound of Clause 8, wherein the enzyme is selected from the group consisting of an alkaline phosphatase, a β-galactosidase and a horseradish peroxidase.
   Clause 11. The compound of Clause 3, wherein the enzyme is glucose-6-phosphate dehydrogenase (G6PD).
   Clause 12. An antibody that specifically binds to the compound of Formula 1 of any one of Clauses 1-10.
   Clause 13. A compound of Formula 2:

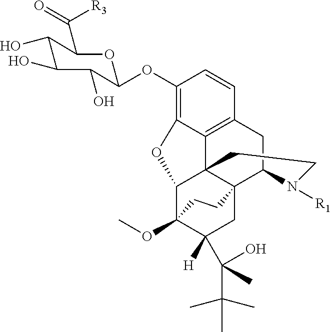

wherein:
R₁ is selected from H, -cyclopropyl methyl, and Y—Z;
R₃ is selected from OH, NH₂, NHR⁴, and Y—Z;
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—;
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R⁴ is selected from an immunogenic carrier, a protein, an enzyme, and a label,
and salts thereof.
   Clause 14. The compound of Clause 13, wherein the linking group comprises 1 to 15 carbon atoms and/or 0 to 6 heteroatoms.
   Clause 15. The compound of Clause 13, wherein the linking group is selected from the group consisting of —(CH₂)ₙC(O)—, —C(O)(CH₂)ₙ—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ—, —(CH₂)ₙNHC(O)—, —(CH₂)ₙNHC(O)(CH₂)ₙ—, —(CH₂)ₙNHC(O)(CH₂)ₙO(CH₂)ₙNHC(O)(CH₂)ₙ—, —(CH₂)ₙNHC(O)(CH₂)ₙNHC(O)(CH₂)ₙ—, —NH(CH₂)ₙC(O)—, —(CH₂)ₙ—, —C(O)NH(CH₂CH₂O)ₘ(CH₂)ₙNHC(O)(CH₂)ₙ—, and —(CH₂)ₙ (heterocyclyl)S(CH₂)ₙC(O)—, each m is independently an integer from 1 to 10, and each n is independently an integer from 1 to 10, and salts thereof.
   Clause 16. The compound of any one of Clauses 13-15, wherein Z is a protein.
   Clause 17. The compound of Clause 16, wherein the protein is an immunogenic carrier selected from the group consisting of a hemocyanin, a globulin, and an albumin.
   Clause 18. The compound of Clause 17, wherein the immunogenic carrier is bovine serum albumin (BSA) or keyhole limpet hemocyanin (KLH).
   Clause 19. The compound of Clause 13, wherein Z is an immunogenic carrier and the immunogenic carrier is a polysaccharide.
   Clause 20. The compound of any one of Clauses 13-15, wherein Z is a label.
   Clause 21. The compound of Clause 20, wherein the label is an enzyme.
   Clause 22. The compound of Clause 21, wherein the enzyme is selected from the group consisting of an alkaline phosphatase, a β-galactosidase and a horseradish peroxidase.
   Clause 23. The compound of Clause 21, wherein the enzyme is glucose-6-phosphate dehydrogenase (G6PD).
   Clause 24. An antibody that specifically binds to the compound of Formula 2 of any one of Clauses 13-23.
   Clause 25. The antibody of Clause 12 or Clause 24, wherein the antibody competes for binding to the compounds of Formula 1 and 2 with an antibody comprising: (SEQUENCES in Table 1)
variable heavy chains (V_{H}) polypeptides comprising
   a V_{H} CDR1 comprising the amino acid sequence (SEQ ID NO: 1-23);
   a V_{H} CDR2 comprising the amino acid sequence (SEQ ID NOs: 24-46); and
   a V_{H} CDR3 comprising the amino acid sequence (SEQ ID NOs: 47-69); and
a variable light chain (V_{L}) polypeptide comprising
   a V_{L} CDR1 comprising the amino acid sequence (SEQ ID NOs: 70-92);
   a V_{L} CDR2 comprising the amino acid sequence (SEQ ID NOs: 93-115); and
   a V_{L} CDR3 comprising the amino acid sequence (SEQ ID NOs: 116-138).
      Clause 26. The antibody of Clause 25, wherein the antibody comprises a variable heavy chain (V_{H}) polypeptides comprising amino acid sequences having 70% or greater identity to the amino acid sequence set forth in SEQ IDS NO: 139-161.
      Clause 27. The antibody of any one of Clauses 25-26, wherein the antibody is selected from the group consisting of: an IgG, Fv, single chain antibody, scFv, Fab, F(ab′)₂, and Fab′.
      Clause 28. The antibody of any one of Clauses 25-27, wherein the antibody is an IgG.
      Clause 29. The antibody of Clause 28, wherein the antibody is an IgG1.
      Clause 30. The antibody of any one of Clauses 27-29, wherein the antibody is a Fab.
      Clause 31. The antibody of any one of Clauses 27-29, wherein the antibody is a single chain antibody.
      Clause 32. The antibody of Clause 31, wherein the antibody is an scFv.
      Clause 33. The antibody of any one of Clauses 25-32, wherein the antibody is a monoclonal antibody.
      Clause 34. The antibody of Clause 25-32, wherein the antibody is a polyclonal antibody.
      Clause 35. The antibody of Clause 34, wherein the antibody is a rabbit polyclonal antibody.
      Clause 36. The antibody of any one of Clauses 24-35, wherein the antibody further specifically has 0.1% cross reactivity with structurally similar opiates in FIG. 2
      Clause 37. A nucleic acid encoding a variable heavy chain (V_{H}) polypeptide, a variable light chain (V_{L}) polypeptide, or both, of an antibody of any one of Clauses 25-36.
      Clause 38. An expression vector comprising the nucleic acid of Clause 37.
      Clause 39. A cell comprising the nucleic acid of Clause 37 or the expression vector of Clause 38.
      Clause 40. A cell comprising:
a first nucleic acid encoding a variable heavy chain (V_{H}) polypeptide of the antibody of any one of Clauses 25-36, and
a second nucleic acid encoding a variable light chain (V_{L}) polypeptide of the antibody of any one of Clauses 25-36.
   Clause 41. The cell of Clause 40, comprising:
a first expression vector comprising the first nucleic acid; and
a second expression vector comprising the second nucleic acid.
   Clause 42. A method of making an antibody, comprising culturing the cell of any one of Clauses 40-41 under conditions suitable for the cell to express the antibody, wherein the antibody is produced.
   Clause 43. A composition comprising:
the compounds of Formula 1 and Formula 2 of any one of the preceding Clauses; or
the antibody of any one of the preceding Clauses; or
the nucleic acid of any one of the preceding Clauses; or
the expression vector of any one of the preceding Clauses; or
the cell of any one of the preceding Clauses; or
any combination thereof.
   Clause 44. The composition of Clause 43, wherein the composition is present in a liquid medium.
   Clause 45. The composition of Clause 43, wherein the composition is present in a lyophilized form.
   Clause 46. A method for determining an amount of a buprenorphine analyte in a medium, the method comprising:
contacting in a medium a sample suspected of containing at least one buprenorphine analyte and metabolite, and
   the antibodies of any one of Clauses 25-36 and
determining the presence or absence of a complex comprising the buprenorphine analyte and metabolites and the antibody,
wherein the presence of the complex indicates the presence of the buprenorphine analyte in the sample.
   Clause 47. The method according to Clause 46, wherein the medium further comprises the compounds of Formula 1, Formula 2, and the compound according to any one of Clauses to 1-23.
   Clause 48. The method according to any one of Clauses 46-47, wherein the determining comprises detecting the presence of an enzymatic reaction product of the compound.
   Clause 49. A kit for determining an amount of at least one buprenorphine analyte in a sample, the kit comprising:
the antibody of any one of Clauses 25-26; and
instructions for using the antibody to determine an amount of buprenorphine analyte in a sample.
   Clause 50. The kit of Clause 49, wherein the antibody specifically has a cross reactivity of less than 0.1% to structurally similar opiates FIG. 2.
   Clause 51. The kit of Clause 49, further comprising instructions for determining an amount of the buprenorphine with cross reactivity of less than 0.1% to naloxone, morphine, codeine, acetyl morphine, oxycodone, and structurally similar opioids in FIG. 2.
   Clause 52. The kit of Clause 49, wherein the antibody is present in a lyophilized form.
   Clause 53. The kit of any one of Clauses 49-52, further comprising a compound of Formula 1, or Formula 2.
   Clause 54. The kit of Clause 53, wherein Z is a label.
   Clause 55. The kit of Clause 54, wherein the label is an enzyme.
   Clause 56. The kit of Clause 55, wherein the enzyme is selected from the group consisting of: an alkaline phosphatase, a β-galactosidase, and a horseradish peroxidase.
   Clause 57. The kit of Clause 55, wherein the enzyme is glucose-6-phosphate dehydrogenase (G6PD).
   Clause 58. The kit of any one of Clauses 53-57, wherein the compound is present in a lyophilized form.
   Clause 59. A kit for determining an amount of buprenorphine analyte in a sample, the kit comprising:
the compound of Formula 1 of any one of Clauses 1-11; and
instructions for using the compound to determine an amount of at least one buprenorphine analyte in a sample.
   Clause 60. The kit of Clause 59, wherein Z is a label.
   Clause 61. The kit of Clause 60, wherein the label is an enzyme.
   Clause 62. The kit of Clause 61, wherein the enzyme is selected from the group consisting of an alkaline phosphatase, a β-galactosidase, and a horseradish peroxidase.
   Clause 63. The kit of Clause 61, wherein the enzyme is glucose-6-phosphate dehydrogenase (G6PD).
   Clause 64. The kit of any one of Clauses 59-63, wherein the compound is present in lyophilized form.
   Clause 65. The kit of any one of Clauses 59-64, further comprising the antibody of any one of Clauses 25-36.
   Clause 66. The kit of Clause 65, wherein the antibody specifically has a cross reactivity of 0.1% with structurally similar opiates.
   Clause 67. The kit of any one of Clauses 59-66, wherein the antibody is present in lyophilized form.

The following examples are put forth to provide those of ordinary skill in the art with a complete disclosure and description of how to make and use the embodiments, and are not intended to limit the scope of what the inventors regard as their invention, nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used.

### Example 1—Synthesis of BUP-G and NBUP-G

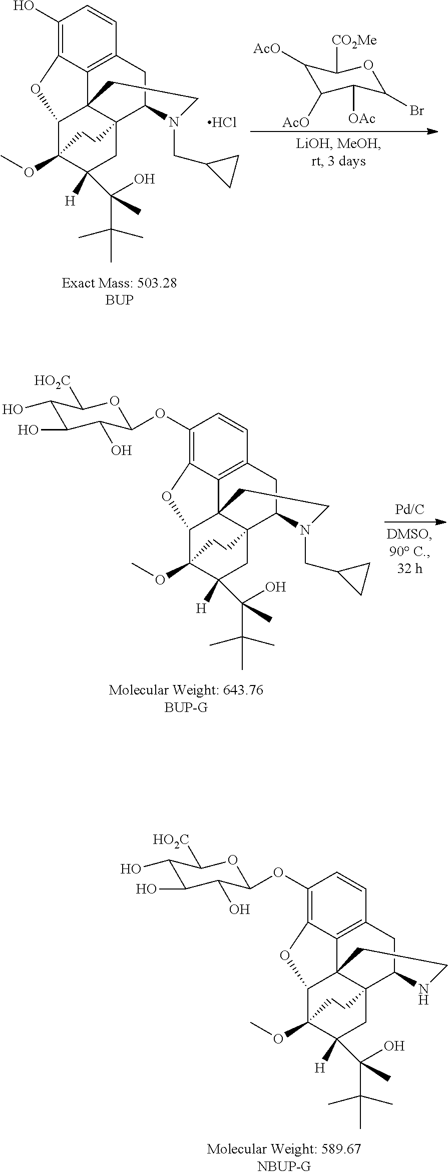 [SMILES: CO[C@@]12CC[C@@]3(C[C@@H]1[C@](C)(O)C(C)(C)C)[C@H]1Cc4ccc(O)c5c4[C@@]3(CCN1CC1CC1)[C@H]2O5 ; Cl ; COC(=O)C1OC(Br)C(OC(C)=O)C(OC(C)=O)C1OC(C)=O ; CO[C@@]12CC[C@@]3(C[C@@H]1[C@](C)(O)C(C)(C)C)[C@H]1Cc4ccc(OC5OC(C(=O)O)C(O)C(O)C5O)c5c4[C@@]3(CCN1CC1CC1)[C@H]2O5 ; CO[C@@]12CC[C@@]3(C[C@@H]1[C@](C)(O)C(C)(C)C)[C@H]1Cc4ccc(OC5OC(C(=O)O)C(O)C(O)C5O)c5c4[C@@]3(CCN1)[C@H]2O5]

Synthesis of (2S,3S,4S,5R,6S)-6-(((4R,4aS,6R,7R,7aR,12bS)-3-(cyclopropylmethyl)-6-((S)-2-hydroxy-3,3-dimethylbutan-2-yl)-7-methoxy-1,2,3,4,5,6,7,7a-octahydro-4a,7-ethano-4,12-methanobenzofuro[3,2-e]isoquinolin-9-yl)oxy)-3,4,5-trihydroxytetrahydro-2H-pyran-2-carboxylic acid (BUP-G): To a stirred solution of buprenorphine hydrochloride (BUP, 1.0 g, 1.98 mmol) in methanol (30 mL) was added LiOH·H₂O (2.5 g, 59.4 mmol), and the reaction mixture was maintained for 1 h at room temperature. To this reaction mixture was added acetobromo-alpha-D-glucuronic acid methyl ester (7.9 g, 19.8 mmol) portion-wise over 3 d. After complete consumption of starting material as analyzed by LCMS, the reaction mixture was quenched with saturated NH₄Cl (70 mL), and the pH was adjusted to 5-6 by adding acetic acid. Solvent was removed under high vacuum, and the residue was purified by chromatography on a C18 column using MeCN:H₂O to give the product BUP-G as a white solid (0.8 g, 69% yield).

### Step 2

Synthesis of (2S,3S,4S,5R,6S)-3,4,5-trihydroxy-6-(((4R,4aS,6R,7R,7aR,12bS)-6-((S)-2-hydroxy-3,3-dimethylbutan-2-yl)-7-methoxy-1,2,3,4,5,6,7,7a-octahydro-4a,7-ethano-4,12-methanobenzofuro[3,2-e]isoquinolin-9-yl)oxy)tetrahydro-2H-pyran-2-carboxylic acid (NBUP-G): To a stirred solution of compound BUP-G (400 mg, 0.62 mmol) in DMSO (8 mL) was added Pd/C (800 mg, 1.5 mmol, 10%). The reaction mixture was stirred for 32 h at room temperature. After complete consumption of starting material, analyzed by LCMS, purification by C18 column using MeCN:H₂O afforded product NBUP-G as a white solid (114 mg, 31% yield). LCMS, desired mass=[589.67], observed mass=[590.4] [M+H⁺].

### Example 2—Synthesis of NBUP N-Ethyl Bromoacetamide, Hapten 1

### Step 1

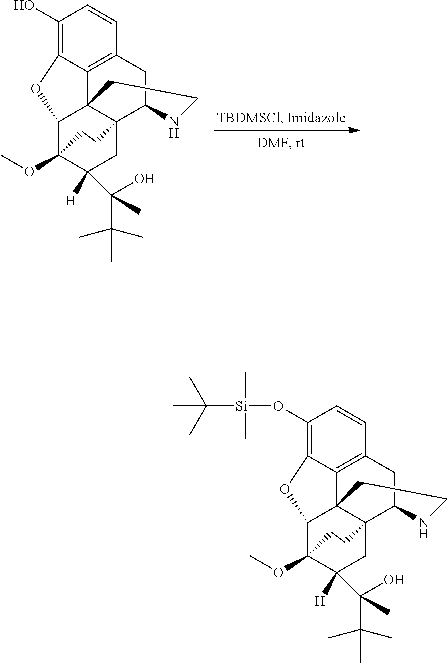 [SMILES: CO[C@@]12CC[C@@]3(C[C@@H]1[C@](C)(O)C(C)(C)C)[C@H]1Cc4ccc(O)c5c4[C@@]3(CCN1)[C@H]2O5 ; CO[C@@]12CC[C@@]3(C[C@@H]1[C@](C)(O)C(C)(C)C)[C@H]1Cc4ccc(O[Si](C)(C)C(C)(C)C)c5c4[C@@]3(CCN1)[C@H]2O5]

Norbuprenorphine (1.54 g, 3.72 mmol), imidazole (0.914 g, 13.4 mmol), and TBSCl (0.656 g, 4.36 mmol) were dissolved in DMF (19 mL). The reaction mixture was stirred at room temperature for 30 min. When judged complete (LCMS), water (5 mL) was added and the resulting mixture stirred. A precipitate formed and was collected by filtration. The precipitate was dissolved in DCM (150 mL), and the solution was washed with water (3×250 ml) and then brine (2×300 ml). The organic layer was dried with MgSO₄, filtered, and concentrated to afford the silyl protected norbuprenorphine as a yellow oil (1.68 g, 86% yield).

### Step 2

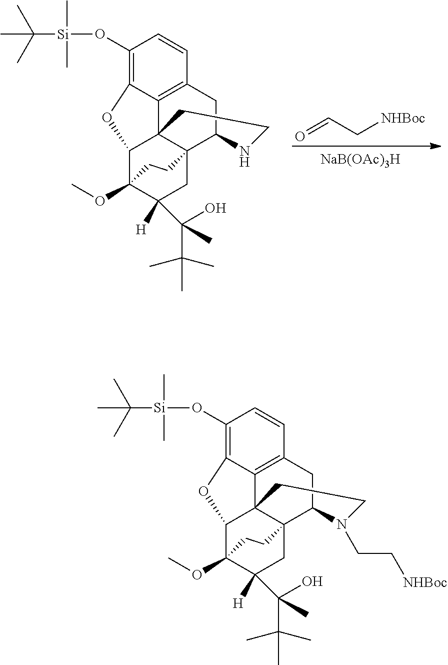

Silyl protected norbuprenorphine (1.27 g, 2.4 mmol), N-Boc-aminoacetaldehyde (0.328 g, 2.64 mmol) and sodium triacetoxyborohydride (1.19 g, 7.2 mmol) were dissolved in DCE (30 mL). The reaction mixture was stirred for 30 min. When judged to be complete (LCMS), water (200 mL) was added, and then the mixture was extracted with CH₂Cl₂ (3×100 mL). The combined organic phases were washed with brine (300 mL) and then dried with MgSO₄, filtered, and concentrated. The crude product was dissolved in DCM and purified by flash column chromatography (0-5% MeOH/CH₂Cl₂ gradient) to afford the reductive amination product as a white solid/powder (1.44 g, 89% yield).

### Step 3

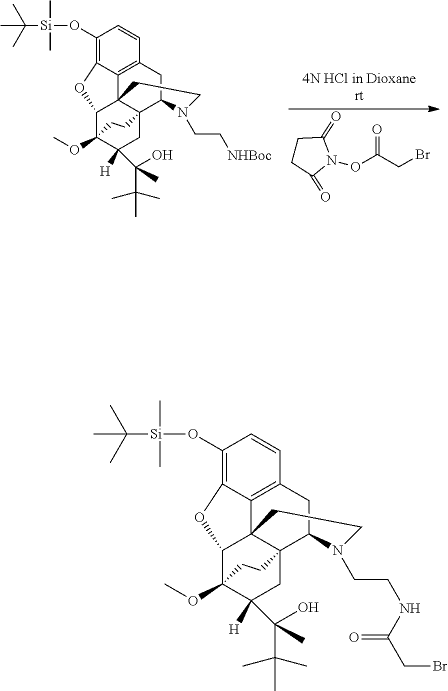 [SMILES: CO[C@@]12CC[C@@]3(C[C@@H]1[C@](C)(O)C(C)(C)C)[C@H]1Cc4ccc(O[Si](C)(C)C(C)(C)C)c5c4[C@@]3(CCN1CCNC(=O)OC(C)(C)C)[C@H]2O5 ; O=C(CBr)ON1C(=O)CCC1=O ; CO[C@@]12CC[C@@]3(C[C@@H]1[C@](C)(O)C(C)(C)C)[C@H]1Cc4ccc(O[Si](C)(C)C(C)(C)C)c5c4[C@@]3(CCN1CCNC(=O)CBr)[C@H]2O5]

To the protected amine isolated above (280 mg, 0.42 mmol) was added in HCl in dioxanes (16.3 mL). The reaction mixture was stirred for 30 min. When the reaction was judged complete, the mixture was concentrated. The residue was redissolved in DCM (50 mL) and concentrated to dryness three times. The resulting yellow oil was carried forward to the next step without further purification (330 mg).
To a solution of the crude amine isolated above (43 mg, 0.0754 mmol) in DCM (100 mL) was added bromoacetic acid N-hydroxysuccinimide ester (23 mg, 0.0981 mmol) and diisopropylethylamine (29 mL, 0.226 mmol). The reaction mixture was stirred for 30 min. Once judged complete (LCMS), water (300 mL) was added. The mixture was extracted with DCM (3×50 mL), and the combined organic phases were washed with brine (2×100 mL). The organic layer was dried with NaSO₄, filtered, and concentrated to give the amide product as a clear wax (57 mg).

### Step 4

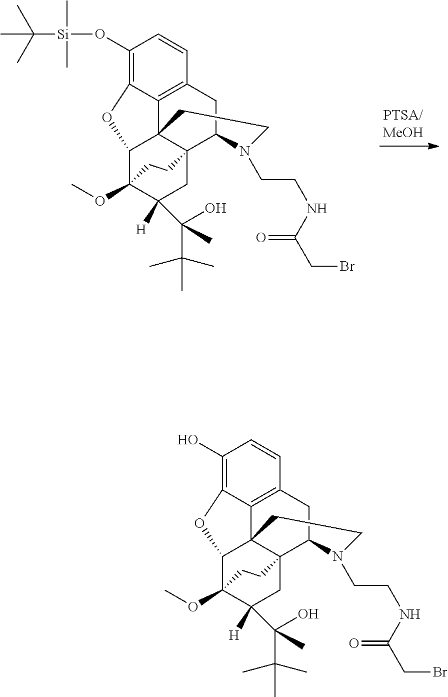 [SMILES: CO[C@@]12CC[C@@]3(C[C@@H]1[C@](C)(O)C(C)(C)C)[C@H]1Cc4ccc(O[Si](C)(C)C(C)(C)C)c5c4[C@@]3(CCN1CCNC(=O)CBr)[C@H]2O5 ; CO[C@@]12CC[C@@]3(C[C@@H]1[C@](C)(O)C(C)(C)C)[C@H]1Cc4ccc(O)c5c4[C@@]3(CCN1CCNC(=O)CBr)[C@H]2O5]

To a solution of the intermediate obtained above (57 mg, 0.0823 mmol) in MeOH (100 mL) was added TsOH (56.7 mg, 0.33 mmol). The reaction mixture was stirred for 3 h. When the reaction was judged complete (LCMS), saturated sodium bicarbonate solution was added to give a pH of 7. The resulting mixture was washed with DCM (3×100 mL), and the combined organic phases were washed with brine (300 mL), dried with NaSO₄, filtered, and concentrated. The residue was dissolved in DMF and purified by reversed phase preparative HPLC (MeCN/aqueous NH₄HCO₃). The product was obtained as a white powder (11 mg, 23% yield). LCMS: [C₂₉H₄₁N₂O₅], calc'd mass=[577.55], observed mass=[577.3] [M+H⁺].

### Example 3—Synthesis of NBUP-G N-Ethyl Bromoacetamide, Hapten 2

### Step1

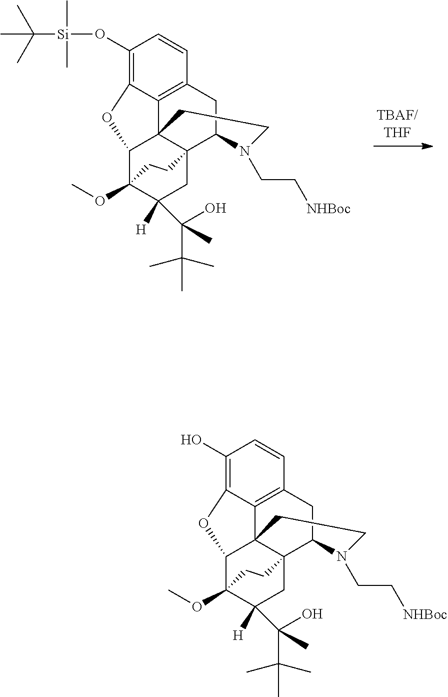 [SMILES: CO[C@@]12CC[C@@]3(C[C@@H]1[C@](C)(O)C(C)(C)C)[C@H]1Cc4ccc(O[Si](C)(C)C(C)(C)C)c5c4[C@@]3(CCN1CCNC(=O)OC(C)(C)C)[C@H]2O5 ; CO[C@@]12CC[C@@]3(C[C@@H]1[C@](C)(O)C(C)(C)C)[C@H]1Cc4ccc(O)c5c4[C@@]3(CCN1CCNC(=O)OC(C)(C)C)[C@H]2O5]

To a 1000 mL round bottom flask was charged Compound (2 g, 2.99 mmol) in THF (500 mL). Then TBAF (940 mg, 3.58 mmol) was added slowly. The reaction mixture was allowed to stir for 1 h then checked for completion via LCMS. Once completed, the reaction was concentrated via rotary evaporator and purified via flash chromatography (0-5% MeOH in DCM). All clean fractions were combined and dried to give Compound, as a light-yellow solid (2 g). LCMS: [C₃₂H₄₈N₂O₆], desired mass=[556.73], observed mass=[557.60] [M+H⁺].

### Step 2

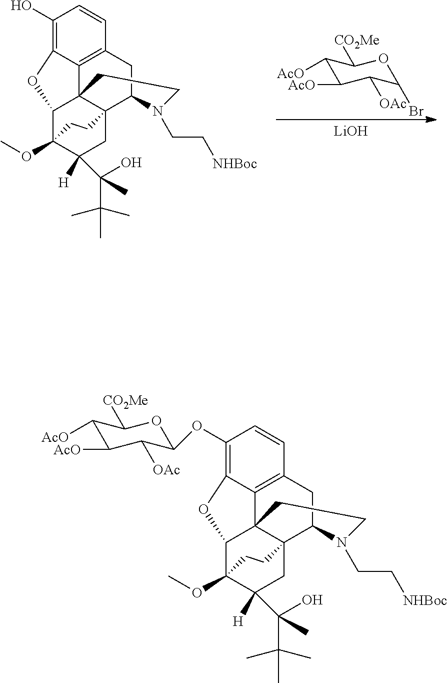 [SMILES: CO[C@@]12CC[C@@]3(C[C@@H]1[C@](C)(O)C(C)(C)C)[C@H]1Cc4ccc(O)c5c4[C@@]3(CCN1CCNC(=O)OC(C)(C)C)[C@H]2O5 ; COC(=O)C1OC(Br)C(OC(C)=O)C(OC(C)=O)C1OC(C)=O ; "LiOH" ; COC(=O)C1OC(Oc2ccc3c4c2O[C@H]2[C@]5(OC)CC[C@@]6(C[C@@H]5[C@](C)(O)C(C)(C)C)[C@@H](C3)N(CCNC(=O)OC(C)(C)C)CC[C@]426)C(OC(C)=O)C(OC(C)=O)C1OC(C)=O]

To solution of the desilylated product obtained above (0.60 g, 1.08 mmol) in methanol (7 mL) was added LiOH monohydrate (0.18 g, 4.31 mmol). The reaction mixture was cooled to ~0° C., and then acetobromo-D-glucuronic acid methyl ester (0.64 g, 1.62 mmol) was added. The solution was stirred at room temperature for 1 h. Then additional LiOH monohydrate (0.18 g, 4.31 mmol) was added, and the reaction mixture was stirred at room temperature for 30 min. Additional acetobromo-D-glucuronic acid methyl ester (0.47 g, 1.20 mmol) was added, and the reaction mixture was stirred at room temperature for an additional 1 h. Repeated addition of LiOH monohydrate (total addition: 1.81 g, 43.2 mmol) and acetobromo-D-glucuronic acid methyl ester (total addition: 5.0 g, 12.42 mmol) was continued until the estimated conversion by LCMS was ~95%. The mixture then was evaporated, and the residue was purified by reversed phase prep-HPLC (MeCN/aqueous 0.1% TFA gradient) and lyophilized to give the protected glucuronide as a white solid (0.64 g, 80%). LCMS: [C₃₈H₅₆N₂O₁₂], desired mass=[732.86], observed mass=[733.40] [M+H⁺]. 1H NMR (500 MHz, DMSO-d6): δ 8.48 (s, 1H), 6.95 (d, J=8.3 Hz, 1H), 6.67 (d, J=8.2 Hz, 1H), 5.48 (s, 1H), 5.07 (d, J=7.2 Hz, 1H), 4.67 (s, 1H), 3.84 (s, 1H), 3.68 (s, 1H), 3.43 (s, 3H), 3.22 (dd, J=7.3, 4.8 Hz, 2H), 3.19 (s, 1H), 2.94 (s, 2H), 2.80 (d, J=13.9 Hz, 1H), 2.17 (d, J=21.1 Hz, 3H), 1.90 (d, J=11.2 Hz, 1H), 1.77 (q, J=9.9, 9.9, 7.6 Hz, 1H), 1.56-1.43 (m, 2H), 1.24 (s, 3H), 0.97 (s, 10H), 0.62 (s, 1H).

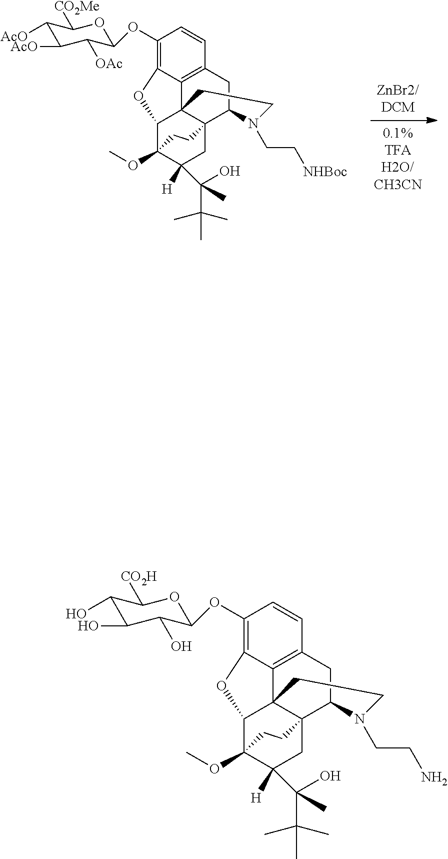 [SMILES: COC(=O)C1OC(Oc2ccc3c4c2O[C@H]2[C@]5(OC)CC[C@@]6(C[C@@H]5[C@](C)(O)C(C)(C)C)[C@@H](C3)N(CCNC(=O)OC(C)(C)C)CC[C@]426)C(OC(C)=O)C(OC(C)=O)C1OC(C)=O ; "0.1% TFAH2O/CH3CN" ; "ZnBr2/DCM" ; CO[C@@]12CC[C@@]3(C[C@@H]1[C@](C)(O)C(C)(C)C)[C@H]1Cc4ccc(OC5OC(C(=O)O)C(O)C(O)C5O)c5c4[C@@]3(CCN1CCN)[C@H]2O5]

### Step 3

To a solution of the protected glucuronide obtained above (0.62 g, 0.85 mmol) in DCM (11.0 mL) was added zinc bromide (0.77 g, 3.41 mmol). The reaction mixture was stirred at room temperature. After 3 d, LCMS analysis showed 50% conversion. Additional zinc bromide (0.13 g, 0.6 mmol) was added, and after another 2 d of stirring at room temperature, a third charge of zinc bromide (0.22 g, 1.02 mmol) was added. In total, the reaction mixture was stirred for 7 d. The reaction mixture was evaporated. DMSO (~20 mL) was added, and the mixture was filtered. The filtrate was purified by reversed phase prep-HPLC (MeCN/aqueous 0.1% TFA gradient) and lyophilized to afford the free amine as a white solid (0.4 g, 73%). LCMS: [C₃₃H₄₈N₂O₁₀], desired mass=[632.74], observed mass=[633.60] [M+H⁺]. 1H NMR (500 MHz, DMSO-d6): δ 6.87 (d, J=8.3 Hz, 1H), 6.53 (d, J=8.4 Hz, 1H), 5.58 (s, 1H), 5.08 (s, 1H), 4.90 (s, 1H), 4.84 (d, J=7.3 Hz, 1H), 4.39 (s, 1H), 3.15 (s, 2H), 2.91-2.77 (m, 1H), 2.69 (d, J=6.1 Hz, 1H), 2.62 (t, J=1.9, 1.9 Hz, 1H), 2.35-2.23 (m, 2H), 2.09 (dt, J=17.8, 8.4, 8.4 Hz, 1H), 2.02-1.88 (m, 1H), 1.70 (t, J=13.1, 13.1 Hz, 1H), 1.51 (d, J=11.6 Hz, 2H), 1.24 (s, 3H), 0.95 (s, 9H), 0.54 (s, 1H).

### Step 4

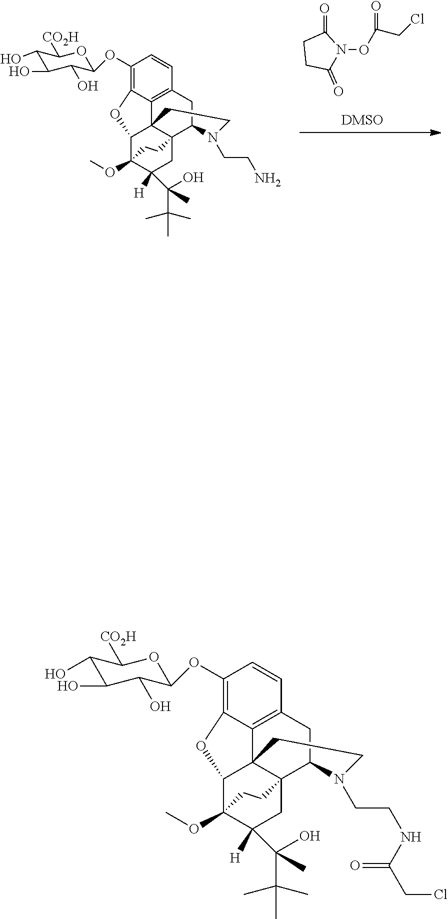

The free amine isolated above (50 mg, 0.079 mmol) was dissolved in DMSO (0.6 mL), and then N-(Chloroacetoxy)succinimide was added. The reaction mixture was stirred at room temperature for 15 min. LCMS analysis showed complete consumption of starting material. The reaction mixture was purified by reversed phase prep-HPLC and lyophilized to afford the target chloroacetamide as a white solid (48 mg, 86%). LCMS: [C₃₅H₄₉ClN₂O₁₁], desired mass=[709.22], observed mass=[709.50] [M+H⁺]. 1H NMR (500 MHz, DMSO-d6): δ 7.95 (t, J=5.5, 5.5 Hz, 1H), 6.83 (d, J=8.2 Hz, 1H), 6.54 (d, J=8.2 Hz, 1H), 553 (s, 1H), 5.26 (d, J=4.9 Hz, 1H), 5.08 (s, 1H), 4.97 (d, J=7.4 Hz, 1H), 4.43 (d, J=2.1 Hz, 1H), 4.01 (d, J=2.8 Hz, 2H), 3.52 (d, J=9.8 Hz, 1H), 3.43 (s, 3H), 3.17 (dt, J=15.1, 6.3, 6.3 Hz, 4H), 2.41 (dt, J=12.4, 6.2, 6.2 Hz, 1H), 2.27 (dd, J=18.8, 6.6 Hz, 1H), 2.21-2.13 (m, 1H), 2.07 (t, J=9.6, 9.6 Hz, 1H), 1.91 (td, J=12.6, 12.6, 5.3 Hz, 1H), 1.71 (t, J=12.9, 12.9 Hz, 1H), 1.54 (dd, J=22.8, 9.9 Hz, 2H), 1.30-1.25 (m, 1H), 1.24 (s, 3H), 1.07 (td, J=12.4, 12.3, 6.0 Hz, 1H), 0.94 (s, 9H), 0.53 (t, J=12.4, 12.4 Hz, 1H).

### Example 4—Synthesis of BUP O-Ethyl Bromoacetamide Hapten 3

### Step 1.

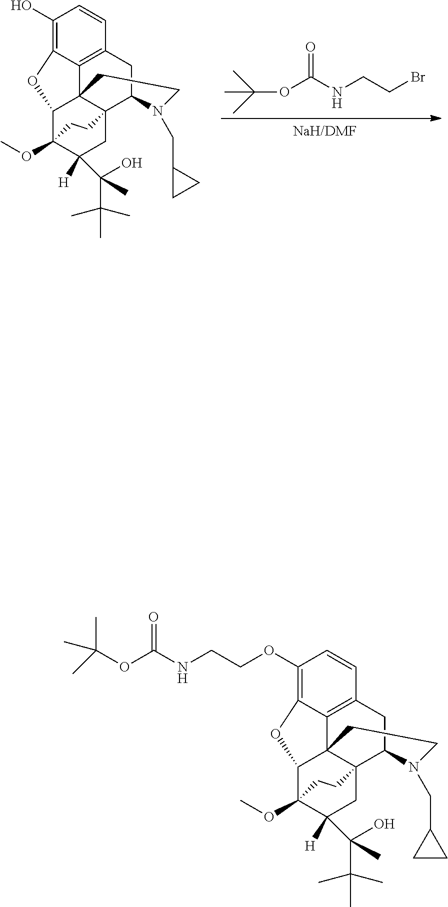

Synthesis of (1S,2S,6S,14R,15R,16R)-16-[(S)-1-Hydroxy-1,2,2-trimethylpropyl]-5-(cyclopropylmethyl)-15-methoxy-11-[2-(tert-butoxycarbonylamino)ethoxy]-13-oxa-5-azahexacyclo[13.2.2.1^{2,8}.0^{1,6}.0^{2,14}.0^{12,20}]icosa-8(20),9,11-triene. Buprenorphine HCl (500 mg, 0.9919 mmol, 1 equiv.) was dissolved in anhydrous DMF (50 mL). NaH (380 mg, 14.58 mmol, 14.7 eq) was added, and the reaction mixture was stirred at room temperature for 20 min. 2-(Boc-amino)ethylbromide (1.7 g, 7.59 mmol, 7.6 eq) was added to the reaction mixture, and stirring was continued at room temperature for 3 h. When the reaction was complete (by LCMS), water was added carefully to the reaction mixture, and the mixture was stirred for 15 min. Solvent was removed in vacuo
Water was added, and the mixture was extracted with ethyl acetate (3×50 mL), dried with anhydrous Na₂SO₄, and concentrated. Product was purified by flash column chromatography (40 g column, 0 to 35% ethyl acetate in hexanes). Fractions were collected and concentrated to obtain the product as a white solid (605 mg, quantitative yield).
MS (ESI+) m/z 610.82 for [M+H]+[C36H54N2O6]+: 611.5; (found), LCMS tR=6.14 min (ELSD).

### Step 2.

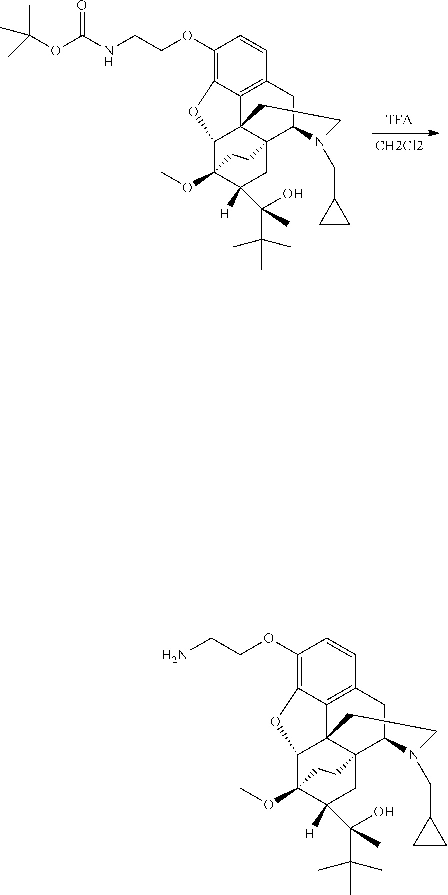

Synthesis of(S)-2-{(1S,2S,6S,14R,15R,16R)-11-(2-Aminoethoxy)-5-(cyclopropylmethyl)-15-methoxy-13-oxa-5-azahexacyclo[13.2.2.1^{2,8}.0^{1,6}.0^{2,14}.0^{12,20}]icosa-8(20),9,11-trien-16-yl}-3,3-dimethyl-2-butanol. The ether obtained above (605 mg, 0.99 mmol, 1 equiv.) was dissolved in anhydrous DCM (20 mL). TFA (1.7 mL, 21.79 mmol, 22 equiv.) was added to the reaction mixture, and the reaction mixture was stirred at room temperature for 2 h. When the reaction was complete (by LCMS), solvent and most of the TFA was removed in vacuo. Purification by flash column chromatography (25 g column, 0 to 20% MeOH in DCM) afforded the free amine produce as a clear solid (505 mg, quantitative yield).
MS (ESI+) m/z 510.71 for [M+H]+[C31H46N2O4]+: 511.8; (found), LCMS, tR=4.58 min (ELSD).

### Step 3.

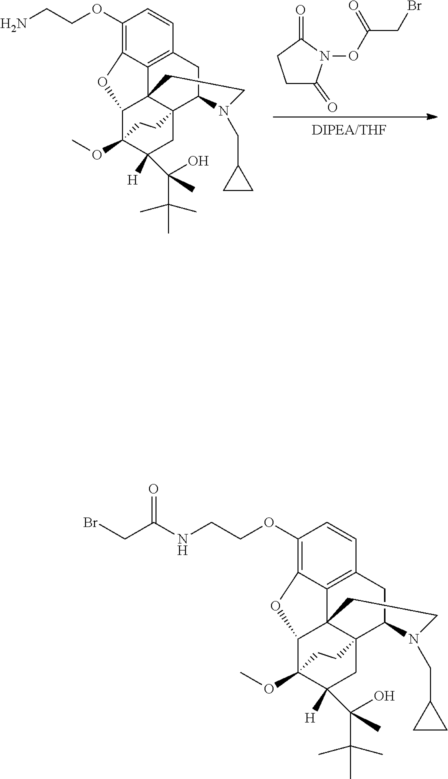

Synthesis of 1-(2-{(1S,2S,6S,14R,15R,16R)-16-[(S)-1-Hydroxy-1,2,2-trimethylpropyl]-5-(cyclopropylmethyl)-15-methoxy-13-oxa-5-azahexacyclo[13.2.2.1^{2,8}.0^{1,6}.0^{2,14}.0^{12,20}]icosa-8(20),9,11-trien-11-yloxy}ethylamino)-2-bromo-1-ethanone. The free amine obtained above (640 mg, 1.253 mmol, 1 equiv.) was dissolved in anhydrous THF (30 mL). DIPEA (5.0 mL, 34.8 mmol, 27.9 equiv.) was added to the reaction mixture. After stirring for 15 min, bromoacetic acid-N-hydroxysuccinimide (591.5 mg, 2.506 mmol, 2.0 equiv.) was added. The reaction mixture was stirred at room temperature for 3 h. When the reaction was complete (by LCMS), solvent was removed in vacuo. Purification by flash column chromatography (25 g column, 0 to 100% ethyl acetate in hexanes) afforded the product as a clear oil (241 mg, 39% yield).
MS (ESI+) m/z 631.64 for [M+H]+[C33H47BrN2O5]+: 631.8; (found), LCMS, tR=5.48 min (ELSD).

### Example 5—Synthesis of NBUP O-Ethyl Bromoacetamide Hapten 4

### Step 1.

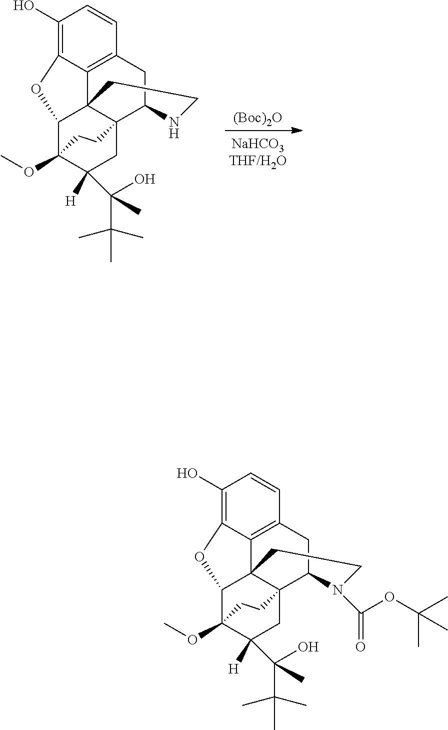 [SMILES: "(Boc)2O" ; CO[C@@]12CC[C@@]3(C[C@@H]1[C@](C)(O)C(C)(C)C)[C@H]1Cc4ccc(O)c5c4[C@@]3(CCN1)[C@H]2O5 ; "NaHCO3THF/H2O" ; CO[C@@]12CC[C@@]3(C[C@@H]1[C@](C)(O)C(C)(C)C)[C@H]1Cc4ccc(O)c5c4[C@@]3(CCN1C(=O)OC(C)(C)C)[C@H]2O5]

Synthesis of tert-Butyl (1S,2S,6S,14R,15R,16R)-16-[(S)-1-hydroxy-1,2,2-trimethylpropyl]-11-hydroxy-15-methoxy-13-oxa-5-azahexacyclo[13.2.2.1^{2,8}.0^{1,6}.0^{2,14}.0^{12,20}]icosa-8(20),9,11-triene-5-carboxylate. The secondary amine obtained above (1.54 g, 3.72 mmol, 1 equiv.) was dissolved in THF: H₂O (v/v: 10:1, 94 mL). NaHCO₃ (3.75 g, 44.69 mmol, 12.0 equiv.) and tBoc₂O (2.44 g, 11.17 mmol, 3 equiv.) were added. The reaction mixture was stirred at room temperature for 2 h. When the reaction was complete (by LCMS), solvent was removed in vacuo. Water was added, and the mixture was extracted with DCM (4×50 mL). The combined organic phases were washed with water (100 mL), dried with anhydrous Na₂SO₄, and concentrated. Purification by flash chromatography (80 g column, 0 to 60% ethyl acetate in hexanes) afforded the Boc protected amine as a white solid (1.54 g, 80% yield).

### Step 2.

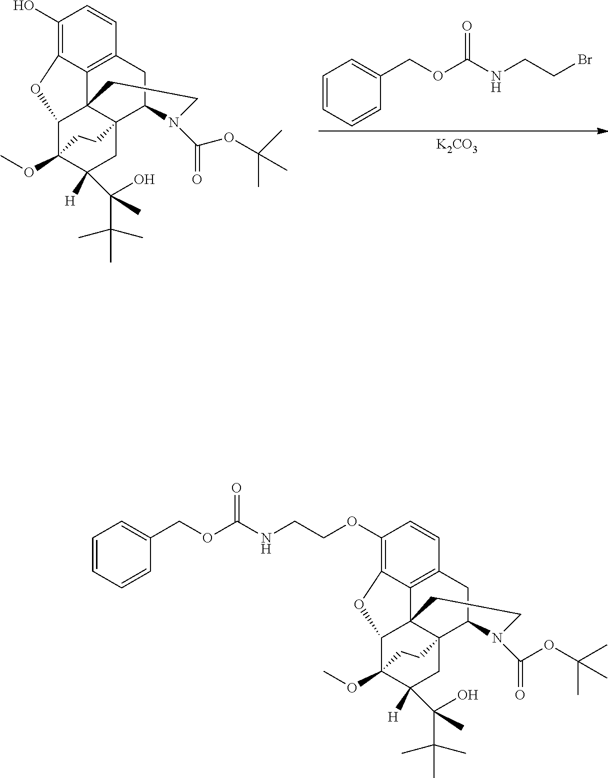

Synthesis of tert-Butyl (1,2S,6S,14R,15R,16R)-16-[(S)-1-hydroxy-1,2,2-trimethylpropyl]-11-[2-(benzyloxycarbonylamino)ethoxy]-15-methoxy-13-oxa-5-azahexacyclo[13.2.2.1^{2,8}.0^{1,6}.0^{2,14}.0^{12,20}]icosa-8(20),9,11-triene-5-carboxylate. The Boc protected amine obtained above (1.18 g, 2.30 mmol, 1 equiv.) was dissolved in anhydrous DMF (41 mL). K₂CO₃ (4.445 g, 32.161 mmol, 14 equiv.) and benzyl-(2-bromoethyl) carbamate (3.69 g, 32.16 mmol, 6.23 eq) were added. The reaction mixture was stirred at room temperature for 3 h. When the reaction was complete (by LCMS), solid was removed by filtration, and solvent was removed in vacuo. Water was added, and the mixture was extracted with ethyl acetate (3×50 mL). The combined organic phases were washed with water (100 mL), dried with anhydrous Na₂SO₄, and concentrated. Purification by column chromatography (40 g column, 0 to 50% ethyl acetate in hexanes) afforded the phenolic ether product as a white solid (1.28 g, 81% yield).
MS (ES+) m/z 690.86 for [M+H]+[C40H54N2O8]+: 691.3 (found), LCMS, tR=8.17 min (ELSD). Step 3

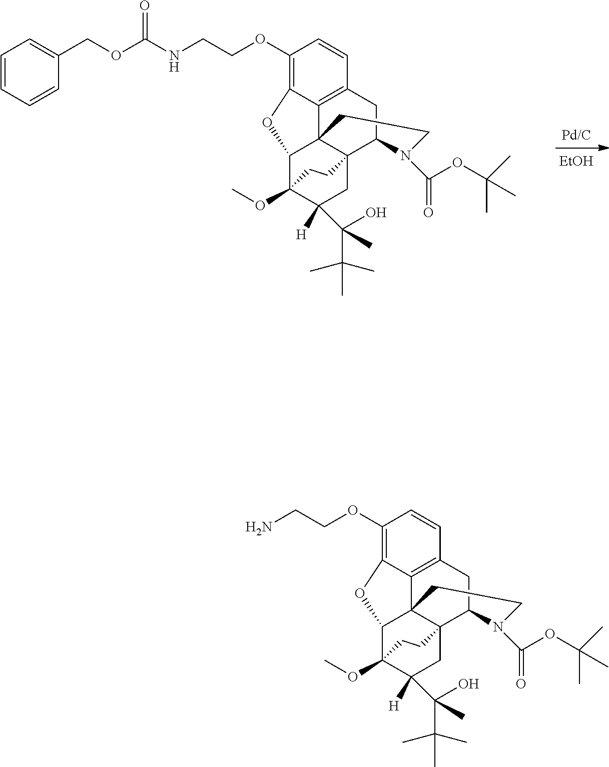 [SMILES: "EtOH" ; CO[C@@]12CC[C@@]3(C[C@@H]1[C@](C)(O)C(C)(C)C)[C@H]1Cc4ccc(OCCNC(=O)OCc5ccccc5)c5c4[C@@]3(CCN1C(=O)OC(C)(C)C)[C@H]2O5 ; "Pd/C" ; CO[C@@]12CC[C@@]3(C[C@@H]1[C@](C)(O)C(C)(C)C)[C@H]1Cc4ccc(OCCN)c5c4[C@@]3(CCN1C(=O)OC(C)(C)C)[C@H]2O5]

Synthesis of tert-Butyl (1S,2S,6S,14R,15R,16R)-16-[(S)-1-hydroxy-1,2,2-trimethylpropyl]-11-(2-aminoethoxy)-15-methoxy-13-oxa-5-azahexacyclo[13.2.2.1^{2,8}.0^{1,6}.0^{2,14}.0^{12,20}]icosa-8(20),9,11-triene-5-carboxylate. The ether obtained above (1.28 g, 1.85 mmol, 1 equiv.) was dissolved in EtOH (30 mL). Pd/C (10%, wet, 256 mg, 0.37 mmol, 0.2 equiv.) was added, and the reaction mixture was shaken under a H₂ atmosphere (60 psi) for 5 h. When the reaction was complete (by LCMS), solid was removed by filtration, and solvent was removed in vacuo. Purification by column chromatography (40 g column, 0 to 20% MeOH in DCM) afforded the free amine as a white solid (0.92 g, 89% yield).
MS (ES+) m/z 556.73 for [M+H]+[C40H54N2O8]+: 557.2; (found), LCMS tR=2.61 min (ELSD).

### Step 4

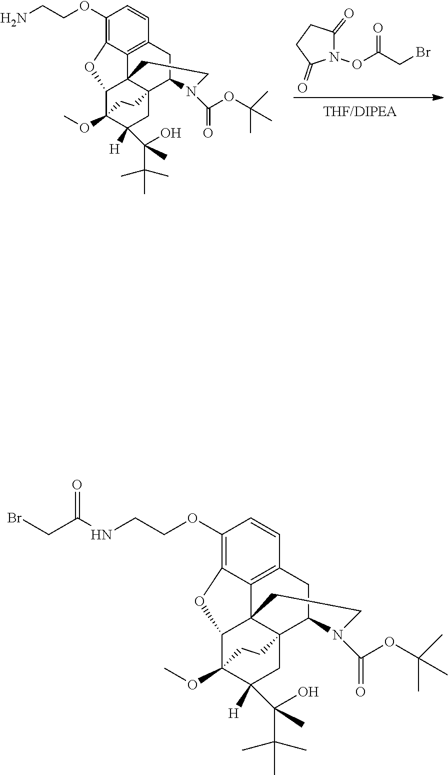 [SMILES: CO[C@@]12CC[C@@]3(C[C@@H]1[C@](C)(O)C(C)(C)C)[C@H]1Cc4ccc(OCCN)c5c4[C@@]3(CCN1C(=O)OC(C)(C)C)[C@H]2O5 ; O=C(CBr)ON1C(=O)CCC1=O ; CO[C@@]12CC[C@@]3(C[C@@H]1[C@](C)(O)C(C)(C)C)[C@H]1Cc4ccc(OCCNC(=O)CBr)c5c4[C@@]3(CCN1C(=O)OC(C)(C)C)[C@H]2O5]

Synthesis of tert-Butyl (1S,2S,6S,14R,15R,16R)-16-[(S)-1-hydroxy-1,2,2-trimethylpropyl]-11-[2-(2-bromoacetylamino)ethoxy]-15-methoxy-13-oxa-5-azahexacyclo[13.2.2.1^{2,8}.0^{1,6}.0^{2,14}.0^{12,20}]icosa-8(20), 9,11-triene-5-carboxylate. The free amine obtained above (0.92 g, 1.65 mmol, 1 equiv.) was dissolved in anhydrous THF (30 mL). DIPEA (1.15 mL, 6.61 mmol, 4 equiv.) was added to the reaction mixture. After stirring at room temperature for 10 min, bromoacetic acid-N-hydroxysuccinimide (585 mg, 2.48 mmol, 1.5 equiv.) was added. The reaction mixture was stirred at room temperature for 3 h. When the reaction was complete (by LCMS), solvent was removed in vacuo. Purification by flash column chromatography (40 g column, 0 to 80% ethyl acetate in hexanes) afforded the amide product as a white solid (770 mg, 69% yield).
MS (ES+) m/z 677.66 for [M+2H]+[C34H49BrN2O7]+: 679.6; (found), LCMS tR=3.4 min (ELSD).

### Step 5

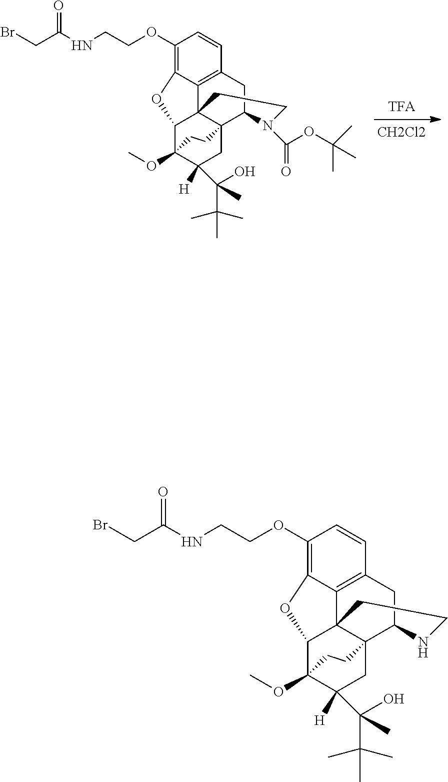

Synthesis of 1-(2-{(1S,2S,6S,14R,15R,16R)-16-[(S)-1-Hydroxy-1,2,2-trimethylpropyl]-15-methoxy-13-oxa-5-azahexacyclo[13.2.2.1^{2,8}.0^{1,6}.0^{2,14}.0^{12,20}]icosa-8(20),9,11-trien-11-yloxy}ethylamino)-2-bromo-1-ethanone. The amide obtained above (200 mg, 0.295 mmol, 1 equiv.) was dissolved in anhydrous DCM (5 ml). TFA (0.5 mL, 6.5 mmol, 22 equiv.) was added, and the reaction mixture was stirred at room temperature for 2 h. When the reaction was complete (by LCMS), solvent and most of the TFA was removed in vacuo. The product was purified by trituration with diethyl ether (5×15 mL) and then stored under vacuum overnight to give a white solid (152 mg, 89% yield).
MS (ES+) m/z 577.55 for [M+2H]+[C34H49BrN2O7]+: 579.4; (found), LCMS tR=2.02 min (ELSD).

### Example 6

### Preparation of Hapten-S-BSA Conjugates

The lysine groups in BSA were thiolated by reaction with SATA at pH 9.0 in carbonate buffer. Hydrolysis by hydroxylamine gave the thiolated BSA-SH which was reacted with the Haptens 1, 2, 3 and 4 haloacetamides to give the Hapten-S-BSA conjugates. The conjugates were purified by dialysis using a 10 kD Slide-A-Lyzer™ MWCO into 2×2 L of phosphate buffer at pH 7.4.
The Hapten-S-BSA conjugates was used to screen B-cells and monoclonal antibodies by indirect ELISA to screen for antibodies from rabbit bleeds. (FIG. 23).

### Example 7

### Preparation of BSA-NH—CO-Glucuronide Haptens

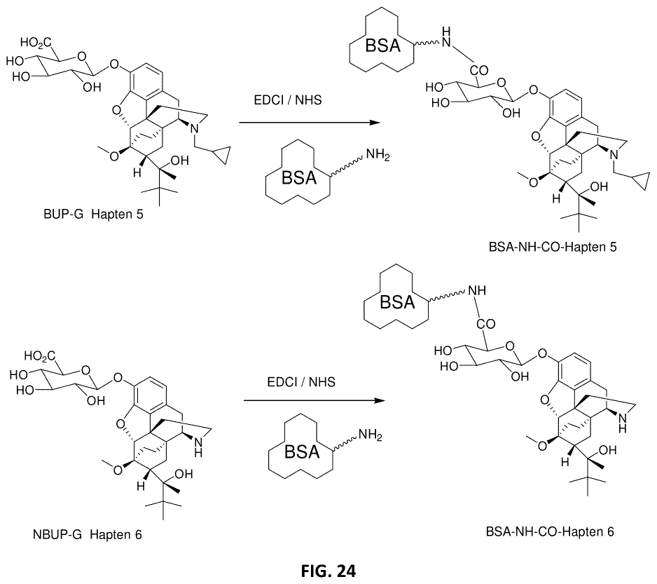

The lysine groups in BSA were coupled to the carboxyl group on the glucuronide haptens using carbodiimide activation with N-hydroxysuccinimide ester at pH 5.5 MES buffer. Adjusted pH to 7.4 with phosphate and purified the conjugates purified by dialysis using a 10 kD Slide-A-Lyzer™ MWCO into 2×2 L of phosphate buffer at pH 7.4.
The BSA-NH—CO-Glucuronide conjugates was used to screen B-cells and monoclonal antibodies by indirect ELISA to screen for antibodies from rabbit bleeds. (FIG. 24).

### Example 8

### Preparation of KLH-S-Hapten Immunogens

The BUP and NBUP haloacetamides (1 through 4 of the haptens) were conjugated to KLH where thiol groups were chemically introduced to KLH. N-Succinimidyl-S-acetylthioacetate (SATA) was reacted with the primary amines of KLH, which added protected sulfhydryls. Deprotection of acetyl protected sulfhydryls with hydroxyl amine produced the desired thiolated SH-KLH (FIG. 21). Conjugation of the haloacetamide haptens with SH-KLH resulted in immunogen KLH-S-Haptens (FIG. 21) which were utilized for immunizing rabbits in several rounds.

### a) Preparation of SH-KLH

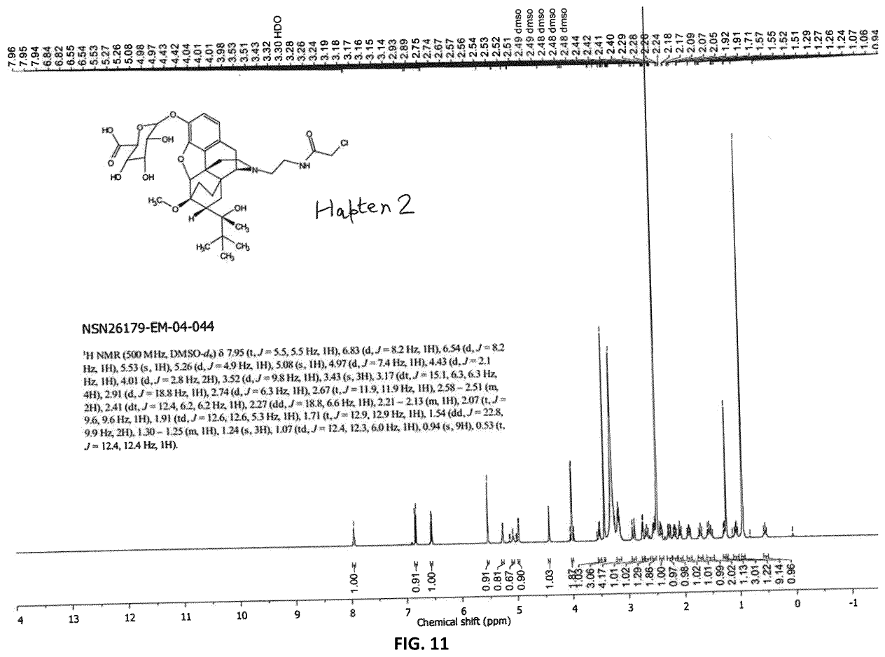

Lyophilized KLH (20 mg) was reconstituted with deionized water and pH adjusted to 8.6 with 1.0 M carbonate-bicarbonate buffer. A solution of N-Succinimidyl S-acetylthioacetate was prepared (4.67 mg was dissolved in 92 μL of DMF to a concentration of 220 mM) and slowly added to the KLH solution over 4 hrs. The reaction mixture was stirred at room temperature while N-Succinimidyl S-acetylthioacetate was being added, then stirred in the cold-room (4° C.) for an additional 16 hours.
Deacylation to generate a sulfhydryl for use in cross-linking was accomplished by adding 200 μL of a deacetylation solution (0.7 M hydroxylamine solution in 12.5 mM NaH₂PO₄—Na₂HPO₄ buffer, pH 7). Contents were mixed and reaction incubated for 2 hours at room temperature resulting in the product SH-KLH (see FIG. 11). EDTA was added at the end of this reaction to a concentration of 1 mM.

### b) Preparation of KLH-S-Hapten Immunogens

Dithiothreitol solution was added to the above SH-KLH solution for a total concentration of 1 mM to minimize disulfide bond formation. The pH was adjusted to 7.2 with 1 M carbonate-bicarbonate buffer. To the SH-KLH solution, 146 μL of the hapten BUP-X-L-bromoacetamide dissolved in DMF (9.6 mg dissolved in 0.2 mL DMF) was added slowly over 4 to 5 hrs. The reaction was continued overnight at 4° C. The mixture was purified by dialysis using a 10,000 MWCO Slide-A-Lyzer® Dialysis Cassette (Pierce) in 2×2 liter NaH₂PO₄—Na₂HPO₄ buffer 12.5 mM, pH 7.0, at 2-8° C. This procedure yielded 4 different immunogens from the Haptens 1-4, KLH-S-Hapten 1, KLH-S-Hapten 2, KLH-S-Hapten 3, and KLH-S-Hapten 4. (FIG. 21).

### Example 9

### Preparation of G6PD-S-Hapten Enzyme Conjugates

Hapten haloacetamide prepared as described in Examples 1-5 is designed for proteins containing cysteine groups such as mutant G6PD (see U.S. Pat. Nos. 6,455,288, 6,090,567, 6,033,890) or introduction of thiol-groups to wild type G6PD similarly as described in Example 8 by chemical reactions.
Hapten (0.02 mmol) was dissolved in DMF (0.21 mL). The solution was stirred at room-temperature for 30 minutes. This hapten solution for conjugation was used as described below.
Dithiothreitol solution was added to mutant G6PD at a concentration of 2 mM to reduce cysteine thiol groups connected in disulfide bonds to sulfhydryl groups. The resulting enzyme solution (0.9 mg, 1.5 mL) was adjusted to pH 7.2 with 1 M carbonate bi-carbonate buffer and mixed with approximately 340 fold molar excess (0.07 mL) of haloacetamide hapten. The reaction mixture was allowed to stir gently at 4° C. for 16 hours. Excess hapten-haloacetamide was separated from the enzyme-hapten conjugate by passing the reaction mixture over a column of Sephadex G-50 in 12.5 mM NaH₂PO₄—Na₂HPO₄ buffer, pH 7.0. The column fractions containing the enzyme-hapten conjugate are pooled by measuring absorption at 280 nm to give conjugate Hapten-S-G6PD. The conjugation process was monitored by measuring percentage deactivation and percentage maximum inhibition with BUP, NBUP, BUP-G and NBUP-G antibodies. The unreacted hapten separated by applying the conjugate to a desalting column and removed by gel filtration using HiTrap™ desalting and elution buffer consisting of phosphate (12.5 mM, pH 7.2 with 0.1 mM EDTA) at room temperature. Column fractions giving enzymatic activity greater than 500 ΔA/min were pooled. The rG6PD conjugate was placed in an 8 mL vial with cap and stored refrigerated.

### Example 10

### Preparation of Polyclonal Antibodies to the Haptens

Twelve female white New Zealand rabbits were immunized by injecting subcutaneously 200 μg/rabbit of KLH-S-Hapten immunogens as prepared in Example 8, emulsified in Complete Freund's adjuvant. The rabbits were boosted every four weeks after the initial injection with 100 μg/rabbit of the same immunogen emulsified in Incomplete Freund's Adjuvant. One hundred and thirty-four days after the initial immunization, bleeds containing polyclonal antibodies from each rabbit were obtained from the central ear artery. The anti-serum from these bleeds containing KLH-S-Hapten antibodies were evaluated in a homogeneous assay format by measuring maximum antibody inhibition of enzyme conjugates G6PD-S-Hapten and modulation in the presence of BUP, NBUP, BUP-G and NBUP-G.
From these experiments rabbits yielding the optimal antibodies were selected to isolate PBMC's as a source of B-cells for cloning from rabbits immunized with the respective KLH-S-Hapten immunogen. Screening 72 (6 immunogens×12 rabbits each) polyclonal antisera immunized with the six haptens immunogens against six hapten enzyme conjugates was performed to select the clones that gave the maximum separation (delta mA/min) in a BUP, NBUP, BUP-G and NBUP-G assay with minimal cross reactivity to structurally similar opioids as shown in FIG. 2.

### Example 11

### Preparation Rabbit Monoclonal Antibody

Rabbit recombinant monoclonal antibodies were prepared using single B-cell screening strategy for efficiently sampling the natural antibody repertoire of immunized rabbits. This technique is generally applicable to produce monoclonal antibodies to BUP, NBUP, BUP-G and NBUP-G as described herein.

Rabbit Peripheral blood mononuclear cells (PBMC's) from immunized animals were used as a source of B-cells. PBMC's were isolated from approximately 40 mL whole blood from each rabbit using standard density gradient centrifugation procedure. The PBMC's were diluted in PBS and theoretically dispensing single cell per well into forty 96-well plates the same day and cultured.

Resulting supernatants from each well were tested by indirect ELISA against Hapten-S-BSA antigen. Forty 96-well microtiter plates were coated with 0.1 μg/well BSA-S-Hapten in 0.1 M carbonate buffer, pH 9.5 and stored over night at 4° C. The plates were emptied and then blocked with 3% skimmed milk powder in PBST with shaking for 1 hr at RT. After flicking off the blocking solution the plates were rinsed with PBST. Twenty-five μL of PBST were added to each well followed by adding 25 μL of cell supernatant to all the wells and incubated for 1 hr at 37° C. in an incubator. Plates were washed 5× with PBST with a total wash time of 30 min. Afterwards, 100 μL/well secondary antibody 1:10,000 (v/v) goat anti-rabbit IgGFc-HRP conjugate in PBST was added and plates were incubated for 1 hour at 37° C. under constant shaking. Plates were washed 5 times with PBST, total wash time of 30 min. TMB substrate was added at 50 μL/well, plates. After a five minute incubation in the dark for color to develop, the reaction was stopped by the addition of 50 μL of 1 N HCl. Color was read via a microplate reader at 450 nm, and data was transferred to a computer for analysis. Supernatants that bound the BSA-S-Hapten conjugate (produced color in the wells) were considered positives. A total of 26 B cells were selected for cloning and expression.

For each ELISA well with an antigen-specific antibody, mRNA was isolated from the corresponding PBMC culture well and divided to separately synthesize cDNA from the variable regions of the rabbit genes, IgH and IgK. After two rounds of PCR to amplify, the cDNA was seamlessly ligated into separate mammalian expression vectors with a constant IgG region of the heavy chain or the constant IgK region of the light chain, respectively. Ligation mixtures were transformed into E. coli to select correct expression constructs and cultured to isolate plasmid. The expression constructs were co-transfected into HEK293 cells. Transfected cells were cultured 2 days to secrete the recombinant antibody. The antigen binding property of the recombinantly expressed antibodies were assessed by indirect ELISA against BSA-S-Hapten antigens as described above. The clones selected were further used for evaluation in the homogeneous enzyme immunoassay format as described on Examples 12 and 15. DNA sequencing was performed for selected rabbit monoclonal antibodies and translated with a standard code to provide protein sequence data for all heavy chain and kappa chain variable region sequences.

Table 1 lists the sequences of the 23 clones from the screening and testing of antibodies derived from 26 B cells (three redundant and identical sequences excluded).

### Example 12

### Reagents and Assays

BUP antibodies and enzyme conjugates may be advantageously used in accordance with the present disclosure in a homogeneous assay format to detect a BUP analyte in samples. Antibodies may be evaluated by known methods, such as, conjugate inhibition, conjugate modulation, calibration, cross-reactivity and spike-recovery. For these purposes, cloned antibody is added into the antibody diluent to prepare the antibody reagent. The antibody reagent includes antibody as prepared above, buffer, salts, stabilizers, preservatives, NAD⁺, and glucose-6-phosphate. Enzyme conjugate G6PD-S-Hapten is added into the conjugate diluent to prepare the enzyme conjugate reagent. The enzyme conjugate reagent includes the conjugate, buffer, stabilizers, salts, and preservatives.

A clinical chemistry analyzer useful to evaluate antibodies and enzyme conjugates in a homogeneous enzyme immunoassay format is the Beckman Coulter AU480 (Beckman Coulter, Brea, CA). The Beckman AU480 is an automated biochemistry spectrophotometer analyzer used by medical laboratories to process biological fluid specimens, such as urine, cerebrospinal fluid, oral fluids, plasma and serum. The analyzer is capable of maintaining a constant temperature, pipetting samples, mixing reagents, measuring light absorbance at several wavelengths and timing the reaction accurately for a kinetic reaction.

A homogeneous enzyme immunoassay is conducted using a liquid, ready-to-use, two reagent assay as described above. Typically, 2-15 μL BUP analyte-containing sample is incubated with 75-150 μL antibody reagent (R1) followed by the addition of the 50-100 μL enzyme conjugate reagent (R2). The kinetic reaction of the conversion of NAD+ to NADH is spectrophotometrically measured at 340 nm with a secondary wavelength correction at 405 nm and the rate reported as mA/min (milliabsorbance units per minute).

The assay is a homogeneous enzyme immunoassay technique used for the analysis of BUP, NBUP, BUP-G and NBUP-G in biological fluids. The assay is based on competition between the analytes BUP, NBUP, BUP-G and NBUP-G in the specimen and BUP, NBUP, BUP-G and NBUP-G derivatives of the current invention labeled with the enzyme glucose-6-phosphate dehydrogenase (G6PD) for antibody binding sites. Enzyme activity decreases upon binding to the antibody, so the analyte concentration in the sample can be measured in terms of enzyme activity. Active enzyme converts nicotinamide adenine dinucleotide (NAD⁺) to NADH, resulting in an absorbance change that is measured spectrophotometrically at 340 nm. Endogenous serum G6PD does not interfere because the coenzyme NAD⁺ functions only with the bacterial (Leuconostoc mesenteroides) enzyme employed in the assay. The change in the absorbance at 340 nm can be measured spectrophotometrically and is proportional to the enzyme conjugate activity which in turn is related to analyte concentration (see FIG. 27).

### Example 13

### Antibody Screening and Calibration Using BUP Analyte

BUP, NBUP, BUP-G and NBUP-G analyte antibodies and enzyme conjugates G6PD-S-Hapten 1, G6PD-S-Hapten 2, G6PD-S-Hapten 3, G6PD-S-Hapten 4, G6PD-S-Hapten 5, G6PD-S-Hapten 6 were used in a homogeneous assay format to generate calibration curves using BUP standards as described in Example 12. Antibody reagents were prepared as described in Examples 8 through 11 using KLH-Hapten1, KLH-Hapten2, KLH-Hapten3, Hapten 4-G6PD, KLH-Hapten 5 and KLH-Hapten 6 generated rabbit polyclonals to select for the antibody-enzyme conjugate pair for the sensitivity (curve separation in mA/min) and specificity (cross reactivity to structurally similar opioids). Antibodies meeting specifications were then used for B-cell based recombinant monoclonal production. A set of 26 B cell clones were identified and their sequence determined. Enzyme conjugates with Haptens are disclosed, G6PD-S-Haptens were used to prepare enzyme conjugate reagents. A 6-point calibration curve was generated on the Beckman AU480 clinical chemistry analyzer as described in Examples 12. The cloned monoclonal antibodies were further tested in the homogeneous format to select pairs, antibodies and enzyme conjugates that have the optimal analytical performance with respect to LOD and LOQ and no cross reactivity to structurally similar opioids.

Approximately 25-30 ml of heparinized whole blood from Rabbits producing the optimal antibody titers and specificity towards the BUP, NBUP, BUP-G and NBUP-G were collected, and the peripheral blood mononuclear cells (PBMC's) were isolated from the whole blood and cultured. In vitro culture (40×96 wells per rabbit) of the B-cells and a screening of the supernatants by indirect ELISA against Hapten-BSA antigen were performed. Antigen-positive B cells from the rabbits were selected for cloning, expression, and further screening. In brief, mRNA was isolated from selected B cells producing response to ELISA binding with BSA-Haptens, cDNA was synthesized, and 2 rounds of PCR were performed to prepare the antibody variable region cDNA for cloning. Rabbit IgG heavy and kappa light chain variable region cDNA's were each cloned into mammalian expression vectors with a rabbit heavy chain and light chain constant region, respectively. Expression constructs were co-transfected into HEK 293 cells, and cell culture supernatants were assayed by indirect ELISA against Hapten-BSA antigens. The B-cells were successfully cloned and expressed in HEK 293 culture media. From the 26 B-cells successfully expressed, some clones were selected to proceed to the 20 mL stage to produce larger quantities of rabbit recombinant antibody for further characterization.

### Example 15

### Antibodies and Calibration Using BUP

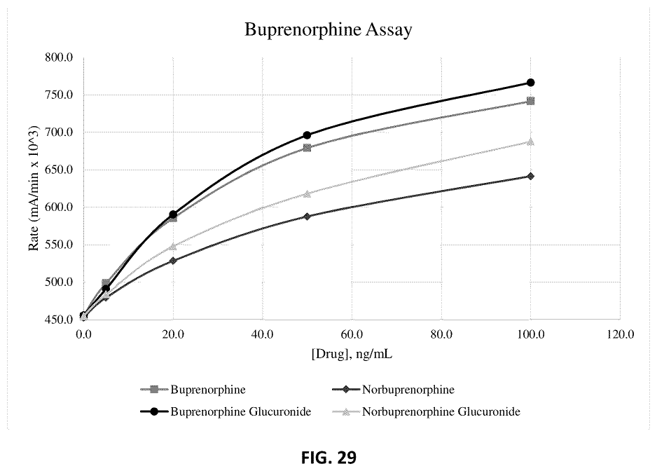

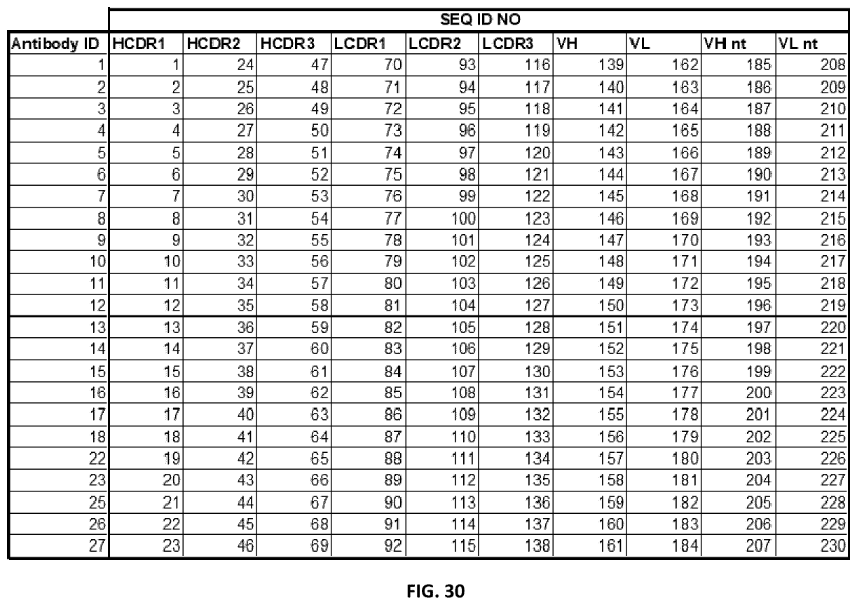

BUP, NBUP, BUP-G and NBUP-G analyte antibodies and enzyme conjugates (G6PD-S-Haptens) were used in a homogeneous assay format to generate calibration curves using standards prepared using BUP as described in Example 7. Antibodies were used to prepare reagents and used in homogeneous assay format to detect BUP. The BUP was quantified from a calibration curve generated from standards prepared using BUP, NBUP, BUP-G and NBUP-G (FIG. 29). Enzyme conjugate G6PD-S-Hapten was used to prepare the conjugate reagent. A 6-point calibration curve was generated on the Beckman AU480 clinical chemistry analyzer as described in Example 8. Typical calibration curves and dose-response curves shown in FIG. 29. Spike-recovery experiments were performed as described and results are shown in Table 12.

### Example 16

### Spike-Recovery

Known amounts of BUP analyte stock solution (mM) were added (spike) into synthetic matrix to achieve concentrations of 0.0, 5.0, 20.0, 50.0 and 100.0 ng/ml. These samples were quantified in triplicate by the homogeneous enzyme immunoassay to confirm concentration (recovery) of the spiked samples on the Beckman AU480 as described in Example 8. The samples were quantified using a separately prepared set of standards by generating a 6-point calibration curve. Calibration curve was generated using standards prepared using the same analyte as the analyte being quantified in the sample being tested. The Enzyme Conjugate Reagent contained conjugate G6PD-S-Hapten and the Antibody Reagents in the diluent. The BUP analyte concentration recovered in the spike-recovery experiments were compared to the known concentration.

In another experiment, known amounts of BUP were analyzed in the homogeneous enzyme immunoassay to confirm concentration (recovery) of the spiked samples on the Beckman AU480 as described in Example 12. Calibration curve was generated using standards prepared using the same analyte as the analyte being quantified in the sample being tested. The Enzyme Conjugate Reagent contained conjugate Hapten-S-G6PD and the Antibody Reagents were prepared containing antibody clone selected from Table 1. The BUP-analyte concentration recovered in the spike-recovery experiments were compared to the known concentration, results in Table 12.

### Calibrators and Controls

The calibrator/control diluent is prepared in a processed urine matrix formulation with pH 6.0±0.20 containing EDTA, NaN₃ and antifoam-3. Buprenorphine solution was obtained from United States Cerilliant Corporation, (B-902, Buprenorphine 100.0 μg/mL in methanol). The calibrator levels for the Buprenorphine assay are 0.0, 5.0, 20.0, 50.0 and 100.0 ng/ml. The control levels (3.0/7.0 ng/ml for 5.0 ng/ml cutoff) were prepared by dilution. The drug containing levels were prepared gravimetrically from a stock solution of 100.0 μg/ml buprenorphine in methanol.

TABLE 7
Buprenorphine Calibrators and Control Levels.

| | | |
|---|---|---|
| ARK Buprenorphine | Calibrator Conc. | 0.0, 5.0, 20.0, |
| Assay | (ng/mL) | 50.0, and 100 |
| (Project 86) | Control Conc. (ng/mL) | Low 3.0 and High 7.0 |

### Specific Performance Characteristics

The antibody reagent was prepared using monoclonal antibody reagents (adjusted concentration of 200 μg/mL) and another monoclonal antibody (adjusted concentration of 200 μg/mL), with loading of 0.52 μL and 0.43 μL loaded per 1 g of antibody diluent, respectively. The enzyme reagent was prepared using mutant enzyme conjugate with the loading of 4.13 μL per 1 mL of enzyme diluent to give a max rate of 803 (mA/min×10³).

### Calibration Curve

A calibration curve was generated on the AU480 by assaying each calibrator levels in duplicate. Typical calibrator rates are shown in Table 8 and the calibrator separation is shown in Table 9. FIG. 4 shows a typical calibration curve shape from 0.0 ng/ml to 100.0 ng/ml.

TABLE 8
Typical Calibrator Rates on AU480 (Semiquantitative mode).

| Calibrator Conc. (ng/mL) | Calibrator Rate (mA/min × 10³) |
|---|---|
| 0 | 430.3 |
| 5.0 | 474.7 |
| 20.0 | 561.4 |
| 50.0 | 654.6 |
| 100.0 | 723.7 |

TABLE 9
Typical Calibrator Separation on AU480 (Semiquantitative mode).

| Calibrator Conc. (ng/mL) | Calibrator Separation (mA/min × 10³) |
|---|---|
| 0.0-5.0 | 44.4 |
| 5.0-20.0 | 86.7 |
| 20.0-50.0 | 93.2 |
| 50.0-100.0 | 69.1 |
| Total Separation | 293.4 |

FIG. 27 shows a typical calibration curve of a Buprenorphine Assay generated on a Beckman AU480 clinical analyzer, according to embodiments of the present invention.

### Precision

Preliminary precision was evaluated using in-house cutoff and controls. A calibration curve was generated, and 10 replicates of the cutoff and QC samples were assayed for semi-quantitative protocols. This procedure was repeated twice to yield 20 replicates for each control level. The study was performed with cutoff 5.0 ng/ml. The root mean square standard deviation (RMS) and coefficient of variation percentage (% CV) was calculated for each control level, shown in Table 10.

TABLE 10
Preliminary Precision on AU480, Semi-Quantitative

| | Mean | | | |
|---|---|---|---|---|
| QC Samples | (ng/mL) | RMS SD | % CV | N |
| Low 3.0 ng/mL | 2.97 | 0.214 | 7.2 | 20 |
| Cutoff 5.0 ng/mL | 4.86 | 0.190 | 3.9 | 20 |
| High 7.0 ng/mL | 7.20 | 0.328 | 4.6 | 20 |

### Qualitative Mode: Histogram Overlap Analysis

A calibration curve was generated, and 20 replicates of each QC sample were assayed for qualitative protocols. Histogram overlap analysis was performed using a study protocol that included the 20 replicates of Negative control, Cutoff, and Positive control. The Histogram Overlap Program worksheet calculated the probability that random observations from a higher concentration sample will be less than or equal to random observations from a lower concentration sample. The program adjusts the separation based on the SD and the “N” value to generate a worst-case probability. The qualitative overlap histograms on AU 680 are shown in FIG. 26. Result shows less than 5% overlap from negative and positive controls from cutoff 5.0 ng/ml.

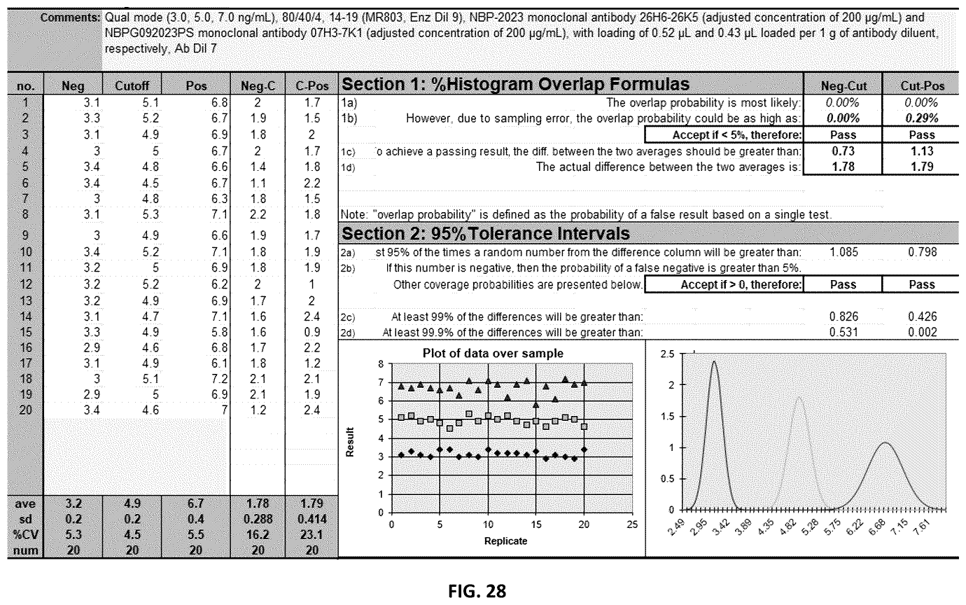

FIG. 28 shows a histogram for Preliminary Precision (Qualitative) with cutoff 5.0 ng/mL, according to embodiments of the present invention.

### 5-Day Precision

5-Day Precision Evaluation (Total Precision and Within-Laboratory Precision) was performed and analyzed with Analyse It. The study involved two runs per day; four replicates of each control level per run. Tables 11a and 11b below summarize the precision and the 5-day total precision ranged from 5.4% to 10.4% CV.

TABLE 11a
5-Day Precision Evaluation, cutoff 5.0 ng/mL

| | | | Within Run | | Between Day | | Total | |
|---|---|---|---|---|---|---|---|---|
| QC Samples | | Mean | | CV | | CV | | CV |
| (ng/mL) | N | (ng/mL) | SD | (%) | SD | (%) | SD | (%) |
| 3.0 | 40 | 2.96 | 0.260 | 8.8 | 0.180 | 6.1 | 0.307 | 10.4 |
| 5.0 | 40 | 4.92 | 0.230 | 4.7 | 0.203 | 4.1 | 0.299 | 6.1 |
| 7.0 | 40 | 7.17 | 0.322 | 4.5 | 0.236 | 3.3 | 0.388 | 5.4 |

TABLE 11b
Buprenorphine Precision (Qualitative), cutoff 5.0 ng/mL

| | | | Qualitative Precision |
|---|---|---|---|
| Cal Matrix | Relative % | # of | Results |
| (ng/mL) | Cutoff | Results | (Negative/Positive) |
| 3.0 | −40 | 40 | 40/0 |
| 5.0 | Cutoff | 40 | 23/17 |
| 7.0 | +40 | 40 | 0/40 |

### Analytical Recovery/Linearity

Linearity of Buprenorphine Assay was evaluated according to Data Collection Guideline 0735-0001-17. Linearity studies were performed by testing concentrations of buprenorphine across the assay calibration range (0.0 to 100.0 ng/ml). Proportional dilutions of 125 ng/ml buprenorphine linearity sample were made proportionally with pooled human urine negative at concentrations of LOQ, 5, 10, 20, 30,40, 50, 60, 70, 80, 90, and 100 ng/ml. Two separately calibrated runs with three replicates of each sample per run were assayed (N=6). The percentage recoveries ranged from 94% to 103.4% from LOQ (2.0 ng/ml) to 100.0 ng/ml (Table 12).

TABLE 12
Analytical Recovery

| QC Samples | Mean | | CV | Recovery | |
|---|---|---|---|---|---|
| (ng/mL) | (ng/mL) | SD | (%) | (%) | N |
| 2.0 (LOQ) | 2.07 | 0.344 | 16.7 | 103.3 | 6 |
| 5.0 | 4.70 | 0.522 | 11.1 | 94.0 | 6 |
| 10.0 | 9.45 | 0.737 | 7.8 | 96.5 | 6 |
| 20.0 | 19.30 | 0.369 | 1.9 | 97.3 | 6 |
| 30.0 | 29.20 | 1.875 | 6.4 | 99.5 | 6 |
| 40.0 | 39.78 | 1.620 | 4.1 | 98.1 | 6 |
| 50.0 | 49.07 | 2.667 | 5.4 | 103.4 | 6 |
| 60.0 | 62.07 | 2.136 | 3.4 | 99.2 | 6 |
| 70.0 | 69.42 | 3.587 | 5.2 | 102.7 | 6 |
| 80.0 | 82.18 | 3.560 | 4.3 | 1004 | 6 |
| 90.0 | 90.33 | 4.692 | 5.2 | 101.1 | 6 |
| 100.0 | 101.10 | 7.758 | 7.7 | 100.9 | 6 |

### Limit of Quantification

The LOQ of the ARK Buprenorphine Assay is defined as the lowest concentration for which acceptable inter-assay precision and recovery is observed. A set of samples were prepared by spiking buprenorphine into negative urine to give a theoretical concentration of 2.0, 3.0 and 4.0 ng/ml. Eight replicates of the test samples were assayed from five calibration curves. This was repeated 5 times to yield 40 replicates of the test samples. Shown in table 13 is the summary of results.

TABLE 13
Summary of Limit of Quantitation Results

| | | | | | | 95% Confidence | |
|---|---|---|---|---|---|---|---|
| Nominal | | Grand | | | | Interval | |
| Concentration | | Mean | % | RMS | % | Lower | Upper |
| (ng/mL) | N | (ng/mL) | Recovery | SD | CV | (−2SD) | (+2SD) |
| 2.0 | 40 | 2.12 | 105.9 | 0.169 | 8.0 | 1.779 | 2.456 |
| 3.0 | 40 | 3.03 | 101.0 | 0.229 | 7.6 | 2.572 | 3.488 |
| 4.0 | 40 | 3.81 | 95.2 | 0.285 | 7.5 | 3.237 | 4.378 |

The Buprenorphine Assay has an LOQ of 2.0 ng/mL.

### Cross-Reactivity

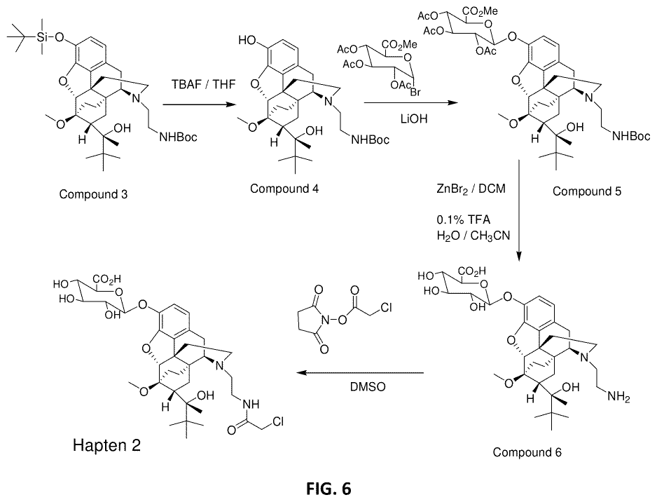

The cross-reactivity of norbuprenorphine, buprenorphine glucuronide, norbuprenorphine glucuronide, and several undesirable compounds were tested with ARK Buprenorphine assay in semi-quantitative mode (Tables 15 and 16). The calibration curves of buprenorphine, norbuprenorphine, buprenorphine glucuronide, and norbuprenorphine glucuronide were obtained (FIG. 6). Norbuprenorphine at 10.0 ng/ml corresponds to 5.0 ng/ml cutoff. Buprenorphine glucuronide at 5.8 ng/ml corresponds to 5.0 ng/ml cutoff. Norbuprenorphine glucuronide at 8.5 ng/ml corresponds to 5.0 ng/ml cutoff. All undesirable compounds showed no cross-reactivity with ARK Buprenorphine Assay.

TABLE 15
Specificity of Norbuprenorphine, Buprenorphine Glucuronide,
Norbuprenorphine Glucuronide, Semi-quantitative.

| | Conc. (ng/mL) corresponds to | % Cross- |
|---|---|---|
| Compound | 5.0 ng/mL of buprenorphine | reactivity |
| Norbuprenorphine | 10.0 | 50.0 |
| Buprenorphine | 5.8 | 86.2 |
| glucuronide | | |
| Norbuprenorphine | 8.5 | 58.8 |
| glucuronide | | |

FIG. 29 shows a graph of calibration curves of Buprenorphine (BUP), Norbuprenorphine (NBUP), Buprenorphine Glucuronide (BUP-G), and Norbuprenorphine Glucuronide (NBUP-G) generated on a Beckman AU480 clinical analyzer using reagents according to embodiments of the present invention.

TABLE 16
Buprenorphine Assay specificity, undesirable
compounds, semi-quantitative.

ARK Buprenorphine Assay

| | Conc. | Mean |
|---|---|---|
| Cross-reactants | (ng/mL) | (ng/mL) |
| 6-Acetylcodeine | 100,000 | 0.4 |
| 6-Acetylmorphine | 100,000 | 0 |
| ciprofloxacin | 100,000 | 0.1 |
| Codeine | 100,000 | 0 |
| Codeine-6-β-Glucuronide | 100,000 | 0 |
| Desmethyl Ofloxacin | 100,000 | 0 |
| Dextromethorphan | 100,000 | 0 |
| Diacetylmorphine (Heroin) | 100,000 | 0 |
| Dihydrocodeine | 100,000 | 0 |
| EDDP | 100,000 | 0 |
| EMDP | 100,000 | 0.2 |
| Ethyl morphine | 100,000 | 0 |
| Fentanyl | 100,000 | 0 |
| Hydrocodone | 100,000 | 0 |
| Hydromorphone | 100,000 | 0 |
| Hydromorphone Glucuronide | 100,000 | 0 |
| Levallorphan | 100,000 | 0 |
| Levorphanol | 100,000 | 0 |
| Meperidine | 100,000 | 0 |
| Methadone | 100,000 | 0.5 |
| Morphine | 100,000 | 0 |
| Morphine-3-Glucuronide | 100,000 | 0 |
| Morphine-6-Glucuronide | 100,000 | 0 |
| Nalbuphine | 100,000 | 0 |
| Nalorphine | 100,000 | 0 |
| Naloxegol | 100,000 | 0 |
| Naloxone | 100,000 | 0 |
| Naltrexone | 100,000 | 0 |
| Norcodeine | 100,000 | 0.3 |
| Norhydrocodone | 100,000 | 0 |
| Normorphine | 100,000 | 0 |
| Noroxycodone | 100,000 | 0 |
| Noroxymorphone | 100,000 | 0.1 |
| Norpropoxyphene | 100,000 | 0.4 |
| Ofloxacin | 100,000 | 0 |
| Oxycodone | 100,000 | 0 |
| Oxymorphone | 100,000 | 0 |
| Oxymorphone-3-β-Glucuronide | 100,000 | 0 |
| Pentazocine (Talwin) | 100,000 | 0 |
| Propoxyphene | 100,000 | 0 |
| Tapentadol | 100,000 | 0 |
| Thebaine | 100,000 | 0 |
| Tilidine | 100,000 | 0 |
| Tramadol | 100,000 | 0 |
| Dextroamphetamine | 100,000 | 0 |
| Diclofenac | 100,000 | 0 |
| Methamphetamine | 100,000 | 0 |
| Methoxetamine | 100,000 | 0 |
| N-desmethyltramadol | 100,000 | 0 |
| N-Desmethylvenlafaxine | 100,000 | NA |
| Norfentanyl | 100,000 | 0 |
| Norketamine | 100,000 | 0 |
| O-desmethyltramadol | 100,000 | 0 |
| O-Desmethylvenlafaxine | 100,000 | 0 |
| Phencyclidine | 100,000 | 0 |
| Venlafaxine | 100,000 | 0 |

### Method Comparison

All patient samples were analyzed in semi-quantitative and applied qualitatively to determine positive and negative for buprenorphine in urine samples.

A total of sixty-three (63) negative urine samples (from UCSF) were tested with Buprenorphine assay. All samples were negative. The data is shown in Table 17.

TABLE 17
Sixty-three (63) true negative urine samples
in Semi-Quantitative and Qualitative modes.

| | | ARK | | | ARK |
|---|---|---|---|---|---|
| | ARK | Quali- | | ARK | Quali- |
| Sam- | Semi- | tative | Sam- | Semi- | tative |
| ple | Quantitative | POS/ | ple | Quantitative | POS/ |
| ID | (ng/mL) | NEG | ID | (ng/mL) | NEG |
| 32016 | 0.7 | NEG | 0401.23 | 0.1 | NEG |
| 32024 | 0.1 | NEG | 0401.24 | 0.4 | NEG |
| 32025 | 0.4 | NEG | 0401.25 | 1.1 | NEG |
| 32038 | 1.1 | NEG | 0401.26 | 0.4 | NEG |
| 32058 | 0.4 | NEG | 30602 | 0.1 | NEG |
| 31427 | 0.2 | NEG | 30607 | 0.7 | NEG |
| 31442 | 0.3 | NEG | 0401.12 | 0.4 | NEG |
| 31454 | 0.3 | NEG | 0401.13 | 0.2 | NEG |
| 31456 | 0.5 | NEG | 0401.14 | 0.7 | NEG |
| 31475 | 0.3 | NEG | 0401.15 | 1.3 | NEG |
| 31476 | 0.4 | NEG | 0401.16 | 0.1 | NEG |
| 32008 | 0.3 | NEG | 0401.17 | 0.2 | NEG |
| 32009 | 2.7 | NEG | 0401.18 | 0.3 | NEG |
| 30643 | 0.4 | NEG | 0401.19 | 1.6 | NEG |
| 30645 | 1 | NEG | 0401.20 | 0.4 | NEG |
| 30654 | 0.4 | NEG | 032032 | 0.4 | NEG |
| 30665 | 0.5 | NEG | 0401.1 | 0.4 | NEG |
| 31401 | 0.3 | NEG | 0401.2 | 0 | NEG |
| 31421 | 0 | NEG | 0401.4 | 0.2 | NEG |
| 31422 | 0.2 | NEG | 0401.5 | 0.8 | NEG |
| 31423 | 0.1 | NEG | 0401.6 | 0.5 | NEG |
| 31424 | 0.4 | NEG | 0401.7 | 0.1 | NEG |
| 30615 | 0.4 | NEG | 0401.8 | 1.2 | NEG |
| 30620 | 0.9 | NEG | 0401.9 | 0.5 | NEG |
| 30621 | 0.6 | NEG | 0401.10 | 0.4 | NEG |
| 30622 | 0.7 | NEG | 0401.26 | 0.4 | NEG |
| 30632 | 1.0 | NEG | 0401.28 | 0.4 | NEG |
| 30633 | 0.2 | NEG | 0401.29 | 0.4 | NEG |
| 30640 | 0.7 | NEG | 0401.30 | 0.3 | NEG |
| 30642 | 0.5 | NEG | 0401.31 | 0.4 | NEG |
| 0401.21 | 1.2 | NEG | 032006 | 0.9 | NEG |

### Semi-Quantitative Mode

Accelerated container reagent stability was performed according to Data Collection Guideline Number 0735-0001-10. Two separately calibrated analytical runs with 10 replicates of each control per run are performed. The mean buprenorphine measurement, root mean square standard deviation (RMS SD) and % CV are calculated.

### Qualitative Mode

Accelerated container reagent stability was performed according to Data Collection Guideline Number 0735-0001-10. One calibrated run (Cal A/Cutoff) and assay 20 replicates of Cutoff (5.0 ng/ml), LOW control (3.0 ng/ml) and HIGH control (7.0 ng/mL).

The preceding merely illustrates the principles of the embodiments of the present disclosure. It will be appreciated that those skilled in the art will be able to devise various arrangements which, although not explicitly described or shown herein, embody the principles of the embodiments and are included within its spirit and scope. Furthermore, all examples and conditional language recited herein are principally intended to aid the reader in understanding the principles of the embodiments and the concepts contributed by the inventors to furthering the art, and are to be construed as being without limitation to such specifically recited examples and conditions. Moreover, all statements herein reciting principles, aspects, and embodiments of the present disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

1. A compound of Formula 1: 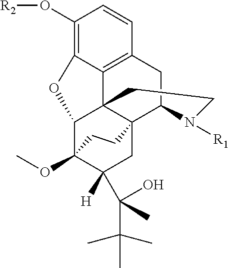
wherein:
R₁ is —Y—Z; and
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—;
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R₂ is H, glucuronic acid, glucuronide salt, or a protecting group,
and salts thereof.

2. The compound of claim 1, wherein the linking group comprises 1 to 15 carbon atoms and/or 0 to 6 heteroatoms.

3. The compound of claim 1, wherein the linking group is selected from the group consisting of —(CH₂)ₙC(O)—, —C(O)(CH₂)ₙ—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ—, —(CH₂)ₙNHC(O)—, —(CH₂)ₙNHC(O)(CH₂)ₙ—, —(CH₂)ₙNHC(O)(CH₂)ₙO(CH₂)ₙNHC(O)(CH₂)ₙ—, —(CH₂)ₙNHC(O)(CH₂)ₙNHC(O)(CH₂)ₙ—, —NH(CH₂)ₙC(O)—, —(CH₂)ₙ—, —C(O)NH(CH₂CH₂O)ₘ(CH₂)ₙNHC(O)(CH₂)ₙ—, and —(CH₂)ₙ (heterocyclyl)S(CH₂)ₙC(O)—, each m is independently an integer from 1 to 10, and each n is independently an integer from 1 to 10, and salts thereof.

4. The compound of claim 1, wherein Z is a protein.

5. The compound of claim 4, wherein the protein is an immunogenic carrier selected from the group consisting of a hemocyanin, a globulin, and an albumin.

6. The compound of claim 5, wherein the immunogenic carrier is bovine serum albumin (BSA) or keyhole limpet hemocyanin (KLH).

7. The compound of claim 1, wherein Z is an immunogenic carrier and the immunogenic carrier is a polysaccharide.

8. The compound of claim 1, wherein Z is a label.

9. The compound of claim 8, wherein the label is an enzyme.

10. The compound of claim 9, wherein the enzyme is selected from the group consisting of an alkaline phosphatase, a β-galactosidase and a horseradish peroxidase.

11. The compound of claim 9, wherein the enzyme is glucose-6-phosphate dehydrogenase (G6PD).

12. A kit for determining buprenorphine and its metabolites norbupronorphine, buprenorphine glucuronide and norbupronorphine gluconide in a sample, comprising of mixture containing an antibody derived from claim 1 and an enzyme conjugate derived from claim 1.

13. A compound of the Formula 2: 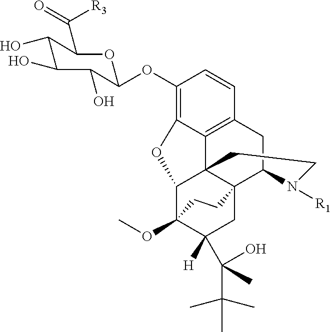
R₁ is selected from H, -cyclopropyl methyl, and Y—Z;
R₃ is selected from OH, NH2, NHR4, and Y—Z—R4
Y is a linking group selected from —(CH₂)ₙCO—, —(CH₂)ₙNHCO—, —(CH₂)ₙ—O—(CH₂)ₙ—CO—, —(CH₂)ₙ—CO—(CH₂)ₙCO—, —(CH₂)ₙ—CO—(CH₂)ₙ—NHCO—, and —(CH₂)ₙ—S—(CH₂)ₙ—CO—;
Z is selected from hydrogen, OH, SH, S-acyl, O-alkyl, halogen, NH₂, epoxy, maleimidyl, haloacetamide, carboxyl, activated carboxyl, an alkyne, an azide, an immunogenic carrier, a protein, and a label; and
R4 is selected from an immunogenic carrier, a protein, an enzyme, and a label,
and salts thereof.

14. The compound of claim 13, wherein the linking group comprises 1 to 15 carbon atoms and/or 0 to 6 heteroatoms.

15. The compound of claim 13, wherein the linking group is selected from the group consisting of —(CH₂)ₙC(O)—, —C(O)(CH₂)ₙ—, —C(O)(CH₂)ₙNHC(O)—, —C(O)(CH₂)ₙNHC(O)(CH₂)ₙ—, —(CH₂)ₙSCH₂C(O)—, —(CH₂)ₙC(O)NH(CH₂)ₙ—, —(CH₂)ₙNHC(O)—, —(CH₂)ₙNHC(O)(CH₂)ₙ—, —(CH₂)ₙNHC(O)(CH₂)NO(CH₂)ₙNHC(O)(CH₂)ₙ—, —(CH₂)ₙNHC(O)(CH₂)ₙNHC(O)(CH₂)ₙ—, —NH(CH₂)ₙC(O)—, —(CH₂)ₙ—, —C(O)NH(CH₂CH₂O)ₘ(CH₂)ₙNHC(O)(CH₂)ₙ—, and —(CH₂)ₙ (heterocyclyl)S(CH₂)ₙC(O)—, each m is independently an integer from 1 to 10, and each n is independently an integer from 1 to 10, and salts thereof.

16. The compound of claim 13, wherein Z is a protein.

17. The compound of claim 16, wherein the protein is an immunogenic carrier selected from the group consisting of a hemocyanin, a globulin, and an albumin.

18. The compound of claim 17, wherein the immunogenic carrier is bovine serum albumin (BSA) or keyhole limpet hemocyanin (KLH).

19. The compound of claim 13, wherein Z is an immunogenic carrier and the immunogenic carrier is a polysaccharide.

20. The compound of claim 13, wherein Z is a label.

21. The compound of claim 20, wherein the label is an enzyme.

22. The compound of claim 21, wherein the enzyme is selected from the group consisting of an alkaline phosphatase, a β-galactosidase and a horseradish peroxidase.

23. The compound of claim 21, wherein the enzyme is glucose-6-phosphate dehydrogenase (G6PD).

24. (canceled)

25. An antibody that specifically binds to the compounds of Formula 1 or Formula 2, wherein the antibody comprises:
a variable heavy chain (V_{H}) polypeptide comprising:
a V_{H} CDR1 comprising the amino acid sequence selected from any one of SEQ ID NOs: 1-23,
a V_{H} CDR2 comprising the amino acid sequence selected from any one of SEQ ID NOs: 24-46, and
a V_{H} CDR3 comprising the amino acid sequence selected from any one of SEQ ID NOs: 47-69; and
a variable light chain (V_{L}) polypeptide comprising:
a V_{L} CDR1 comprising the amino acid sequence selected from any one of SEQ ID NOs: 70-92, and
a V_{L} CDR2 comprising the amino acid sequence selected from any one of SEQ ID NOs: 93-115, and
a V_{L} CDR3 comprising the amino acid sequence selected from any one of SEQ ID NOs: 116-138.

26. The antibody of claim 25, wherein the antibodies comprise:
a variable heavy chain (V_{H}) polypeptide comprising an amino acid sequence having 95% or greater identity to an amino acid sequence set forth in any one of SEQ ID NOs: 139-161; and
a variable light chain (V_{L}) polypeptide comprising an amino acid sequence having 95% or greater identity to an amino acid sequence set forth in any one of SEQ ID NOs: 162-184.

27. The antibody of claim 25, wherein the antibody comprises:
a variable heavy chain (V_{H}) polypeptide comprising an amino acid sequence having 99% or greater identity to an amino acid sequence set forth in any one of SEQ ID NOs: 139-161; and
a variable light chain (V_{L}) polypeptide comprising an amino acid sequence having 99% or greater identity to an amino acid sequence set forth in any one of SEQ ID NOs: 162-184.

28. The antibody of claim 25, wherein the antibody is a monoclonal antibody.

29. The antibody of claim 25, wherein the antibody is a single chain antibody.

30. The antibodies of claim 25, wherein the antibody further specifically has 50-100% cross reactivity to norbuprenorphine, buprenorphine glucuronide and norbuprenorphine glucuronide.

31. A method for determining an amount of a buprenorphine analyte in a medium, the method comprising:
contacting in a medium
a sample suspected of containing at least one buprenorphine analyte, and
the antibody of claim 25; and
determining the presence or absence of a complex comprising the buprenorphine analyte and the antibody,
wherein the presence of the complex indicates the presence of the buprenorphine analyte in the sample.
